(19) United States
(12) Patent Application Publication
(10) Pub. No.: US 2026/0355325 A1
(43) Pub. Date: Nov. 12, 2026

(54) DIMER COMPOUNDS

(71) Applicant: KESMALEA THERAPEUTICS LIMITED, London (GB)

(72) Inventor: Harry FINCH, London (GB)

(73) Assignee: KESMALEA THERAPEUTICS LIMITED, London (GB)

(21) Appl. No.: 19/158,362

(22) PCT Filed: Feb. 20, 2024
(86) PCT No.: PCT/GB2024/050471

§ 371 Date: Aug. 20, 2025

(30) Foreign Application Priority Data
Feb. 20, 2023 (GB) 2302402.9

(51) Int. Cl. A61K 47/55; A61K 47/54; A61P 35/00

(52) U.S. Cl. CPC A61K 47/55; A61K 47/545; A61P 35/00

(57) ABSTRACT

A pharmaceutical composition comprising a preformed and reversible dimer compound and optionally a pharmaceutically acceptable excipient for use in targeted protein degradation, which may be for use as a medicament.

### FIELD OF THE INVENTION

The present invention relates to a pharmaceutical composition comprising a dimer compound for use in targeted protein degradation.

### BACKGROUND OF THE INVENTION

The balance of protein synthesis and protein degradation is responsible for the abundance of any protein in the human body. Regulation of protein degradation has therefore, emerged as an effective method of controlling individual protein abundance in cells and is largely mediated by the ubiquitin-proteasome system (UPS). The UPS is crucial to cellular functioning and protein quality control by identifying and destroying misfolded or otherwise aberrant proteins that may be toxic to cells. Dysregulation of protein degradation pathways is the cause of many diseases, such as cancer, neurological disorders and proteostatic disease.

A number of protein targets that have key roles in these diseases have proven to be intractable and difficult to target using conventional small molecules. This may be due to broad active sites, shallow pockets that are difficult to occupy with small molecules or smooth surfaces that present few active sites for binding. Thus, these protein targets have remained as a great therapeutic interest.

Current technologies involve proteolysis targeting chimeras (PROTACs) and click-formed proteolysis targeting chimeras (CLIPTACs). Both technologies incorporate a ligand for the target protein, a linker and an E3 ubiquitin ligase recruiting group which bring together the target protein and ubiquitinating machinery for protein degradation. However, PROTACs are pre-formed and administered as single, whole and typically large molecules, and as such, they are often poorly permeable and poorly orally bioavailable. CLIPTACs are formed by two monomer compounds that combine via a click reaction after administration. However, it is very difficult to control when and where the click reaction occurs, and as such due to the irreversible nature of the bond formed, CLIPTACs often self-assemble extracellularly, and may fail to enter cells and degrade the target protein. To facilitate this, the two monomer CLIPTACs are administered separately and sequentially with a sufficient time gap between administrations to ensure high enough delivery to the target tissue and followed by cell penetration so that significant quantities of monomer are present intracellularly in the target cell before dimer formation to become functionally active. Therefore, the utilities and administration methods (particularly oral administration) of PROTACs and CLIPTACs are limited from a therapeutic treatment perspective and there exists a need for a novel and effective technology with improved cell permeability and bioavailability (particularly orally) properties.

The patent applications WO2022/031772 and WO2022/031774 disclose therapeutic compositions primarily comprising two precursor monomers that in principle, are expected to form a dimer in vivo for targeted degradation of BET domain proteins. These applications aim to address the above problems by administration of individual monomers which bind reversibly to form a heterodimer which can subsequently bind to their targets once inside the cell. However, administration as two precursor monomers as a therapeutic product, either given separately, sequentially, or simultaneously is a suboptimal solution due to more complex drug product administration, management and supply chain, increased manufacturing and packaging costs. The drug substance and formulated solid product may also be more stable stored as the dimer.

### SUMMARY OF THE INVENTION

The present invention addresses the above problems by providing a preformed heterobifunctional dimer which is reversible. Due to the reversible nature of the bonds, the dimer compound will dissociate into monomers in vitro and in vivo, allowing them to enter cells, and then re-form as the dimer within the cell to then bind to target proteins and elicit ubiquitination and consequent proteasomal degradation of a target protein. Synthesizing and purifying a single compound for pharmaceutical use is more efficient, cost-effective and easier to gain regulatory approval than two monomer compounds. Dimer compositions of the present invention have been shown to be effective in eliciting ubiquitination and protein degradation as shown by the in vitro assays in the Examples.

Accordingly, in a first aspect of the invention there is a method of manufacturing a dimer compound, or a pharmaceutically acceptable salt or prodrug thereof, wherein the dimer compound comprises:
a. a first portion comprising a target protein ligand that binds to an intracellular target protein;
b. a second portion comprising a ligand that binds to an E3 ubiquitin ligase; and
c. a linker portion covalently coupling the first and second portions;
   wherein the linker portion comprises reversible covalent boronic ester bonds, the method comprising the step of:
i. reacting a first compound comprising the first portion and a second compound comprising the second portion, wherein the first and second compounds further comprise one of and different functional groups selected from a boronic acid derivative and a diol,
ii. isolating, and optionally characterising, the dimer compound.

In a second aspect of the invention there is a pharmaceutical composition comprising a dimer compound, or a pharmaceutically acceptable salt or prodrug thereof, and optionally a pharmaceutically acceptable excipient, wherein the dimer compound comprises:
a. a first portion comprising a target protein ligand that binds to an intracellular target protein;
b. a second portion comprising a ligand that binds to an E3 ubiquitin ligase; and
c. a linker portion covalently coupling the first and second portions;
   wherein the linker portion comprises reversible covalent boronic ester bonds;
wherein the composition comprises substantially no monomer compound, wherein the monomer compound comprises:
d. the first portion or the second portion; and
e. a boronic acid derivative or a diol functional group.

In a third aspect of the invention there is a method of treating a disease comprising administering a pharmaceutical composition of the second aspect or the dimer compound obtained by the first aspect, wherein the disease is selected from cancer, neurological disorders, hepatitis, ulcerative colitis, gastritis, autoimmunity, restenosis, stroke, heart failure, neurodegenerative conditions such as Alzheimer's disease, Parkinson's disease, Huntington's disease, myotonic dystrophy, and amyotrophic lateral sclerosis, AIDS, ischemia such as traumatic brain injury, spinal cord injury, cerebral ischemia, cerebral ischemia/reperfusion (l/R) injury, acute and chronic CNS injury ischemia, stroke or myocardial infarction, degenerative diseases of the musculoskeletal system such as osteoporosis, autoimmune diseases, connective tissue disorders, inflammatory diseases or proteostatic disease.

### DETAILED DESCRIPTION OF THE INVENTION

Unless indicated otherwise, all technical and scientific terms used herein will have their common meaning as understood by one of ordinary skills in the art to which this invention pertains.

The present invention relates to synthesizing dimer compounds and their utility in a pharmaceutical composition comprising, such as consisting of, said dimer compound and at least one pharmaceutically acceptable excipient for use in protein degradation. As such, the dimers and compositions have utility in the treatment of diseases. The dimer compound comprises a reversible covalent bond formed between two monomers and is capable of eliciting ubiquitination and subsequent protein degradation when delivered intracellularly.

In the first aspect of the invention there is a method of manufacturing a dimer compound or a pharmaceutically acceptable salt or prodrug thereof as outlined in the summary of invention. “Different functional groups” means that the first and second compounds have complementary functional groups selected from those listed (boronic acid derivative and diol). For example, if the first compound comprises the boronic acid derivative functional group, then the second compound must comprise the diol functional group. Alternatively, if the first compound comprises the diol functional group, then the second compound must comprise the boronic acid derivative functional group. As such, during the reaction of step (i) the first and second compound will react and form reversible boronic ester bonds via the boronic acid derivative and diol functional groups.

Suitably the reaction of step (i) of the first and second compounds occurs in an organic solvent, under dehydrating conditions, optionally using 4 A molecular sieves. Typical polar solvents include pyridine, THF, dichloromethane, acetonitrile, dimethylformamide, acetone and DMF. Preferably the solvent is THF.

Suitably the reaction of step (i) of the first and second compounds occurs in a reaction vessel. Reaction vessels are well-known in the art, (e.g., flasks, beakers, test tubes, vials, and other such containers).

The term “isolating” is known in the field to refer to separating a substance from a mixture.

The term “characterising” is known in the field to refer to methods used to identify or quantify chemicals or materials, or to characterize their physical properties.

Suitably the dimer compound is in the form of a solid, such as a powder.

Suitably the reaction of step (i) of the first and second compounds occurs at a temperature of between 10 and 100° C., preferably between 10 and 80° C., more preferably between 10 and 60° C., for example between 15 and 45° C., such as between 15 and 35° C., preferably between 20 and 30° C.

The first and second compounds require time to react. As such, suitably the step (i) occurs for at least 10 hours, preferably at least 12 hours, more preferably at least 14 hours.

Suitably the method further comprises the step of purifying the isolated dimer compound. Such purification may be done by one or more of well-known techniques in the field such as washing, filtering, and chromatography.

In another embodiment there is the dimer compound obtainable by the aforementioned method of the first aspect. Said dimer compound may be for use as a medicament. Specifically, in another embodiment, said dimer compound is for use in the treatment or prevention of cancer, neurological disorders, hepatitis, ulcerative colitis, gastritis, autoimmunity, restenosis, stroke, heart failure, neurodegenerative conditions such as Alzheimer's disease, Parkinson's disease, Huntington's disease, myotonic dystrophy, and amyotrophic lateral sclerosis, AIDS, ischemia such as traumatic brain injury, spinal cord injury, cerebral ischemia, cerebral ischemia/reperfusion (I/R) injury, acute and chronic CNS injury ischemia, stroke or myocardial infarction, degenerative diseases of the musculoskeletal system such as osteoporosis, autoimmune diseases, connective tissue disorders, inflammatory diseases or proteostatic disease.

Suitably the method further comprises the step of mixing the isolated or purified dimer compound with a pharmaceutically acceptable excipient to make a pharmaceutical composition. Suitably an effective amount of the dimer compound is in the formed pharmaceutical composition.

Suitably the method further comprises the step of processing the isolated or purified dimer compound into an orally administrable form, for example, compressing into a tablet or pellet, or encapsulating within a capsule.

The term “comprises” or “comprising” will take its usual meaning in the art, namely indicating that the component includes but is not limited to the relevant features (i.e. including, among other things). As such, the term “comprises” will include references to the component consisting essentially of (such as consisting of) the relevant features. The term “consists of” or “consisting” will take its usual meaning in the art, namely indicating that the component includes and is limited to the relevant features.

The term “comprises substantially no” indicates that the composition comprises an insignificant amount of the named feature. Suitably, the composition comprises less than 5 wt % of the named feature, preferably less than 4 wt %, more preferably less than 3 wt %, more preferably less than 2 wt %, most preferably less than 1 wt %. For example, “a composition comprising substantially no monomer” means that the composition comprises less than 5 wt %, preferably less than 4 wt %, more preferably less than 3 wt %, more preferably less than 2 wt %, most preferably less than 1 wt % of monomer.

Where appropriate, the following definitions and embodiments apply to both the dimer (or pharmaceutical composition) manufactured via the first aspect and the pharmaceutical composition of the second aspect. Suitably, the pharmaceutical composition comprises substantially no second dimer compound. Suitably, the composition comprises less than 5 wt %, preferably less than 4 wt %, more preferably less than 3 wt %, even more preferably less than 2 wt %, most preferably less than 1 wt % of a second dimer compound.

The term “dimer” or “dimer compound” refers to a molecular complex comprising two molecules (“monomers”) bonded together via the linker portion. The dimers of the present invention are bifunctional, i.e., they bind to targets and elicit protein degradation.

The term “monomer” or “monomer compound” refers to a molecule that can bind to another molecule to form a dimer. In the present invention, the monomer comprises a target protein ligand that binds to an intracellular target protein or a ligand that binds to an E3 ubiquitin ligase, and one of a boronic acid derivative or a diol functional group.

The term “linker portion” (or “linker”) refers to a flexible or conformational restricted chain or a series of atoms that is used to link two molecules (i.e., the “monomers” defined above) of interest together by reversible, chemical bonds. This linker portion is central in the dimer linking the two “monomers” together, rather than at the outer parts of the dimer that bind to the target protein or an E3 ubiquitin ligase.

The linker is of a length appropriate to bring together the target protein and the ubiquitinating ligase and thereby elicit the ubiquitination of the protein of interest and its subsequent degradation. It is therefore to be understood that the linker serves as a spacer, physically separating the target protein and ligase ligands to a degree sufficient to ensure that binding with their respective targets when as a dimer is not rendered mutually exclusive as a result of steric inhibition. In the absence of a linker, or if the linker is too short, the interaction between the target and ligase ligands of the dimer and their respective targets could be disrupted. However, it will also be understood that the linker should also not be too long, since in such cases the bound E3 ligase might not be in sufficiently close spatial proximity to the target protein to trigger its ubiquitination. Those skilled in the art will therefore appreciate that the length of the linker is preferably optimized by reference inter alia to target and E3 ligase binding efficiency as well as target protein ubiquitination.

In some embodiments, the linker may be 5-25 bonds in length, for example 5-20, 5-19, 5-18, 5-17, 5-16, 5-15, 5-14, 5-13, 5-12, 5-11, 5-10, 5-9, 5-8, 5-7 or 5-6 bonds in length. In other embodiments, the linker may be 5-25 bonds in length, for example 6-25, 7-25, 8-25, 9-25, 10-25, 11-25, 12-25, 13-25, 14-25, 15-25, 16-25, 17-25, 18-25, 18-25, 19-25 or 20-25 bonds in length.

The term “reversible” refers to bonds formed via a reversible chemical reaction in which the reactants react to form the products and simultaneously the products again form the reactants. Specifically, the term “reversible covalent boronic ester bonds” refers to bonds formed as a result of the reaction between a boronic acid derivative and a diol which can dissociate back to form the reactants. For example, the dimer of the invention may dissociate into the two monomers (e.g., in vivo) used to form said dimer. The boronic ester bonds (i.e., Boron-Oxygen) are well-known in the field and recognised by the following general formula:

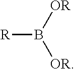

The term “treatment” or “treating” as used herein, refers to therapeutic (curative) treatment including amelioration. Treatment also includes stopping the disease from developing or slowing further progression of the disease. For example, treatment may include preventing symptoms from worsening. The term “prevention” as used herein, refers to prophylaxis treatment i.e., action taken to prevent disease.

Suitably the pharmaceutical composition comprises an effective amount of the dimer compound. As used herein, an “effective amount” of a compound or composition defines an amount that can be administered to a subject without excessive toxicity, irritation, allergic response, or other problem or complication, commensurate with a reasonable benefit/risk ratio, but one that is sufficient to provide the desired effect, e.g., the treatment or prophylaxis manifested by a permanent or temporary improvement in the subject's condition. The amount will vary from subject to subject, depending on the age and general condition of the individual, mode of administration and other factors. Thus, while it is not possible to specify an exact effective amount, those skilled in the art will be able to determine an appropriate “effective” amount in any individual case using routine experimentation and background general knowledge. A therapeutic result in this context includes eradication or lessening of symptoms, reduced pain or discomfort, prolonged survival, improved mobility and other markers of clinical improvement. A therapeutic result need not be a complete cure.

A “pharmaceutical composition” is a solid or liquid composition in a form, concentration and level of purity suitable for administration to a patient (e.g., a human or animal patient) upon which administration it can elicit the desired physiological changes. Pharmaceutical compositions are typically sterile and/or non-pyrogenic. The term non-pyrogenic as applied to the pharmaceutical compositions of the invention defines compositions which do not elicit undesirable inflammatory responses when administered to a patient. Preferably the pharmaceutical composition is in a solid form.

The term “pharmaceutically acceptable excipient” refers to any diluent, carrier or excipient, such as fillers or binders, that is compatible with the other ingredients of the composition, and which is not deleterious to the recipient. The pharmaceutically acceptable excipient can be selected on the basis of the desired route of administration, in accordance with standard pharmaceutical practices.

The term “pharmaceutically acceptable salt” refers to salts prepared from pharmaceutically acceptable non-toxic acids or bases including inorganic acids and bases and organic acids and bases. Pharmaceutically acceptable acids include both inorganic acids such as hydrochloric, sulphuric, phosphoric, diphosphoric, hydrobromic or nitric acid and organic acids such as citric, fumaric, maleic, malic, ascorbic, succinic, tartaric, benzoic, acetic, methanesulfonic, ethanesulfonic, salicylic, stearic, benzenesulfonic or p-toluenesulfonic acid. Pharmaceutically acceptable bases include alkali metal (e.g. sodium or potassium) and alkali earth metal (e.g. calcium or magnesium) hydroxides and organic bases such as alkyl amines, aryl amines or heterocyclic amines.

The term “pharmaceutically acceptable prodrug” as used herein refers to a biologically inactive compound which can be metabolized in the body to produce a drug. Suitably, the dimer is administered in the form of a prodrug which is converted to the active form during absorption in the body. Suitably the dimer prodrug of the present invention is an ester, carbonate, carbamate, amide, phosphate or oxime prodrug.

The term “ubiquitination” refers to an enzymatic process, wherein a ubiquitin protein binds to a target protein intracellular, leading to consequent degradation of the target protein via the ubiquitin-proteasome pathway.

The term “protein degradation” or “proteasomal degradation” refers to the degradation of unnecessary, abnormal and targeted proteins. The accumulation of these proteins is implicated in the pathogenesis of many diseases.

The terms “patient” and “subject” are used interchangeably and refer to a subject of treatment or prevention with the pharmaceutical composition comprising a dimer compound. Preferably, the subject is human.

The term “halo” or “halogen” as used herein refers to fluorine, chlorine, bromine, or iodine.

Preferably the halo is fluorine.

The term “alkyl” as used herein refers to an aliphatic hydrocarbon group, which may be straight or branched, missing at least one hydrogen such that a bonding position is available, i.e., an alkyl group, such as a C₁-C₈-alkyl group, preferably C₁-C₄-alkyl group, such as a C₁-C₂-alkyl group. Examples include methyl, ethyl, n-propyl and t-butyl. It may be monovalent, e.g., propyl, or divalent, e.g. propylene. A monovalent alkyl group may also be described by —CₙH₂ₙ₊₁ and a divalent alkyl group may also be described by —(CH₂)ₙ—, where n is independently selected from 1 to 8 for each substituent if not specified herein.

As used herein “haloalkyl” means an alkyl group as defined above which is substituted with up to 8 halogen atoms or more preferably up to 4 halogens. For example, they may be substituted by 1, 2, 3 or 4 halogen atoms. Preferably, the halogen is fluorine. Preferably the haloalkyl is selected from —CF₃, —CHF₂, and —CH₂F.

The term “cycloalkyl” refers to an aliphatic (i.e., non-aromatic) alkyl as defined above in the form of a monocyclic or multi-cyclic ring system, preferably of 5 to about 10 carbon atoms, preferably 5 or 6 carbons. Examples of suitable cycloalkyls are cyclopropyl, cyclobutyl, cyclobutenyl, cyclopentyl, cyclopentenyl, cyclohexyl.

The term “heterocycloalkyl” is a cycloalkyl as defined above, in which one or more of the carbons in the ring system is substituted with nitrogen, oxygen, or sulfur, such as azetidine, pyrrolidine, piperidine, piperazine, morpholine, dioxane, tetrahydrofuran and thiomorpholine.

The term “aryl” as used herein refers to an aromatic monocyclic or multi-cyclic ring system, preferably of 5 to about 10 carbon atoms, preferably 5 or 6 carbons. Preferably the aryl (or “aromatic ring”) is a monocyclic, such as benzene, toluene, ethylbenzene or xylene.

The term “heteroaryl” is an aryl as defined above, in which one or more of the carbons in the ring system is substituted with nitrogen, oxygen, or sulfur, such as pyridine, oxazole, isoxazole, triazole or thiophene.

The alkyls, haloalkyls, cycloalkyls, heterocycloalkyls, aryls and heteroaryls defined above may not be substituted or may be substituted i.e., one or more (preferably one or two, such as one) of the hydrogen atoms may be independently substituted, preferably with a single alternative atom or a small molecular group, preferably an alkyl, haloalkyl, halo, preferably alkyl or halo.

Suitably, the dimer compound comprises reversible covalent boronic ester bonds formed from the reaction between a boronic acid derivative and a diol, such as a boronic acid and a diol. Any suitable boronic acid derivative may be utilised in the invention to form the dimer. It is understood in the field that a boronic acid derivative is a compound comprising the functional group —B(OH)(OR) (in other words, one Boron atom bonded to both an OH and to an OR). It will also be understood in the context of the invention and the description that the boronic acid derivatives utilised in the invention will react with a diol to form reversible covalent boronic ester bonds. Preferably, the boronic acid derivative is a boronic acid.

It is known and understood that boronic acids have the general formula —R—B(OH)₂. It will be understood to the skilled person that the descriptions herein and above for the boronic acids refers to the R group of this formula, for example, the “aryl boronic acid” means that the R group of the general boronic acid formula is an aryl group. It will also be understood in the context of the invention and the description that this boronic acid moiety is at the terminus of one of the monomers and its linker, therefore the R group is also connected to the remainder of the monomer. In particular, the boronic acid derivative is selected from the list consisting of an alkyl boronic acid, haloalkyl boronic acid, cycloalkyl boronic acid, heterocycloalkyl boronic acid, aryl boronic acid, and a heteroaryl boronic acid. Preferably, the boronic acid is an aryl boronic acid or an alkyl boronic acid.

Suitably, the dimer compound comprises reversible covalent boronic ester bonds formed from the reaction between a boronic acid derivative and a diol. Any suitable diol may be utilised in the invention to form the dimer. Suitably the skilled person will appreciate that the two alcohol groups need to be in close proximity to each other in order to both react with the boronic acid derivative, i.e., the diol is a cis-1,2-diol, 1,3-diol, preferably a cis-1,2-diol.

Suitably the diol is an aromatic diol or an aliphatic diol, preferably an aliphatic diol. Suitably the aromatic diol is an aryl diol or a heteroaryl diol. Preferably the aliphatic diol is an alkyl diol, a cycloalkyl diol, a heterocycloalkyl diol. The cycloalkyl and heterocycloalkyl diols may be bridged cyclics, for example the diol may be cis-1,2-pinanediol or cis-2,3-norbornanediol.

“Bridged” is known in the field to mean a bicyclic molecule in which a one or more atoms spans (bridges) another ring of atoms.

It is known and understood that diols have the general formula —R—(OH)₂. In the present invention the R group comprises at least two carbon atoms and preferably the two OH groups are attached to two different adjacent carbon atoms. It will be understood to the skilled person that the description above for the diols refers to the R group of this formula, for example, the “cycloalkyl diol” means that the R group of the general diol formula is a cycloalkyl group. It will also be understood in the context of the invention and the description that this diol moiety is at the terminus of one of the monomers, therefore the R group is also connected to the remainder of the monomer and its linker.

The term “ligand” as used herein defines a binding partner for a biological target molecule in vivo (for example, an enzyme or receptor). The ligands for use according to the invention are preferably small molecules that can be functionalized by the addition of a bioorthogonal click reactive moiety via a linker.

The term “small molecule” as used herein refers to a molecule with a low molecular weight, such as less than 900 Daltons, preferably less than 700 Daltons, more preferably less than 600 Daltons. Suitably, the target protein ligand of the first portion is non-peptidic, preferably a small molecule.

Suitably, the target protein ligand of the first portion comprises a peptide, preferably an oligopeptide.

As used herein, the term “peptide” defines organic compounds comprising two or more amino acids covalently joined by peptide bonds. The corresponding adjectival term “peptidic” is to be interpreted accordingly. Peptides may be referred to with respect to the number of constituent amino acids, i.e., a dipeptide contains two amino acid residues, a tripeptide contains three, etc. Peptides containing ten or fewer amino acids may be referred to as oligopeptides, while those with more than ten amino acid residues are polypeptides. Such peptides may also include any of the modifications and additional amino and carboxy groups.

The target protein ligands of the invention target binding with many different molecules, including, but not limited to: (1) G-protein coupled receptors; (2) nuclear receptors; (3) voltage gated ion channels; (4) ligand gated ion channels; (5) receptor tyrosine kinases; (6) growth factors; (7) proteases; (8) sequence specific proteases; (9) phosphatases; (10) protein kinases; (11) tumor suppressor genes; (12) cytokines; (13) chemokines; (14) viral proteins; (15) cell division proteins; (16) scaffold proteins; (17) DNA repair proteins; (18) ubiquitin ligases and ubiquitin complexes; (19) histone modifying enzymes; (20) apoptosis regulators; (21) chaperone proteins; (22) serine/threonine protein kinases: (23) cyclin dependent kinases; (24) growth factor receptors; (25) proteasome; (26) signaling protein complexes; (27) protein/nucleic acid transporters; (28) viral capsids; (29) viral proteins; (30) chromatin remodeling proteins; (31) extracellular matrix proteins; (32) cell adhesion proteins; (33) transmembrane proteins; (34) DNA modifying enzymes; (35) RNA modifying enzymes; (36) hormones; (37) transmembrane receptors; (38) intracellular receptors; (39) DNA binding proteins; (40) transcription factors; (41) oncogenes; (42) RNA binding proteins; (43) immune system proteins; and (44) multi-component protein complexes.

Specific target proteins may be selected from AR (Androgen Receptor), ERK1/2, ERK5, A-Raf, B-Raf, C-Raf, c-Mos, Tpl2/Cot, MEK, MKK1, MKK2, MKK3, MKK4, MKK5, MKK6, MKK7, TYK2, JNK1, JNK2, JNK3, MEKK1, MEKK2, MEKK3, MEKK4, ASK1, ASK2, MLK1, MLK2, MLK3, p38 a, p38 β, p38 Y, p38 δ, BRD2, BRD3, BRD4, phosphatidyl inositol-3 kinase (PI3K), AKT, Protein kinase A (PKA), Protein Kinase B (PKB), Protein kinase C (PKC), mTOR, PDK-1, p70 S6 kinase, forkhead translocation factor, MELK, eIF4E, Hsp90, Hsp70, Hsp60, topoisomerase type I, topoisomerase type II, DNMT1, DNMT3A, DNMT3B, Cdk1 1, Cdk2, Cdk3, Cdk4, Cdk5, Cdk6, Cdk7, α-tubulin, β-tubulin, γ-tubulin, δ-tubulin, ε-Tubulin, Janus Kinases (JAK1, JAK2, JAK3), ABL1, ABL2, EGFR, EPH A1, EPHA2, EPHA3, EPHA4, EPHA5, EPHA6, EPHA7, EPHA8, EPHA10, EPHB1, EPHB2, EPHB3, EPHB4, EPHB6, EZH2, HER2/neu, Her3, Her4, ALK, FGFR1, FGFR2, FGFR3, FGFR4, IGF1 R, INSR, INSRR, VEGFR-1, VEGFR-2, VEGFR-3, FLT-3, FLT4.PDGFRA, PDGFRB, CSF1 R, Axl, IRAK4, SCFR, Fyn, MuSK, BTK, CSK, PLK4, Fes, MER, c-MET, LMTK2, FRK, ILK, Lck, TIE1, FAK, PTK6, TNNI3, ROSCCK4, ZAP-70, c-Src, Tec, Lyn, TrkA.TrkB, TrkC, RET, ROR1, ROR2, ACK1, Syk, SMARCA2, MDM2, HRas, KRas, NRas, ROCK, PI3K, BACE1, BACE2, CTSD, CTSE, NAPSA, PGC, Renin, MMSET, Aurora A kinase, Aurora B kinase, Aurora C kinase, farnesyltransferase, telomerase, adenylyiy cyclase, cAMP phosphodiesterase, PARP1, PARP2, PARP4, PARP-5a, PARP-5b, PKM2, Keapl, Nrf2, TNF, TRAIL, OX40L Lymphotoxin-alpha, IFNAR1, IFNAR2, IFN-α, IFN-β, IFN-γ, IFNLR1, CCL3, CCL4, CCL5, IL1 a, IL1 β, IL-2, IL-2, IL-4, IL-5, IL-6, IL-7, IL-9, IL-9, IL-10, IL-1 1, IL-12, IL-13, IL-15, IL-17, Bcl-2, Bcl-xL, Bax, HCV helicase, E1, E2, p7, NS2, NS3, NS4A, NS4B, NS5A, NS5B, NF-KB1, NF-B2, RelA, RelB, c-Rel, RIP1, ACE, HIV protease, HIV integrase, Gag, Pol, gp160, Tat, Rev, Nef, Vpr, Vif, Vpu, RNA polymerase, GABA transaminase, Reverse transcriptase, DNA polymerase, prolactin, ACTH, ANP, insulin, PDE, AMPK, iNOS, HDAC1, HDAC2, HDAC3, HDAC4, HDAC5, HDAC6, HDAC7, HDAC8, HDAC9, HDAC10, HDAC1 1, lactase, amylase lysozyme, neuraminidase, invertase, chitinase, hyaluronidase, maltase, sucrase, phosphatase, phosphorylases, P, Histidine decarboxylase, PTEN, histone lysine demethylase (KDM), GCN5, PCAF, Hat1, ATF-2, Tip60, MOZ, MORF, HB01, p300, CBP, SRC-1, SRC-3, ACTR, TIF-2, TAF1, TFIIIC, protein O-mannosyl-transferase 1 (POMT1), amyloid β and Tau.

Suitably, the target protein ligand of the first portion is for an intracellular target protein, wherein the target protein is a BET domain protein binding moiety, such as Bromodomain-containing 2 (BRD2), Bromodomain-containing 3 (BRD3), Bromodomain-containing 4 (BRD4), NRF2 and c-Myc. Preferably the target protein is a BET domain protein containing BD1 and BD2, particularly BRD3 and BRD4.

Suitably, the target protein ligand of the first portion is for an intracellular target protein, wherein the target protein is a BTK protein.

Suitably, the target protein ligand of the first portion is for an intracellular target protein, wherein the target protein is an AR protein.

Suitably, the ligand of the second portion comprises a peptide or an antibody of an E3 ubiquitin ligase.

Suitably, the ligand of the second portion is non-peptidic, preferably a small molecule.

Suitably, the ligand of the second portion for an E3 ubiquitin ligase is selected from the list consisting of, but not limited to, Cereblon, (CRBN), Von Hippel-Lindau (VHL), mouse double minute 2 (mdm2), Cullin-RING, Cellular inhibitor of apoptosis protein 1 (cIAP1), RNF4 (RING finger protein 4), RNF114 (RING finger protein 114), DCAF16 (DDB1 and CUL4 associated factor 16), DCAF15 (DDB1 And CUL4 Associated Factor 15), DCAF11 (DDB1 And CUL4 Associated Factor 11), FEM1B (Fem-1 Homolog B) (KEAP1 (Kelch-like ECH-associated protein 1). Preferably, the E3 ubiquitin ligase is CRBN or VHL.

In a preferred embodiment, the first portion comprising the target protein ligand that binds to an intracellular target protein is the diol-containing moiety, i.e., the diol-containing monomer used to form the dimer comprises said first portion comprising the target protein ligand that binds to an intracellular target protein.

In a preferred embodiment, the second portion comprising a ligand that binds to an E3 ubiquitin ligase is the boronic acid derivative-containing moiety, i.e., the boronic acid derivative-containing monomer used to form the dimer comprises said second portion comprising a ligand that binds to an E3 ubiquitin ligase.

Suitably, the dimer compound comprises a linker portion, wherein the linker portion is of a length sufficient to physically separate the first and second portions to an extent whereby binding of the ligands with their respective targets is not mutually exclusive as a result of steric inhibition and/or such that the E3 ubiquitin ligase bound to the ligand of the second portion is sufficiently close to the target protein bound to the ligand of the first portion to trigger ubiquitination of the target protein.

Typical dimer compounds of the present disclosure include, but are not limited to: (2S,4R)-1-((2S)-2-(3-(2-((3aR,4R,6R,7aS)-3a-(2-(2-((6S)-4-(4-chlorophenyl)-2,3,9-trimethyl-6H-thieno[3,2-f][1,2,4]triazolo[4,3-a][1,4]diazepin-6-yl)acetamido)ethyl)-5,5-dimethylhexahydro-4,6-methanobenzo[d][1,3,2]dioxaborol-2-yl)phenyl)propanamido)-3,3-dimethylbutanoyl)-4-hydroxy-N-(4-(4-methylthiazol-5-yl)benzyl)pyrrolidine-2-carboxamide, 4-(2-((3aR,4R,6R,7aS)-3a-(3-(2-((S)-4-(4-chlorophenyl)-2,3,9-trimethyl-6H-thieno[3,2-f][1,2,4]triazolo[4,3-a][1,4]diazepin-6-yl)acetamido)propyl)-5,5-dimethylhexahydro-4,6-methanobenzo[d][1,3,2]dioxaborol-2-yl)phenyl)-N-(2-(2,6-dioxopiperidin-3-yl)-1,3-dioxoisoindolin-4-yl)butanamide,
(2S,4R)-1-((2S)-2-(3-(2-((3aR,4R,6R,7aS)-3a-(2-(2-((6S)-4-(4-chlorophenyl)-2,3,9-trimethyl-6H-thieno[3,2-f][1,2,4]triazolo[4,3-a][1,4]diazepin-6-yl)acetamido)ethyl)-5,5-dimethylhexahydro-4,6-methanobenzo[d][1,3,2]dioxaborol-2-yl)-3-fluorophenyl)propanamido)-3,3-dimethylbutanoyl)-4-hydroxy-N-(4-(4-methylthiazol-5-yl)benzyl)pyrrolidine-2-carboxamide,
4-(2-((3aR,4R,6R,7aS)-3a-(2-(2-((S)-4-(4-chlorophenyl)-2,3,9-trimethyl-6H-thieno[3,2-f][1,2,4]triazolo[4,3-a][1,4]diazepin-6-yl)acetamido)ethyl)-5,5-dimethylhexahydro-4,6-methanobenzo[d][1,3,2]dioxaborol-2-yl)phenyl)-N-(2-(2,6-dioxopiperidin-3-yl)-1,3-dioxoisoindolin-4-yl)butanamide, (2S,4R)-1-((2S)-2-(3-(2-((3aR,4R,6R,7aS)-3a-(3-(2-((6S)-4-(4-chlorophenyl)-2,3,9-trimethyl-6H-thieno[3,2-f][1,2,4]triazolo[4,3-a][1,4]diazepin-6-yl)acetamido)propyl)-5,5-dimethylhexahydro-4,6-methanobenzo[d][1,3,2]dioxaborol-2-yl)phenyl)propanamido)-3,3-dimethylbutanoyl)-4-hydroxy-N-(4-(4-methylthiazol-5-yl)benzyl)pyrrolidine-2-carboxamide,
(2S,4R)-1-((2S)-2-(3-(2-((3aR,4R,6R,7aS)-3a-(2-(2-((6S)-4-(4-chlorophenyl)-2,3,9-trimethyl-6H-thieno[3,2-f][1,2,4]triazolo[4,3-a][1,4]diazepin-6-yl)acetamido)ethyl)-5,5-dimethylhexahydro-4,6-methanobenzo[d][1,3,2]dioxaborol-2-yl)phenyl)propanamido)-3,3-dimethylbutanoyl)-4-hydroxy-N—((S)-1-(4-(4-methylthiazol-5-yl)phenyl)ethyl)pyrrolidine-2-carboxamide,
(2S,4R)-1-((2S)-2-(3-(2-((3aR,4R,6R,7aS)-3a-(3-(2-((6S)-4-(4-chlorophenyl)-2,3,9-trimethyl-6H-thieno[3,2-f][1,2,4]triazolo[4,3-a][1,4]diazepin-6-yl)acetamido)propyl)-5,5-dimethylhexahydro-4,6-methanobenzo[d][1,3,2]dioxaborol-2-yl)phenyl)propanamido)-3,3-dimethylbutanoyl)-4-hydroxy-N—((S)-1-(4-(4-methylthiazol-5-yl)phenyl)ethyl)pyrrolidine-2-carboxamide,
2-((S)-4-(4-chlorophenyl)-2,3,9-trimethyl-6H-thieno[3,2-f][1,2,4]triazolo[4,3-a][1,4]diazepin-6-yl)-N-(3-((3aS,4S,6S,7aR)-2-(2-(2-(1-(4-(2,4-dioxotetrahydropyrimidin-1(2H)-yl)phenyl)azetidin-3-yl)ethyl)phenyl)-5,5-dimethyltetrahydro-4,6-methanobenzo[d][1,3,2]dioxaborol-3a(4H)-yl)propyl)acetamide,
(2S,4R)-1-(2-(3-((2-(3a-(3-(2-((S)-4-(4-chlorophenyl)-2,3,9-trimethyl-6H-thieno[3,2-f][1,2,4]triazolo[4,3-a][1,4]diazepin-6-yl)acetamido)propyl)hexahydro-4,7-methanobenzo[d][1,3,2]dioxaborol-2-yl)benzyl)oxy)isoxazol-5-yl)-3-methylbutanoyl)-4-hydroxy-N—((S)-1-(4-(4-methylthiazol-5-yl)phenyl)ethyl)pyrrolidine-2-carboxamide,
(2S,4R)—N-(4-chloro-2-((3aR,4R,6R,7aS)-3a-(2-(2-((S)-4-(4-chlorophenyl)-2,3,9-trimethyl-6H-thieno[3,2-f][1,2,4]triazolo[4,3-a][1,4]diazepin-6-yl)acetamido)ethyl)-5,5-dimethylhexahydro-4,6-methanobenzo[d][1,3,2]dioxaborol-2-yl)benzyl)-4-hydroxy-1-(3-methyl-2-(3-methylisoxazol-5-yl)butanoyl)pyrrolidine-2-carboxamide,
(2S,4R)—N-(4-chloro-2-(((3aS,4R,6R,7aS)-2-(2-(2-(2-((S)-4-(4-chlorophenyl)-2,3,9-trimethyl-6H-thieno[3,2-f][1,2,4]triazolo[4,3-a][1,4]diazepin-6-yl)acetamido)ethyl)phenyl)-5,5-dimethyltetrahydro-4,6-methanobenzo[d][1,3,2]dioxaborol-3a(4H)-yl)methoxy)benzyl)-4-hydroxy-1-(3-methyl-2-(3-methylisoxazol-5-yl)butanoyl)pyrrolidine-2-carboxamide,
(2S,4R)—N-(4-chloro-2-(3-((3aR,4R,6R,7aS)-3a-(2-(2-((S)-4-(4-chlorophenyl)-2,3,9-trimethyl-6H-thieno[3,2-f][1,2,4]triazolo[4,3-a][1,4]diazepin-6-yl)acetamido)ethyl)-5,5-dimethylhexahydro-4,6-methanobenzo[d][1,3,2]dioxaborol-2-yl)propoxy)benzyl)-4-hydroxy-1-((R)-3-methyl-2-(3-methylisoxazol-5-yl)butanoyl)pyrrolidine-2-carboxamide,
(2S,4R)-1-(2-(3-((2-((3aS,4S,6S,7aR)-3a-(2-(2-((S)-4-(4-chlorophenyl)-2,3,9-trimethyl-6H-thieno[3,2-f][1,2,4]triazolo[4,3-a][1,4]diazepin-6-yl)acetamido)ethyl)-5,5-dimethylhexahydro-4,6-methanobenzo[d][1,3,2]dioxaborol-2-yl)benzyl)oxy)isoxazol-5-yl)-3-methylbutanoyl)-4-hydroxy-N-(4-(4-methylthiazol-5-yl)benzyl)pyrrolidine-2-carboxamide, 4-((3aR,4R,6R,7aS)-3a-(2-(2-(4-(4-amino-3-(4-phenoxyphenyl)-1H-pyrazolo[3,4-d]pyrimidin-1-yl)piperidin-1-yl)ethoxy)ethyl)-5,5-dimethylhexahydro-4,6-methanobenzo[d][1,3,2]dioxaborol-2-yl)-N-(3-((1-(2,6-dioxopiperidin-3-yl)-3-methyl-2-oxo-2,3-dihydro-1H-benzo[d]imidazol-5-yl)oxy)propyl)-N,3-dimethylbenzamide,
4-((3aR,4R,6R,7aS)-3a-(2-(2-(4-(4-amino-3-(4-phenoxyphenyl)-1H-pyrazolo[3,4-d]pyrimidin-1-yl)piperidin-1-yl)ethoxy)ethyl)-5,5-dimethylhexahydro-4,6-methanobenzo[d][1,3,2]dioxaborol-2-yl)-N-(4-(2-(2,6-dioxopiperidin-3-y)-1-oxoisoindolin-4-yl)but-3-yn-1-yl)-3-methylbenzamide,
4-((3aR,4R,6R,7aS)-3a-(2-(2-(4-(4-amino-3-(4-phenoxyphenyl)-1H-pyrazolo[3,4-d]pyrimidin-1-yl)piperidin-1-yl)-2-oxoethoxy)ethyl)-5,5-dimethylhexahydro-4,6-methanobenzo[d][1,3,2]dioxaborol-2-yl)-N-(3-((1-(2,6-dioxopiperidin-3-yl)-3-methyl-2-oxo-2,3-dihydro-1H-benzo[d]imidazol-5-yl)oxy)propyl)-N,3-dimethylbenzamide,
4-((3aR,4R,6R,7aS)-3a-(2-(2-(4-(4-amino-3-(4-phenoxyphenyl)-1H-pyrazolo[3,4-d]pyrimidin-1-yl)piperidin-1-yl)-2-oxoethoxy)ethyl)-5,5-dimethylhexahydro-4,6-methanobenzo[d][1,3,2]dioxaborol-2-yl)-N-(4-(2-(2,6-dioxopiperidin-3-yl)-1-oxoisoindolin-4-yl)but-3-yn-1-yl)-3-methylbenzamide,
4-((3aR,4R,6R,7aS)-3a-(2-(2-(4-(4-amino-3-(4-phenoxyphenyl)-1H-pyrazolo[3,4-d]pyrimidin-1-yl)piperidin-1-yl)-2-oxoethoxy)ethyl)-5,5-dimethylhexahydro-4,6-methanobenzo[d][1,3,2]dioxaborol-2-yl)-N-(3-((2-(2,6-dioxopiperidin-3-yl)-1-oxoisoindolin-4-yl)oxy)propyl)-N,3-dimethylbenzamide,
3-((3aR,4R,6R,7aS)-3a-(2-(2-(4-(4-amino-3-(4-phenoxyphenyl)-1H-pyrazolo[3,4-d]pyrimidin-1-yl)piperidin-1-yl)-2-oxoethoxy)ethyl)-5,5-dimethylhexahydro-4,6-methanobenzo[d][1,3,2]dioxaborol-2-yl)-N-(3-((2-(2,6-dioxopiperidin-3-yl)-1-oxoisoindolin-4-yl)oxy)propyl)-N,4-dimethylbenzamide,
4-((3aR,4R,6R,7aS)-3a-(2-(2-(4-(4-amino-3-(4-phenoxyphenyl)-1H-pyrazolo[3,4-d]pyrimidin-1-yl)piperidin-1-yl)-2-oxoethoxy)ethyl)-5,5-dimethylhexahydro-4,6-methanobenzo[d][1,3,2]dioxaborol-2-yl)-N-(2-((2-(2,6-dioxopiperidin-3-yl)-1-oxoisoindolin-4-yl)oxy)ethyl)-N,3-dimethylbenzamide,
4-(3-(4-(2-((3aR,4R,6R,7aS)-2-(2-(3-((2-(2,6-dioxopiperidin-3-yl)-1-oxoisoindolin-4-yl)oxy)propyl)-6-methyl-1-oxoisoindolin-5-yl)-5,5-dimethyltetrahydro-4,6-methanobenzo[d][1,3,2]dioxaborol-3a(4H)-yl)ethoxy)phenyl)-4,4-dimethyl-5-oxo-2-thioxoimidazolidin-1-yl)-2-(trifluoromethyl)benzonitrile,
(2S,4R)—N-(4-chloro-2-((3aR,4R,6R,7aS)-3a-(2-((4-((trans-4-(3-chloro-4-cyanophenoxy)cyclohexyl)carbamoyl)phenyl)amino)ethyl)-5,5-dimethylhexahydro-4,6-methanobenzo[d][1,3,2]dioxaborol-2-yl)benzyl)-4-hydroxy-1-((S)-3-methyl-2-(3-methylisoxazol-5-yl)butanoyl)pyrrolidine-2-carboxamide,
4-((3aR,4R,6R,7aS)-3a-(3-((4-((trans-4-(3-chloro-4-cyanophenoxy)cyclohexyl)carbamoyl)phenyl)amino)propyl)-5,5-dimethylhexahydro-4,6-methanobenzo[d][1,3,2]dioxaborol-2-yl)-N-(3-((2-(2,6-dioxopiperidin-3-yl)-1-oxoisoindolin-4-yl)oxy)propyl)-N,3-dimethylbenzamide, and
3-((3aR,4R,6R,7aS)-3a-(3-((4-((trans-4-(3-chloro-4-cyanophenoxy)cyclohexyl)carbamoyl)phenyl)amino)propyl)-5,5-dimethylhexahydro-4,6-methanobenzo[d][1,3,2]dioxaborol-2-yl)-N-(3-((2-(2,6-dioxopiperidin-3-yl)-1-oxoisoindolin-4-yl)oxy)propyl)-N,4-dimethylbenzamide,
   or a pharmaceutically acceptable salt or prodrug thereof.

Suitably, the pharmaceutical composition comprises a dimer compound and a pharmaceutically acceptable excipient, wherein the dimer compound is capable of eliciting ubiquitination and consequent proteasomal degradation of the target protein.

Suitably, the pharmaceutical compositions of the invention may be useful in the treatment of a wide range of diseases where protein knockdown is indicated. Such diseases include proteostatic diseases and diseases arising from undesired intracellular protein activity (for example, in the treatment of cancer, where the target protein may be an oncoprotein).

As such, suitably, the pharmaceutical compositions is for use in the treatment or prevention of a disease such as cancer, neurological disorders, hepatitis, ulcerative colitis, gastritis, autoimmunity, restenosis, stroke, heart failure, neurodegenerative conditions such as Alzheimer's disease, Parkinson's disease, Huntington's disease, myotonic dystrophy, and amyotrophic lateral sclerosis, AIDS, ischemia such as traumatic brain injury, spinal cord injury, cerebral ischemia, cerebral ischemia/reperfusion (l/R) injury, acute and chronic CNS injury ischemia, stroke or myocardial infarction, degenerative diseases of the musculoskeletal system such as osteoporosis, autoimmune diseases, connective tissue disorders, inflammatory diseases or proteostatic disease.

As used herein, the term “neurological disorders” are diseases of the central and peripheral nervous system, including but not limited to epilepsy, neuromuscular disorders, autism, ADD, brain tumors, and cerebral palsy.

As used herein, the term “proteostatic disease” is used to define a set of diseases mediated, at least in part, by deficiencies in proteostasis. The term therefore covers aggregative and misfolding proteostatic diseases, including in particular neurodegenerative disorders (e.g. Parkinson's disease, Alzheimer's disease and Huntington's disease).

The skilled practitioner would understand that the compound of the invention is formulated as a pharmaceutical composition comprising a dimer compound and a pharmaceutically acceptable excipient. In one embodiment, the dimer compound is the only active agent in the composition. By only active agent, it is meant that the composition does not contain other components which may be used in the treatment of diseases, such as cancer, neurological disorders and proteostatic disease.

Alternatively, the dimer may be used in combination with a second active agent which works via a different mechanism or has a different structure as the dimer but is targeting the same disease. As such, in one embodiment the pharmaceutical composition comprises said second active agent.

Suitably, the dimer or pharmaceutical composition comprising a dimer compound and a pharmaceutically acceptable excipient is used in a method of selectively inducing the degradation of an intracellular target protein in vivo; and/or selectively inhibiting the activity of an intracellular target protein in vivo; and/or selectively ubiquitinating an intracellular target protein in vivo; and/or selectively targeting an intracellular target protein for degradation by the endogenous ubiquitin proteasome system (UPS) in vivo.

In the present invention, the dimer or pharmaceutical composition may be administered in a variety of dosage forms. In one embodiment, the composition may be formulated in a format suitable for oral, rectal, parenteral, intranasal or transdermal administration or administration by inhalation or by suppository.

Preferably, the dimer or pharmaceutical composition is administered orally, for example as a tablet, aqueous or oily suspension. Preferably, the composition is formulated such that it is suitable for oral administration, for example as a tablet or capsule.

Tablets and capsules may be prepared with binding agents, for example, syrup, acacia, gelatin, sorbitol, tragacanth, celluloses or polyvinylpyrrolidone; fillers, such as lactose, sucrose, corn starch, calcium phosphate, sorbitol, or glycine; lubricants, such as magnesium stearate, talc, polyethylene glycol, or silica; and surfactants, such as sodium lauryl sulfate. Liquid compositions may contain conventional additives such as suspending agents, for example sorbitol syrup, methyl cellulose, sugar syrup, gelatin, carboxymethyl-cellulose, or edible fats; emulsifying agents and surfactants such as lecithin, or acacia; vegetable oils such as almond oil, coconut oil, cod liver oil, or peanut oil; preservatives such as butylated hydroxy anisole (BHA) and butylated hydroxytoluene (BHT). Liquid compositions may be encapsulated in, for example, gelatin to provide a unit dosage form.

In a preferred embodiment, the dimer or pharmaceutical composition is formulated as a solid in order to maintain the improved stability of the dimer compound.

In the third aspect of the invention there is a method of treating or preventing a disease comprising administering a dimer or pharmaceutical composition of the first or second aspects, wherein the disease is selected from cancer, neurological disorders, hepatitis, ulcerative colitis, gastritis, autoimmunity, restenosis, stroke, heart failure, neurodegenerative conditions such as Alzheimer's disease, Parkinson's disease, Huntington's disease, myotonic dystrophy, and amyotrophic lateral sclerosis, AIDS, ischemia such as traumatic brain injury, spinal cord injury, cerebral ischemia, cerebral ischemia/reperfusion (l/R) injury, acute and chronic CNS injury ischemia, stroke or myocardial infarction, degenerative diseases of the musculoskeletal system such as osteoporosis, autoimmune diseases, connective tissue disorders, inflammatory diseases or proteostatic disease. This aspect may have any of the features described above for the first aspect. Suitably an effective amount of the dimer compound is administered. This method of treating or preventing may have any of the features described above for the first and second aspects.

There is also provided a use of the dimer of the first aspect for the manufacture of a medicament for the treatment of the diseases of the third aspect.

### EXAMPLES

The present invention is illustrated by way of the following examples.

The starting materials, particularly those that have not been assigned an Intermediate or Example number, are generally available from commercial sources or are readily prepared using methods well known to those skilled in the art.

### Abbreviations

Abbreviations as used herein will be known to those skilled in the art. In particular, the following abbreviations may be used herein.

| | |
|---|---|
| ACN | Acetonitrile |
| AcOH | Acetic acid |
| Ac₂O | Acetic anhydride |
| AIBN | 2,2′-azobis(2-methylpropionitrile) |
| atm | atmosphere(s) |
| aq. | aqueous |
| boc | Tert-butyloxycarbonyl |
| boc₂O | Tert-butyloxycarbonyl anhydride |
| CDI | Carbodiimidazole |
| CMBP | Cyanomethylenetributylphosphorane |
| DCM | Dichloromethane |
| DIAD | Diisopropyl azodicarboxylate |
| DIEA | N,N-Diisopropylethylamine |
| DMAP | 4-dimethylaminopyridine |
| DMF | N,N-Dimethylformamide |
| DMSO | Dimethyl sulfoxide |
| dppf | 1,1′-Ferrocenediyl-bis(diphenylphosphine) |
| DPBS | Dulbecco's Phosphate-Buffered Saline |
| EDCI | 1-(3-Dimethylaminopropyl)-3-ethylcarbodiimide |
| | hydrochloride |
| EtOAc/EA | Ethyl Acetate |
| eq. | equivalent(s) |
| FA | Formic Acid |
| g | gram(s) |
| HATU | O-(7-Azabenzotriazol-1-yl)-N,N,N′,N′-tetramethyluronium |
| | hexafluorophosphate |
| HOBT | 1-Hydroxybenzotriazole |
| Int. | Intermediate |
| i.p. | Intraperitoneal |
| IPA | Isopropyl alcohol |
| KOAc | Potassium acetate |
| LDA | Lithium diisopropylamide |
| MeCN | Acetonitrile |
| MeNH₂ | Methylamine |
| MeOH | Methanol |
| MeONa | Sodium methoxide |
| mg | milligram(s) |
| mmol | millimole(s) |
| NBS | N-bromosuccinimide |
| nm | nanometer(s) |
| TsCl | 4-methylbenzene-1-sulfonyl chloride |
| NMI | 1-Methylimidazole |
| NMO | 4-methylmorpholine N-oxide |
| NMP | 1-methyl-2-pyrrolidinone |
| Pd(dppf)Cl₂ | Bis(diphenylphosphino)ferrocene]dichloropalladium(II) |
| Pd(dppf)Cl₂•DCM | 1,1′-bis(diphenylphosphino) ferrocene-palladium(II) |
| | dichloride dichloromethane complex |
| Pd(OAc)₂ | Palladium acetate |
| PE | Petroleum Ether |
| PPh₃ | Triphenylphosphine |
| r.t. | Room temperature |
| RT | Retention Time |
| t-BuOK | Potassium tert-butoxide |
| TCFH | N,N,N′,N′-Tetramethylchloroformamidinium |
| TEA | Triethylamine |
| TFA | Trifluoroacetic acid |
| THF | Tetrahydrofuran |
| TsCl | 4-Methylbenzene-1-sulfonyl chloride |
| TsOH | p-Toluenesulfonic acid |
| wt. | Weight |
| XPhos | 2-Dicyclohexylphosphino-2′,4′,6′-triisopropylbiphenyl |

### Synthesis of Example Compounds

### Example 1

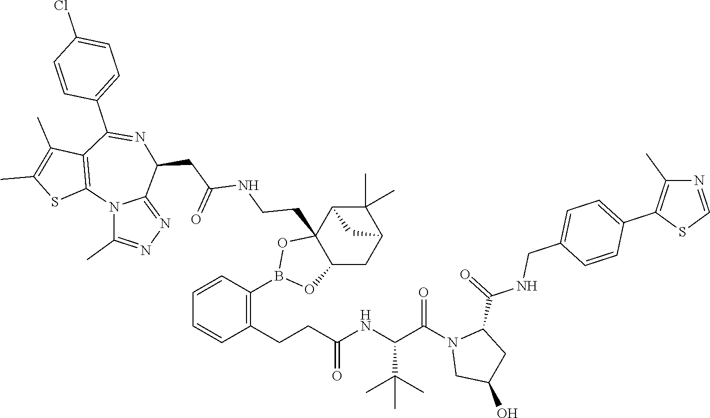 [SMILES: Cc1ncsc1-c1ccc(CNC(=O)[C@@H]2C[C@@H](O)CN2C(=O)[C@@H](NC(=O)CCc2ccccc2B2O[C@H]3C[C@H]4C[C@H](C4(C)C)[C@@]3(CCNC(=O)C[C@@H]3N=C(c4ccc(Cl)cc4)c4c(sc(C)c4C)-n4c(C)nnc43)O2)C(C)(C)C)cc1]

### (a) Synthesis of Int. 1

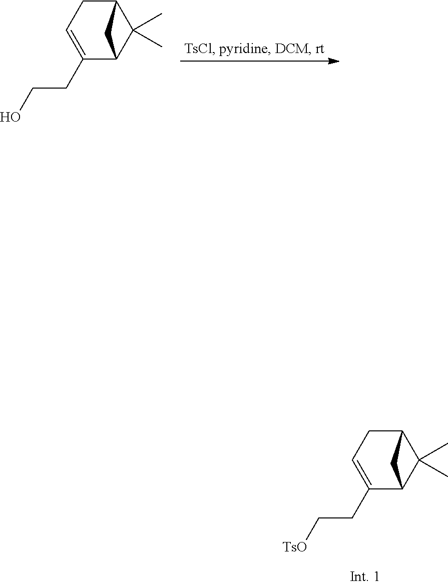 [SMILES: CC1(C)[C@H]2CC=C(CCO)[C@@H]1C2 ; Cc1ccc(S(=O)(=O)OCCC2=CC[C@H]3C[C@@H]2C3(C)C)cc1]

Into a 250 mL round-bottom flask were added 2-[(1R,5S)-6,6-dimethylbicyclo[3.1.1]hept-2-en-2-yl]ethanol (10 g, 60.2 mmol, 1 equiv), TsCl (12.61 g, 66.2 mmol, 1.1 equiv), pyridine (9.51 g, 120.3 mmol, 2 equiv), and DCM (100 mL) at r.t. The resulting mixture was stirred for 16 h at r.t. Upon completion of the reaction, the mixture was poured into water (200 mL) and extracted with CHCl₃ (3×500 mL). The combined organic layers were washed with brine (3×100 mL) and dried over anhydrous Na₂SO₄. After filtration, the filtrate was concentrated under reduced pressure. The residue was purified by silica gel column chromatography eluting with PE/EA (10:1) to afford 2-[(1R,5S)-6,6-dimethylbicyclo[3.1.1]hept-2-en-2-yl]ethyl 4-methylbenzenesulfonate (13.5 g, 34% yield) as a colorless oil.

LCMS: (ES, m/z): [M+H+17]⁺=338 (LCMS condition: Column: Shim-pack Scepter C18-120; Mobile phase A: Water/6.5 mM NH₄HCO₃; Mobile phase B: Acetonitrile; Flow rate: 1.5 mL/min; RT: 1.397 min)

### (b) Synthesis of Int. 2

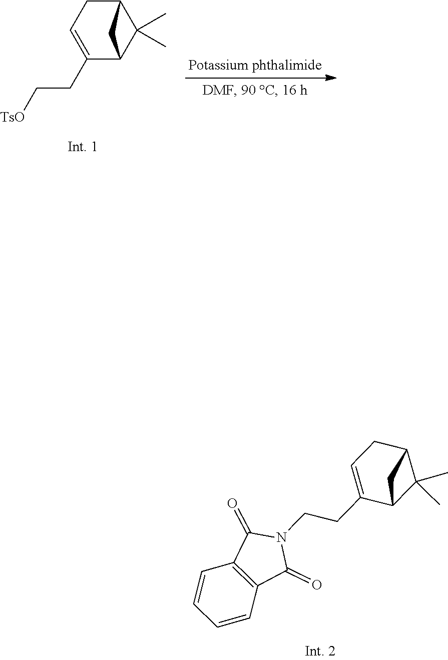 [SMILES: Cc1ccc(S(=O)(=O)OCCC2=CC[C@H]3C[C@@H]2C3(C)C)cc1 ; CC1(C)[C@H]2CC=C(CCN3C(=O)c4ccccc4C3=O)[C@@H]1C2]

Into a 500 mL round-bottom flask were added 2-[(1R,5S)-6,6-dimethylbicyclo[3.1.1]hept-2-en-2-yl]ethyl 4-methylbenzenesulfonate (13.5 g, 42.1 mmol, 1 equiv), potassium 1,3-dioxoisoindolin-2-ide (11.70 g, 63.2 mmol, 1.50 equiv), and DMF (120 mL) at r.t. The mixture was stirred at 90° C. for 16 h. After cooling to r.t., the mixture was poured into water (300 mL) and extracted with EtOAc (3×300 mL). The combined organic layers were washed with brine (3×300 mL), dried over anhydrous Na₂SO₄. After filtration, the filtrate was concentrated under reduced pressure. The residue was purified by silica gel column chromatography eluting with PE/EA (5:1) to afford 2-{2-[(1R,5S)-6,6-dimethylbicyclo[3.1.1]hept-2-en-2-yl]ethyl}isoindole-1,3-dione (12 g, 94% yield) as a colourless oil.

LCMS: (ES, m/z): [M+H]⁺=296 (LCMS condition: Column: Shim-pack Scepter; Mobile phase A: Water/5 mM NH₄HCO₃; Mobile phase B: Acetonitrile; Flow rate: 1.5 mL/min; RT: 1.351 min

### (c) Synthesis of Int. 3

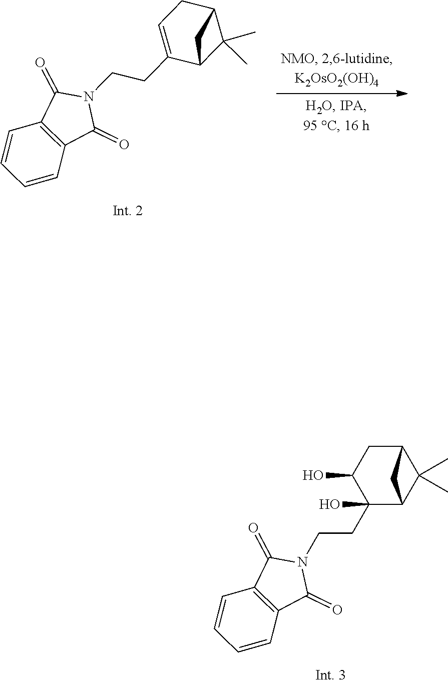 [SMILES: CC1(C)[C@H]2CC=C(CCN3C(=O)c4ccccc4C3=O)[C@@H]1C2 ; CC1(C)[C@@H]2C[C@H]1[C@](O)(CCN1C(=O)c3ccccc3C1=O)[C@@H](O)C2]

Into a 250 mL round-bottom flask were added 2-[(1R,5S)-6,6-dimethylbicyclo[3.1.1]hept-2-en-2-yl]ethanol (12 g, 72.2 mmol, 1 equiv), NMO (10.99 g, 93.8 mmol, 1.30 equiv), 2,6-lutidine (9.28 g, 86.6 mmol, 1.20 equiv), K₂OsO₂(OH)₄ (2.66 g, 7.217 mmol, 0.10 equiv), 2-propanol (60 mL), and H₂O (12 mL) at r.t. The mixture was stirred at 95° C. under a N₂ atmosphere for 16 h. The mixture was neutralized to pH 7 with HCl (aq.) and extracted with EtOAc (3×300 mL). The combined organic layers were washed with brine (3×100 mL) and dried over anhydrous Na₂SO₄. After filtration, the filtrate was concentrated under reduced pressure. The residue was purified by silica gel column chromatography eluting with PE/EA (1:1) to afford (1R,2R,3S,5R)-2-(2-hydroxyethyl)-6,6-dimethylbicyclo[3.1.1]heptane-2,3-diol (8.8 g, 60% yield) as an off-white solid.

LCMS: (ES, m/z): [M−H]⁻=328 (LCMS condition: Column: Shim-pack Scepter C18-120; Mobile phase A: Water/6.5 mM NH₄HCO₃; Mobile phase B: Acetonitrile; Flow rate: 1.5 mL/min; RT: 0.984 min)

### (d) Synthesis of Int. 4

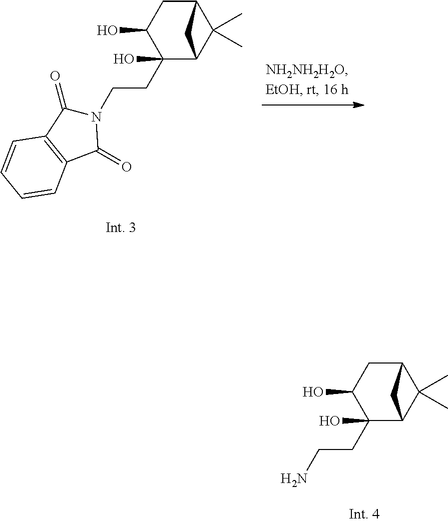 [SMILES: CC1(C)[C@@H]2C[C@H]1[C@](O)(CCN1C(=O)c3ccccc3C1=O)[C@@H](O)C2 ; CC1(C)[C@@H]2C[C@H]1[C@](O)(CCN)[C@@H](O)C2]

Into a 250 mL round-bottom flask were added 2-{2-[(1R,2R,3S,5R)-2,3-dihydroxy-6,6-dimethylbicyclo[3.1.1]heptan-2-yl]ethyl}isoindole-1,3-dione (3 g, 9.11 mmol, 1 equiv), NH₂NH₂·H₂O (0.46 g, 9.19 mmol, 1.01 equiv), and ethanol (100 mL) at r.t. The resulting mixture was stirred at r.t. for 16 h. The mixture was concentrated under reduced pressure to afford (1R,2R,3S,5R)-2-(2-aminoethyl)-6,6-dimethylbicyclo[3.1.1]heptane-2,3-diol (3.6 g, crude product) which was used without purification.

LCMS: (ES, m/z): [M+H]⁺=200 (LCMS condition: Column: Shim-pack Scepter C18-12; Mobile phase A: Water/6.5 mM NH₄HCO₃; Mobile phase B: Acetonitrile; Flow rate: 1.5 mL/min; RT: 0.847 min)

### (e) Synthesis of Int. 5

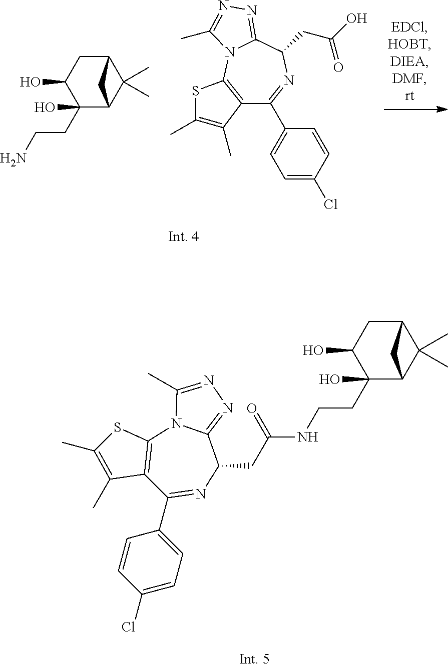

Into a 250 mL round-bottom flask were added (1R,2R,3S,5R)-2-(2-aminoethyl)-6,6-dimethylbicyclo[3.1.1]heptane-2,3-diol (2.00 g, 10.028 mmol, 2.01 equiv), (S)-2-(4-(4-chlorophenyl)-2,3,9-trimethyl-6H-thieno[3,2-f][1,2,4]triazolo[4,3-a][1,4]diazepin-6-yl)acetic acid (2 g, 4.99 mmol, 1.00 equiv), EDCI (1.24 g, 6.49 mmol, 1.30 equiv), HOBT (0.88 g, 6.49 mmol, 1.3 equiv), DIEA (1.93 g, 14.97 mmol, 3 equiv), and DMF (100 mL) at r.t. The resulting mixture was stirred at r.t. for 2 h. Upon completion of the reaction, the mixture was poured into water (300 mL) and extracted with EtOAc (3×300 mL). The combined organic layers were washed with brine (3×300 mL) and dried over anhydrous Na₂SO₄. After filtration, the filtrate was concentrated under reduced pressure. The residue was purified by silica gel column chromatography eluting with DCM/MeOH (10:1) to afford 2-((S)-4-(4-chlorophenyl)-2,3,9-trimethyl-6H-thieno[3,2-f][1,2,4]triazolo[4,3-a][1,4]diazepin-6-yl)-N-(2-((1R,2R,3S,5R)-2,3-dihydroxy-6,6-dimethylbicyclo[3.1.1]heptan-2-yl)ethyl)acetamide(2.05 g, 69% yield) as a white solid.

LCMS: (ES, m/z): [M+H]⁺=582 (LCMS condition: Column: HALO 90A CIS; Mobile phase A: water/0.1% FA; Mobile phase B: ACN/0.1% FA; Flow rate: 1.2 mL/min.; RT: 0.936 min.)

### (f) Synthesis of Int. 6

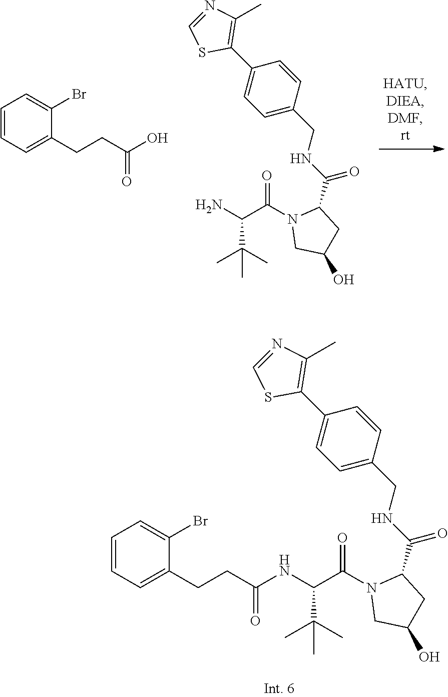 [SMILES: O=C(O)CCc1ccccc1Br ; Cc1ncsc1-c1ccc(CNC(=O)[C@@H]2C[C@@H](O)CN2C(=O)[C@@H](N)C(C)(C)C)cc1 ; Cc1ncsc1-c1ccc(CNC(=O)[C@@H]2C[C@@H](O)CN2C(=O)[C@@H](NC(=O)CCc2ccccc2Br)C(C)(C)C)cc1]

Into a 250 mL round-bottom flask were added 3-(2-bromophenyl)propanoic acid (4 g, 17.462 mmol, 1 equiv), (2S,4R)-1-[(2S)-2-amino-3,3-dimethylbutanoyl]-4-hydroxy-N-{[4-(4-methyl-1,3-thiazol-5-yl)phenyl]methyl}pyrrolidine-2-carboxamide (7.52 g, 17.5 mmol, 1 equiv), HATU (9.96 g, 26.2 mmol, 1.5 equiv), DIEA (6.77 g, 52.4 mmol, 3 equiv), and DMF (100 mL) at r.t. The resulting mixture was stirred for 16 h at r.t. Upon completion of the reaction, the mixture was poured into water (300 mL) and extracted with EtOAc (3×500 mL). The combined organic layers were washed with brine (3×100 mL), dried over anhydrous Na₂SO₄. After filtration, the filtrate was concentrated under reduced pressure. The residue was purified by silica gel column chromatography eluting with DCM/MeOH (10:1) to afford (2S,4R)-1-[(2S)-2-[3-(2-bromophenyl)propanamido]-3,3-dimethylbutanoyl]-4-hydroxy-N-{[4-(4-methyl-1,3-thiazol-5-yl)phenyl]methyl}pyrrolidine-2-carboxamide (10.5 g, 94% yield) as a yellow semi-solid.

LCMS: (ES, m/z): [M+H]+=643 (LCMS condition: Column: Shim-pack Scepter C18-120; Mobile phase A: Water/6.5 mM NH₄HCO₃; Mobile phase B: Acetonitrile; Flow rate: 1.5 mL/min; RT: 0.981 min)

### (g) Synthesis of Int. 7

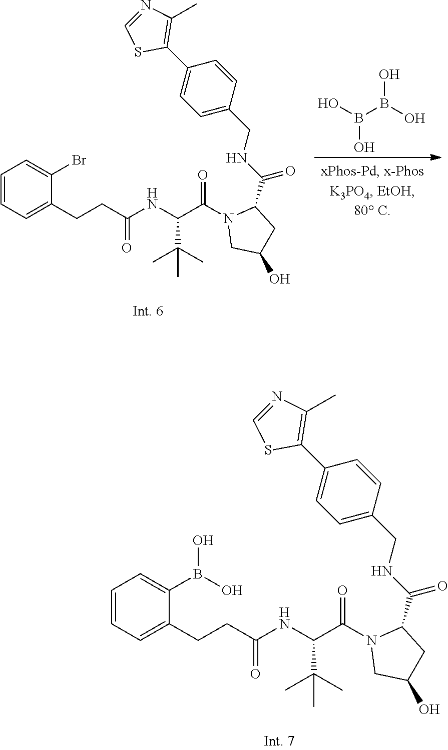 [SMILES: Cc1ncsc1-c1ccc(CNC(=O)[C@@H]2C[C@@H](O)CN2C(=O)[C@@H](NC(=O)CCc2ccccc2Br)C(C)(C)C)cc1 ; OB(O)B(O)O ; Cc1ncsc1-c1ccc(CNC(=O)[C@@H]2C[C@@H](O)CN2C(=O)[C@@H](NC(=O)CCc2ccccc2B(O)O)C(C)(C)C)cc1]

A mixture of (2S,4R)-1-[(2S)-2-[3-(2-bromophenyl)propanamido]-3,3-dimethylbutanoyl]-4-hydroxy-N-{[4-(4-methyl-1,3-thiazol-5-yl)phenyl]methyl}pyrrolidine-2-carboxamide (3.3 g, 5.143 mmol, 1 equiv), tetrahydroxydiborane (3.49 g, 39.0 mmol, 5 equiv), XPhos (0.37 g, 0.779 mmol, 0.1 equiv), XPhos Pd G3 (0.66 g, 0.779 mmol, 0.1 equiv), and K₃PO₄ (3.28 g, 15.43 mmol, 3 equiv) in EtOH was stirred for 16 h at 80° C. under a nitrogen atmosphere. After cooling to r.t., the mixture was poured into water (100 mL) and extracted with EtOAc (3×300 mL). The combined organic layers were washed with brine (3×100 mL), dried over anhydrous Na₂SO₄. After filtration, the filtrate was concentrated under reduced pressure. The residue was purified by silica gel column chromatography eluting with DCM/MeOH (10:1) to afford crude product. The crude product (2.5 g) was purified by Prep-HPLC with the following conditions (Column: XBridge Prep OBD C18 Column, 30*150 mm, 5 μm; Mobile Phase A: Water (10 mmol/L NH₄HCO₃+0.05% NH₃/H₂O), Mobile Phase B: ACN; Flow rate: 60 mL/min; Gradient: 25% B to 55% B in 8 min; Wavelength: 254 nm/220 nm; RT (min): 6.57) to afford 2-(2-{[(2S)-1-[(2S,4R)-4-hydroxy-2-({[4-(4-methyl-1,3-thiazol-5-yl)phenyl]methyl}carbamoyl)pyrrolidin-1-yl]-3,3-dimethyl-1-oxobutan-2-yl]carbamoyl}ethyl)phenylboronic acid (1.37 g, 43% yield) as a white solid.

LCMS: (ES, m/z): [M−H]⁻=605 (LCMS condition: Column: Shim-pack Scepter C18-120; Mobile phase A: Water/5 mM NH₄HCO₃; Mobile phase B: Acetonitrile; Flow rate: 1.5 mL/min; RT: 1.358 min)

### (h) Synthesis of Example 1

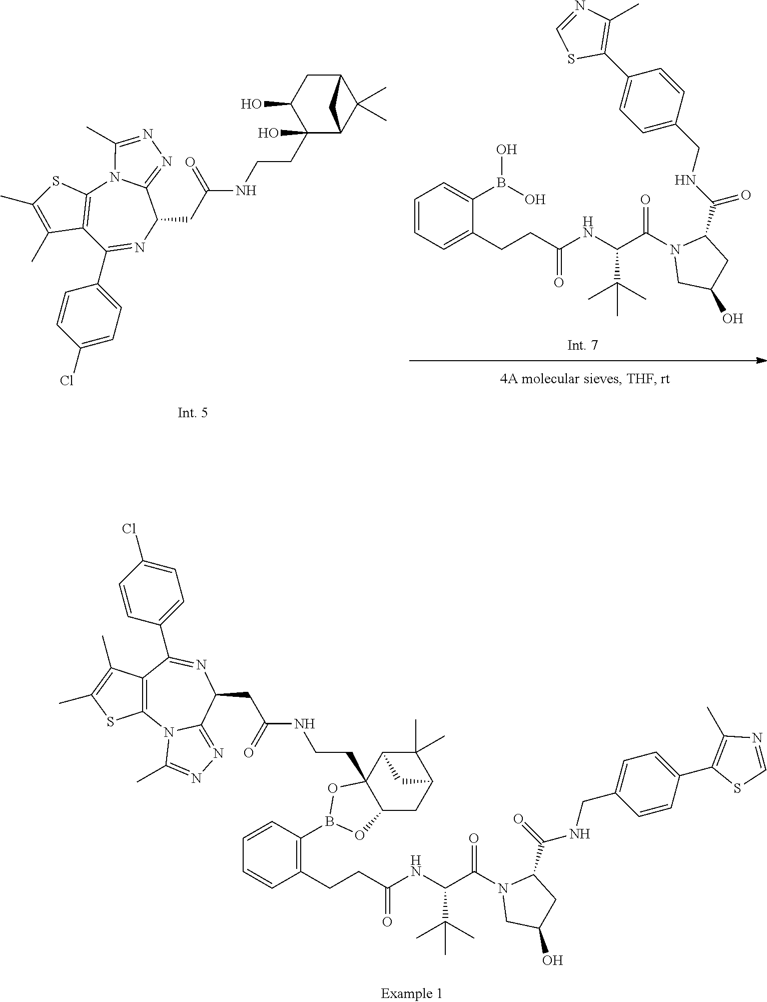

Into a 250 mL round-bottom flask were added 2-((S)-4-(4-chlorophenyl)-2,3,9-trimethyl-6H-thieno[3,2-f][1,2,4]triazolo[4,3-a][1,4]diazepin-6-yl)-N-(2-((1R,2R,3S,5R)-2,3-dihydroxy-6,6-dimethylbicyclo[3.1.1]heptan-2-yl)ethyl)acetamide (150 mg, 0.258 mmol, 1 equiv), 4A molecular sieves (900 mg), and 2-(2-{[(2S)-1-[(2S,4R)-4-hydroxy-2-({[4-(4-methyl-1,3-thiazol-5-yl)phenyl]methyl}carbamoyl)pyrrolidin-1-yl]-3,3-dimethyl-1-oxobutan-2-yl]carbamoyl}ethyl)phenylboronic acid (156.28 mg, 0.258 mmol, 1 equiv) and THF (75 mL) at r.t. The mixture was stirred for 16 h at r.t. The resulting mixture was filtered and the filter cake was washed with THF (3×30 mL). The filtrate was concentrated under reduced pressure. The crude product (450 mg) was purified by Prep-ACHIRAL-SFC with the following conditions (Column: DAICEL DCpak P4VP 3*25 cm, 5 μm; Mobile Phase A: CO₂, Mobile Phase B: MeOH (1% 2M NH₃/MeOH); Flow rate: 65 mL/min; Gradient: isocratic 30% B; Column Temperature (° C.): 35; Back Pressure (bar): 100; Wavelength: 220 nm; RT (min): 4.55; Sample Solvent: MeOH; Injection Volume: 2.5 mL) to afford (2S,4R)-1-((S)-2-(3-(2-((3aR,4R,6R,7aS)-3a-(2-(2-((S)-4-(4-chlorophenyl)-2,3,9-trimethyl-6H-thieno[3,2-f][1,2,4]triazolo[4,3-a][1,4]diazepin-6-yl)acetamido)ethyl)-5,5-dimethylhexahydro-4,6-methanobenzo[d][1,3,2]dioxaborol-2-yl)phenyl)propanamido)-3,3-dimethylbutanoyl)-4-hydroxy-N-(4-(4-methylthiazol-5-yl)benzyl)pyrrolidine-2-carboxamide (146.8 mg, 49% yield) as a white solid.

LCMS: (ES, m/z): [M+H]⁺=1153 (LCMS condition: Column: Shim-pack Scepter C18-120; Mobile phase A: Water/5 mM NH₄HCO₃; Mobile phase B: Acetonitrile; Flow rate: 1.5 mL/min; RT: 0.847 min)

### Example 2

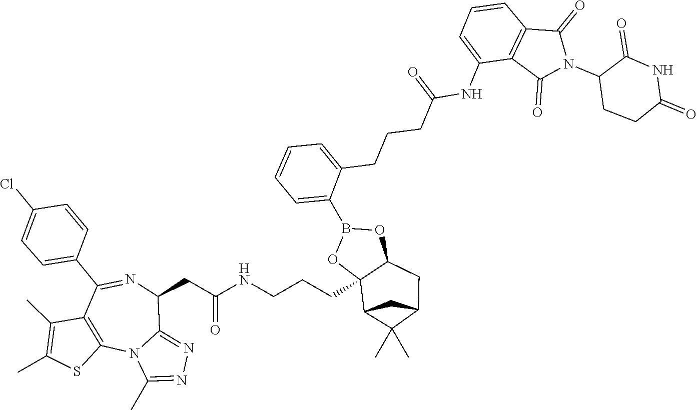 [SMILES: Cc1sc2c(c1C)C(c1ccc(Cl)cc1)=N[C@@H](CC(=O)NCCC[C@]13OB(c4ccccc4CCCC(=O)Nc4cccc5c4C(=O)N(C4CCC(=O)NC4=O)C5=O)O[C@H]1C[C@H]1C[C@@H]3C1(C)C)c1nnc(C)n1-2]

### (a) Synthesis of Int. 1 as Described in Example 1

### (b) Synthesis of Int. 9

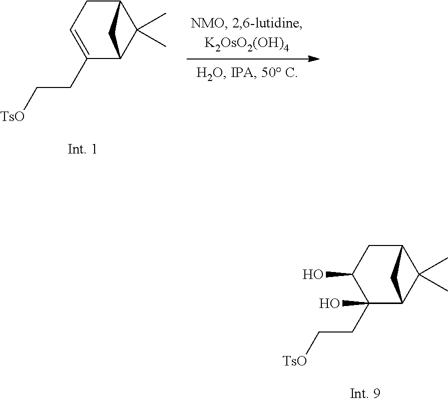 [SMILES: Cc1ccc(S(=O)(=O)OCCC2=CC[C@H]3C[C@@H]2C3(C)C)cc1 ; Cc1ccc(S(=O)(=O)OCC[C@@]2(O)[C@@H]3C[C@H](C[C@@H]2O)C3(C)C)cc1]

Into a 250 mL 3-necked round-bottom flask were added 2-[(1R,5S)-6,6-dimethylbicyclo[3.1.1]hept-2-en-2-yl]ethyl 4-methylbenzenesulfonate (5 g, 15.6 mmol, 1 equiv), NMO (2.38 g, 20.3 mmol, 1.3 equiv), 2,6-lutidine (2.01 g, 18.7 mmol, 1.20 equiv), K₂OsO₂(OH)₄ (0.57 g, 1.56 mmol, 0.1 equiv), 2-Propanol (50 mL), and H₂O (10 mL). The resulting mixture stirred at 50° C. under N₂ for 2 h. After cooling to r.t., the mixture was poured into water (200 mL), and then the resulting mixture was extracted with EtOAc (3×500 mL). The combined organic layers were washed with brine (3×300 mL) and dried over anhydrous Na₂SO₄. After filtration, the filtrate was concentrated under reduced pressure. The residue was purified by reverse phase flash with the following conditions (eluting with 0 to 100% acetonitrile/0.1% aq. NH₃) to afford 2-[(1R,2R,3S,5R)-2,3-dihydroxy-6,6-dimethylbicyclo[3.1.1]heptan-2-yl]ethyl 4-methylbenzenesulfonate (3.00 g, 51% yield) as a yellow oil.

LCMS: (ES, m/z): [M+H+17]⁺=372 (LCMS condition: Column: Shim-pack Scepter C18-120; Mobile phase A: Water/5 mM NH₄HCO₃; Mobile phase B: Acetonitrile; Flow rate: 1.5 mL/min; RT: 1.361 min)

### (c) Synthesis of Int. 10

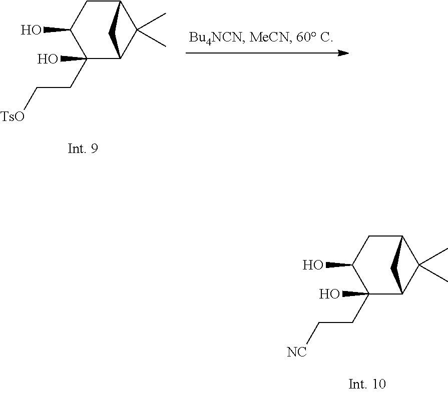 [SMILES: Cc1ccc(S(=O)(=O)OCC[C@@]2(O)[C@@H]3C[C@H](C[C@@H]2O)C3(C)C)cc1 ; CC1(C)[C@@H]2C[C@H]1[C@](O)(CCC#N)[C@@H](O)C2]

Into a 40 mL vial were added 2-[(1R,2R,3S,5R)-2,3-dihydroxy-6,6-dimethylbicyclo[3.1.1]heptan-2-yl]ethyl 4-methylbenzenesulfonate (1.00 g, 2.82 mmol, 1 equiv), Bu₄NCN (1.51 g, 5.64 mmol, 2 equiv), and MeCN (20 mL). The resulting mixture stirred at 60° C. for 16 h. After cooling to r.t., the mixture was concentrated under reduced pressure to afford 3-[(1R,2R,3S,5R)-2,3-dihydroxy-6,6-dimethylbicyclo[3.1.1]heptan-2-yl]propanenitrile (2.10 g) as an yellow oil, the crude product was directly used in the next step without further purification.

LCMS: (ES, m/z): [M+H]⁺=210 (LCMS condition: Column: Shim-pack Scepter C18-120; Mobile phase A: Water/5 mM NH₄HCO₃; Mobile phase B: Acetonitrile; Flow rate: 1.5 mL/min; RT: 0.643 min)

### (d) Synthesis of Int. 11

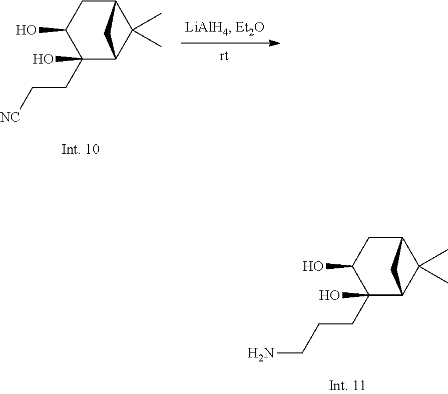

Into a 100 mL round-bottom flask were added 3-[(1R,2R,3S,5R)-2,3-dihydroxy-6,6-dimethylbicyclo[3.1.1]heptan-2-yl]propanenitrile (2.00 g, 9.56 mmol, 1 equiv), lithium aluminum hydride (1.09 g, 28.7 mmol, 3 equiv), and diethyl ether (40 mL) at 0° C. The resulting mixture was warmed to r.t. and stirred for 4 h. The reaction was quenched by the addition of water/ice (20 mL) at 0° C. After filtration, the filtrate was concentrated under reduced pressure to afford (1R,2R,3S,5R)-2-(3-aminopropyl)-6,6-dimethylbicyclo[3.1.1]heptane-2,3-diol (1.66 g, 81% yield) as an off-white semi-solid.

LCMS: (ES, m/z): [M+H]⁺=214 (LCMS condition: Column: Shim-pack Scepter C18-120; Mobile phase A: Water/5 mM NH₄HCO₃; Mobile phase B: Acetonitrile; Flow rate: 1.5 mL/min; RT: 0.618 min)

### (e) Synthesis of Int. 12

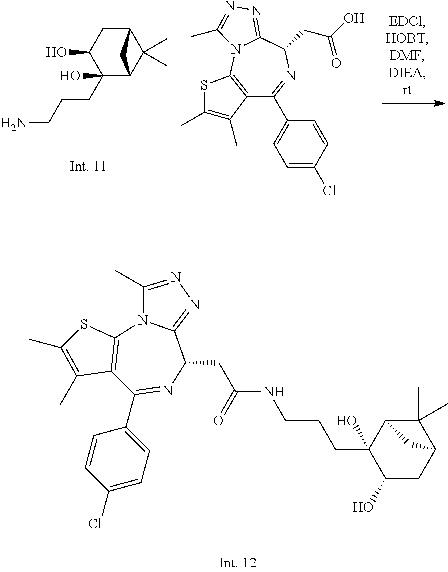

Into a 500 mL round-bottom flask were added (1R,2R,3S,5R)-2-(3-aminopropyl)-6,6-dimethylbicyclo[3.1.1]heptane-2,3-diol (4.80 g, 22.5 mmol, 1 equiv), (S)-2-(4-(4-chlorophenyl)-2,3,9-trimethyl-6H-thieno[3,2-f][1,2,4]triazolo[4,3-a][1,4]diazepin-6-yl)acetic acid (1.80 g, 4.50 mmol, 0.2 equiv), EDCI (5.61 g, 29.3 mmol, 1.3 equiv), HOBT (3.95 g, 29.2 mmol, 1.3 equiv), DIEA (872 g, 67.5 mmol, 3 equiv), and DMF (200 mL). The resulting mixture was stirred at 25° C. for 16 h. Upon completion of the reaction, the mixture was poured into water (200 mL) and then the resulting mixture was extracted with EtOAc (3×500 mL). The combined organic layers were washed with brine (3×300 mL) and dried over anhydrous Na₂SO₄. After filtration, the filtrate was concentrated under reduced pressure. The crude product was purified by reverse phase flash with the following conditions (Column: XBridge Prep OBD C18 Column, 30×150 mm, 5 μm; Mobile Phase A: Water (10 mmol/L NH₄HCO₃+0.05% NH₃), Mobile Phase B: ACN; Flow rate: 60 mL/min mL/min; Gradient: 30% B to 60% B in 8 min; Wave Length: 254 nm/220 nm; RT1 (min): 6.9) to afford 2-((S)-4-(4-chlorophenyl)-2,3,9-trimethyl-6H-thieno[3,2-f][1,2,4]triazolo[4,3-a][1,4]diazepin-6-yl)-N-(3-((1R,2R,3S,5R)-2,3-dihydroxy-6,6-dimethylbicyclo[3.1.1]heptan-2-yl)propyl)acetamide(807 mg, 6% yield) as a white solid.

LCMS: (ES, m/z): [M−H]⁻=594 (LCMS condition: Column: Shim-pack Scepter C18-120; Mobile phase A: Water/5 mM NH₄HCO₃; Mobile phase B: Acetonitrile; Flow rate: 1.5 mL/min; RT: 1.336 min)

### (f) Synthesis of Int. 13

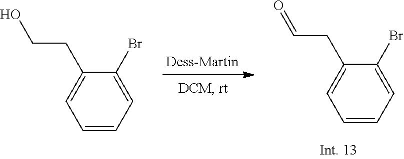 [SMILES: OCCc1ccccc1Br ; O=CCc1ccccc1Br]

Into a 500 mL round-bottom flask were added 2-(2-bromophenyl)ethanol (10.0 g, 49.7 mmol, 1 equiv), DCM (200 mL) and Dess-Martin periodinane (25.3 g, 59.7 mmol, 1.2 equiv) at 0° C. The resulting mixture was stirred for 3 h at 17° C. The reaction was filtered through a pad of Celite and then the Celite was washed with DCM (100 mL). Sat. NaHCO₃ solution (200 mL) was added to the filtrate and the phases were separated. The organic phase was washed with sat. NaHCO₃ solution (200 mL) and brine (100 mL), dried over anhydrous magnesium sulfate, and concentrated under reduced pressure. The residue was purified by silica gel column chromatography, eluted with DCM/PE (1:5) to afford 2-(2-bromophenyl)acetaldehyde (5.20 g, 41% yield) as a yellow oil.

LCMS: (ES, m/z): none (LCMS condition: Column: Shim-pack Scepter C18-120; Mobile phase A: Water/5 mM NH₄HCO₃; Mobile phase B: Acetonitrile; Flow rate: 1.5 mL/min; RT: 0.951 min)

### (g) Synthesis of Int. 14

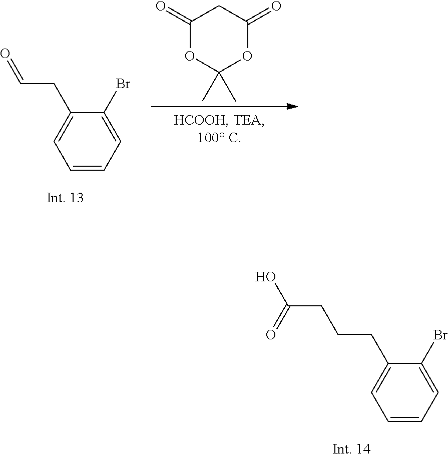

Into a 40 mL microwave tube were added 2-(2-bromophenyl)acetaldehyde (2.60 g, 13.1 mmol, 1 equiv), 6.0 mL TEAF (TEAF:TEA (10 mL) was added to FA (6.8 mL) at 0° C.), and meldrum's acid (1.88 g, 13.1 mmol, 1 equiv) at 17° C. The resulting mixture was stirred for 16 h at 100° C. The reaction was quenched with water/ice at 17° C. and the resulting mixture was extracted with EtOAc (3×100 mL). The combined organic layers were washed with brine (100 mL) and dried over anhydrous Na₂SO₄. After filtration, the filtrate was concentrated under reduced pressure. The residue was purified by reverse-phase flash chromatography with the following conditions: column, C18 silica gel; mobile phase, MeCN in water (0.05% FA), 40 to 60% gradient in 10 min; detector, UV 220 nm to afford 4-(2-bromophenyl)butanoic acid (1.85 g, 58% yield) as an off-white solid.

LCMS: (ES, m/z): [M−H]⁻=241 (LCMS condition: Column: Shim-pack Scepter C18-120; Mobile phase A: Water/5 mM NH₄HCO₃; Mobile phase B: Acetonitrile; Flow rate: 1.5 mL/min; RT: 0.565 min)

### (h) Synthesis of Int. 15

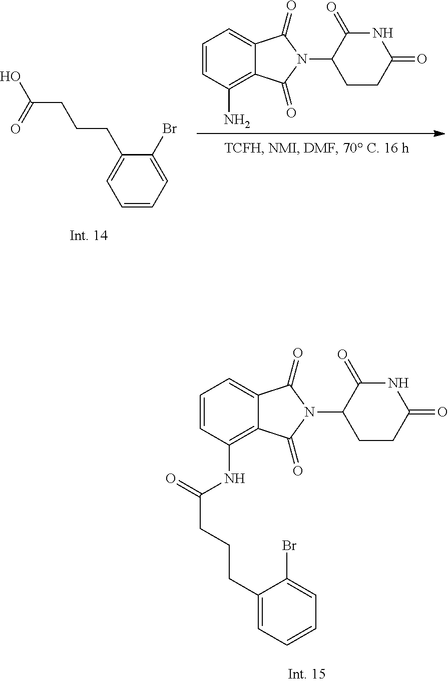 [SMILES: O=C(O)CCCc1ccccc1Br ; Nc1cccc2c1C(=O)N(C1CCC(=O)NC1=O)C2=O ; O=C1CCC(N2C(=O)c3cccc(NC(=O)CCCc4ccccc4Br)c3C2=O)C(=O)N1]

Into a 40 mL vial were added 4-(2-bromophenyl)butanoic acid (1.44 g, 5.92 mmol, 1 equiv), DMF (14 mL), pomalidomide (2.42 g, 8.89 mmol, 1.5 equiv), TCFH (2.5 g, 8.89 mmol, 1.5 equiv), and NMI (1.46 g, 17.8 mmol, 3 equiv) at 16° C. and the reaction was stirred for 14 h at 70° C. The reaction was cooled to 16° C. and quenched with water. The resulting mixture was extracted with EtOAc (3×100 mL). The combined organic layers were washed with brine (100 mL), dried over anhydrous Na₂SO₄. After filtration, the filtrate was concentrated under reduced pressure. The residue was purified by reverse-phase flash chromatography with the following conditions: column, C18 silica gel; mobile phase, MeCN in water (0.1% FA), 30 to 70% gradient in 20 min; detector, UV 254 nm to afford 4-(2-bromophenyl)-N-[2-(2,6-dioxopiperidin-3-yl)-1,3-dioxoisoindol-4-yl]butanamide (racemic) (1.30 g, 44% yield) as an off-white solid.

LCMS: (ES, m/z): [M−H]⁻=496 (LCMS condition: Column: HALO 90A C18; Mobile phase A: water/0.1% FA; Mobile phase B: ACN/0.11% FA; Flow rate: 1.5 mL/min; RT: 1.125 min)

### (i) Synthesis of Int. 16

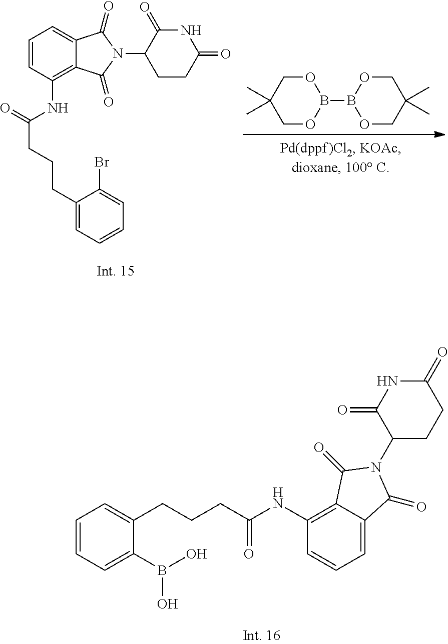

Into a 100 mL round-bottom flask were added 4-(2-bromophenyl)-N-[2-(2,6-dioxopiperidin-3-yl)-1,3-dioxoisoindol-4-yl]butanamide (racemic) (2.80 g, 5.62 mmol, 1 equiv), 2-(5,5-dimethyl-1,3,2-dioxaborinan-2-yl)-5,5-dimethyl-1,3,2-dioxaborinane (2.54 g, 11.2 mmol, 2 equiv), dioxane (30 mL), KOAc (1.65 g, 16.9 mmol, 3 equiv) and Pd(dppf)Cl₂ (411 mg, 0.562 mmol, 0.1 equiv) at 16° C. The reaction was stirred for 2 h at 100° C. under N₂. The resulting mixture was cooled to 16° C., quenched with water, and extracted with EtOAc (3×100 mL). The combined organic layers were washed with brine (100 mL) and dried over anhydrous Na₂SO₄. After filtration, the filtrate was concentrated under reduced pressure. The residue was purified by reverse-phase flash chromatography with the following conditions: column, C18 silica gel; mobile phase, MeCN in water (0.05% FA), 40 to 80% gradient in 250 min; detector, UV 254 nm. The crude product was further purified by prep-HPLC with the following conditions (Column: Xselect CSH Prep C18 Column, 30×150 mm, 5 μm; Mobile Phase A: water (0.1% FA), Mobile Phase B: ACN; Flow rate: 60 mL/min; Gradient: 26 to 56% B in 10 min; Wave Length: 254 nm/220 nm; RT (min): 8.18) to afford 1.9 g crude product as a light yellow solid. The yellow solid was then re-purified by prep-HPLC using the following conditions (Column: WelFlash C18-I, Regular C18 20-40 μm, 330 g; Mobile Phase A: water (0.1% FA), Mobile Phase B: 20 mm NaOH+10% ACN; Flow rate: 100 mL/min; Gradient: 25 to 55% B in 30 min; Wave Length: 254 nm/220 nm; Rt1 (min): 25) to afford 2-(3-{[2-(2,6-dioxopiperidin-3-yl)-1,3-dioxoisoindol-4-yl]carbamoyl}propyl)phenylboronic acid (racemic) (1.01 g, 58% yield) as a light yellow solid.

LCMS: (ES, m/z): [M−H]⁻=462 (LCMS condition: Column: HALO 90A C18; Mobile phase A: water/0.1% FA; Mobile phase B: ACN/0.1% FA; Flow rate: 1.2 mL/min; RT: 1.356 min)

### (j) Synthesis of Example 2

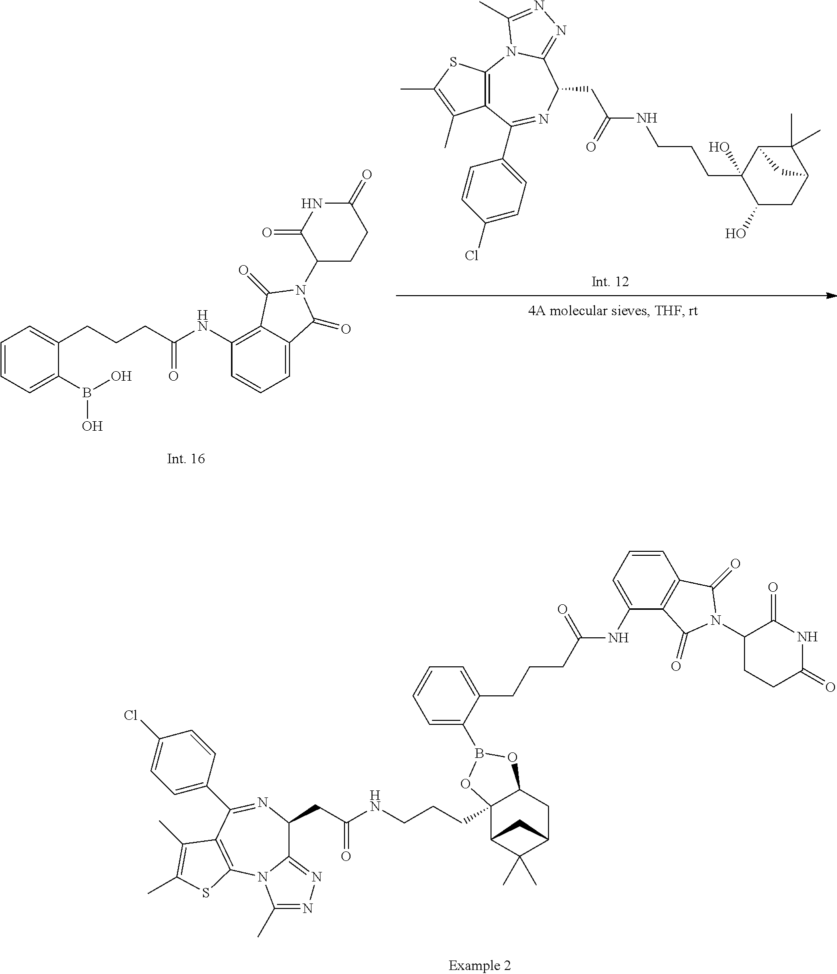

Into a 250 mL round-bottom flask were added 2-(3-{[2-(2,6-dioxopiperidin-3-yl)-1,3-dioxoisoindol-4-yl]carbamoyl}propyl)phenylboronic acid (racemic) (112 mg, 0.242 mmol, 1 equiv), 2-((S)-4-(4-chlorophenyl)-2,3,9-trimethyl-6H-thieno[3,2-f][1,2,4]triazolo[4,3-a][1,4]diazepin-6-yl)-N-(3-((1R,2R,3S,5R)-2,3-dihydroxy-6,6-dimethylbicyclo[3.1.1]heptan-2-yl)propyl)acetamide(144 mg, 0.242 mmol, 1 equiv), 4A molecular sieves (300 mg), and THF (50 mL). The reaction was stirred at 25° C. for 16 h and then filtered. The filter cake was washed with THE (3×30 mL) and the filtrate was concentrated under reduced pressure. The crude product was purified by Prep-Achiral-SFC with the following conditions (Column: DAICEL DCpak P4VP 3*25 cm, 5 μm; Mobile Phase A: CO₂, Mobile Phase B: MeOH (1%-2M-NH₃-MeOH); Flow rate: 65 mL/min; Gradient: isocratic 38% B) Column Temperature (° C.): 35; Back Pressure (bar): 100; Wave Length: 220 nm; RT (min): 6.52; Sample Solvent: MeOH; Injection Volume: 3 mL; Number Of Runs: 4) to afford 4-(2-((3aR,4R,6R,7aS)-3a-(3-(2-((S)-4-(4-chlorophenyl)-2,3,9-trimethyl-6H-thieno[3,2-f][1,2,4]triazolo[4,3-a][1,4]diazepin-6-yl)acetamido)propyl)-5,5-dimethylhexahydro-4,6-methanobenzo[d][1,3,2]dioxaborol-2-yl)phenyl)-N-(2-(2,6-dioxopiperidin-3-yl)-1,3-dioxoisoindolin-4-yl)butanamide (mixture of diastereoisomers) (133 mg, 53% yield) as a white solid.

LCMS: (ES, m/z): [M+H]⁺=1023 LCMS condition: Column: Shim-pack Scepter C18-120: Mobile phase A: Water/5 mM NH₄HCO₃; Mobile phase B: Acetonitrile; Flow rate: 1.5 mL/min; RT: 1.092 min)

### Example 3

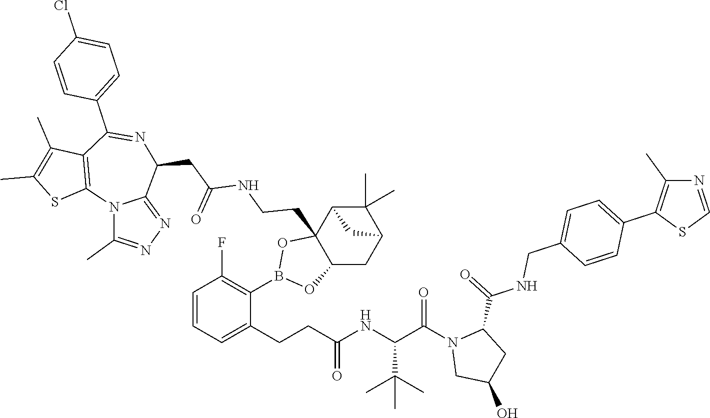 [SMILES: Cc1ncsc1-c1ccc(CNC(=O)[C@@H]2C[C@@H](O)CN2C(=O)[C@@H](NC(=O)CCc2cccc(F)c2B2O[C@H]3C[C@H]4C[C@H](C4(C)C)[C@@]3(CCNC(=O)C[C@@H]3N=C(c4ccc(Cl)cc4)c4c(sc(C)c4C)-n4c(C)nnc43)O2)C(C)(C)C)cc1]

### (a) Synthesis of Int. 1-5 as Described in Example 1

### (b) Synthesis of Int. 18

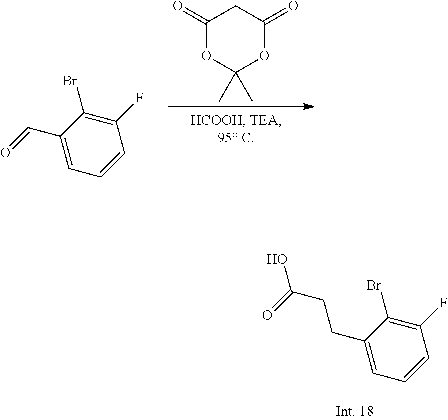

To a 50 mL flask containing formic acid (6.8 mL, 44.8 mmol) at 0° C. was slowly added NEt₃ (10 mL, 62.1 mmol). To the mixture was added 2-bromo-3-fluorobenzaldehyde (3.5 g, 17.241 mmol, 1 equiv) and Meldrum's acid (2.48 g, 17.2 mmol, 1 equiv) in portions and the resulting mixture was stirred overnight at 95° C. After the completion of reaction, the mixture was basified to pH 11 with aqueous NaOH and extracted with EA (3×100 mL). The aqueous layer was acidified to pH 2 with 4 M HCl and was left to crystallize. After filtration, the crystals were washed with water and dried to afford 3-(2-bromo-3-fluorophenyl)propanoic acid (1.63 g, 37% yield) as a white solid.

LCMS: (ES, m/z): [M−H]⁻=245 (LCMS condition: Column: HALO 90A C18; Mobile phase A: water/0.1% FA; Mobile phase B: ACN/0.1% FA; Flow rate: 1.2 mL/min; RT: 0.801 min)

### (c) Synthesis of Int. 19

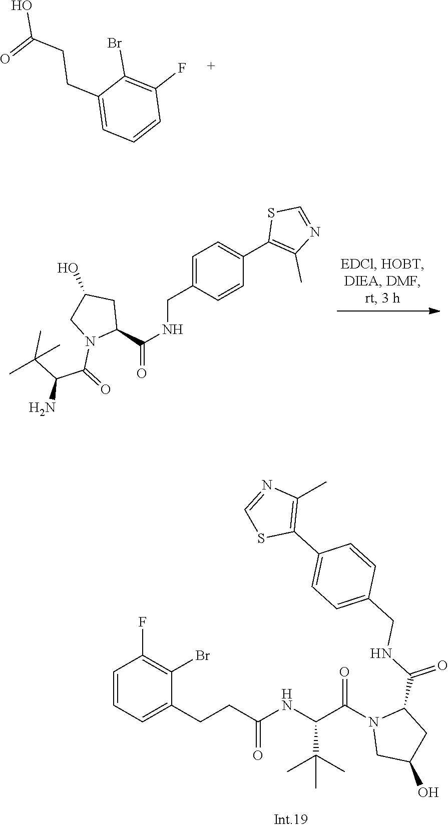 [SMILES: O=C(O)CCc1cccc(F)c1Br ; Cc1ncsc1-c1ccc(CNC(=O)[C@@H]2C[C@@H](O)CN2C(=O)[C@@H](N)C(C)(C)C)cc1 ; Cc1ncsc1-c1ccc(CNC(=O)[C@@H]2C[C@@H](O)CN2C(=O)[C@@H](NC(=O)CCc2cccc(F)c2Br)C(C)(C)C)cc1]

To a stirred solution of 3-(2-bromo-3-fluorophenyl)propanoic acid (1.5 g, 6,071 mmol, 1 equiv) and (2S,4R)-1-[(2S)-2-amino-3,3-dimethylbutanoyl]-4-hydroxy-N-{[4-(4-methyl-1,3-thiazol-5-yl)phenyl]methyl}pyrrolidine-2-carboxamide (3.14 g, 7.285 mmol, 1.2 equiv) in anhydrous DMF (15 mL) was added EDCI (1.75 g, 9.107 mmol, 1.5 equiv), HOBT (1.23 g, 9.107 mmol, 1.5 equiv) and DIEA (2.35 g, 18.213 mmol, 3 equiv) at r.t and stirred for 3 h. After completion of reaction, the mixture was quenched by addition of water (100 mL). The aqueous layer was extracted with EtOAc (3×150 mL). The combined organic extracts were washed with brine (150 mL), dried over anhydrous sodium sulfate and concentrated under reduced pressure. The crude material was purified by silica column chromatography eluting with 0 to 10% MeOH in DCM gradient to afford desired compound (2S,4R)-1-[(2S)-2-[3-(2-bromo-3-fluorophenyl)propanamido]-3,3-dimethylbutanoyl]-4-hydroxy-N-{[4-(4-methyl-1,3-thiazol-5-yl)phenyl]methyl}pyrrolidine-2-carboxamide (2.1 g, 52% yield) as a light yellow solid.

LCMS: (ES, m/z): [M+H]⁺=659 (LCMS condition: Column: HALO 90A C18; Mobile phase A: water/0.1% FA; Mobile phase B: ACN/0.1% FA; Flow rate: 1.2 mL/min; RT: 0.879 min)

### (d) Synthesis of Int. 20

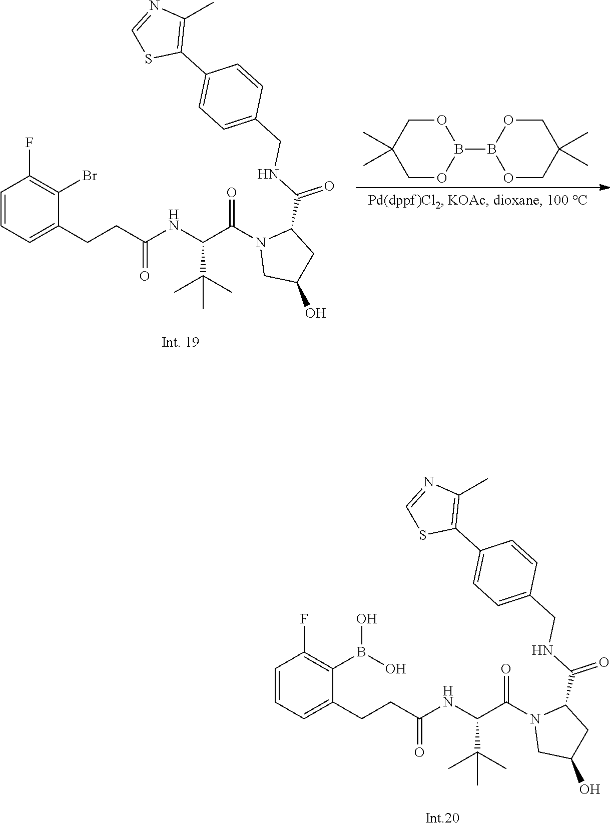

A solution of (2S,4R)-1-[(2S)-2-[3-(2-bromo-3-fluorophenyl)propanamido]-3,3-dimethylbutanoyl]-4-hydroxy-N-{[4-(4-methyl-1,3-thiazol-5-yl)phenyl]methyl}pyrrolidine-2-carboxamide (2 g, 2.91 mmol, 1 equiv) and 2-(5,5-dimethyl-1,3,2-dioxaborinan-2-yl)-5,5-dimethyl-1,3,2-dioxaborinane (1.32 g, 5.82 mmol, 2 equiv) in 1,4-dioxane (5 mL) was treated with Pd(dppf)Cl₂·CH₂Cl₂ (237.12 mg, 0.291 mmol, 0.10 equiv) and KOAc (857.01 mg, 8.73 mmol, 3 equiv) under a nitrogen atmosphere. The mixture was stirred at 100° C. for 2 h. After completion of the reaction, the mixture was filtered, the filter cake was washed with DCM and the filtrate was concentrated under reduced pressure. The crude product was purified by silica column chromatography eluting with 0 to 10% MeOH in DCM gradient to afford a black solid (1.9 g). This was further purified by Prep-HPLC with the following conditions (Column: WelFlash C18-I, Regular C18 20-40 μm, 120 g; Mobile Phase A: Water (0.1% FA), Mobile Phase B: ACN; Flow rate: 60 mL/min; Gradient: 20% to 50% B in 30 min; Wavelength: 254 nm/220 nm; RT (min): 25) to afford 2-fluoro-6-(2-{[(2S)-1-[(2S,4R)-4-hydroxy-2-({[4-(4-methyl-1,3-thiazol-5-yl)phenyl]methyl}carbamoyl)pyrrolidin-1-yl]-3,3-dimethyl-1-oxobutan-2-yl]carbamoyl}ethyl)phenylboronic acid (830.5 mg, 45% yield) as a white solid.

LCMS: (ES, m/z): [M−H]⁻=623 (LCMS condition: Column: Shim-pack Scepter C18-120; Mobile phase A: Water/5 mM NH₄HCO₃; Mobile phase B: Acetonitrile; Flow rate: 1.5 mL/min; RT: 1.357 min)

### (e) Synthesis of Example 3

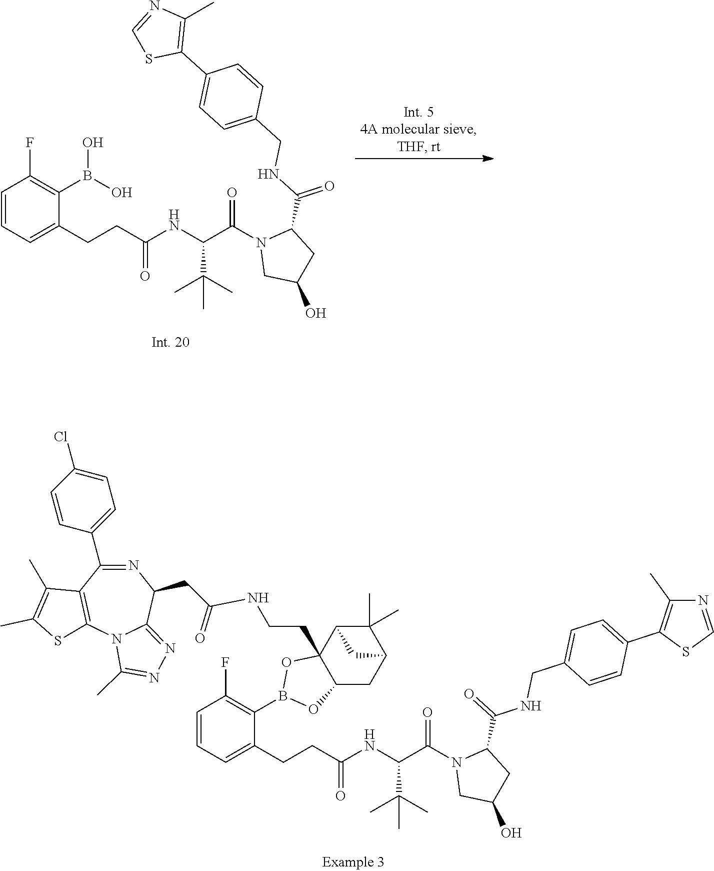 [SMILES: Cc1ncsc1-c1ccc(CNC(=O)[C@@H]2C[C@@H](O)CN2C(=O)[C@@H](NC(=O)CCc2cccc(F)c2B(O)O)C(C)(C)C)cc1 ; Cc1ncsc1-c1ccc(CNC(=O)[C@@H]2C[C@@H](O)CN2C(=O)[C@@H](NC(=O)CCc2cccc(F)c2B2O[C@H]3C[C@H]4C[C@H](C4(C)C)[C@@]3(CCNC(=O)C[C@@H]3N=C(c4ccc(Cl)cc4)c4c(sc(C)c4C)-n4c(C)nnc43)O2)C(C)(C)C)cc1]

To a stirred solution of 2-((S)-4-(4-chlorophenyl)-2,3,9-trimethyl-6H-thieno[3,2-f][1,2,4]triazolo[4,3-a][1,4]diazepin-6-yl)-N-(2-((1R,2R,3S,5R)-2,3-dihydroxy-6,6-dimethylbicyclo[3.1.1]heptan-2-yl)ethyl)acetamide(140 mg, 0.238 mmol, 1 equiv) in THF (2 mL) was added 2-fluoro-6-(2-{[(2S)-1-[(2S,4R)-4-hydroxy-2-({[4-(4-methyl-1,3-thiazol-5-yl)phenyl]methyl}carbamoyl)pyrrolidin-1-yl]-3,3-dimethyl-1-oxobutan-2-yl]carbamoyl}ethyl)phenylboronic acid (148.69 mg, 0.238 mmol, 1 equiv) in portions at 25° C. The mixture was stirred for 16 h at 25° C. After the completion of reaction, the mixture was filtered, and the filtrate was concentrated under reduced pressure. The crude product was purified by Prep-HPLC with the following conditions (Column: XBridge Prep OBD C18 Column, 30×150 mm, 5 μm; Mobile Phase A: Water (10 mmol/L NH₄HCO₃), Mobile Phase B: ACN; Flow rate: 60 mL/min; Gradient: 56% to 66% B in 7 min; Wavelength: 254 nm/220 nm; RT (min): 6.8) to afford 2-((S)-4-(4-chlorophenyl)-2,3,9-trimethyl-6H-thieno[3,2-f][1,2,4]triazolo[4,3-a][1,4]diazepin-6-yl)-N-(2-((1R,2R,3S,5R)-2,3-dihydroxy-6,6-dimethylbicyclo[3.1.1]heptan-2-yl)ethyl)acetamide (129.1 mg, 46% yield) as a white solid.

LCMS: (ES, m/z): [M+H]⁺=1170 (LCMS condition: Column: Shim-pack Scepter C18-120; Mobile phase A: Water/5 mM NH₄HCO₃; Mobile phase B: Acetonitrile; Flow rate: 1.5 mL/min.; RT: 1.393 min)

### Example 4

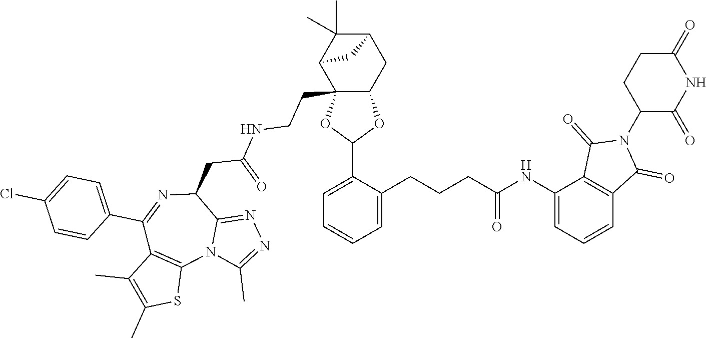

### (a) Synthesis of Int. 1-5 and 13-16 as Described in Examples 1 and 2 Respectively

### (b) Synthesis of Example 4

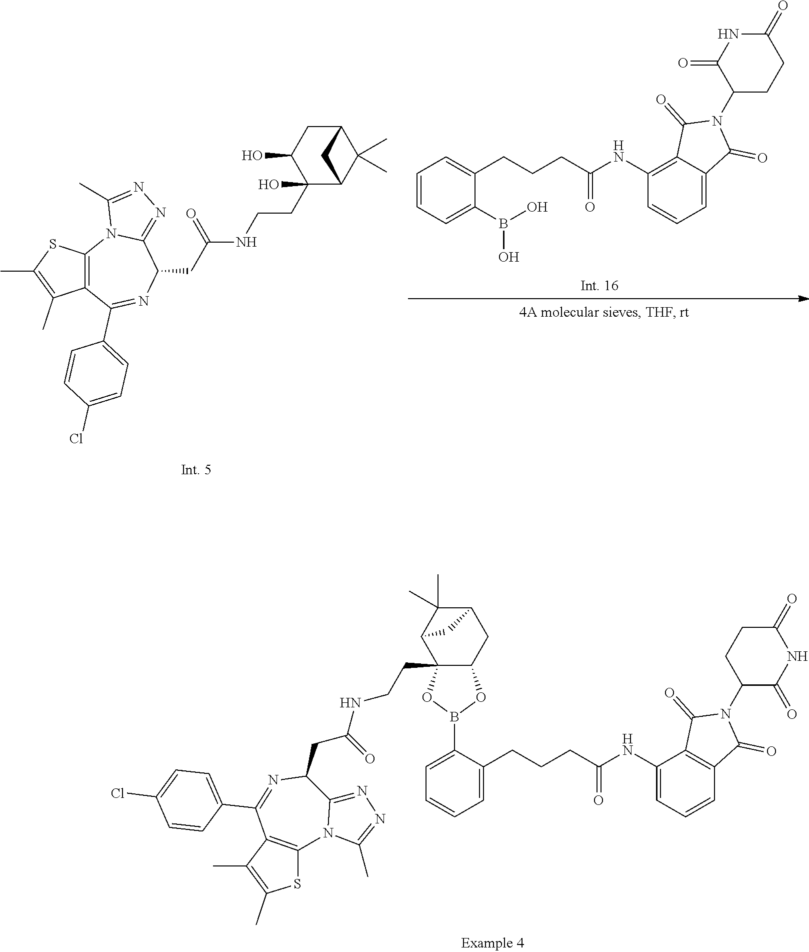

A solution of 2-((S)-4-(4-chlorophenyl)-2,3,9-trimethyl-6H-thieno[3,2-f][1,2,4]triazolo[4,3-a][1,4]diazepin-6-yl)-N-(2-((1R,2R,3S,5R)-2,3-dihydroxy-6,6-dimethylbicyclo[3.1.1]heptan-2-yl)ethyl)acetamide(150 mg, 0.258 mmol, 1 equiv), 4A molecular sieves (450 mg), and 2-(3-{[2-(2,6-dioxopiperidin-3-yl)-1,3-dioxoisoindol-4-yl]carbamoyl}propyl)phenylboronic acid (racemic) (119 mg, 0.258 mmol, 1 equiv) in THF was stirred for 16 h at 25° C. The resulting mixture was filtered, the filter cake was washed with THF (3×20 mL), and the filtrate was concentrated under reduced pressure. The resulting mixture was purified by Prep-SFC with the following conditions (Column: GreenSepBasic, 4.6*×100 mm, 3 μm; Mobile Phase B: MeOH (1% 2M NH₃); Gradient: isocratic/b B) to afford crude product (150 mg) as a light-yellow solid. The crude product (150 mg) was purified by Prep-HPLC with the following conditions (Column: Xselect CSH Prep C18 Column, 30*×150 mm, 5 μm; Mobile Phase A: Water (0.1% FA), Mobile Phase B: ACN; Flow rate: 60 mL/min mL/min; Gradient: 60% B to 90% B in 10 min; Wave Length: 254 nm/220 nm; RT (min): 9) to afford 4-(2-((3aR,4R,6R,7aS)-3a-(2-(2-((S)-4-(4-chlorophenyl)-2,3,9-trimethyl-6H-thieno[3,2-f][1,2,4]triazolo[4,3-a][1,4]diazepin-6-yl)acetamido)ethyl)-5,5-dimethylhexahydro-4,6-methanobenzo[d][1,3,2]dioxaborol-2-yl)phenyl)-N-(2-(2,6-dioxopiperidin-3-yl)-1,3-dioxoisoindolin-4-yl)butanamide (mixture of diastereoisomers) (103 mg, 39% yield) as a white solid.

LCMS: (ES, m/z): [M+H]⁺=1009 (LCMS condition: Column: Shim-pack Scepter C18-120; Mobile phase A: Water/5 mM NH₄HCO₃; Mobile phase B: Acetonitrile; Flow rate: 1.5 mL/min; RT: 1.068 min)

### Example 5

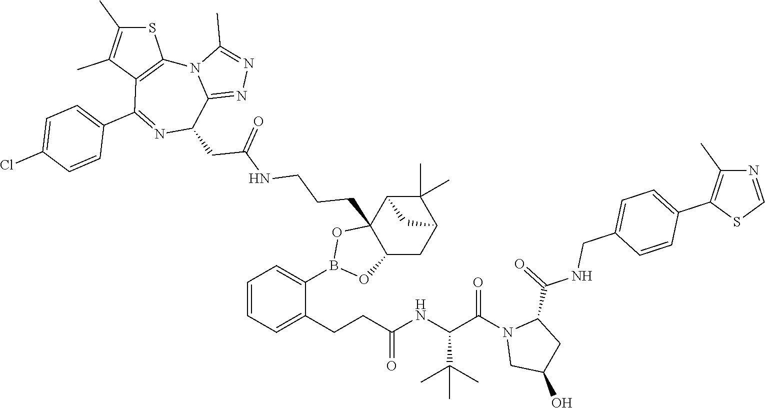 [SMILES: Cc1ncsc1-c1ccc(CNC(=O)[C@@H]2C[C@@H](O)CN2C(=O)[C@@H](NC(=O)CCc2ccccc2B2O[C@H]3C[C@H]4C[C@H](C4(C)C)[C@@]3(CCCNC(=O)C[C@@H]3N=C(c4ccc(Cl)cc4)c4c(sc(C)c4C)-n4c(C)nnc43)O2)C(C)(C)C)cc1]

(a) Synthesis of Int. 6, 7 then 1 as Described in Example 1 and Synthesis of Int. 9-12 as Described in Example 2

### (b) Synthesis of Example 5

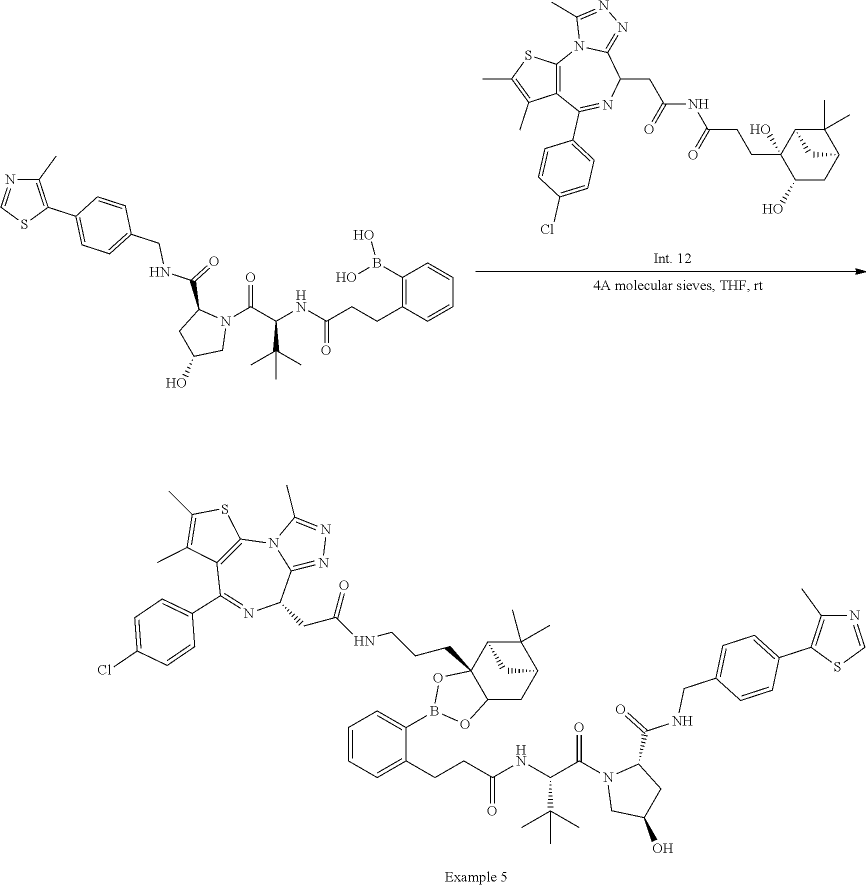

Into a 250 mL round-bottom flask were added 2-(2-{[(2S)-1-[(2S,4R)-4-hydroxy-2-({[4-(4-methyl-1,3-thiazol-5-yl)phenyl]methyl}carbamoyl)pyrrolidin-1-yl]-3,3-dimethyl-1-oxobutan-2-yl]carbamoyl}ethyl)phenylboronic acid (120 mg, 0.198 mmol, 1 equiv), 2-((S)-4-(4-chlorophenyl)-2,3,9-trimethyl-6H-thieno[3,2-f][1,2,4]triazolo[4,3-a][1,4]diazepin-6-yl)-N-(3-((1R,2R,3S,5R)-2,3-dihydroxy-6,6-dimethylbicyclo[3.1.1]heptan-2-yl)propyl)acetamide (118 mg, 0.198 mmol, 1 equiv), 4A molecular sieves (300 mg) and THF (60 mL) and the reaction was stirred at 25° C. for 16 h. The resulting mixture was filtered, the filter cake was washed with THF (3×30 mL), and the filtrate was concentrated under reduced pressure. The crude product (320 mg) was purified by Prep-HPLC with the following conditions (Column: XBridge Prep OBD C18 Column, 19×250 mm, 5 μm; Mobile Phase A: Water (10 mmol/L NH₄HCO₃+0.05% NH₃), Mobile Phase B: ACN (1% 2 mM NH₃-MeOH); Flow rate: 25 mL/min mL/min; Gradient: 68% B to 98% B in 12 min; Wave Length: 254 nm/220 nm; RT (min): 11.5) to afford (2S,4R)-1-((S)-2-(3-(2-((3aR,4R,6R,7aS)-3a-(3-(2-((S)-4-(4-chlorophenyl)-2,3,9-trimethyl-6H-thieno[3,2-f][1,2,4]triazolo[4,3-a][1,4]diazepin-6-yl)acetamido)propyl)-5,5-dimethylhexahydro-4,6-methanobenzo[d][1,3,2]dioxaborol-2-yl)phenyl)propanamido)-3,3-dimethylbutanoyl)-4-hydroxy-N-(4-(4-methylthiazol-5-yl)benzyl)pyrrolidine-2-carboxamide (105 mg, 46% yield) as a white solid.

LCMS: (ES, m/z): [M+H]⁺=1166 (LCMS condition: Column: Shim-pack Scepter C18-120; Mobile phase A: Water/5 mM NH₄HCO₃; Mobile phase B: Acetonitrile; Flow rate: 1.5 mL/min; RT: 1.574 min)

### Example 6

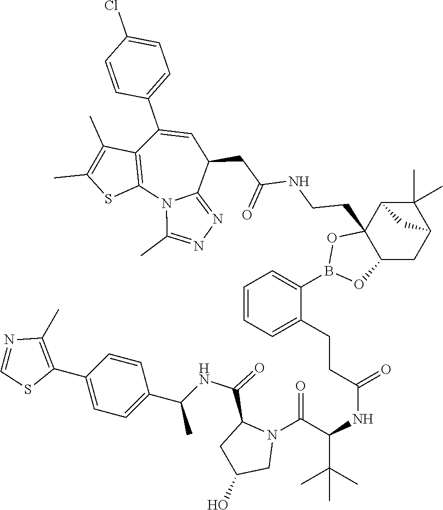 [SMILES: Cc1ncsc1-c1ccc([C@H](C)NC(=O)[C@@H]2C[C@@H](O)CN2C(=O)[C@@H](NC(=O)CCc2ccccc2B2O[C@H]3C[C@H]4C[C@H](C4(C)C)[C@@]3(CCNC(=O)C[C@@H]3C=C(c4ccc(Cl)cc4)c4c(sc(C)c4C)-n4c(C)nnc43)O2)C(C)(C)C)cc1]

### (a) Synthesis of Int. 1-5 as Described in Example 1

### (b) Synthesis of Int. 24

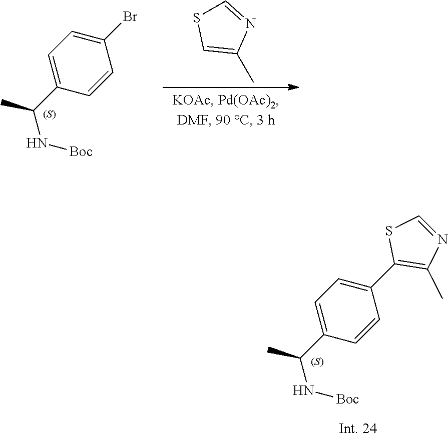

Into a 250 mL 3-necked round-bottom flask were added tert-butyl N-[(1S)-1-(4-bromophenyl)ethyl]carbamate (5.0 g, 16.656 mmol, 1 equiv), 4-methylthiazole (3.30 g, 33.283 mmol, 2 equiv), DMF (50 mL), KOAc (3.27 g, 33.319 mmol, 2 equiv), and Pd(OAc)₂ (0.19 g, 0.846 mmol, 0.05 equiv) at 16° C. The resulting mixture was stirred for 3 h at 90° C. under a nitrogen atmosphere and then quenched with water at 16° C. The resulting mixture was extracted with EtOAc (3×50 mL). The combined organic layers were washed with brine (3×50 mL) and dried over anhydrous Na₂SO₄. After filtration, the filtrate was concentrated under reduced pressure. The residue was purified by silica gel column chromatography eluting with PE/EA (1:1) to afford tert-butyl N-[(1S)-1-[4-(4-methyl-1,3-thiazol-5-yl)phenyl]ethyl]carbamate (4.5 g, 74% yield) as a yellow solid.

LCMS: (ES, m/z): [M+H]+=319 (LCMS condition: Column: Shim-pack Scepter C18-120; Mobile phase A: Water/5 mM NH₄HCO₃ Mobile phase B: Acetonitrile; Flow rate: 1.5 mL/min; RT: 1.353 min)

### (c) Synthesis of Int. 25

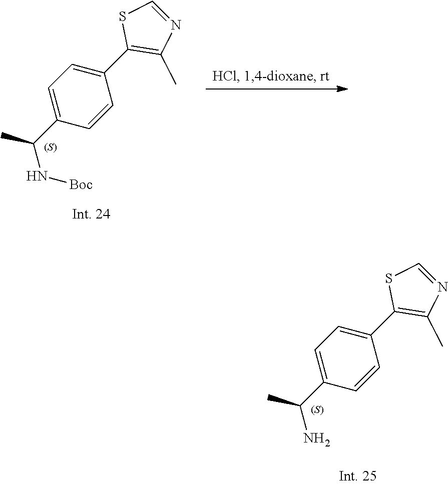

Into a 100 mL round-bottom flask were added tert-butyl N-[(1S)-1-[4-(4-methyl-1,3-thiazol-5-yl)phenyl]ethyl]carbamate (4.5 g, 14.131 mmol, 1 equiv), 1,4-dioxane (10 mL), and HCl in 1,4-dioxane (50 mL, 4 M) at 16° C. The resulting mixture was stirred for 1 h at 16° C. The resulting mixture was concentrated under reduced pressure to give 1-[4-(4-methyl-1,3-thiazol-5-yl)phenyl]ethanamine (3.3 g, 93% yield) as a yellow solid.

LCMS: (ES, m/z): [M+H−17]⁺=202 (LCMS condition: Column: Shim-pack Scepter C18-120; Mobile phase A: Water/5 mM NH₄HCO₃; Mobile phase B: Acetonitrile; Flow rate: 1.5 mL/min; RT: 0.634 min)

### (d) Synthesis of Int. 26

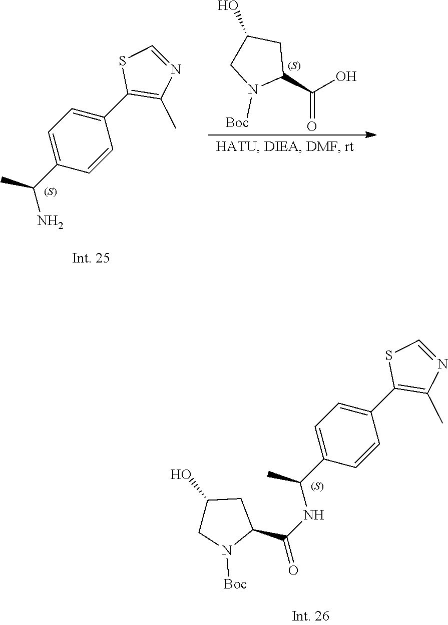

Into a 100 mL round-bottom flask were added (1S)-1-[4-(4-methyl-1,3-thiazol-5-yl)phenyl]ethanamine (3.3 g, 15.115 mmol, 1.2 equiv), (2S,4R)-1-(tert-butoxycarbonyl)-4-hydroxypyrrolidine-2-carboxylic acid (2.91 g, 12.596 mmol, 1 equiv), DMF (30 mL), HATU (7.18 g, 18.894 mmol, 1.5 equiv), and DIEA (4.88 g, 37.788 mmol, 3 equiv) and the resulting mixture was stirred for 1 h at 16° C. The reaction was quenched with water at 16° C. and the resulting mixture was extracted with EtOAc (3×100 mL). The combined organic layers were washed with brine (2×100 mL) and dried over anhydrous Na₂SO₄. After filtration, the filtrate was concentrated under reduced pressure. The residue was purified by silica gel column chromatography eluting with PE/EA (1:1) to afford tert-butyl (2S,4R)-4-hydroxy-2-{[(1S)-1-[4-(4-methyl-1,3-thiazol-5-yl)phenyl]ethyl]carbamoyl}pyrrolidine-1-carboxylate (2.8 g, 51% yield) as a light yellow solid.

LCMS: (ES, m/z): [M−H]⁻=430 (LCMS condition: Column: Shim-pack Scepter C18-120; Mobile phase A: Water/5 mM NH₄HCO₃; Mobile phase B: Acetonitrile; Flow rate: 1.5 mL/min; RT: 0.801 min)

### (e) Synthesis of Int. 27

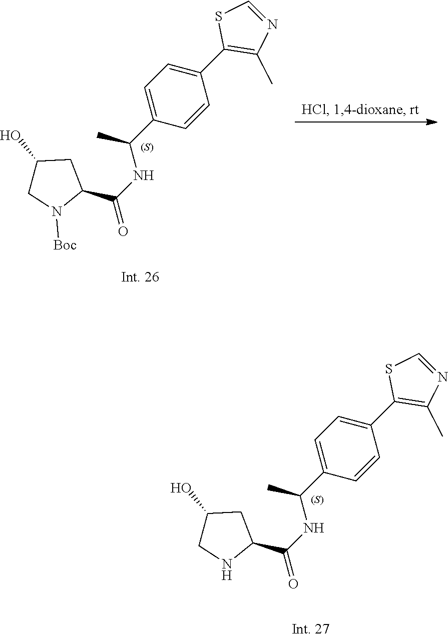

Into a 100 mL round-bottom flask were added tert-butyl (2S,4R)-4-hydroxy-2-{[(1S)-1-[4-(4-methyl-1,3-thiazol-5-yl)phenyl]ethyl]carbamoyl}pyrrolidine-1-carboxylate (2.8 g, 6.488 mmol, 1 equiv), 1,4-dioxane (10 mL) and HCl in 1,4-dioxane (0.79 mL, 4 M). The mixture was stirred for 1 h at 16° C. and was concentrated under reduced pressure. This afforded (2S,4R)-4-hydroxy-N-[(1S)-1-[4-(4-methyl-1,3-thiazol-5-yl)phenyl]ethyl]pyrrolidine-2-carboxamide (2.1 g, 98% yield) as a yellow green solid.

LCMS: (ES, m/z): [M+H]⁺=332 (LCMS condition: Column: Shim-pack Scepter C18-120; Mobile phase A: Water/5 mM NH₄HCO₃; Mobile phase B: Acetonitrile; Flow rate: 1.5 mL/min)

### (f) Synthesis of Int. 28

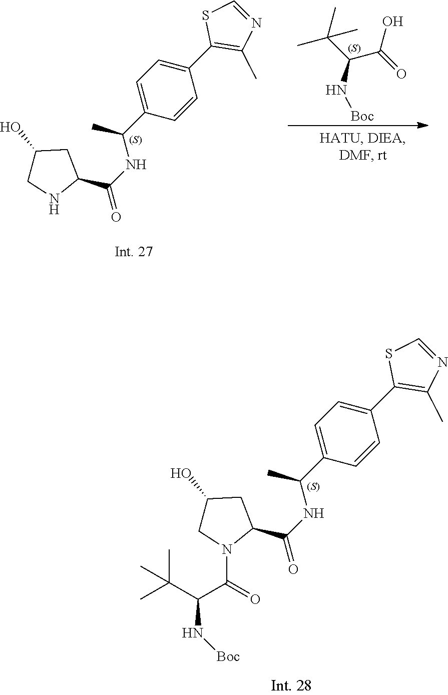

Into a 100 mL round-bottom flask were added (2S,4R)-4-hydroxy-N-[(1S)-1-[4-(4-methyl-1,3-thiazol-5-yl)phenyl]ethyl]pyrrolidine-2-carboxamide (2.5 g, 7.543 mmol, 1.2 equiv), (2S)-2-[(tert-butoxycarbonyl)amino]-3,3-dimethylbutanoic acid (1.45 g, 6.286 mmol, 1 equiv), DMF (25 mL), HATU (3.59 g, 9.429 mmol, 1.5 equiv), and DIEA (2.44 g, 18.858 mmol, 3 equiv) and the mixture stirred for 1 h at 16° C. The reaction was quenched with water at 16° C. The residue was purified by reversed-phase flash chromatography on a C18 column eluting with a 5% to 60% gradient of MeCN in water (10 mmol/L NH₄HCO₃) over 40 min with UV (254 nm) detection. This resulted in tert-butyl N-[(2S)-1-[(2S,4R)-4-hydroxy-2-{[(1S)-1-[4-(4-methyl-1,3-thiazol-5-yl)phenyl]ethyl]carbamoyl}pyrrolidin-1-yl]-3,3-dimethyl-1-oxobutan-2-yl]carbamate (509.4 mg, 15% yield) as a white solid.

LCMS: (ES, m/z): [M+H]⁺=545 (LCMS condition: Column: Shim-pack Scepter C18-120; Mobile phase A: Water/5 mM NH₄HCO₃; Mobile phase B: Acetonitrile; Flow rate: 1.5 mL/min; RT: 0.965 min)

### (g) Synthesis of Int. 29

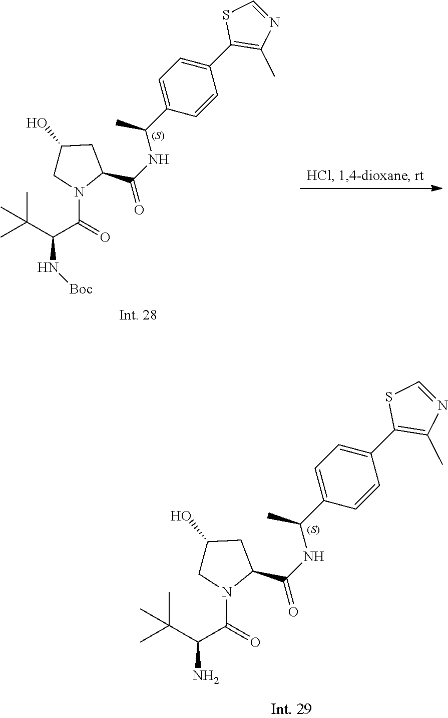

Into a 40 mL vial were added tert-butyl N-[(2S)-1-[(2S,4R)-4-hydroxy-2-{[(15)-1-[4-(4-methyl-1,3-thiazol-5-yl)phenyl]ethyl]carbamoyl}pyrrolidin-1-yl]-3,3-dimethyl-1-oxobutan-2-yl]carbamate (500 mg, 0.918 mmol, 1 equiv), 1,4-dioxane (1 mL) and HCl in 1,4-dioxane (4 mL, 4 M). The mixture was stirred for 1 h at 16° C. and concentrated under reduced pressure. This afforded (2S,4R)-1-[(2S)-2-amino-3,3-dimethylbutanoyl]-4-hydroxy-N-[(1S)-1-[4-(4-methyl-1,3-thiazol-5-yl)phenyl]ethyl]pyrrolidine-2-carboxamide (400 mg, 96% yield) as an off-white solid.

LCMS: (ES, m/z): [M+H]⁺=445 (LCMS condition: Column: HALO 90A C18; Mobile phase A: water/0.1% FA; Mobile phase B: ACN/0.1% FA; Flow rate: 1.2 mL/min; RT: 0.561 min) (h) Synthesis of Int. 30

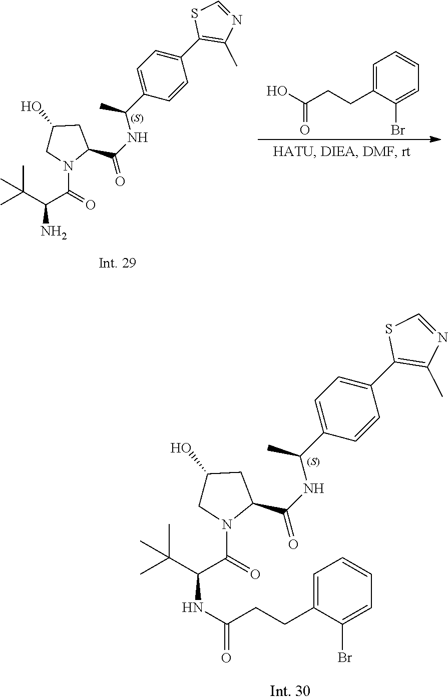

Into a 40 mL vial were added (2S,4R)-1-[(25)-2-amino-3,3-dimethylbutanoyl]-4-hydroxy-N-[(1S)-1-[4-(4-methyl-1,3-thiazol-5-yl)phenyl]ethyl]pyrrolidine-2-carboxamide (400 mg, 0.900 mmol, 1.2 equiv), 3-(2-bromophenyl)propanoic acid (171.75 mg, 0.750 mmol, 1 equiv), DMF (0.5 mL), HATU (427.62 mg, 1.125 mmol, 1.5 equiv) and DIEA (290.71 mg, 2.250 mmol, 3 equiv), and the resulting mixture was stirred for 1 h at 16° C. The reaction was quenched with water at 16° C. and extracted with EtOAc (3×50 mL). The combined organic layers were washed with brine (3×50 mL) and dried over anhydrous Na₂SO₄. After filtration, the filtrate was concentrated under reduced pressure. The residue was purified by silica gel column chromatography eluting with PE/EA (5:1) to afford (2S,4R)-1-[(2S)-2-[3-(2-bromophenyl)propanamido]-3,3-dimethylbutanoyl]-4-hydroxy-N-[(1S)-1-[4-(4-methyl-1,3-thiazol-5-yl)phenyl]ethyl]pyrrolidine-2-carboxamide (490 mg, 98% yield) as an off-white solid.

LCMS: (ES, m/z): [M+H]⁺=655 (LCMS condition: Column: HALO 90A C18; Mobile phase A: water/0.1% FA; Mobile phase B: ACN/0.1% FA; Flow rate: 1.2 mL/min; RT: 0.911 min)

### (i) Synthesis of Int. 31

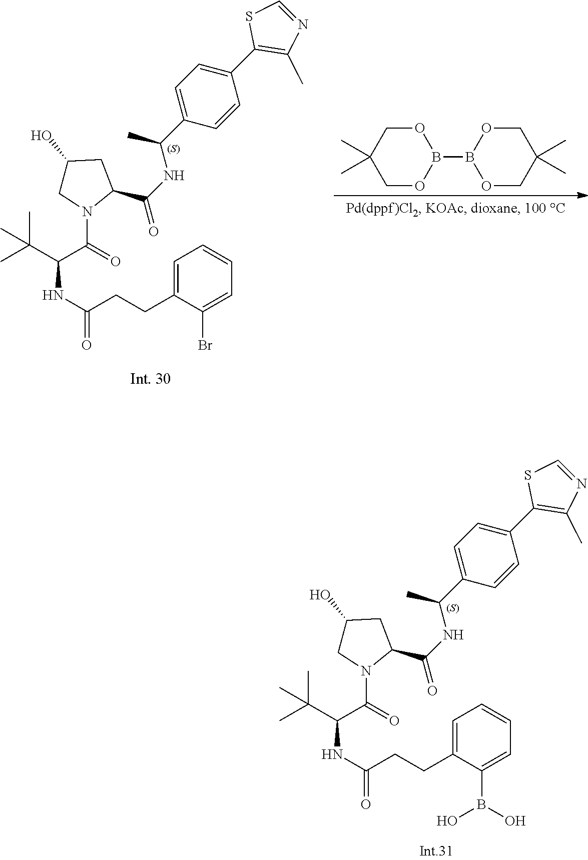

Into a 40 mL vial were added (2S,4R)-1-[(2S)-2-[3-(2-bromophenyl)propanamido]-3,3-dimethylbutanoyl]-4-hydroxy-N-[(1S)-1-[4-(4-methyl-1,3-thiazol-5-yl)phenyl]ethyl]pyrrolidine-2-carboxamide (200 mg, 0.305 mmol, 1 equiv), 2-(5,5-dimethyl-1,3,2-dioxaborinan-2-yl)-5,5-dimethyl-1,3,2-dioxaborinane (137.81 mg, 0.610 mmol, 2 equiv), dioxane (2 mL), KOAc (89.81 mg, 0.915 mmol, 3 equiv), and Pd(dppf)Cl₂ (22.32 mg, 0.030 mmol, 0.1 equiv) at 16° C. The resulting mixture was stirred for 1.5 h at 100° C. under a nitrogen atmosphere. The reaction was quenched with water at 16° C. and was extracted with EtOAc (3×50 mL). The combined organic layers were washed with brine (2×30 mL) and dried over anhydrous Na₂SO₄. After filtration, the filtrate was concentrated under reduced pressure. The crude product (259 mg) was purified by Prep-HPLC with the following conditions (Column: XselectCSH Prep OBD C18 Column, 30×150 mm, 5 μm; Mobile Phase A: water (0.1% FA), Mobile Phase B: ACN; Flow rate: 60 mL/min; Gradient: 28 to 58% B over 8 min; Wave Length: 254 nm/220 nm; RT (min): 6.4) to afford 2-(2-{[(2S)-1-[(2S,4R)-4-hydroxy-2-{[(1S)-1-[4-(4-methyl-1,3-thiazol-5-yl)phenyl]ethyl]carbamoyl}pyrrolidin-1-yl]-3,3-dimethyl-1-oxobutan-2-yl]carbamoyl}ethyl)phenylboronic acid (107.1 mg, 56% yield) as a white solid.

LCMS: (ES, m/z): [M−H]⁻=619 (LCMS condition: Column: HALO 90A C18; Mobile phase A: water/0.1% FA; Mobile phase B: ACN/0.1% FA; Flow rate: 1.2 mL/min; RT: 0.763 min)

### (j) Synthesis of Example 6

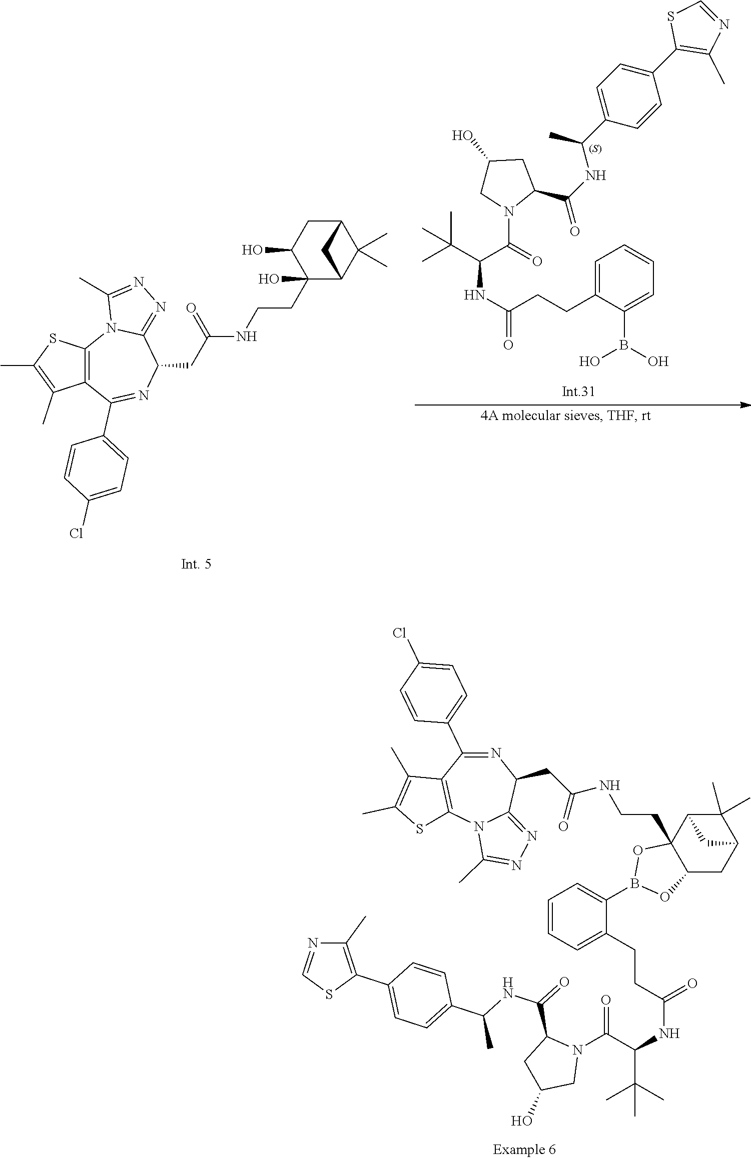

Into a 20 mL vial were added 2-((S)-4-(4-chlorophenyl)-2,3,9-trimethyl-6H-thieno[3,2-f][1,2,4]triazolo[4,3-a][1,4]diazepin-6-yl)-N-(2-((1R,2R,3S,5R)-2,3-dihydroxy-6,6-dimethylbicyclo[3.1.1]heptan-2-yl)ethyl)acetamide(45 mg, 0.077 mmol, 1 equiv), 2-(2-{[(2S)-1-[(2S,4R)-4-hydroxy-2-{[(15)-1-[4-(4-methyl-1,3-thiazol-5-yl)phenyl]ethyl]carbamoyl}pyrrolidin-1-yl]-3,3-dimethyl-1-oxobutan-2-yl]carbamoyl}ethyl)phenylboronic acid (47.97 mg, 0.077 mmol, 1 equiv), 4A molecular sieves (135 mg), and THF (5 mL) and the mixture was stirred for 16 h at r.t. The mixture was filtered, and the filter cake was washed with MeOH (3×20 mL). The filtrate was concentrated under reduced pressure. The crude product (106 mg) was purified by Prep-ACHIRAL-SFC with the following conditions (Column: Viridis BEH Prep 2-EP OBD Column 3×15 cm, 5 μm; Mobile Phase A: CO₂, Mobile Phase B: MeOH (1% 2M NH₃ in MeOH); Flow rate: 65 mL/min; Gradient: isocratic 25% B; Column Temperature (° C.): 35; Back Pressure (bar): 100; Wave Length: 220 nm; RT (min): 4.71; Sample Solvent: MeOH; Injection Volume: 3 mL; Number Of Runs: 4) to afford (2S,4R)-1-((S)-2-(3-(2-((3aR,4R,6R,7aS)-3a-(2-(2-((S)-4-(4-chlorophenyl)-2,3,9-trimethyl-6H-thieno[3,2-f][1,2,4]triazolo[4,3-a][1,4]diazepin-6-yl)acetamido)ethyl)-5,5-dimethylhexahydro-4,6-methanobenzo[d][1,3,2]dioxaborol-2-yl)phenyl)propanamido)-3,3-dimethylbutanoyl)-4-hydroxy-N—((S)-1-(4-(4-methylthiazol-5-yl)phenyl)ethyl)pyrrolidine-2-carboxamide (39.7 mg, 44% yield) as a white solid.

LCMS: (ES, m/z): [M+H]⁺=1166 (LCMS condition: Column: Shim-pack Scepter C18-120; Mobile phase A: Water/5 mM NH₄HCO₃; Mobile phase B: Acetonitrile; Flow rate: 1.5 mL/min; RT: 1.118 min)

### Example 7

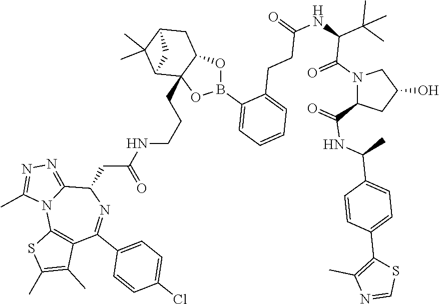 [SMILES: Cc1ncsc1-c1ccc([C@H](C)NC(=O)[C@@H]2C[C@@H](O)CN2C(=O)[C@@H](NC(=O)CCc2ccccc2B2O[C@H]3C[C@H]4C[C@H](C4(C)C)[C@@]3(CCCNC(=O)C[C@@H]3N=C(c4ccc(Cl)cc4)c4c(sc(C)c4C)-n4c(C)nnc43)O2)C(C)(C)C)cc1]

### (a) Synthesis of Int. 29 and 9 as Described in Examples 6 and 2 Respectively

### (b) Synthesis of Example 7

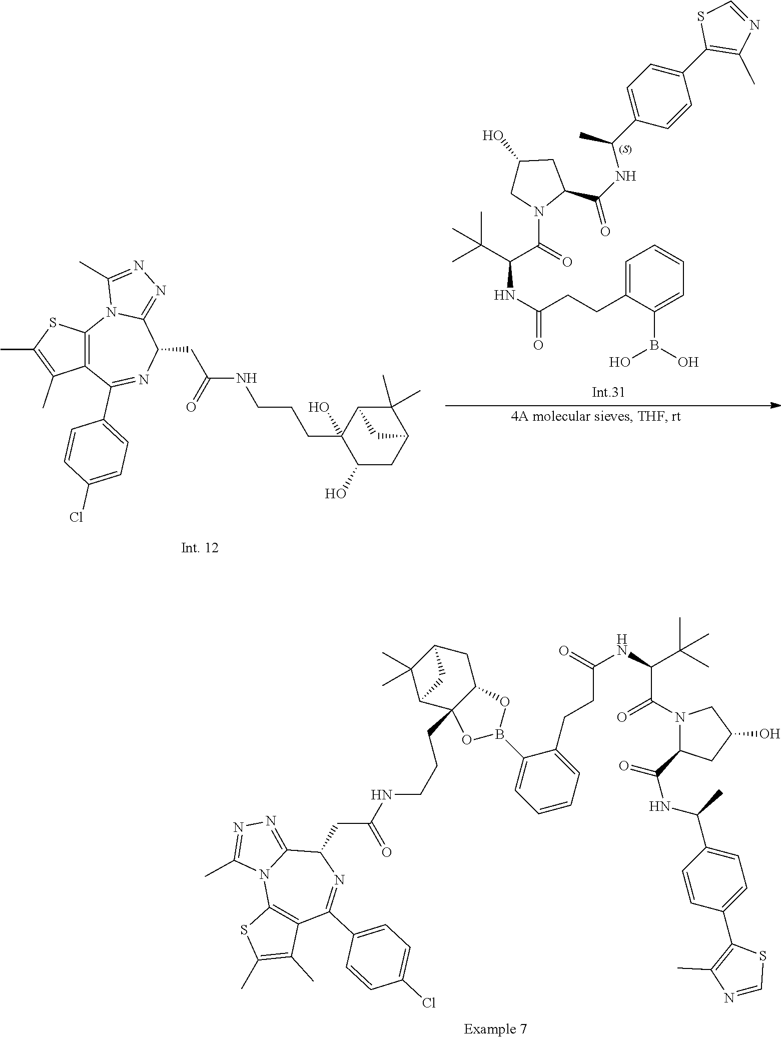

Into an 8 mL vial were added 2-((S)-4-(4-chlorophenyl)-2,3,9-trimethyl-6H-thieno[3,2-f][1,2,4]triazolo[4,3-a][1,4]diazepin-6-yl)-N-(3-((1R,2R,3S,5R)-2,3-dihydroxy-6,6-dimethylbicyclo[3.1.1]heptan-2-yl)propyl)acetamide(50 mg, 0.084 mmol, 1 equiv), 4A molecular sieves (150 mg, 154 mmol, 3 equiv), THF (0.5 mL), and 2-(2-{[(2S)-1-[(2S,4R)-4-hydroxy-2-{[(1S)-1-[4-(4-methyl-1,3-thiazol-5-yl)phenyl]ethyl]carbamoyl}pyrrolidin-1-yl]-3,3-dimethyl-1-oxobutan-2-yl]carbamoyl}ethyl)phenylboronic acid (52.04 mg, 0.084 mmol, 1 equiv) and the mixture was stirred for 16 h at 16° C. The mixture was filtered, and the filter cake was washed with THF (3×25 mL). The filtrate was concentrated under reduced pressure. The residue was purified by Achiral SFC with the following conditions (Column: Viridis BEH Prep 2-EP OBD Column 3×15 cm, 5 μm; Mobile Phase A: CO₂, Mobile Phase B: IPA (1% 2 M NH₃ in MeOH); Flow rate: 65 mL/min; Gradient: isocratic 35% B; Column Temperature (° C.): 35; Back Pressure(bar): 100; Wave Length: 220 nm; RT (min): 4.96; Sample Solvent: EtOH; Injection Volume: 2 mL; Number Of Runs: 4) to afford (2S,4R)-1-((S)-2-(3-(2-((3aR,4R,6R,7aS)-3a-(3-(2-((S)-4-(4-chlorophenyl)-2,3,9-trimethyl-6H-thieno[3,2-f][1,2,4]triazolo[4,3-a][1,4]diazepin-6-yl)acetamido)propyl)-5,5-dimethylhexahydro-4,6-methanobenzo[d][1,3,2]dioxaborol-2-yl)phenyl)propanamido)-3,3-dimethylbutanoyl)-4-hydroxy-N—((S)-1-(4-(4-methylthiazol-5-yl)phenyl)ethyl)pyrrolidine-2-carboxamide (42.8 mg, 42% yield) as a white solid.

LCMS: (ES, m/z): [M+H]⁺=1180 (LCMS condition: Column: Shim-pack Scepter C18-120; Mobile phase A: Water/5 mM NH₄HCO₃; Mobile phase B: Acetonitrile; Flow rate: 1.5 mL/min; RT: 0.984 min)

### Example 8

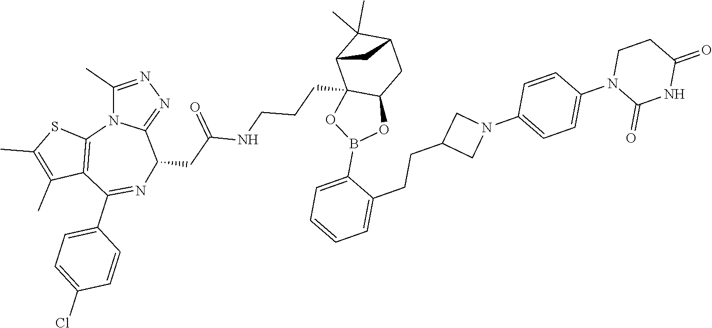 [SMILES: Cc1sc2c(c1C)C(c1ccc(Cl)cc1)=N[C@@H](CC(=O)NCCC[C@@]13OB(c4ccccc4CCC4CN(c5ccc(N6CCC(=O)NC6=O)cc5)C4)O[C@@H]1C[C@@H]1C[C@H]3C1(C)C)c1nnc(C)n1-2]

### (a) Synthesis of Int. 32

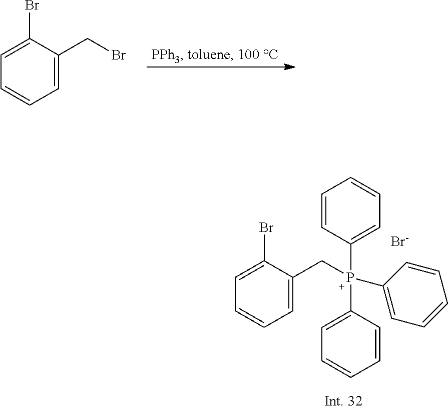

To a stirred solution of 1-bromo-2-(bromomethyl)benzene (10.0 g, 40.011 mmol, 1 eq.) in toluene (100 mL) at r.t. was added triphenylphosphine (11.5 g, 44.012 mmol, 1.1 eq.). The reaction was stirred for 16 h at 100° C. The mixture was filtered through a Celite pad and washed with n-hexane (3×100 mL). The filter cake was collected to afford [(2-bromophenyl)methyl]triphenylphosphanium bromide (20.38 g, yield=99%) as a white solid.

LCMS: (ES, m/z): [M+H]⁺=431 (LCMS condition: Column: HALO 90A C18; Mobile phase A: water (containing 0.05% TFA); Mobile phase B: MeCN (containing 0.05% TFA); Flow rate: 1.5 mL/min.; RT: 0.769 min.)

### (b) Synthesis of Int. 33

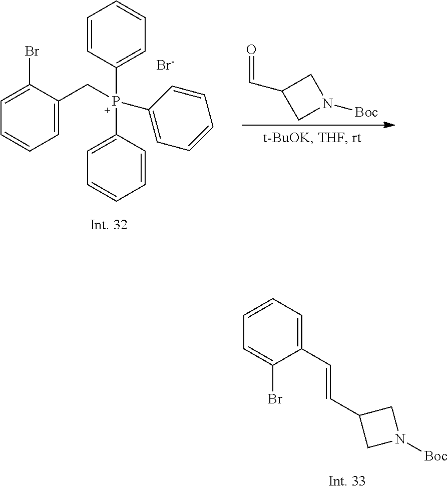

A solution of [(2-bromophenyl)methyl]triphenylphosphanium bromide (16.6 g, 32.393 mmol, 1.2 eq.) in THF (100 mL) under nitrogen was treated with potassium 2-methylpropan-2-olate (4.0 g, 35.112 mmol, 1.30 eq.) for 30 min at 25° C. The mixture was cooled to 0° C. and tert-butyl 3-formylazetidine-1-carboxylate (5.0 g, 26.994 mmol, 1.00 eq.) in THF (25 mL) added dropwise. The mixture was stirred for 16 h at 25° C. under nitrogen. The reaction was quenched with water (100 mL) at 25° C. and the mixture extracted with EtOAc (3×200 mL).

The combined organic extracts were washed with brine (3×50 mL) and dried over anhydrous Na₂SO₄. After filtration, the filtrate was concentrated under reduced pressure and the residue purified by silica gel column chromatography eluting with PE/EtOAc (4:1) to afford tert-butyl (E)-3-(2-bromostyryl)azetidine-1-carboxylate (4.7 g, yield=31%) as a light-yellow oil.

LCMS: (ES, m/z): [M−C₄H₈]⁺=282 (LCMS condition: Column: HALO 90A C18; Mobile phase A: water (containing 0.1% formic acid); Mobile phase B: MeCN (containing 0.1% formic acid); Flow rate: 1.2 mL/min.; RT: 1.205 min.)

### (c) Synthesis of Int. 34

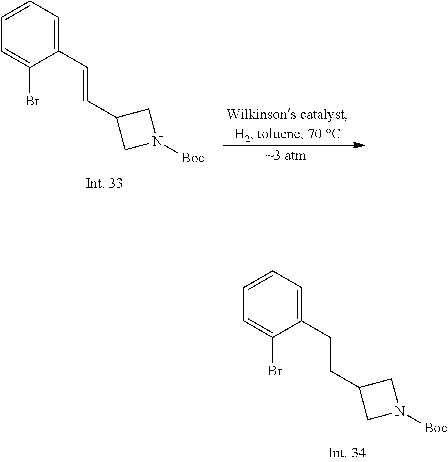 [SMILES: CC(C)(C)OC(=O)N1CC(/C=C/c2ccccc2Br)C1 ; CC(C)(C)OC(=O)N1CC(CCc2ccccc2Br)C1]

To a solution of tert-butyl 3-[(E)-2-(2-bromophenyl)ethenyl]azetidine-1-carboxylate (4.7 g, 13.895 mmol, 1 eq.) in toluene (75 mL) was added Wilkinson's catalyst (1.5 g, 1.667 mmol, 1.2 eq.) in a pressure tank. The mixture was purged with nitrogen for 2 min and pressurized to 3 atm with hydrogen at 70° C. for 16 h. The reaction mixture was cooled to r.t. and filtered. The filtrate was concentrated under reduced pressure and the residue purified by silica gel column chromatography eluting with PE/EtOAc (5:1) to afford tert-butyl 3-[2-(2-bromophenyl)ethyl]azetidine-1-carboxylate (4.37 g, yield=89%) as a yellow oil.

LCMS: (ES, m/z): [M−C₄H₈]⁺=284 (LCMS condition: Column: HALO 90A C18; Mobile phase A: water (containing 0.1% formic acid); Mobile phase B: MeCN (containing 0.1% formic acid); Flow rate: 1.2 mL/min.; RT: 1.206 min.)

### (d) Synthesis of Int. 35

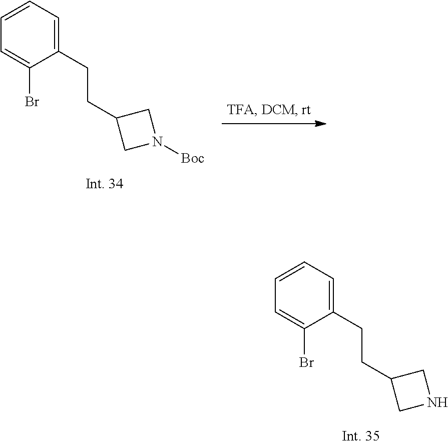 [SMILES: CC(C)(C)OC(=O)N1CC(CCc2ccccc2Br)C1 ; Brc1ccccc1CCC1CNC1]

Into a 50 mL round-bottom flask were added tert-butyl 3-[2-(2-bromophenyl)ethyl]azetidine-1-carboxylate (2.0 g, 5.878 mmol, 1 eq.), DCM (20 mL) and TFA (5 mL) at 25° C. The reaction was stirred for 30 min at 25° C. The mixture was concentrated under reduced pressure and diluted with water (20 mL). The mixture was neutralized to pH 8 with saturated NaHCO₃ (aq.) and extracted with DCM (3×20 mL). The organic extracts were combined and concentrated under reduced pressure to afford 3-(2-bromophenethyl)azetidine (1.1 g, yield=78%) as a yellow oil.

LCMS: (ES, m/z): [M+H]⁺=240 (LCMS condition: Column: HALO 90A C18; Mobile phase A: water (containing 0.1% formic acid); Mobile phase B: MeCN (containing 0.1% formic acid); Flow rate: 1.2 mL/min.; RT: 0.523 min.)

### (e) Synthesis of Int. 36

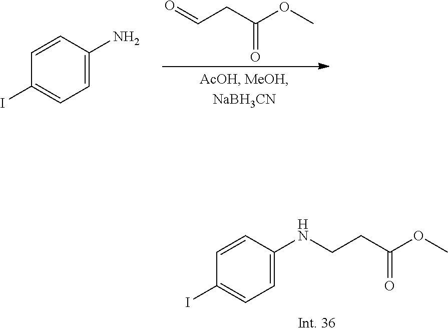

Into a 500 mL 3-necked round-bottom flask were added 4-iodoaniline (14.3 g, 65.289 mmol, 1 eq.), MeOH (143 mL), methyl 3-oxopropanoate (20.0 g, 195.867 mmol, 3 eq.) and AcOH (11.76 g, 195.867 mmol, 3 eq.) at 25° C. The reaction was stirred for 1 h at 60° C. under nitrogen. The mixture was cooled to 25° C. and NaBH₃CN (12.31 g, 195.867 mmol, 3 eq.) added in portions at under nitrogen. The reaction was stirred for 16 h at 60° C. under nitrogen. The reaction was cooled to 25° C., quenched with water and extracted with EtOAc (3×500 mL). The combined organic extracts were washed with brine (3×100 mL) and dried over anhydrous Na₂SO₄. After filtration, the filtrate was concentrated under reduced pressure and the residue purified by reversed-phase flash chromatography on a C18 column using a 40 to 50% gradient of MeCN in water (containing 10 mmol/L NH₄HCO₃) over 10 min to afford methyl 3-[(4-iodophenyl)amino]propanoate (15.0 g, yield=73%) as a yellow solid. LCMS: (ES, m/z): [M+H]⁺=306 (LCMS condition: Column: Kinetex EVO C18; Mobile phase A: water (containing 6.5 mM NH₄HCO₃ and ammonia hydroxide (pH=10)); Mobile phase B: MeCN; Flow rate: 1.5 mL/min; RT: 0.851 min)

### (f) Synthesis of Int. 37

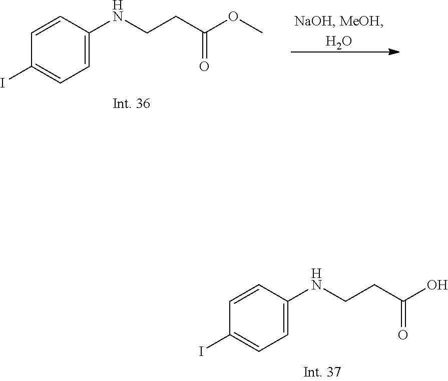

Into a 500 mL round-bottom flask were added methyl 3-[(4-iodophenyl)amino]propanoate (15.0 g, 49.162 mmol, 1 eq.), MeOH (120 mL), water (40 mL), and LiOH (11.8 g, 491.620 mmol, 10 eq.). The reaction was stirred for 16 h at 25° C. The mixture was diluted with water and concentrated under reduced pressure. The residue was acidified to pH 3 with 2 N HCl (aq.) and extracted with EtOAc (3×200 mL). The combined organic extracts were washed with brine (3×200 mL) and dried over anhydrous Na₂SO₄. After filtration, the filtrate was concentrated under reduced pressure to afford 3-[(4-iodophenyl)amino]propanoic acid (10.6 g, yield=72%) as a yellow solid.

LCMS: (ES, m/z): [M+H]⁺=292 (LCMS condition: Column: HALO 90A C18; Mobile phase A: water (containing 0.1% formic acid); Mobile phase B: MeCN (containing 0.1% formic acid); Flow rate: 1.2 mL/min.; RT: 1.355 min.)

### (g) Synthesis of Int. 38

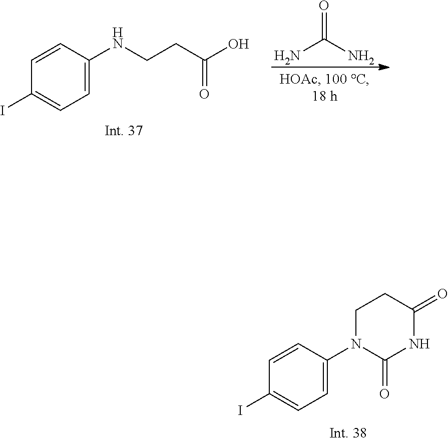

Into a 250 mL round-bottom flask were added 3-[(4-iodophenyl)amino]propanoic acid (10.6 g, 36.415 mmol, 1 eq.), AcOH (106 mL, 1849.863 mmol, 50.80 eq.) and urea (6.6 g, 109.231 mmol, 3.00 eq.) at 25° C. The reaction was stirred for 18 h at 100° C. The mixture was concentrated under vacuum and the residue purified by reversed-phase flash chromatography on a CIS column using a 40 to 50% gradient of MeCN in water (containing 0.1%/a formic acid) over 10 min to afford 1-(4-iodophenyl)dihydropyrimidine-2,4(1H,3H)-dione (1.5 g, yield=12%) as a brown solid.

LCMS: (ES, m/z): [M+H]⁺=317 (LCMS condition: Column: HALO 90A C18; Mobile phase A: water (containing 0.1% formic acid); Mobile phase B: MeCN (containing 0.1% formic acid); Flow rate: 1.2 mL/min.; RT: 1.209 min.)

### (h) Synthesis of Int. 39

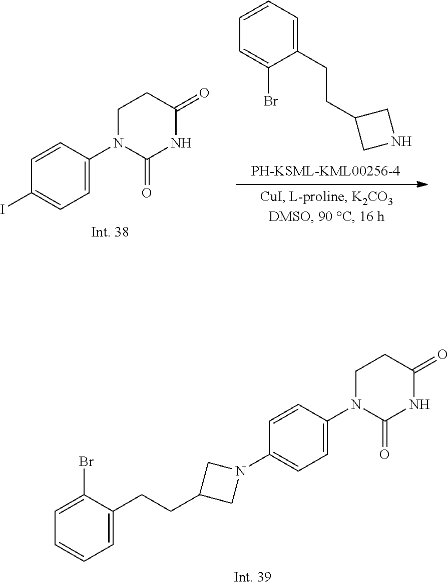 [SMILES: O=C1CCN(c2ccc(I)cc2)C(=O)N1 ; Brc1ccccc1CCC1CNC1 ; O=C1CCN(c2ccc(N3CC(CCc4ccccc4Br)C3)cc2)C(=O)N1]

Into a 40 mL vial were added 1-(4-iodophenyl)dihydropyrimidine-2,4(1H,3H)-dione (930 mg, 2.942 mmol, 1 eq.), DMSO (10 mL), 3-[2-(2-bromophenyl)ethyl]azetidine (706 mg, 2.942 mmol, 1 eq.), CuI (280 mg, 1.471 mmol, 0.5 eq.), K₂CO₃ (2033 mg, 14.710 mmol, 5 eq.) and L-proline (169.37 mg, 1.471 mmol, 0.5 eq.) at 25° C. The reaction was stirred for 16 h at 90° C. under nitrogen. The mixture was evaporated, and the residue purified by reversed-phase flash chromatography on a C18 column using a 40 to 60% gradient of MeCN in water (containing 0.1% formic acid) over 10 min to afford 1-(4-(3-(2-bromophenethyl)azetidin-1-yl)phenyl)dihydropyrimidine-2,4(1H,3H)-dione (300 mg, yield=15%) as a brown solid.

LCMS: (ES, m/z): [M+H]⁺=428 (LCMS condition: Column: HALO 90A C18; Mobile phase A: water (containing 0.1% formic acid); Mobile phase B: MeCN (containing 0.1% formic acid); Flow rate: 1.2 mL/min; RT: 1.228 min)

### (i) Synthesis of Int. 40

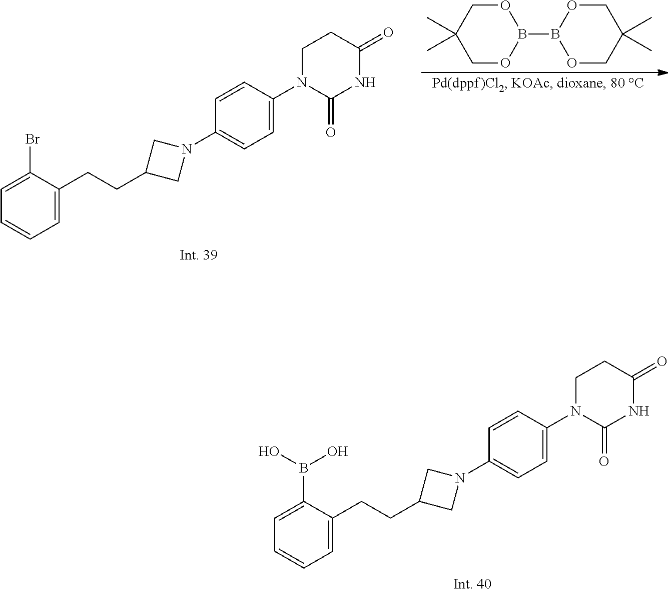

Into a 40 mL vial were added dioxane (12 mL), KOAc (402 mg, 4.098 mmol, 3 eq.), 1-(4-(3-(2-bromophenethyl)azetidin-1-yl)phenyl)dihydropyrimidine-2,4(1H,3H)-dione (585 mg, 1.366 mmol, 1 eq.), 2-(5,5-dimethyl-1,3,2-dioxaborinan-2-yl)-5,5-dimethyl-1,3,2-dioxaborinane (926 mg, 4.098 mmol, 3 eq.), and Pd(dppf)Cl₂ (100 mg, 0.137 mmol, 0.1 eq.) at 25° C. The resulting mixture was stirred for 2 h at 80° C. under nitrogen. The mixture was allowed to cool to r.t. and evaporated. The residue was purified by reversed-phase flash chromatography on a C13 column using a 20 to 40% gradient of MeCN in water (containing 0.1% formic acid) over 10 min. The crude product was purified by preparative HPLC (Column: Xselect CSH Prep OBD C18, 30×150 mm, 5 μm; Mobile Phase A: water (containing 0.1% formic acid), Mobile Phase B: MeCN; Flow rate: 60 mL/min; Gradient: 16 to 37% B over 11 min; Wave Length: 254 and 220 nm; RT (min): 11.4) to afford (2-(2-(1-(4-(2,4-dioxotetrahydropyrimidin-1(2H)-yl)phenyl)azetidin-3-yl)ethyl)phenyl)boronic acid (95.8 mg, yield=18%) as a white solid.

LCMS: (ES, m/z): [M+H]⁺=394 (LCMS condition: Column: HALO 90A C18; Mobile phase A: water (containing 0.1% formic acid); Mobile phase B: MeCN (containing 0.1% formic acid); Flow rate: 1.2 mL/min.; RT: 0.719 min.)

### (j) Synthesis of Int. 41

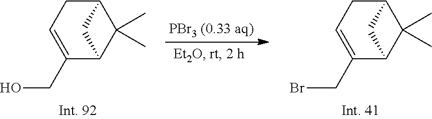

To a stirred solution of [(1S,5R)-6,6-dimethylbicyclo[3.1.1]hept-2-en-2-yl]methanol (20.0 g, 131.374 mmol, 1 eq.) in diethyl ether (200 mL) was added phosphorus tribromide (11.7 g, 43.353 mmol, 0.33 eq.). The reaction was stirred for 2 h at r.t. The mixture was quenched with water (200 mL) and extracted with EtOAc (3×200 mL). The organic extracts were combined, washed with brine (2×200 mL) and dried over Na₂SO₄. After filtration, the filtrate was concentrated under reduced pressure to afford crude (1S,5R)-2-(bromomethyl)-6,6-dimethylbicyclo[3.1.1]hept-2-ene (18.0 g) as a yellow oil which was used in the next step without purification.

### (k) Synthesis of Int. 42

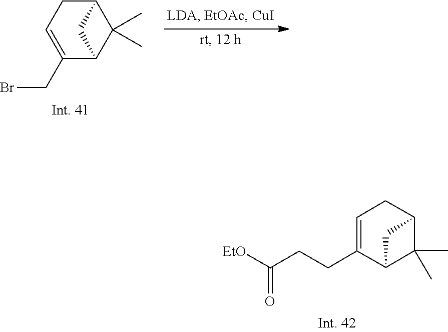 [SMILES: CC1(C)[C@@H]2CC=C(CBr)[C@H]1C2 ; CCOC(=O)CCC1=CC[C@@H]2C[C@H]1C2(C)C]

To a mixture of copper (I) iodide (63.7 g, 334.676 mmol, 4 eq.) and EtOAc (14.7 g, 167.338 mmol, 2 eq.) in THF (180 mL) was added LDA (19.7 g, 184.072 mmol, 2.2 eq.) at −78° C. under nitrogen and the reaction stirred for 1 h at −78° C. (1S,5R)-2-(Bromomethyl)-6,6-dimethylbicyclo[3.1.1]hept-2-ene (18.0 g, 83.669 mmol, 1 eq.) was added and the reaction stirred overnight at r.t. under nitrogen. The mixture was quenched with saturated NH₄Cl (aq.) (150 mL) and extracted with EtOAc (3×200 mL). The organic extracts were combined, washed with brine (2×200 mL) and dried over Na₂SO₄. After filtration, the filtrate was concentrated under reduced pressure and the residue purified by silica gel column chromatography eluting with PE/EtOAc (95:5) to afford ethyl 3-[(1S,5R)-6,6-dimethylbicyclo[3.1.1]hept-2-en-2-yl]propanoate (7.7 g, yield=41%) as a yellow oil.

LCMS: (ES, m/z): [M+H]⁺=223 (LCMS condition: Column: HALO 90A C18; Mobile phase A: water (containing 0.1% formic acid); Mobile phase B: MeCN (containing 0.1% formic acid); Flow rate: 1.2 mL/min.; RT: 1.080 min.)

### (l) Synthesis of Int. 43

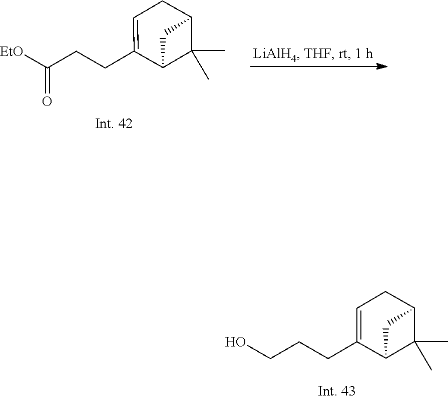 [SMILES: CCOC(=O)CCC1=CC[C@@H]2C[C@H]1C2(C)C ; CC1(C)[C@@H]2CC=C(CCCO)[C@H]1C2]

To a mixture of ethyl 3-[(1S,5R)-6,6-dimethylbicyclo[3.1.1]hept-2-en-2-yl]propanoate (7.7 g, 34.791 mmol, 1 eq.) in THF (80 mL) was added LiAlH₄ (105 mL, 208.746 mmol, 6 eq., 2M, in THF) at 0° C. The reaction was stirred for 2 h at r.t. The mixture was quenched with water (200 mL) and extracted with EtOAc (3×500 mL). The organic extracts were combined, washed with brine (2×100 mL) and dried over Na₂SO₄. After filtration, the filtrate was concentrated under reduced pressure to afford 3-[(1S,5R)-6,6-dimethylbicyclo[3.1.1]hept-2-en-2-yl]propan-1-ol (4.5 g, yield=33%) as a yellow oil.

LCMS: (ES, m/z): [M+CH₃CN+H]⁺=222 (LCMS condition: Column: Kinetex EVO C18; Mobile phase A: water (containing 6.5 mM NH₄HCO₃ and ammonia hydroxide (pH=10)); Mobile phase B: MeCN; Flow rate: 1.5 mL/min.; RT: 0.895 min.)

### (m) Synthesis of Int. 44

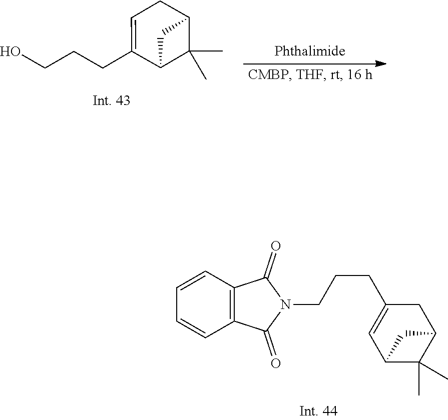 [SMILES: CC1(C)[C@@H]2CC=C(CCCO)[C@H]1C2 ; CC1(C)[C@@H]2CC(CCCN3C(=O)c4ccccc4C3=O)=C[C@H]1C2]

Into a 100 mL 3-necked round-bottom flask were added 3-[(1S,5R)-6,6-dimethylbicyclo[3.1.1]hept-2-en-2-yl]propan-1-ol (4.0 g, 22.186 mmol, 1 eq.), THF (40 mL), 2,3-dihydro-1H-isoindole-1,3-dione (6.5 g, 44.372 mmol, 2 eq.) and CMBP (11.2 g, 46.591 mmol, 2.1 eq.) at 20° C. The reaction was stirred for 4 h under nitrogen. The mixture was quenched with water and extracted with EtOAc (3×200 mL). The organic extracts were combined and concentrated under reduced pressure. The residue was purified by silica gel column chromatography eluting with PE/EtOAc (5:1) to afford 2-(3-((1S,5R)-6,6-dimethylbicyclo[3.1.1]hept-2-en-2-yl)propyl)isoindoline-1,3-dione(2.0 g, yield=20%) as a yellow oil.

LCMS: (ES, m/z): [M+H]⁺=310 (LCMS condition: Column: HALO 90A C18; Mobile phase A: water (containing 0.1% formic acid); Mobile phase B: MeCN (containing 0.1% formic acid); Flow rate: 1.2 mL/min.; RT: 1.016 min.

### (n) Synthesis of Int. 45

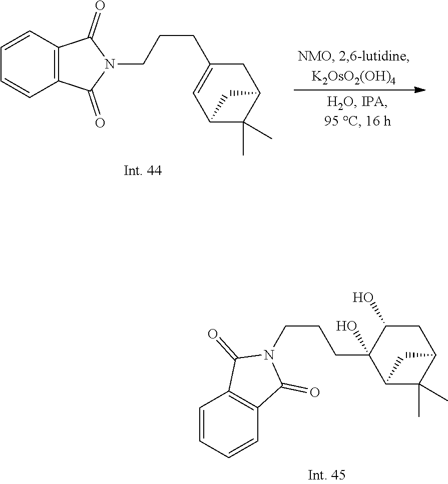 [SMILES: CC1(C)[C@@H]2CC(CCCN3C(=O)c4ccccc4C3=O)=C[C@H]1C2 ; CC1(C)[C@@H]2C[C@@H](O)[C@](O)(CCCN3C(=O)c4ccccc4C3=O)[C@H]1C2]

Into a 100 mL 3-necked round-bottom flask were added 2-(3-((1S,5R)-6,6-dimethylbicyclo[3.1.1]hept-2-en-2-yl)propyl)isoindoline-1,3-dione(2.0 g, 6.464 mmol, 1 eq.), IPA (15 mL), water (3 mL), NMO (2.0 g, 16.806 mmol, 2.6 eq.), 2,6-lutidine (970 mg, 9.050 mmol, 1.40 eq.) and K₂OsO₂(OH)₄ (476 mg, 1.293 mmol, 0.2 eq.) at 20° C. The reaction was stirred for 16 h at 95° C. under nitrogen. The mixture was quenched with water (30 mL) at 20° C. and extracted with EtOAc (3×100 mL). The organic extracts were combined and concentrated under reduced pressure. The residue was purified by silica gel column chromatography eluting with PE/THF (10:1) to afford 2-{3-[(1S,2S,3R,5S)-2,3-dihydroxy-6,6-dimethylbicyclo[3.1.1]heptan-2-yl]propyl}isoindole-1,3-dione (1.0 g, yield=43%) as a light brown oil.

LCMS: (ES, m/z): [M+Na]⁺=366 (LCMS condition: Column: HALO 90A C18; Mobile phase A: water (containing 0.1% formic acid); Mobile phase B: MeCN (containing 0.1% formic acid); Flow rate: 1.2 mL/min.; RT: 0.890 min.)

### (o) Synthesis of Int. 46

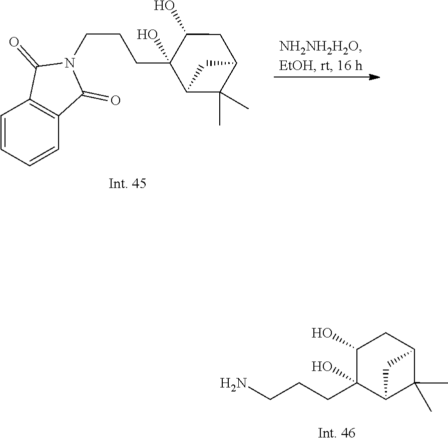 [SMILES: CC1(C)[C@@H]2C[C@@H](O)[C@](O)(CCCN3C(=O)c4ccccc4C3=O)[C@H]1C2 ; CC1(C)[C@@H]2C[C@@H](O)[C@](O)(CCCN)[C@H]1C2]

Into a 50 mL round-bottom flask were added 2-{3-[(1S,2S,3R,5S)-2,3-dihydroxy-6,6-dimethylbicyclo[3.1.1]heptan-2-yl]propyl}isoindole-1,3-dione (700 mg, 2.038 mmol, 1 eq.), NH₂NH₂·H₂O (163.26 mg, 3.261 mmol, 1.6 eq.) and EtOH (20 mL) at r.t. The reaction was stirred for 16 h at r.t. The mixture was concentrated under reduced pressure to afford crude (1S,2S,3R,5S)-2-(3-aminopropyl)-6,6-dimethylbicyclo[3.1.1]heptane-2,3-diol (860 mg) as a yellow solid which was used in the next step without purification.

LCMS: (ES, m/z): [M+H]⁺=214 (LCMS condition: Column: HALO 90A C18; Mobile phase A: water (containing 0.1% formic acid); Mobile phase B: MeCN (containing 0.1% formic acid); Flow rate: 1.2 mL/min.; RT: 0.468 min.)

### (p) Synthesis of Int. 47

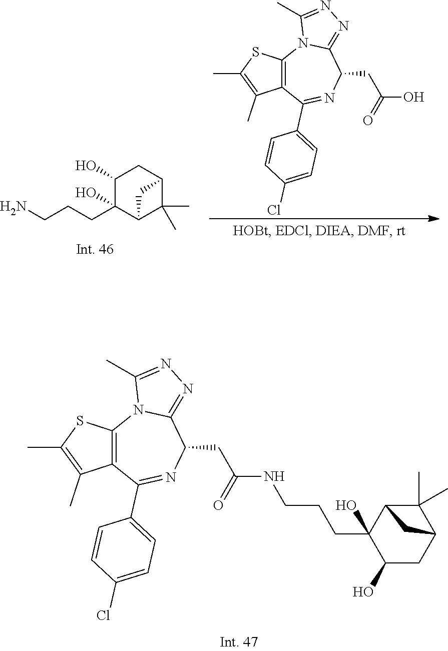

To a solution of (S)-2-(4-(4-chlorophenyl)-2,3,9-trimethyl-6H-thieno[3,2-f][1,2,4]triazolo[4,3-a][1,4]diazepin-6-yl)acetic acid (200 mg, 0.499 mmol, 1 eq.) in DMF (3 mL) was added EDCI (124 mg, 0.649 mmol, 1.3 eq.), HOBT (88 mg, 0.649 mmol, 1.3 eq.) and DIEA (193 mg, 1.497 mmol, 3 eq.) and the mixture stirred for 30 min at r.t. (1S,2S,3R,5S)-2-(3-Aminopropyl)-6,6-dimethylbicyclo[3.1.1]heptane-2,3-diol (213 mg, 0.998 mmol, 2 eq.) was added and the reaction stirred for 1 h at r.t. The mixture was quenched with water (20 mL) and extracted with EtOAc (3×50 mL). The organic extracts were combined, washed with brine (2×20 mL) and dried over Na₂SO₄. After filtration, the filtrate was concentrated under reduced pressure and purified by preparative HPLC (Column: Xselect CSH Prep C18 Column, 30×150 mm, 5 μm; Mobile Phase A: water (containing 0.1% formic acid), Mobile Phase B: MeCN; Flow rate: 60 mL/min; Gradient: 42 to 63% B over 10 min; Wavelength: 254 and 220 nm; RT (min): 8.68) to afford the crude product. The crude product was separated by preparative chiral SFC (Column: CHIRAL ART Cellulose-SZ 3×25 cm, 5 μm; Mobile Phase A: CO₂, Mobile Phase B: MeOH (containing 1% of a 2 M solution of NH₃ in MeOH); Flow rate: 100 mL/min; Gradient: isocratic 48% B; Column Temperature (° C.): 35; Back Pressure (bar): 100; Wave Length: 220 nm; RT peak 1 (min): 8.03; RT peak 2 (min): 11.33; Sample Solvent: MeOH; Injection Volume: 1 mL) to afford 2-((S)-4-(4-chlorophenyl)-2,3,9-trimethyl-6H-thieno[3,2-f][1,2,4]triazolo[4,3-a][1,4]diazepin-6-yl)-N-(3-((1S,2S,3R,5S)-2,3-dihydroxy-6,6-dimethylbicyclo[3.1.1]heptan-2-yl)propyl)acetamide (92.0 mg, yield=31%) as a white solid.

LCMS: (ES, m/z): [M+H]⁺=596 (LCMS condition: Column: HALO 90A C18; Mobile phase A: water (containing 0.1% formic acid); Mobile phase B: MeCN (containing 0.1% formic acid) Flow rate: 1.2 mL/min; RT: 0.962 min)

### (q) Synthesis of Example 8

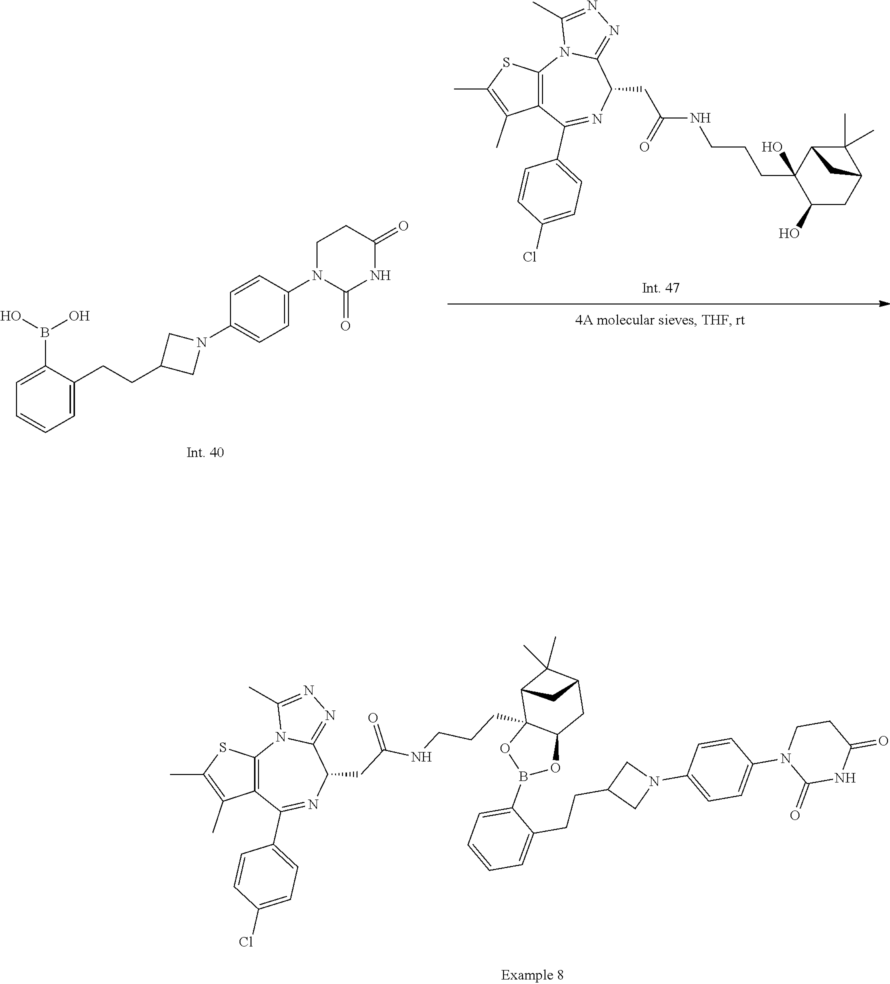

A solution of 2-(2-{1-[4-(2,4-dioxo-1,3-diazinan-1-yl)phenyl]azetidin-3-yl}ethyl)phenylboronic acid (37 mg, 0.094 mmol, 1 eq.), molecular sieves (4 Å) (200 mg), and 2-((S)-4-(4-chlorophenyl)-2,3,9-trimethyl-6H-thieno[3,2-f][1,2,4]triazolo[4,3-a][1,4]diazepin-6-yl)-N-(3-((1S,2S,3R,5S)-2,3-dihydroxy-6,6-dimethylbicyclo[3.1.1]heptan-2-yl)propyl)acetamide (56 mg, 0.094 mmol, 1 eq.) in THF (5 mL) was stirred for 16 h at r.t. under nitrogen. The mixture was filtered, and the filter cake washed with THF (3×5 mL). The filtrate was concentrated under reduced pressure and the residue purified by preparative SFC (Column: Torus Diol OBD 3×25 cm, 5 μm; Mobile Phase A: CO₂, Mobile Phase B: IPA; Flow rate: 75 mL/min; Gradient: isocratic 25% B; Column Temperature (° C.): 35; Back Pressure (bar): 100; Wave Length: 220 nm; RT (min): 5.75; Sample Solvent: MeCN; Injection Volume: 2 mL; Number of runs: 3) to afford 2-((S)-4-(4-chlorophenyl)-2,3,9-trimethyl-6H-thieno[3,2-f][1,2,4]triazolo[4,3-a][1,4]diazepin-6-yl)-N-(3-((3a5,4S,6S,7aR)-2-(2-(2-(1-(4-(2,4-dioxotetrahydropyrimidin-1(2H)-yl)phenyl)azetidin-3-yl)ethyl)phenyl)-5,5-dimethyltetrahydro-4,6-methanobenzo[d][1,3,2]dioxaborol-3a(4H)-yl)propyl)acetamide (56.0 mg, yield=62%) as a white solid.

LCMS: (ES, m/z): [M+H]⁺=953 (LCMS condition: Column: Shim-pack Scepter C18-120; Mobile phase A: Water/MeCN (95:5, v:v) (containing 5 mM NH₄HCO₃); Mobile phase B: MeCN; Flow rate: 1.5 mL/min.; RT: 0.980 min.)

### Example 9

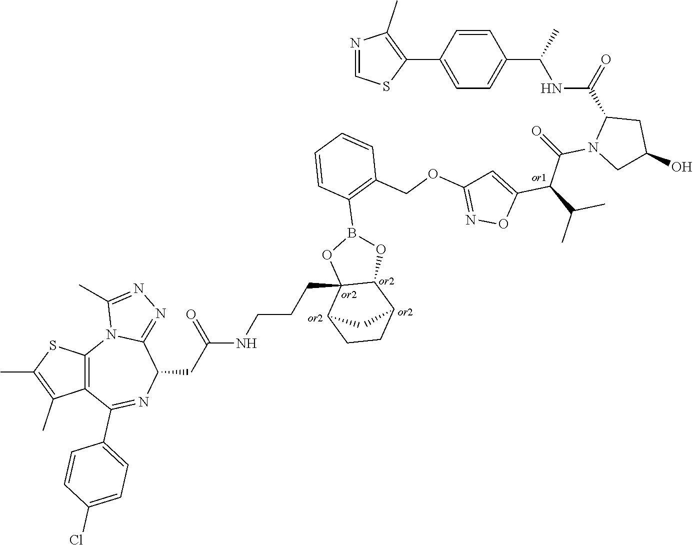 [SMILES: Cc1ncsc1-c1ccc([C@H](C)NC(=O)[C@@H]2C[C@@H](O)CN2C(=O)[C@H](c2cc(OCc3ccccc3B3O[C@@H]4[C@H]5CC[C@H](C5)[C@]4(CCCNC(=O)C[C@@H]4N=C(c5ccc(Cl)cc5)c5c(sc(C)c5C)-n5c(C)nnc54)O3)no2)C(C)C)cc1]

### (a) Synthesis of Int. 48

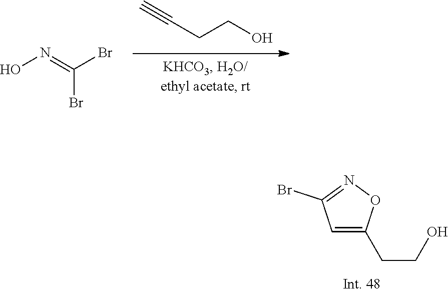

To a stirred solution of but-3-yn-1-ol (28.0 g, 399.342 mmol, 4.05 eq.) and KHCO₃ (30.0 g, 299.753 mmol, 3.04 eq.) in EtOAc (200 mL) and water (20 mL) was added a solution of 1-bromo-N-hydroxymethanecarbonimidoyl bromide (20.0 g, 98.603 mmol, 1 eq.) in EtOAc (50 mL) dropwise at r.t. The reaction was stirred for 16 h at r.t. The mixture was poured into water (300 mL) and extracted with EtOAc (3×500 mL). The combined organic extracts were washed with brine (3×100 mL) and dried over anhydrous Na₂SO₄. After filtration, the filtrate was concentrated under reduced pressure and the residue purified by silica gel column chromatography eluting with PE/EtOAc (4:1) to afford 2-(3-bromoisoxazol-5-yl)ethan-1-ol (14.3 g, yield=75%) as a yellow oil.

LCMS: (ES, m/z): [M+H]⁺=192 (LCMS condition: Column: HALO 90A C18; Mobile phase A: water (containing 0.1% formic acid); Mobile phase B: MeCN (containing 0.1% formic acid) Flow rate: 1.2 mL/min; RT: 0.521 min)

### (b) Synthesis of Int. 49

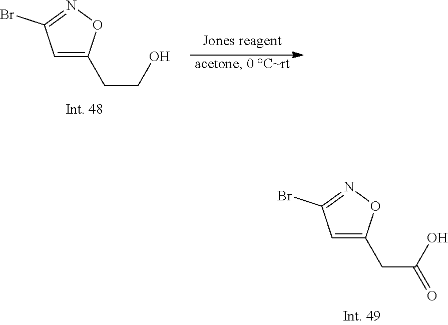 [SMILES: OCCc1cc(Br)no1 ; O=C(O)Cc1cc(Br)no1]

To a stirred solution of 2-(3-bromoisoxazol-5-yl)ethan-1-ol (14.3 g, 74.475 mmol, 1 eq.) in acetone (185 mL, 220.559 mmol) was added Jones reagent (70 mL, 455.787 mmol, 6.12 eq.) dropwise at 0° C. The reaction was stirred for 16 h at r.t. The mixture was poured into water (200 mL) and extracted with EtOAc (3×400 mL). The organic extracts were combined and dried over anhydrous Na₂SO₄. After filtration, the filtrate was concentrated under reduced pressure and the residue purified by reversed-phase flash chromatography on a C18 column using a 0 to 100% gradient of MeCN in water (containing 0.1% formic acid) over 40 min to afford 2-(3-bromoisoxazol-5-yl)acetic acid (15.1 g, yield=92%) as a light yellow solid.

LCMS: (ES, m/z): [M+H]⁺=206 (LCMS condition: Column: HALO 90A C18; Mobile phase A: water (containing 0.1% formic acid); Mobile phase B: MeCN (containing 0.1% formic acid); Flow rate: 1.2 mL/min.; RT: 0.707 min.)

### (c) Synthesis of Int. 50

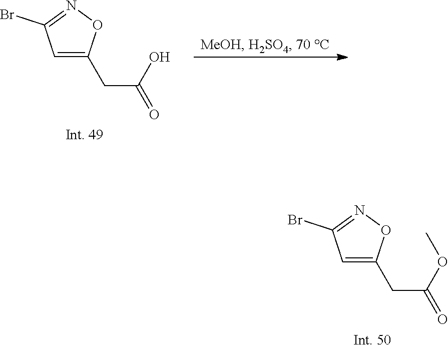

A solution of 2-(3-bromoisoxazol-5-yl)acetic acid (14.0 g, 67.963 mmol, 1 eq.) and H₂SO₄ (7.9 mL, 148.221 mmol) in MeOH (145.0 mL, 3581.331 mmol) was stirred for 2 h at 70° C.

After cooling to r.t., the mixture was concentrated under reduced pressure. The mixture was diluted with water (100 mL) and extracted with EtOAc (3×200 mL). The organic extracts were combined and dried over anhydrous Na₂SO₄. After filtration, the filtrate was concentrated under reduced pressure and the residue purified by reversed-phase flash chromatography on a C18 column using a 10 to 50% gradient of MeCN in water (containing 0.1% formic acid) over 40 min to afford methyl 2-(3-bromoisoxazol-5-yl)acetate (9.3 g, yield=60%) as a white solid.

LCMS: (ES, m/z): [M+H]⁺=220 (LCMS condition: Column: HALO 90A C18; Mobile phase A: water (containing 0.05% TFA); Mobile phase B: MeCN (containing 0.05% TFA); Flow rate: 1.5 mL/min.; RT: 1.008 min.)

### (d) Synthesis of Int. 51

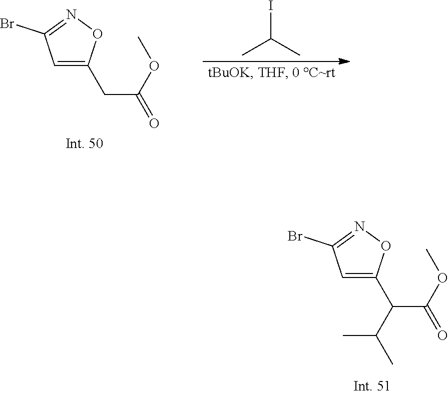

To a stirred mixture of methyl 2-(3-bromoisoxazol-5-yl)acetate (9.3 g, 42.269 mmol, 1 eq.) and t-BuOK (7.1 g, 63.403 mmol, 1.5 eq.) in THF was added 2-iodopropane (9.3 g, 54.950 mmol, 1.30 eq.) dropwise at 0° C. The reaction was stirred for 16 h at r.t. The mixture was concentrated under reduced pressure, diluted with water (200 mL) and extracted with EtOAc (3×500 mL). The organic extracts layers were combined, washed with brine (3×100 mL) and dried over anhydrous Na₂SO₄. After filtration, the filtrate was concentrated under reduced pressure and the residue purified by reversed-phase flash chromatography on a C18 column using a 0 to 100% gradient of MeCN in water (containing 0.1% formic acid) over 40 min to afford methyl 2-(3-bromo-1,2-oxazol-5-yl)-3-methylbutanoate (racemic) (5.0 g, yield=42%) as a yellow oil.

LCMS: (ES, m/z): [M+H]⁺=262 (LCIS condition: Column: HALO 90A C18; Mobile phase A: water (containing 0.1% formic acid); Mobile phase B: MeCN (containing 0.1% formic acid); Flow rate: 1.2 mL/min.; RT: 0.954 min.)

### (e) Synthesis of Int. 52

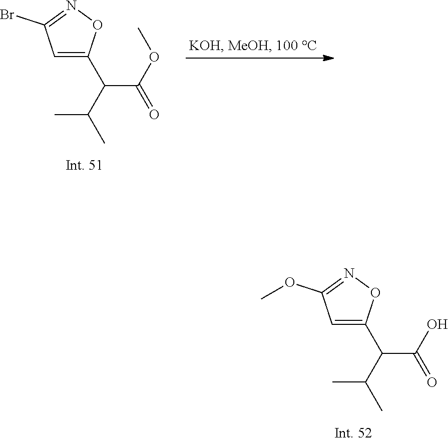

A mixture of methyl 2-(3-bromo-1,2-oxazol-5-yl)-3-methylbutanoate (racemic) (5.0 g, 19.076 mmol, 1 eq.) and KOH (10.7 g, 190.760 mmol, 10 eq.) in MeOH (50 mL) was stirred for 16 h at 100° C. After cooling to r.t., the mixture was concentrated under reduced pressure, diluted with water (100 mL) and washed with EtOAc (2×100 mL). The aqueous layer was acidified to pH 2 with 2 N HCl (aq.) and extracted with EtOAc (3×150 mL). The organic extracts were combined and dried over anhydrous Na₂SO₄. After filtration, the filtrate was concentrated under reduced pressure and the residue purified by reversed-phase flash chromatography on a C18 column using a 0 to 50% gradient of MeCN in water (containing 0.1% formic acid) over 45 min to afford to afford 2-(3-methoxyisoxazol-5-yl)-3-methylbutanoic acid (racemic) (3.0 g, yield=79%) as a yellow oil.

LCMS: (ES, m/z): [M+H]⁺=200 (LCMS condition: Column: HALO 90A C18; Mobile phase A: water (containing 0.1% formic acid); Mobile phase B: MeCN (containing 0.1% formic acid); Flow rate: 1.2 mL/min.; RT: 0.693 min.)

### (f) Synthesis of Int. 53

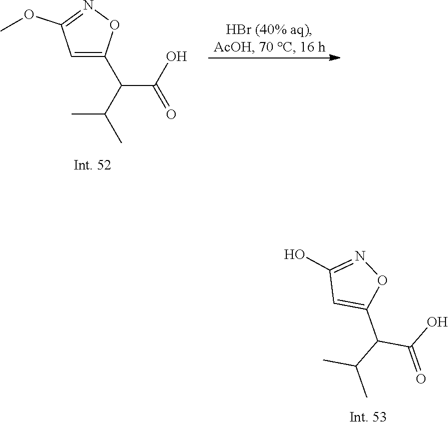

A mixture of 2-(3-methoxyisoxazol-5-yl)-3-methylbutanoic acid (racemic) (3.0 g, 15.060 mmol, 1 eq.) and HBr in water (42 mL) in AcOH (18 mL) was stirred for 16 h at 70° C. The mixture was allowed to cool to r.t. and concentrated under reduced pressure. The residue was purified by reversed-phase flash chromatography on a C18 column using a 0 to 40% gradient of MeCN in water (containing 0.1% formic acid) over 40 min to afford 2-(3-hydroxyisoxazol-5-yl)-3-methylbutanoic acid (racemic) (2.5 g, yield=900%) as a yellow solid.

LCMS: (ES, m/z): [M+H]⁺=186 (LCMS condition: Column: HALO 90A C18; Mobile phase A: water (containing 0.1% formic acid); Mobile phase B: MeCN (containing 0.1% formic acid); Flow rate: 1.2 mL/min.; RT: 0.570 min.)

### (g) Synthesis of Int. 54

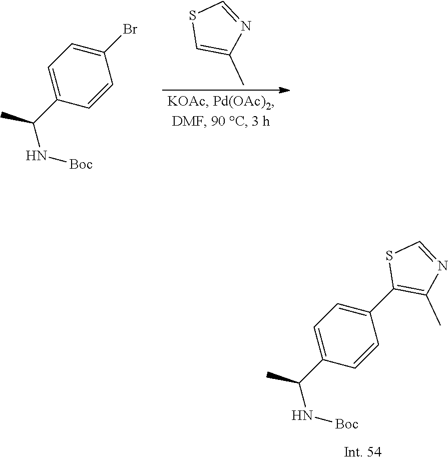

Into a 250 mL 3-necked round-bottom flask were added tert-butyl N-[(1S)-1-(4-bromophenyl)ethyl]carbamate (5.0 g, 16.656 mmol, 1 eq.), 4-methylthiazole (3.30 g, 33.283 mmol, 2 eq.), DMF (50 mL), KOAc (3.27 g, 33.319 mmol, 2 eq.) and Pd(OAc)₂ (0.19 g, 0.846 mmol, 0.05 eq.) at 16° C. The reaction was stirred for 3 h at 90° C. under nitrogen. The reaction was cooled to 16° C., quenched with water and extracted with EtOAc (3×50 mL). The combined organic extracts were washed with brine (3×50 mL) and dried over anhydrous Na₂SO₄. After filtration, the filtrate was concentrated under reduced pressure and the residue purified by silica gel column chromatography eluting with PE/EtOAc (1:1) to afford tert-butyl (S)-(1-(4-(4-methylthiazol-5-yl)phenyl)ethyl)carbamate (4.5 g, yield=74%) as a yellow solid.

LCMS: (ES, m/z): [M+H]⁺=319 (LCMS condition: Column: Shim-pack Scepter C18-120; Mobile phase A: water (containing 5 mM NH₄HCO₃); Mobile phase B: MeCN; Flow rate: 1.5 mL/min; RT: 1.353 min)

### (h) Synthesis of Int. 55

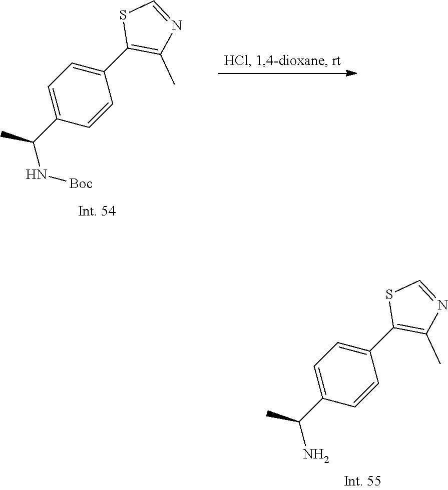 [SMILES: Cc1ncsc1-c1ccc([C@H](C)NC(=O)OC(C)(C)C)cc1 ; Cc1ncsc1-c1ccc([C@H](C)N)cc1]

Into a 100 mL round-bottom flask were added tert-butyl tert-butyl (S)-(1-(4-(4-methylthiazol-5-yl)phenyl)ethyl)carbamate (4.5 g, 14.131 mmol, 1 eq.), 1,4-dioxane (10 mL), and 4 M HCl in 1,4-dioxane (50 mL). The reaction was stirred for 1 h at 16° C. The mixture was concentrated under reduced pressure to afford (S)-1-(4-(4-methylthiazol-5-yl)phenyl)ethan-1-amine (3.3 g, yield=93%) as a yellow solid.

LCMS: (ES, m/z): [M−NH₂]⁺=202 (LCMS condition: Column: Shim-pack Scepter C18-120; Mobile phase A: water (containing 5 mM NH₄HCO₃); Mobile phase B: MeCN; Flow rate: 1.5 mL/min.; RT: 0.634 min.)

### (i) Synthesis of Int. 56

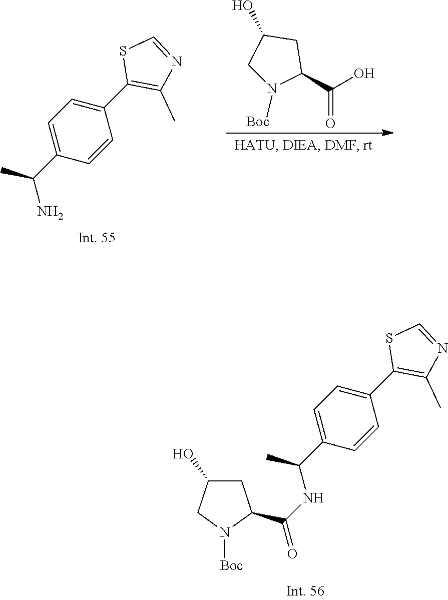 [SMILES: Cc1ncsc1-c1ccc([C@H](C)N)cc1 ; CC(C)(C)OC(=O)N1C[C@H](O)C[C@H]1C(=O)O ; Cc1ncsc1-c1ccc([C@H](C)NC(=O)[C@@H]2C[C@@H](O)CN2C(=O)OC(C)(C)C)cc1]

Into a 100 mL round-bottom flask were added (S)-1-(4-(4-methylthiazol-5-yl)phenyl)ethan-1-amine (3.3 g, 15.115 mmol, 1.2 eq.), (2S,4R)-1-(tert-butoxycarbonyl)-4-hydroxypyrrolidine-2-carboxylic acid (2.9 g, 12.596 mmol, 1 eq.), DMF (30 mL), HATU (7.18 g, 18.894 mmol, 1.5 eq.) and DIEA (4.38 g, 37.733 mmol, 3 eq.). The reaction was stirred for 1 h at 16° C. The mixture was quenched with water and extracted with EtOAc (3×100 mL). The combined organic extracts were washed with brine (2×100 mL) and dried over anhydrous Na₂SO₄. After filtration, the filtrate was concentrated under reduced pressure and the residue purified by silica gel column chromatography eluting with PE/EtOAc (1:1) to afford tert-butyl (2S,4R)-4-hydroxy-2-(((S)-1-(4-(4-methylthiazol-5-yl)phenyl)ethyl)carbamoyl)pyrrolidine-1-carboxylate hydrochloride (2.8 g, yield=51%) as a light yellow solid.

LCMS: (ES, m/z): [M−H]⁻=430 (LCMS condition: Column: Shim-pack Scepter C18-120 Mobile phase A: water (containing 5 mM NH₄HCO₃); Mobile phase B: MeCN; Flow rate: 1.5 mL/min; RT: 0.801 min)

### (j) Synthesis of Int. 57

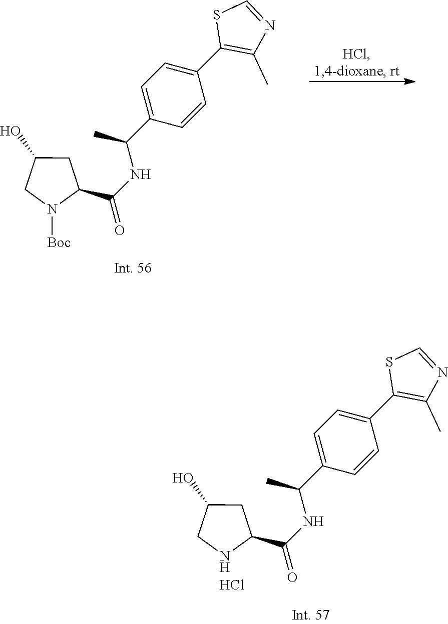 [SMILES: Cc1ncsc1-c1ccc([C@H](C)NC(=O)[C@@H]2C[C@@H](O)CN2C(=O)OC(C)(C)C)cc1 ; Cc1ncsc1-c1ccc([C@H](C)NC(=O)[C@@H]2C[C@@H](O)CN2)cc1 ; Cl]

Into a 100 mL round-bottom flask were added tert-butyl (2S,4R)-4-hydroxy-2-(((S)-1-(4-(4-methylthiazol-5-yl)phenyl)ethyl)carbamoyl)pyrrolidine-1-carboxylate hydrochloride (2.8 g, 6.488 mmol, 1 eq.), 1,4-dioxane (10 mL) and 4 M HCl in 1,4-dioxane (0.79 mL). The reaction was stirred for 1 h at 16° C. The mixture was concentrated under reduced pressure to afford (2S,4R)-4-hydroxy-N-[(1S)-1-[4-(4-methyl-1,3-thiazol-5-yl)phenyl]ethyl]pyrrolidine-2-carboxamide hydrochloride (2.1 g, yield=98%) as a yellow green solid.

LCMS: (ES, m/z): [M+H]⁺=332 (LCMS condition: Column: Shim-pack Scepter C18-120; Mobile phase A: water (containing 5 mM NH₄HCO₃); Mobile phase B: MeCN; Flow rate: 1.5 mL/min; RT: 0.642 min)

### (k) Synthesis of Int. 58

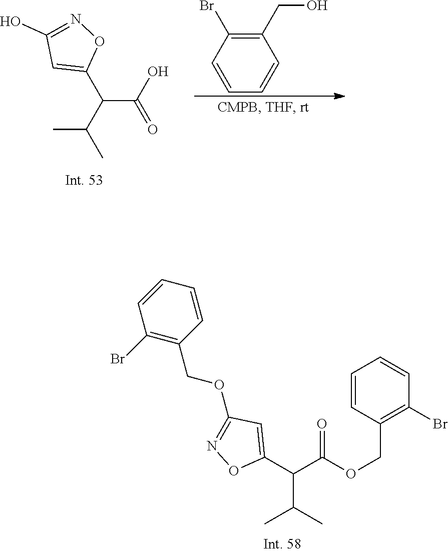 [SMILES: CC(C)C(C(=O)O)c1cc(O)no1 ; OCc1ccccc1Br ; CC(C)C(C(=O)OCc1ccccc1Br)c1cc(OCc2ccccc2Br)no1]

A mixture of 2-(3-hydroxyisoxazol-5-yl)-3-methylbutanoic acid (racemic) (2.0 g, 11.686 mmol, 1 eq.), (2-bromophenyl)methanol (8.66 g, 46.277 mmol, 3.96 eq.), and CMBP (11.34 g, 46.978 mmol, 4.02 eq.) in THF (100 mL) was stirred for 16 h at r.t. under nitrogen. The mixture was diluted with water (100 mL) and extracted with EtOAc (3×200 mL). The organic extracts were combined and dried over anhydrous Na₂SO₄. After filtration, the filtrate was concentrated under reduced pressure and the residue purified by reversed-phase flash chromatography on a C18 column using a 70 to 90% gradient of MeCN in water (containing 0.1% formic acid) over 40 min to afford 2-bromobenzyl 2-(3-((2-bromobenzyl)oxy)isoxazol-5-yl)-3-methylbutanoate (racemic) (2.0 g, yield=32%) as a brown oil.

LCMS: (ES, m/z): [M+H]⁺=524 (LCMS condition: Column: HALO 90A C18; Mobile phase A: water (containing 0.1% formic acid); Mobile phase B: MeCN (containing 0.1/a formic acid); Flow rate: 1.2 mL/min; RT: 1.137 min)

### (l) Synthesis of Int. 59

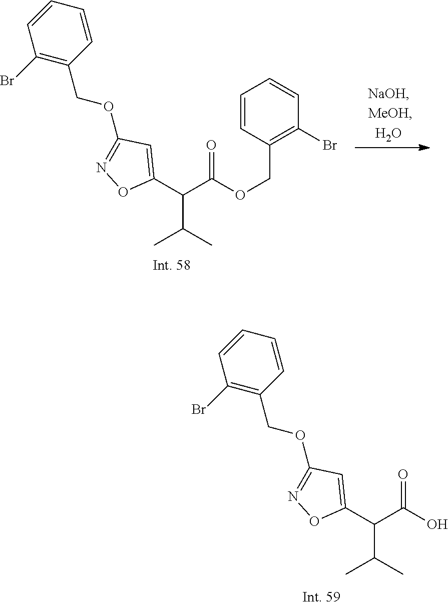 [SMILES: CC(C)C(C(=O)OCc1ccccc1Br)c1cc(OCc2ccccc2Br)no1 ; CC(C)C(C(=O)O)c1cc(OCc2ccccc2Br)no1]

Into a 250 mL round-bottom flask were added 2-bromobenzyl 2-(3-((2-bromobenzyl)oxy)isoxazol-5-yl)-3-methylbutanoate (racemic) (2.0 g, 3.822 mmol, 1 eq.), NaOH (458.66 mg, 11.466 mmol, 3.00 eq.), MeOH (100 mL) and water (20 mL) at 25° C. The reaction was stirred for 16 h at r.t. The mixture was concentrated under reduced pressure and the residue acidified to pH 5 with 1 N HCl (aq.). The mixture was extracted with EtOAc (3×100 mL), organic extracts combined and dried over anhydrous Na₂SO₄. After filtration, the filtrate was concentrated under reduced pressure and the residue was purified by reversed-phase flash chromatography on a C18 column using a 55 to 65% gradient of MeCN in water (containing 0.1% formic acid) over 10 min to afford 2-(3-((2-bromobenzyl)oxy)isoxazol-5-yl)-3-methylbutanoic acid (racemic) (1.2 g, yield=88%) as a light yellow oil.

LCMS: (ES, m/z): [M+H]⁺=356 (LCMS condition: Column: HALO 90A C18; Mobile phase A: water (containing 0.1% formic acid); Mobile phase B: MeCN (containing 0.1% formic acid); Flow rate: 1.2 mL/min; RT: 0.973 min)

### (m) Synthesis of Int. 60

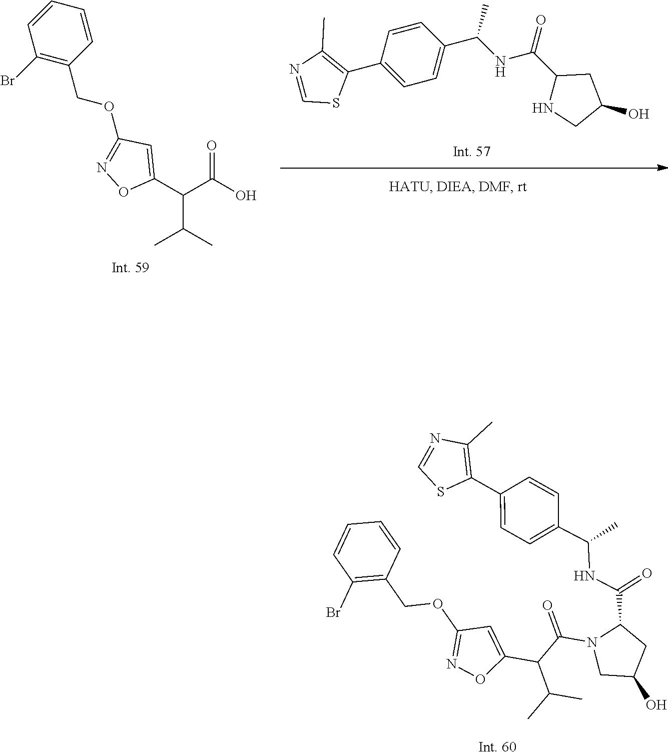 [SMILES: CC(C)C(C(=O)O)c1cc(OCc2ccccc2Br)no1 ; Cc1ncsc1-c1ccc([C@H](C)NC(=O)C2C[C@@H](O)CN2)cc1 ; Cc1ncsc1-c1ccc([C@H](C)NC(=O)[C@@H]2C[C@@H](O)CN2C(=O)C(c2cc(OCc3ccccc3Br)no2)C(C)C)cc1]

Into a 50 mL round-bottom flask were added 2-(3-((2-bromobenzyl)oxy)isoxazol-5-yl)-3-methylbutanoic acid (racemic) (1.2 g, 3.388 mmol, 1 eq.), (2S,4R)-4-hydroxy-N-[(1S)-1-[4-(4-methyl-1,3-thiazol-5-yl)phenyl]ethyl]pyrrolidine-2-carboxamide hydrochloride (1.7 g, 5.082 mmol, 1.5 eq.), HATU (1.93 g, 5.082 mmol, 1.5 eq.), DIEA (8.76 g, 67.760 mmol, 20 eq.) and DMF (20 mL) at r.t. The reaction was stirred for 2 h at r.t. The mixture was purified by reversed-phase flash chromatography on a C18 column using a 60 to 70% gradient of MeCN in water (containing 0.1/a formic acid) over 30 min to afford (2S,4R)-1-(2-(3-((2-bromobenzyl)oxy)isoxazol-5-yl)-3-methylbutanoyl)-4-hydroxy-N—((S)-1-(4-(4-methylthiazol-5-yl)phenyl)ethyl)pyrrolidine-2-carboxamide (mixture of diastereoisomers) (2.0 g, yield=88%) as a yellow solid.

LCMS: (ES, m/z): [M+H]⁺=669 (LCMS condition: Column: Shim-pack Scepter C18-120; Mobile phase A: Water/MeCN (95:5, v:v) (containing 5 mM NH₄HCO₃); Mobile phase B: MeCN; Flow rate: 1.5 mL/min; RT: 1.361 min)

### (n) Synthesis of Int. 61

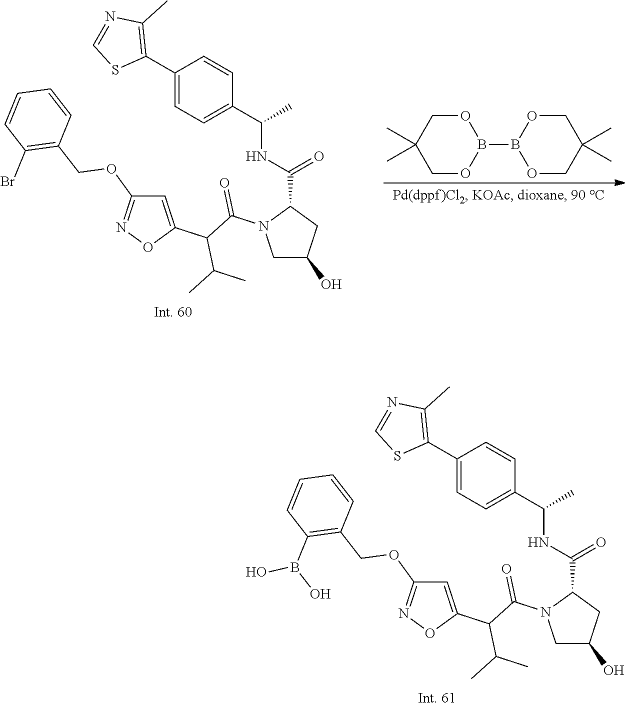

A mixture of (2S,4R)-1-(2-(3-((2-bromobenzyl)oxy)isoxazol-5-yl)-3-methylbutanoyl)-4-hydroxy-N—((S)-1-(4-(4-methylthiazol-5-yl)phenyl)ethyl)pyrrolidine-2-carboxamide (mixture of diastereoisomers) (2.0 g, 2.996 mmol, 1 eq.), 2-(5,5-dimethyl-1,3,2-dioxaborinan-2-yl)-5,5-dimethyl-1,3,2-dioxaborinane (6.77 g, 29.960 mmol, 10 eq.), Pd(dppf)Cl₂·DCM (244.04 mg, 0.300 mmol, 0.1 eq.) and KOAc (2.94 g, 29.96 mmol, 10 eq.) in 1,4-dioxane was stirred for 1 h at 90° C. under nitrogen. The mixture was allowed to cool to r.t., diluted with water (100 mL) and extracted with EtOAc (3×200 mL). The organic extracts were combined and dried over anhydrous Na₂SO₄. After filtration, the filtrate was concentrated under reduced pressure The residue was purified by reversed-phase flash chromatography on a C18 column using a 0 to 100% gradient of MeCN in water (containing 10 mmol/L NH₄HCO₃) over 35 min. The crude product (930 mg) was purified by preparative HPLC (Column: WelFlash C18 20-40 μm, 330 g; Mobile Phase A: water (containing 10 mmol/L NH₄HCO₃), Mobile Phase B: MeCN; Flow rate: 100 mL/min; Gradient: 25 B to 55% B over 30 min; Wave Length: 254 and 220 nm; RT (min): 27.89) to afford (2-(((5-(1-((2S,4R)-4-hydroxy-2-(((S)-1-(4-(4-methylthiazol-5-yl)phenyl)ethyl)carbamoyl)pyrrolidin-1-yl)-3-methyl-1-oxobutan-2-yl)isoxazol-3-yl)oxy)methyl)phenyl)boronic acid (mixture of diastereoisomers) (650 mg, yield=34%) as a white solid.

LCMS: (ES, m/z): [M−H]⁻=631 (LCMS condition: Column: Shim-pack Scepter C18-120; Mobile phase A: Water/MeCN (95:5, v:v) (containing 5 mM NH₄HCO₃); Mobile phase B: MeCN; Flow rate: 1.5 mL/min; RT: 1.167 min)

### (o) Separation of Int. 61 Diastereoisomers

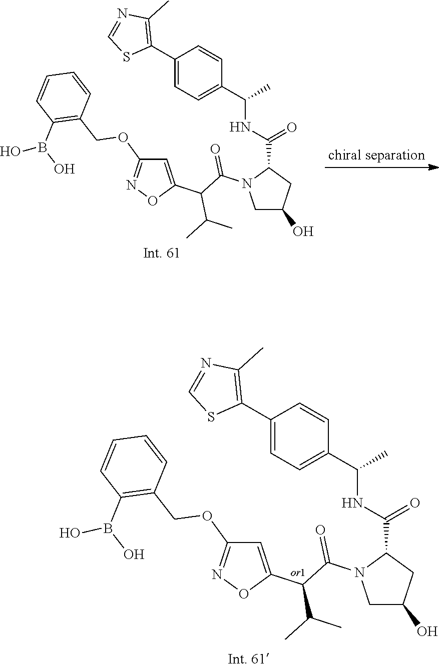 [SMILES: Cc1ncsc1-c1ccc([C@H](C)NC(=O)[C@@H]2C[C@@H](O)CN2C(=O)C(c2cc(OCc3ccccc3B(O)O)no2)C(C)C)cc1 ; Cc1ncsc1-c1ccc([C@H](C)NC(=O)[C@@H]2C[C@@H](O)CN2C(=O)[C@H](c2cc(OCc3ccccc3B(O)O)no2)C(C)C)cc1]

(2-(((5-(1-((2S,4R)-4-Hydroxy-2-(((S)-1-(4-(4-methylthiazol-5-yl)phenyl)ethyl)carbamoyl)pyrrolidin-1-yl)-3-methyl-1-oxobutan-2-yl)isoxazol-3-yl)oxy)methyl)phenyl)boronic acid (mixture of diastereoisomers) (650 mg) was separated by preparative chiral SFC (Column: CHIRAL ART Amylose-C NEO 3×25 cm, 5 μm; Mobile Phase A: CO₂, Mobile Phase B: MeOH (containing 1% of a 2 M solution of NH₃ in MeOH); Flow rate: 90 mL/min; Gradient: isocratic 28% B; Column Temperature (° C.): 35; Back Pressure (bar): 100; Wave Length: 220 nm; RT peak 1 (min): 4.52; RT peak 2 (min): 6.45; Sample Solvent: MeOH; Injection Volume: 1 mL) to afford (2-(((5-(1-((2S,4R)-4-hydroxy-2-(((S)-1-(4-(4-methylthiazol-5-yl)phenyl)ethyl)carbamoyl)pyrrolidin-1-yl)-3-methyl-1-oxobutan-2-yl)isoxazol-3-yl)oxy)methyl)phenyl)boronic acid (second eluting peak) (single diastereoisomer with unknown absolute stereochemistry at the centre indicated in the structure) (244.8 mg, yield=37%) as a white solid.

LCMS: (ES, m/z): [M−H]⁻=631 (LCMS condition: Column: HALO 90A C18; Mobile phase A: water (containing 0.1% formic acid); Mobile phase B: MeCN (containing 0.1% formic acid); Flow rate: 1.2 mL/min; RT: 0.805 min)

### (p) Synthesis of Int. 62

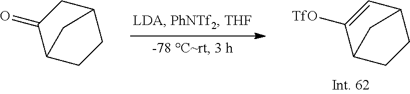 [SMILES: O=C1CC2CCC1C2 ; O=S(=O)(OC1=CC2CCC1C2)C(F)(F)F]

To a stirred mixture of norcamphor (racemic) (5.0 g, 45.390 mmol, 1 eq.) in THF (50 mL) was added LDA (25.0 mL, 183.829 mmol, 4.05 eq.) at −78° C. under nitrogen. The reaction was stirred for 30 min at r.t. under nitrogen. 1,1,1-Trifluoro-N-phenyl-N-(trifluoromethane)sulfonylmethanesulfonamide (8.9 g, 24.965 mmol, 0.55 eq.) was added at −78° C. under nitrogen and the reaction stirred for 3 h warming to r.t. The mixture was quenched with water (100 mL) and extracted with EtOAc (3×100 mL). The organic extracts were combined, washed with brine (2×100 mL) and dried over Na₂SO₄. After filtration, the filtrate was concentrated under reduced pressure and the residue purified by silica gel column chromatography eluting with PE/EtOAc to afford bicyclo[2.2.1]hept-2-en-2-yl trifluoromethanesulfonate (racemic) (10.2 g, yield=88%) as an orange oil.

### (q) Synthesis of Int. 63

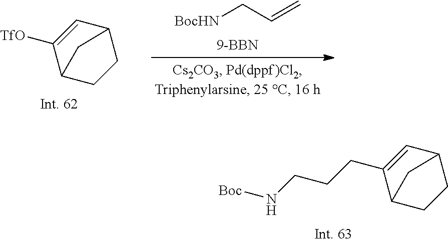 [SMILES: O=S(=O)(OC1=CC2CCC1C2)C(F)(F)F ; C=CCNC(=O)OC(C)(C)C ; CC(C)(C)OC(=O)NCCCC1=CC2CCC1C2]

A mixture of tert-butyl allylcarbamate (4.9 g, 30.9 mmol, 1.5 eq.) and 9-borabicyclo[3.3.1]nonane (82.6 mL, 2.0 eq.) in THF (50 mL) was stirred for 4 h at 25° C. To this mixture was added water (100 mL), bicyclo[2.2.1]hept-2-en-2-yl trifluoromethanesulfonate (racemic) (5.0 g, 20.6 mmol, 1.0 eq.), triphenylarsine (4.0 mL, 0.15 eq.), Cs₂CO₃ (10.0 g, 30.9 mmol, 1.5 eq.), DMF (50 mL) and Pd(dppf)Cl₂ (2.3 g, 3.09 mmol, 0.15 eq.). The resulting mixture was stirred for 16 h at 25° C. The mixture was quenched with saturated NH₄Cl (aq.) (50 mL) and extracted with EtOAc (3×150 mL). The organic extracts were combined, washed with brine (2×150 mL) and dried over Na₂SO₄. After filtration, the filtrate was concentrated under reduced pressure and the residue purified by silica gel column chromatography eluting with PE/EtOAc to afford tert-butyl N-(3-{bicyclo[2.2.1]hept-2-en-2-yl}propyl)carbamate (racemic) (4.1 g, yield=79%) as a yellow oil.

LCMS: (ES, m/z): [M−C₄H₈]⁺=196 (LCMS condition: Column: HALO 90A C18; Mobile phase A: water (containing 0.1% formic acid); Mobile phase B: MeCN (containing 0.1% formic acid); Flow rate: 1.5 mL/min.; RT: 1.048 min)

### (r) Synthesis of Int. 64

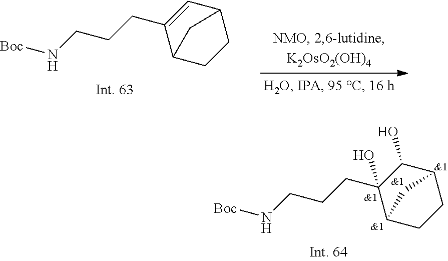 [SMILES: CC(C)(C)OC(=O)NCCCC1=CC2CCC1C2 ; CC(C)(C)OC(=O)NCCC[C@]1(O)[C@@H]2CC[C@@H](C2)[C@H]1O]

A mixture of tert-butyl N-(3-{bicyclo[2.2.1]hept-2-en-2-yl}propyl)carbamate (racemic) (4.1 g, 16.3 mmol, 1.0 eq.), NMO (2.5 g, 21.2 mmol, 1.3 eq.), 2,6-lutidine (2.1 g, 19.5 mmol, 1.2 eq.), K₂OsO₂(OH)₄ (0.6 g, 1.63 mmol, 0.1 eq.) in IPA (20 mL) and water (4 mL) was stirred for 16 h at 95° C. under nitrogen. The reaction was quenched with saturated NH₄Cl (aq.) (60 mL) and extracted with EtOAc (3×60 mL). The organic extracts were combined, washed with brine (2×60 mL) and dried over Na₂SO₄. After filtration, the filtrate was concentrated under reduced pressure and the residue purified by silica gel column chromatography eluting with PE/EtOAc to afford tert-butyl (3-(2,3-cis-exo-dihydroxybicyclo[2.2.1]heptan-2-endo-yl)propyl)carbamate (racemic) (3 g, yield=61%) as a yellow green oil.

LCMS: (ES, m/z): [M+Na]⁺=308 (LCMS condition: Column: HALO 90A C18; Mobile phase A: water (containing 0.1% formic acid); Mobile phase B: MeCN (containing 0.1% formic acid); Flow rate: 1.5 mL/min.; RT: 0.597 min)

### (s) Synthesis of Int. 65

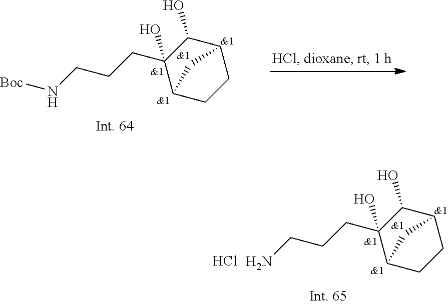 [SMILES: CC(C)(C)OC(=O)NCCC[C@]1(O)[C@@H]2CC[C@@H](C2)[C@H]1O ; Cl ; NCCC[C@]1(O)[C@@H]2CC[C@@H](C2)[C@H]1O]

A mixture of tert-butyl (3-(2,3-cis-exo-dihydroxybicyclo[2.2.1]heptan-2-endo-yl)propyl)carbamate (racemic) (600 mg, 2.10 mmol, 1.0 eq.) and 4 M HCl in 1,4-dioxane (6 mL) was stirred for 30 min at 25° C. The mixture was concentrated under reduced pressure to afford crude 2-endo-(3-aminopropyl)bicyclo[2.2.1]heptane-2,3-cis-exo-diol hydrochloride (racemic) (389 mg) as an off-white solid which was used in the next step without purification.

LCMS: (ES, m/z): [M+H]⁺=186 (LCMS condition: Column: HALO 90A C18; Mobile phase A: water (containing 0.1% formic acid); Mobile phase B: MeCN (containing 0.1% formic acid); Flow rate: 1.2 mL/min.)

### (t) Synthesis of Int. 66

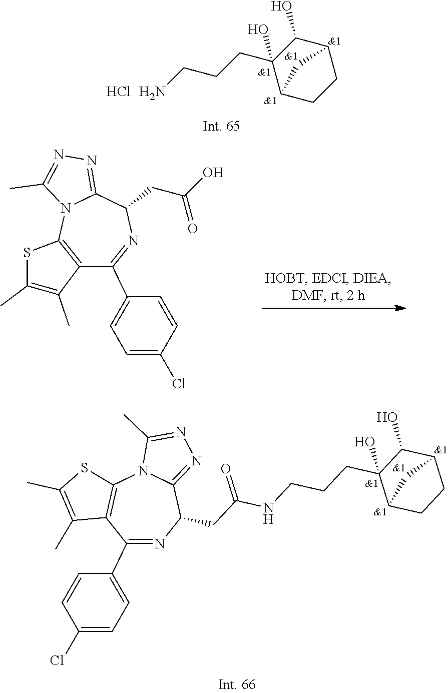 [SMILES: Cl ; NCCC[C@]1(O)[C@@H]2CC[C@@H](C2)[C@H]1O ; Cc1sc2c(c1C)C(c1ccc(Cl)cc1)=N[C@@H](CC(=O)O)c1nnc(C)n1-2 ; Cc1sc2c(c1C)C(c1ccc(Cl)cc1)=N[C@@H](CC(=O)NCCC[C@]1(O)[C@@H]3CC[C@@H](C3)[C@H]1O)c1nnc(C)n1-2]

To a stirred mixture of (S)-2-(4-(4-chlorophenyl)-2,3,9-trimethyl-6H-thieno[3,2-f][1,2,4]triazolo[4,3-a][1,4]diazepin-6-yl)acetic acid (336 mg, 0.84 mmol, 1.0 eq.), HOBT (170 mg, 1.26 mmol, 1.5 eq.) and DIEA (760 mg, 5.88 mmol, 7.0 eq.) in DMF (4 mL) was added EDCI (242 mg, 1.26 mmol, 1.5 eq.). After stirring for 30 min at 0° C., 2-endo-(3-aminopropyl)bicyclo[2.2.1]heptane-2,3-cis-exo-diol hydrochloride (racemic) (389 mg, 2.10 mmol, 2.5 eq.) was added. The reaction was stirred for 2 h at 25° C. The mixture was quenched with water (20 mL) and extracted with EtOAc (3×20 mL). The organic extracts were combined, washed with brine (2×20 mL) and dried over Na₂SO₄. After filtration, the filtrate was concentrated under reduced pressure and the residue purified by preparative HPLC (Column: Xselect CSH Prep C18, 30×150 mm, 5 μm; Mobile Phase A: water (containing 0.1% formic acid), Mobile Phase B: MeCN; Flow rate: 60 mL/min mL/min; Gradient: 36 to 52% B over 10 min; Wave Length: 254 and 220 nm; RT (min): 8.68) to afford 2-((S)-4-(4-chlorophenyl)-2,3,9-trimethyl-6H-thieno[3,2-f][1,2,4]triazolo[4,3-a][1,4]diazepin-6-yl)-N-(3-(2,3-dihydroxybicyclo[2.2.1]heptan-2-yl)propyl)acetamide (mixture of diastereoisomers (175.5 mg, yield=37%) as a white solid.

LCMS: (ES, m/z): [M+H]⁺=568 (LCMS condition: Column: HALO 90A C18; Mobile phase A: water (containing 0.1% formic acid); Mobile phase B: MeCN (containing 0.1% formic acid); Flow rate: 1.2 mL/min.; RT: 1.304 min.)

### (u) Separation of Int. 66 Diastereoisomers

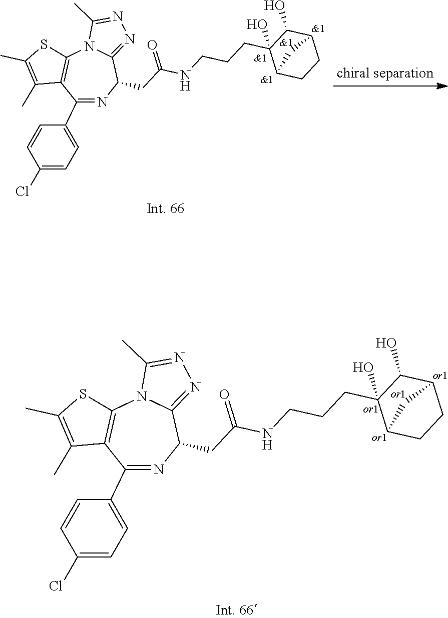 [SMILES: Cc1sc2c(c1C)C(c1ccc(Cl)cc1)=N[C@@H](CC(=O)NCCC[C@]1(O)[C@@H]3CC[C@@H](C3)[C@H]1O)c1nnc(C)n1-2 ; Cc1sc2c(c1C)C(c1ccc(Cl)cc1)=N[C@@H](CC(=O)NCCC[C@]1(O)[C@@H]3CC[C@@H](C3)[C@H]1O)c1nnc(C)n1-2]

2-((S)-4-(4-Chlorophenyl)-2,3,9-trimethyl-6H-thieno[3,2-f][1,2,4]triazolo[4,3-a][1,4]diazepin-6-yl)-N-(3-(2,3-dihydroxybicyclo[2.2.1]heptan-2-yl)propyl)acetamide (mixture of diastereoisomers) (160 mg) was separated by preparative chiral HPLC (Column: Chiralpak IG, 2×25 cm, 5 μm; Mobile Phase A: Hexane (containing 0.1% formic acid), Mobile Phase B: EtOH: DCM (1:1); Flow rate: 20 mL/min; Gradient: isocratic 40% B; Wave Length: 254 and 220 nm; RT peak 2 (min): 13.163; Sample Solvent: EtOH; Injection Volume: 0.4 mL; Number of runs: 12) to afford 2-((S)-4-(4-chlorophenyl)-2,3,9-trimethyl-6H-thieno[3,2-f][1,2,4]triazolo[4,3-a][1,4]diazepin-6-yl)-N-(3-(2,3-dihydroxybicyclo[2.2.1]heptan-2-yl)propyl)acetamide (second eluting peak) (single diastereoisomer with unknown absolute stereochemistry at the centres indicated in the structure) (69.5 mg, yield=44%) as white solid.

LCMS: (ES, m/z): [M+H]⁺=568 (LCMS condition: Column: HALO 90A C18; Mobile phase A: water (containing 0.1% formic acid); Mobile phase B: MeCN (containing 0.1% formic acid); Flow rate: 1.2 mL/min.; RT: 0.871 min.)

### (v) Synthesis of Example 9

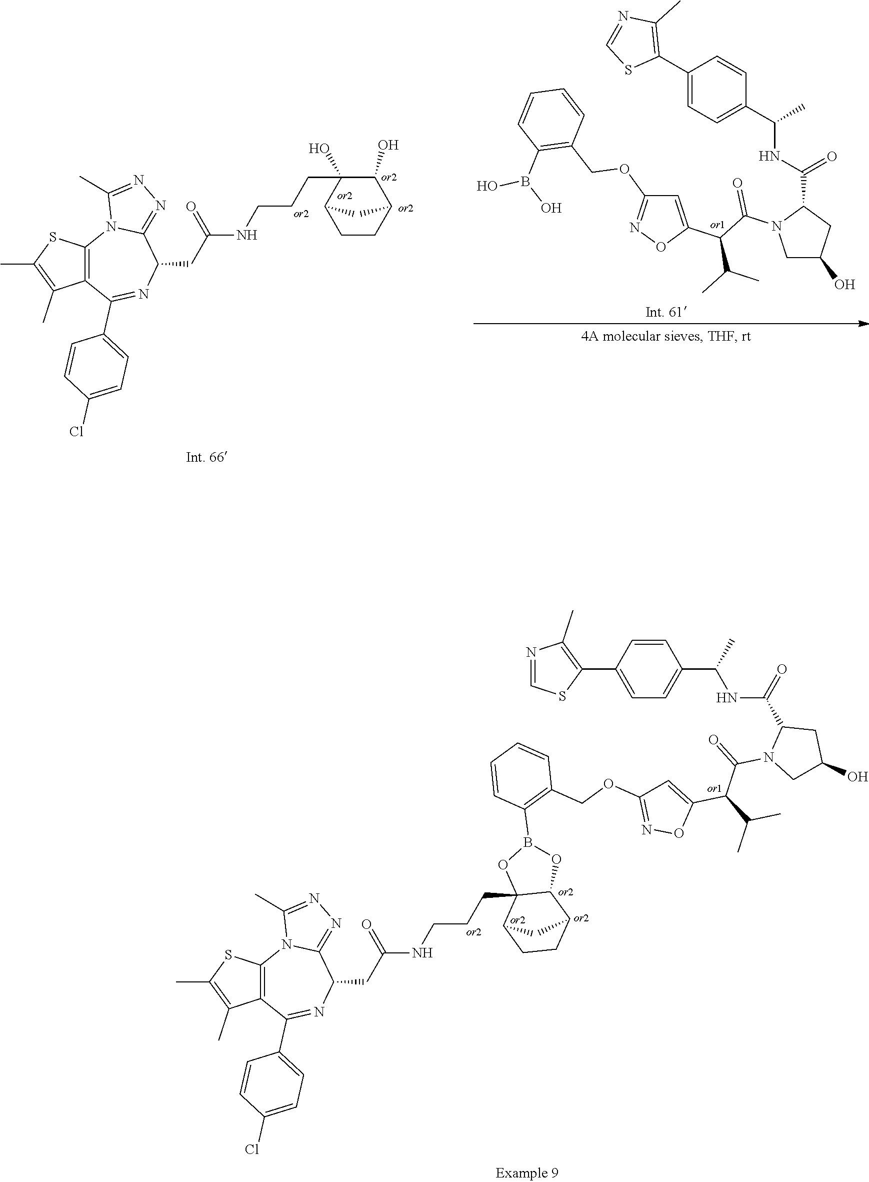

A mixture of 2-((S)-4-(4-chlorophenyl)-2,3,9-trimethyl-6H-thieno[3,2-f][1,2,4]triazolo[4,3-a][1,4]diazepin-6-yl)-N-(3-(2,3-dihydroxybicyclo[2.2.1]heptan-2-yl)propyl)acetamide (single diastereoisomer with unknown absolute stereochemistry at the centres indicated in the structure) (13 mg, 0.032 mmol, 1 eq.), molecular sieves (4 Å) (90 mg) and (2-(((5-(1-((2S,4R)-4-hydroxy-2-(((S)-1-(4-(4-methylthiazol-5-yl)phenyl)ethyl)carbamoyl)pyrrolidin-1-yl)-3-methyl-1-oxobutan-2-yl)isoxazol-3-yl)oxy)methyl)phenyl)boronic acid (single diastereoisomer with unknown absolute stereochemistry at the centre indicated in the structure) (20 mg, 0.032 mmol, 1 eq.) in THF (5 mL) was stirred for 16 h at r.t. under nitrogen. The mixture was filtered, and the filter cake washed with THF (3×5 mL). The filtrate was concentrated under reduced pressure and the residue purified by preparative SFC (Column: Viridis BEH Prep 2-EP OBD 3×15 cm, 5 μm; Mobile Phase A: CO₂, Mobile Phase B: MeOH (containing 1% of a 2 M solution of NH₃ in MeOH); Flow rate: 65 mL/min; Gradient: isocratic 26% B; Column Temperature (C): 35; Back Pressure (bar): 100; Wave Length: 220 nm; RT (min): 3.93; Sample Solvent: MeOH; Injection Volume: 2 mL) to afford (2S,4R)-1-(2-(3-((2-(3a-(3-(2-((S)-4-(4-chlorophenyl)-2,3,9-trimethyl-6H-thieno[3,2-f][1,2,4]triazolo[4,3-a][1,4]diazepin-6-yl)acetamido)propyl)hexahydro-4,7-methanobenzo[d][1,3,2]dioxaborol-2-yl)benzyl)oxy)isoxazol-5-yl)-3-methylbutanoyl)-4-hydroxy-N—((S)-1-(4-(4-methylthiazol-5-yl)phenyl)ethyl)pyrrolidine-2-carboxamide (single diastereoisomer with unknown absolute stereochemistry at the centres indicated in the structure) (14.1 mg, yield=37%) as a white solid.

LCMS: (ES, m/z): [M+H]⁺=1164 (LCMS condition: Column: Shim-pack Scepter C18-120; Mobile phase A: Water/MeCN (95:5, v:v) (containing 5 mM NH₄HCO₃); Mobile phase B: MeCN; Flow rate: 1.5 mL/min.; RT: 0.977 min.)

### Example 10

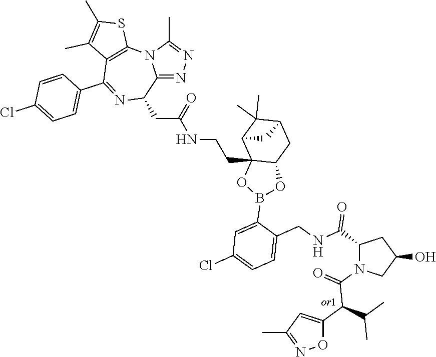 [SMILES: Cc1cc([C@@H](C(=O)N2C[C@H](O)C[C@H]2C(=O)NCc2ccc(Cl)cc2B2O[C@H]3C[C@H]4C[C@H](C4(C)C)[C@@]3(CCNC(=O)C[C@@H]3N=C(c4ccc(Cl)cc4)c4c(sc(C)c4C)-n4c(C)nnc43)O2)C(C)C)on1]

### (a) Synthesis of Int. 2-5 as Described in Example 1

### (b) Synthesis of Int. 67

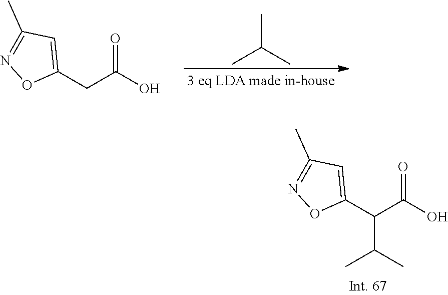

A stirred solution of diisopropylamine (14.4 g, 141.907 mmol, 1.00 eq.) in THF (140 mL) at −78° C. under nitrogen was treated with 2.5 M n-BuLi in hexanes (56.8 mL, 141.907 mmol, 1 eq.) dropwise and the mixture stirred for 1.5 h at −78° C. The reaction was allowed to warm to r.t. and stirred for 30 min. The mixture was at cooled to −78° C. and treated with (3-methyl-1,2-oxazol-5-yl)acetic acid (10.0 g, 70.859 mmol, 1 eq.) in THF (100 mL) dropwise over 30 min. The mixture was stirred for 2.5 h at −78° C. 2-Iodopropane (36.1 g, 212.577 mmol, 3 eq.) was added dropwise over 30 min at −78° C. The mixture was stirred for 30 min at −78° C. and allowed to warm to 25° C. stirring overnight. The mixture was quenched with 1 N HCl (aq.) at 25° C. and extracted with EtOAc (3×300 mL). The combined organic extracts were washed with brine (200 mL) and dried over anhydrous Na₂SO₄. After filtration, the filtrate was concentrated under reduced pressure and the residue purified by silica gel column chromatography eluting with PE/THF (5:1) to afford 3-methyl-2-(3-methyl-1,2-oxazol-5-yl)butanoic acid (racemic) (6 g, yield=46%).

LCMS: (ES, m/z): [M+H]⁺=184 (LCMS condition: Column: HALO 90A C18; Mobile phase A: water (containing 0.1% formic acid); Mobile phase B: MeCN (containing 0.1% formic acid) Flow rate: 1.121 mL/min; RT: 0.521 min

### (c) Synthesis of Int. 68

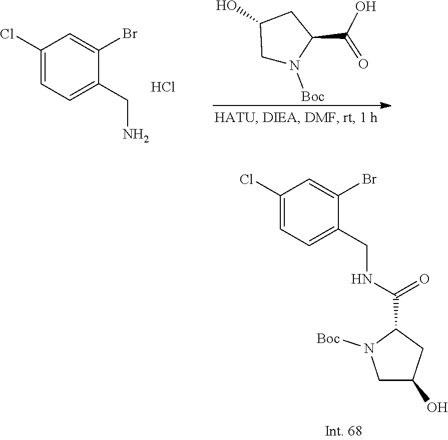 [SMILES: NCc1ccc(Cl)cc1Br ; Cl ; CC(C)(C)OC(=O)N1C[C@H](O)C[C@H]1C(=O)O ; CC(C)(C)OC(=O)N1C[C@H](O)C[C@H]1C(=O)NCc1ccc(Cl)cc1Br]

To a 250 mL round-bottom flask were added 1-(2-bromo-4-chlorophenyl)methanamine hydrochloride (7.5 g, 29.189 mmol, 1 eq.), (2S,4R)-1-(tert-butoxycarbonyl)-4-hydroxypyrrolidine-2-carboxylic acid (6.8 g, 29.189 mmol, 1.00 eq.), HATU (14.4 g, 37.946 mmol, 1.3 eq.), DIEA (37.73 g, 291.890 mmol, 10.00 eq.) and DMF (75.0 mL, 12.922 mmol). The reaction was stirred for 2 h at r.t. The mixture was quenched with water (200 mL) and extracted with EtOAc (3×300 mL). The organic extracts were combined, washed with brine and dried over anhydrous Na₂SO₄. After filtration, the filtrate was concentrated under reduced pressure and the residue purified by reversed-phase flash chromatography on a C18 column using a 10 to 60% gradient of MeCN in water (containing 10 mmol/L NH₄HCO₃) over 40 min to afford tert-butyl (2S,4R)-2-{[(2-bromo-4-chlorophenyl)methyl]carbamoyl}-4-hydroxypyrrolidine-1-carboxylate (11 g, yield=87%) as an off-white solid.

LCMS: (ES, m/z): [M−C₅H₇O₂]⁺=335 (LCMS condition: Column: HALO 90A C18; Mobile phase A: water (containing 0.1% formic acid); Mobile phase B: MeCN (containing 0.1% formic acid); Flow rate: 1.2 mL/min.; RT: 1.224 min.)

### (d) Synthesis of Int. 69

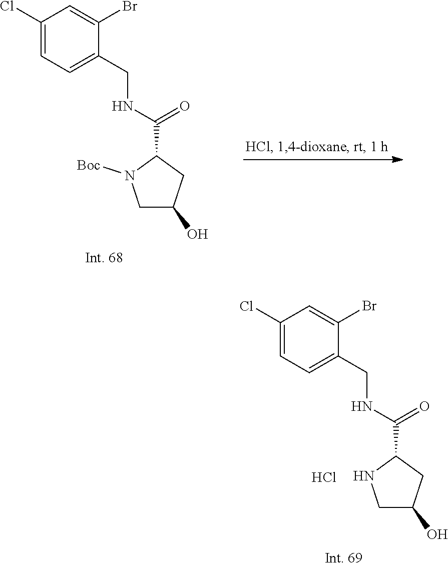 [SMILES: CC(C)(C)OC(=O)N1C[C@H](O)C[C@H]1C(=O)NCc1ccc(Cl)cc1Br ; O=C(NCc1ccc(Cl)cc1Br)[C@@H]1C[C@@H](O)CN1 ; Cl]

To a 250 mL round-bottom flask were added tert-butyl (2S,4R)-2-{[(2-bromo-4-chlorophenyl)methyl]carbamoyl}-4-hydroxypyrrolidine-1-carboxylate (6 g, 13.833 mmol, 1 eq.) and 4 M HCl in 1,4-dioxane (60 mL) at r.t. The reaction was stirred for 2 h at r.t. The mixture was concentrated under reduced pressure to afford crude (2S,4R)—N-[(2-bromo-4-chlorophenyl)methyl]-4-hydroxypyrrolidine-2-carboxamide hydrochloride (5.3 g) as an off-white solid which was used in the next step without purification.

LCMS: (ES, m/z): [M+H]⁺=335 (LCMS condition: Column: Kinetex EVO C18; Mobile phase A: water (containing 6.5 mM NH₄HCO₃ and ammonia hydroxide (pH=10)); Mobile phase B: MeCN; Flow rate: 1.5 mL/min.; RT: 0.634 min.)

### (e) Synthesis of Int. 70

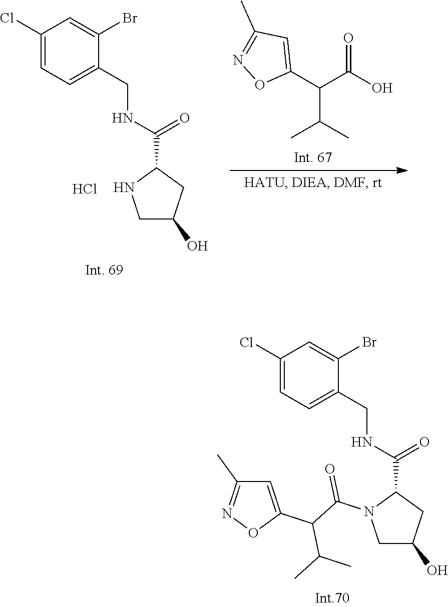 [SMILES: O=C(NCc1ccc(Cl)cc1Br)[C@@H]1C[C@@H](O)CN1 ; Cl ; Cc1cc(C(C(=O)O)C(C)C)on1 ; Cc1cc(C(C(=O)N2C[C@H](O)C[C@H]2C(=O)NCc2ccc(Cl)cc2Br)C(C)C)on1]

To a 40 mL vial were added 3-methyl-2-(3-methyl-1,2-oxazol-5-yl)butanoic acid (racemic) (1.5 g, 8.187 mmol, 1 eq.), (2S,4R)—N-[(2-bromo-4-chlorophenyl)methyl]-4-hydroxypyrrolidine-2-carboxamide hydrochloride (3.3 g, 9.824 mmol, 1.20 eq.), HATU (4.67 g, 12.282 mmol, 1.50 eq.), DIEA (5.29 g, 40.935 mmol, 5 eq.) and DMF (15 mL). The reaction was stirred for 2 h at r.t. The mixture was quenched with water (200 mL) and extracted with EtOAc (3×300 mL). The organic extracts were combined, washed with brine (3×200 mL) and dried over Na₂SO₄. After filtration, the filtrate was concentrated under reduced pressure and the residue purified by reversed-phase flash chromatography on a C18 column using a 10 to 60% gradient of MeCN in water (containing 0.1% formic acid) over 30 min to afford (2S,4R)—N-[(2-bromo-4-chlorophenyl)methyl]-4-hydroxy-1-[3-methyl-2-(3-methyl-1,2-oxazol-5-yl)butanoyl]pyrrolidine-2-carboxamide (mixture of diastereoisomers) (3.3 g, yield=81%) as an off-white solid.

LCMS: (ES, m/z): [M−H]⁻=498 (LCMS condition: Column: HALO 90A C18; Mobile phase A: water (containing 0.1% formic acid); Mobile phase B: MeCN (containing 0.1% formic acid); Flow rate: 1.2 mL/min.; RT: 1.280 min.)

### (f) Synthesis of Int. 71

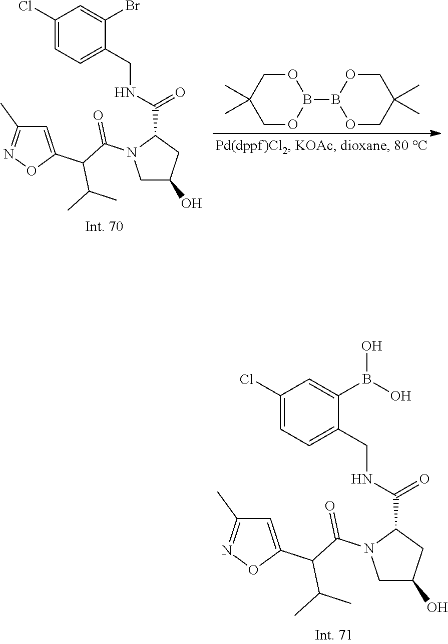

A mixture of (2S,4R)—N-[(2-bromo-4-chlorophenyl)methyl]-4-hydroxy-1-[3-methyl-2-(3-methyl-1,2-oxazol-5-yl)butanoyl]pyrrolidine-2-carboxamide (mixture of diastereoisomers) (1.8 g, 3.609 mmol, 1 eq.), 2-(5,5-dimethyl-1,3,2-dioxaborinan-2-yl)-5,5-dimethyl-1,3,2-dioxaborinane (8.2 g, 36.090 mmol, 10 eq.), Pd(dppf)Cl₂·DCM (323 mg, 0.397 mmol, 0.11 eq.) and KOAc (1.8 g, 18.045 mmol, 5 eq.) in 1,4-dioxane (45 mL, 531.178 mmol, 147.20 eq.) was stirred for 2 h at 80° C. under nitrogen. The mixture was allowed to cool to r.t., filtered, and the filter cake washed with 1,4-dioxane (3×40 mL). The filtrate was concentrated under reduced pressure and the residue purified by reversed-phase flash chromatography on a C18 column using a 10 to 50% gradient of MeCN in water (containing 10 mmol/L NH₄HCO₃) over 30 min. The crude product (1.2 g) was purified by preparative HPLC (Column: Welch Ultimate XB-C18 50×250 mm, 10 μm; Mobile Phase A: Water (containing 10 mmol/L NH₄HCO₃), Mobile Phase B: MeCN; Flow rate: 60 mL/min; Gradient: 26 to 56% B over 20 min; Wave Length: 254 and 220 nm; RT (min): 19.5) to afford 5-chloro-2-({[(2S,4R)-4-hydroxy-1-[3-methyl-2-(3-methyl-1,2-oxazol-5-yl)butanoyl]pyrrolidin-2-yl]formamido}methyl)phenylboronic acid (mixture of diastereoisomers) (400 mg, yield=24%) as a white solid.

LCMS: (ES, m/z): [M−OH]⁺=446 (LCMS condition: Column: Shim-pack Scepter C18-120; Mobile phase A: Water/MeCN (95:5, v:v) (containing 5 mM NH₄HCO₃); Mobile phase B: MeCN; Flow rate: 1.5 mL/min.; RT: 1.339 min.)

### (g) Separation of Int. 71 Diastereoisomers

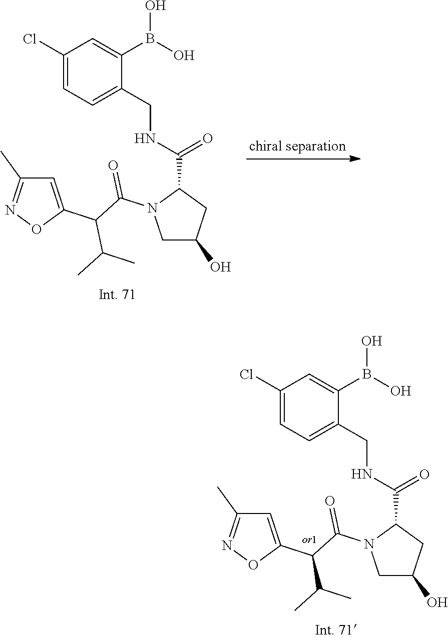 [SMILES: Cc1cc(C(C(=O)N2C[C@H](O)C[C@H]2C(=O)NCc2ccc(Cl)cc2B(O)O)C(C)C)on1 ; Cc1cc([C@@H](C(=O)N2C[C@H](O)C[C@H]2C(=O)NCc2ccc(Cl)cc2B(O)O)C(C)C)on1]

5-Chloro-2-({[(2S,4R)-4-hydroxy-1-[3-methyl-2-(3-methyl-1,2-oxazol-5-yl)butanoyl]pyrrolidin-2-yl]formamido}methyl)phenylboronic acid (mixture of diastereoisomers) (400 mg) was separated by preparative chiral HPLC Column: Chiralpak IF, 2×25 cm, 5 μm; Mobile Phase A: hexane (containing 0.1% formic acid), Mobile Phase B: EtOH: DCM (1:1); Flow rate: 20 mL/min; Gradient: isocratic 20% B; Wave Length: 254 and 220 nm; RT peak 1 (min): 5.393; RT peak 2 (min): 11.95; Sample Solvent: EtOH; Injection Volume: 1.0 mL; Number of runs: 10) to afford (5-chloro-2-(((2S,4R)-4-hydroxy-1-(3-methyl-2-(3-methylisoxazol-5-yl)butanoyl)pyrrolidine-2-carboxamido)methyl)phenyl)boronic acid (second eluting peak) (single diastereoisomer with unknown absolute stereochemistry at the centre indicated in the structure (169.6 mg, yield=42%) as a white solid.

LCMS: (ES, m/z): [M−H]⁻=462 (LCMS condition: Column: HALO 90A C18; Mobile phase A: water (containing 0.1% formic acid); Mobile phase B: MeCN (containing 0.1% formic acid); Flow rate: 1.2 mL/min.; RT: 0.704 min.)

### (h) Synthesis of Example 10

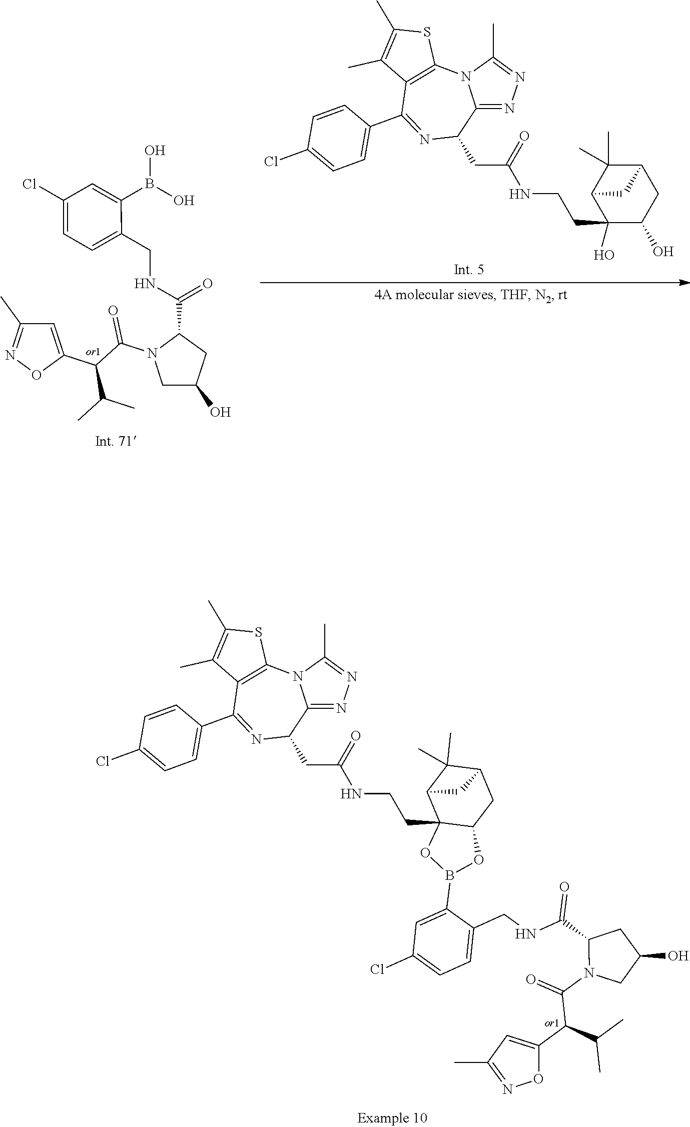 [SMILES: Cc1cc([C@@H](C(=O)N2C[C@H](O)C[C@H]2C(=O)NCc2ccc(Cl)cc2B(O)O)C(C)C)on1 ; Cc1sc2c(c1C)C(c1ccc(Cl)cc1)=N[C@@H](CC(=O)NCC[C@@]1(O)[C@@H]3C[C@H](C[C@@H]1O)C3(C)C)c1nnc(C)n1-2 ; Cc1cc([C@@H](C(=O)N2C[C@H](O)C[C@H]2C(=O)NCc2ccc(Cl)cc2B2O[C@H]3C[C@H]4C[C@H](C4(C)C)[C@@]3(CCNC(=O)C[C@@H]3N=C(c4ccc(Cl)cc4)c4c(sc(C)c4C)-n4c(C)nnc43)O2)C(C)C)on1]

A solution of (5-chloro-2-(((2S,4R)-4-hydroxy-1-(3-methyl-2-(3-methylisoxazol-5-yl)butanoyl)pyrrolidine-2-carboxamido)methyl)phenyl)boronic acid (single diastereoisomer with unknown absolute stereochemistry at the centre indicated in the structure) (18 mg, 0.039 mmol, 1 eq.), molecular sieves (4 Å) (90 mg) and 2-((S)-4-(4-chlorophenyl)-2,3,9-trimethyl-6H-thieno[3,2-f][1,2,4]triazolo[4,3-a][1,4]diazepin-6-yl)-N-(2-((1R,2R,3S,5R)-2,3-dihydroxy-6,6-dimethylbicyclo[3.1.1]heptan-2-yl)ethyl)acetamide (22 mg, 0.039 mmol, 1 eq.) in THF (5 mL) was stirred for 16 h at r.t. under nitrogen. The reaction was filtered, and the filter cake washed with THF (3×5 mL). The filtrate was concentrated under reduced pressure and the residue purified by preparative SFC (Column: Viridis BEH Prep 2-EP OBD 3×15 cm, 5 μm; Mobile Phase A: CO₂, Mobile Phase B: MeOH (containing 1% of a 2 M solution of NH₃ in MeOH); Flow rate: 65 mL/min; Gradient: isocratic 22% B; Column Temperature (° C.): 35; Back Pressure (bar): 100; Wave Length: 220 nm; RT (min): 3.53; Sample Solvent: MeOH; Injection Volume: 2.5 mL) to afford (2S,4R)—N-(4-chloro-2-((3aR,4R,6R,7aS)-3a-(2-(2-((S)-4-(4-chlorophenyl)-2,3,9-trimethyl-6H-thieno[3,2-f][1,2,4]triazolo[4,3-a][1,4]diazepin-6-yl)acetamido)ethyl)-5,5-dimethylhexahydro-4,6-methanobenzo[d][1,3,2]dioxaborol-2-yl)benzyl)-4-hydroxy-1-(3-methyl-2-(3-methylisoxazol-5-yl)butanoyl)pyrrolidine-2-carboxamide (single diastereoisomer with unknown absolute stereochemistry at the centre indicated in the structure) (22.5 mg, yield=57%) as a white solid.

LCMS: (ES, m/z): [M+H]⁺=1009 (LCMS condition: Column: Shim-pack Scepter C18-120; Mobile phase A: Water/MeCN (95:5, v:v) (containing 5 mM NH₄HCO₃); Mobile phase B: MeCN; Flow rate: 1.5 mL/min.; RT: 1.017 min.)

### Example 11

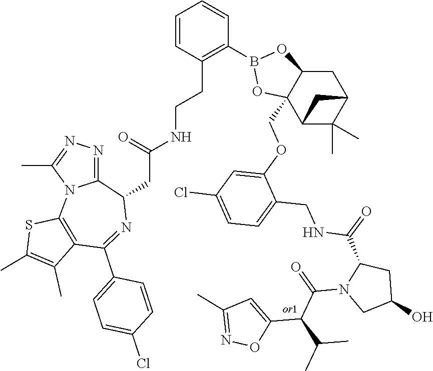 [SMILES: Cc1cc([C@@H](C(=O)N2C[C@H](O)C[C@H]2C(=O)NCc2ccc(Cl)cc2OC[C@]23OB(c4ccccc4CCNC(=O)C[C@@H]4N=C(c5ccc(Cl)cc5)c5c(sc(C)c5C)-n5c(C)nnc54)O[C@H]2C[C@H]2C[C@@H]3C2(C)C)C(C)C)on1]

### (a) Synthesis of Int. 72

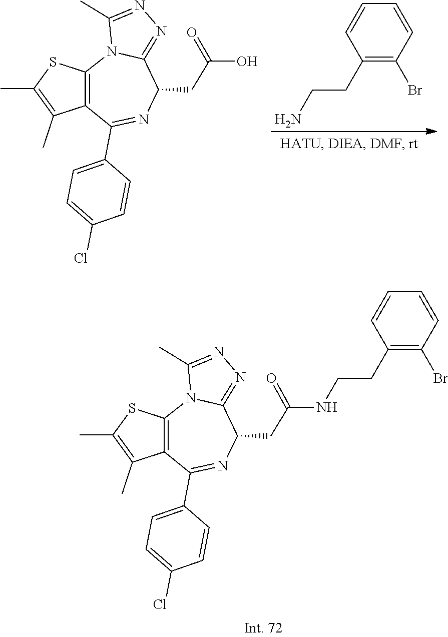 [SMILES: Cc1sc2c(c1C)C(c1ccc(Cl)cc1)=N[C@@H](CC(=O)O)c1nnc(C)n1-2 ; NCCc1ccccc1Br ; Cc1sc2c(c1C)C(c1ccc(Cl)cc1)=N[C@@H](CC(=O)NCCc1ccccc1Br)c1nnc(C)n1-2]

Into a 40 mL vial were added (S)-2-(4-(4-chlorophenyl)-2,3,9-trimethyl-6H-thieno[3,2-f][1,2,4]triazolo[4,3-a][1,4]diazepin-6-yl)acetic acid (500 mg, 1.247 mmol, 1 eq.), DMF (5 mL), 2-(2-bromophenyl)ethanamine (374 mg, 1871 mmol, 1.5 eq.), 2-(2-bromophenyl)ethanamine (374 mg, 1.87 mmol, 1.5 eq.), HATU (711 mg, 1.871 mmol, 1.5 eq.) and DIEA (484 mg, 3.741 mmol, 3 eq.). The resulting mixture was stirred for 1 h at r.t. The mixture was quenched with water at 0° C. and extracted with EtOAc (3×100 mL). The combined organic extracts were washed with brine (3×100 mL) and dried over anhydrous Na₂SO₄. After filtration, the filtrate was concentrated under reduced pressure and the residue purified by reversed-phase flash chromatography on a C18 column using a 30 to 70% gradient of MeCN in water (containing 0.05% formic acid) over 30 min to afford (S)—N-(2-bromophenethyl)-2-(4-(4-chlorophenyl)-2,3,9-trimethyl-6H-thieno[3,2-f][1,2,4]triazolo[4,3-a][1,4]diazepin-6-yl)acetamide (700 mg, yield=96%) as an off-white solid.

LCMS: (ES, m/z): [M+H]⁺=582 (LCMS condition: Column: HALO 90A C18; Mobile phase A: water (containing 0.1% formic acid); Mobile phase B: MeCN (containing 0.1% formic acid); Flow rate: 1.2 mL/min.; RT: 1.033 min.)

### (b) Synthesis of Int. 73

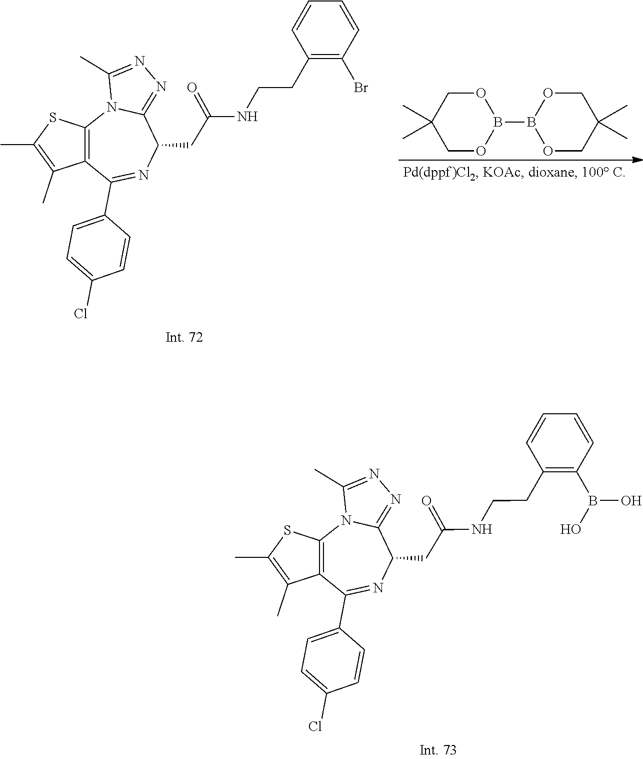

Into a 40 mL vial were added (S)—N-(2-bromophenethyl)-2-(4-(4-chlorophenyl)-2,3,9-trimethyl-6H-thieno[3,2-f][1,2,4]triazolo[4,3-a][1,4]diazepin-6-yl)acetamide (600 mg, 1.029 mmol, 1 eq.), 1,4-dioxane (6 mL), 2-(5,5-dimethyl-1,3,2-dioxaborinan-2-yl)-5,5-dimethyl-1,3,2-dioxaborinane (465 mg, 2.058 mmol, 2 eq.), KOAc (303 mg, 3.087 mmol, 3 eq.) and Pd(dppf)Cl₂·DCM (75 mg, 0.103 mmol, 0.1 eq.) at 16° C. The reaction was stirred for 2 h at 100° C. under nitrogen. The mixture was allowed to cool to r.t., quenched with water at 0° C. and extracted with EtOAc (3×100 mL). The combined organic extracts were washed with brine (100 mL) and dried over anhydrous Na₂SO₄. After filtration, the filtrate was concentrated under reduced pressure and the was purified by reversed-phase flash chromatography on a C18 column using a 60 to 90% gradient of MeCN in water (containing 0.05% formic acid) over 30 min. The crude product was purified by preparative HPLC (Column: XBridge Prep OBD CIS, 30×150 mm, 5 μm; Mobile Phase A: water (containing 10 mmol/L NH₄HCO₃ and 0.05% aqueous NH₃), Mobile Phase B: MeCN; Flow rate: 60 mL/min; Gradient: 56 to 86% B over 8 min; Wave Length: 254 and 220 nm; RT (min): 6.4) to afford (S)-(2-(2-(2-(4-(4-chlorophenyl)-2,3,9-trimethyl-6H-thieno[3,2-f][1,2,4]triazolo[4,3-a][1,4]diazepin-6-yl)acetamido)ethyl)phenyl)boronic acid (276 mg, yield=49%) as a white solid.

LCMS: (ES, m/z): [M+H]⁺=548 (LCMS condition: Column: HALO 90A C18; Mobile phase A: water (containing 0.1% formic acid); Mobile phase B: MeCN (containing 0.1% formic acid); Flow rate: 1.2 mL/min.; RT: 0.852 min.

### (c) Synthesis of Int. 74

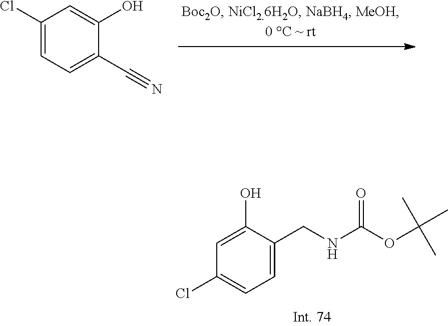 [SMILES: N#Cc1ccc(Cl)cc1O ; CC(C)(C)OC(=O)NCc1ccc(Cl)cc1O]

A solution of 4-chloro-2-hydroxybenzonitrile (13.2 g, 85.954 mmol, 1 eq.) in MeOH (130 mL) was treated with NiCl₂·6H₂O (2.0 g, 8.595 mmol, 0.1 eq.) and Boc₂O (37.5 g, 171.908 mmol, 2 eq.) at 0° C. followed by the addition of NaBH₄ (22.7 g, 601.678 mmol, 7 eq.) in portions at 0° C. The reaction was stirred for 1 h at r.t. To the reaction was added bis(2-aminoethyl)amine (17.7 g, 171.908 mmol, 2 eq.) in portions at r.t. and the mixture stirred for 30 min at r.t. The mixture was quenched with water (100 mL) at 0° C., partially evaporated, and extracted with DCM (3×200 mL). The combined organic extracts were washed with brine (3×100 mL) and dried over anhydrous Na₂SO₄. After filtration, the filtrate was concentrated under reduced pressure and the residue purified by reversed-phase flash chromatography on a C18 column using a 0 to 1000% gradient of MeCN in water (containing mmol/L NH₄HCO₃) over 30 min to afford tert-butyl N-[(4-chloro-2-hydroxyphenyl)methyl]carbamate (13.5 g, yield=52%) as a yellow solid.

LCMS: (ES, m/z): [M−H]⁻=256 (LCMS condition: Column: Shim-pack Scepter C18-120; Mobile phase A: Water/MeCN (95:5, v:v) (containing 5 mM NH₄HCO₃); Mobile phase B: MeCN; Flow rate: 1.5 mL/min; RT: 1.076 min)

### (d) Synthesis of Int. 75

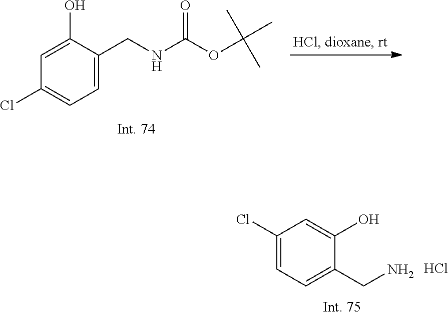 [SMILES: CC(C)(C)OC(=O)NCc1ccc(Cl)cc1O ; NCc1ccc(Cl)cc1O ; Cl]

Into a 250 mL round-bottom flask were added tert-butyl N-[(4-chloro-2-hydroxyphenyl)methyl]carbamate (10.0 g, 38.804 mmol, 1 eq.) and 4 M HCl in 1,4-dioxane (50 mL) at r.t. The resulting mixture was stirred for 2 h at r.t. The resulting mixture was concentrated under reduced pressure to afford 2-(aminomethyl)-5-chlorophenol hydrochloride (6.3 g, yield=98%) as a white solid.

LCMS: (ES, m/z): [M−H]⁻=156 (LCMS condition: Column: Shim-pack Scepter C18-120; Mobile phase A: Water/MeCN (95:5, v:v) (containing 5 mM NH₄HCO₃); Mobile phase B: MeCN; Flow rate: 1.5 mL/min; RT: 0.516 min)

### (e) Synthesis of Int. 76

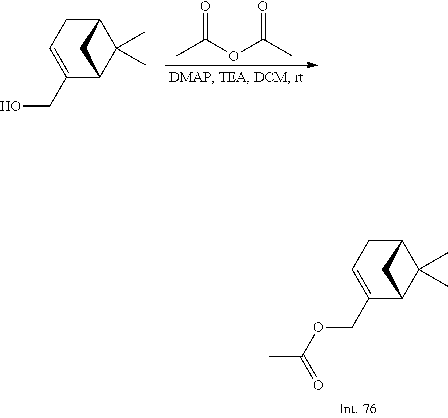

To a stirred solution of [(1R,5S)-6,6-dimethylbicyclo[3.1.1]hept-2-en-2-yl]methanol (3.0 g, 19.706 mmol, 1 eq.), acetic anhydride (2.4 g, 23.647 mmol, 1.2 eq.) and DMAP (24 mg, 0.197 mmol, 0.01 eq.) in DCM (30 mL) was added TEA (4 mL, 29.559 mmol, 1.5 eq.). The reaction was stirred for 16 h at r.t. The mixture was concentrated under reduced pressure and the residue purified by silica gel column chromatography eluting with a gradient of 0 to 100% EtOAc in PE to afford [(1R,5S)-6,6-dimethylbicyclo[3.1.1]hept-2-en-2-yl]methyl acetate (3.6 g, yield=85%) as a colourless oil.

LCMS condition: Column: HALO 90A C18; Mobile phase A: water (containing 0.1% formic acid); Mobile phase B: MeCN (containing 0.1% formic acid); Flow rate: 1.2 mL/min; RT: 1.122 min)

### (f) Synthesis of Int. 77

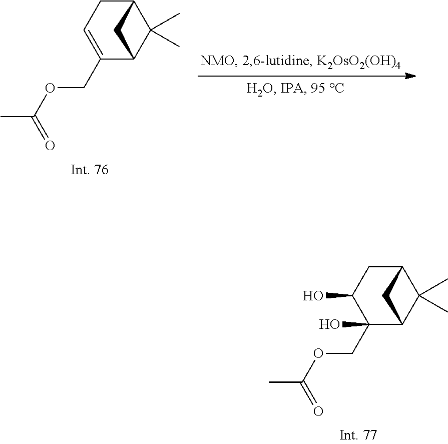 [SMILES: CC(=O)OCC1=CC[C@H]2C[C@@H]1C2(C)C ; CC(=O)OC[C@@]1(O)[C@@H]2C[C@H](C[C@@H]1O)C2(C)C]

To a stirred solution of [(1R,5S)-6,6-dimethylbicyclo[3.1.1]hept-2-en-2-yl]methyl acetate (3.6 g, 18.531 mmol, 1 eq.), NMO (2.6 g, 22.237 mmol, 1.20 eq.) and 2,6-lutidine (2.9 g, 27.796 mmol, 1.5 eq.) in water (5 mL) and IPA (30 mL) was added K₂OsO₂(OH)₄ (682 mg, 1.853 mmol, 0.1 eq.) and the reaction stirred for 16 h at 95° C. The mixture was quenched with water (100 mL) and extracted with EtOAc (3×200 mL). The combined organic extracts were washed with brine (200 mL) and dried over Na₂SO₄. After filtration, the filtrate was concentrated under reduced pressure and the residue was purified by silica gel column chromatography eluting with PE/THF (1:1) to afford [(1R,2S,3S,5R)-2,3-dihydroxy-6,6-dimethylbicyclo[3.1.1]heptan-2-yl]methyl acetate (2.5 g, yield=59%) as a yellow oil.

### (g) Synthesis of Int. 78

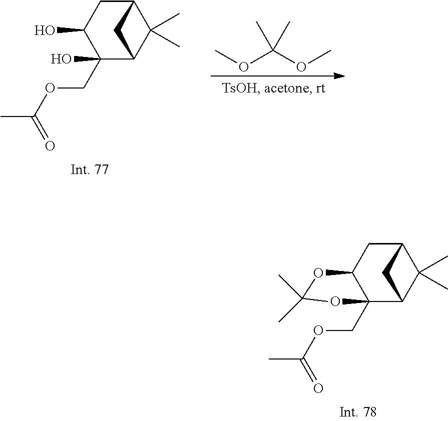

To a stirred solution of [(1R,2S,3S,5R)-2,3-dihydroxy-6,6-dimethylbicyclo[3.1.1]heptan-2-yl]methyl acetate (2.5 g, 10.951 mmol, 1 eq.) in acetone (25 mL) was added TsOH (377 mg, 2.190 mmol, 0.20 eq.) and the reaction stirred for 3 h at r.t. The mixture was concentrated under reduced pressure and the residue purified by silica gel column chromatography eluting with a gradient of 0 to 20% EtOAc in PE to afford ((3aS,4R,6R,7aS)-2,2,5,5-tetramethyltetrahydro-4,6-methanobenzo[d][1,3]dioxol-3a(4H)-yl)methyl acetate (2.0 g, yield=65%) as a colourless oil.

### (h) Synthesis of Int. 79

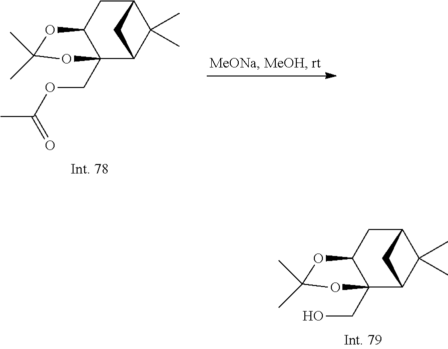

To a stirred solution of ((3aS,4R,6R,7aS)-2,2,5,5-tetramethyltetrahydro-4,6-methanobenzo[d][1,3]dioxol-3a(4H)-yl)methyl acetate (2.0 g, 7.453 mmol, 1 eq.) in MeOH (20 mL) was added MeONa (483 mg, 8.944 mmol, 1.2 eq.) and the reaction stirred for 16 h at r.t. The mixture was concentrated under reduced pressure and the residue purified by silica gel column chromatography eluting with a gradient of 0 to 40% EtOAc in PE to afford ((3aS,4R,6R,7aS)-2,2,5,5-tetramethyltetrahydro-4,6-methanobenzo[d][1,3]dioxol-3a(4H)-yl)methanol (1.3 g, yield=73%) as a colourless oil.

### (i) Synthesis of Int. 80

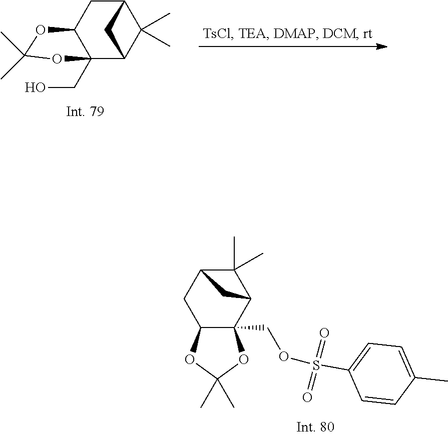

Into a 20 mL vial were added ((3aS,4R,6R,7aS)-2,2,5,5-tetramethyltetrahydro-4,6-methanobenzo[d][1,3]dioxol-3a(4H)-yl)methanol (500 mg, 2.209 mmol, 1 eq.), p-toluenesulfonyl chloride (463 mg, 2.430 mmol, 1.1 eq.), TEA (447 mg, 4.418 mmol, 2 eq.), DMAP (134 mg, 1.105 mmol, 0.5 eq.) and DCM (3 mL) at r.t. The reaction was stirred for 16 h at r.t. The mixture was concentrated under reduced pressure and the residue purified by silica gel column chromatography eluting with PE/THF (1:1) to afford ((3aS,4R,6R,7aS)-2,2,5,5-tetramethyltetrahydro-4,6-methanobenzo[d][1,3]dioxol-3a(4H)-yl)methyl 4-methylbenzenesulfonate (400 mg, yield=40%) as a yellow solid.

LCMS: (ES, m/z): [M+H]⁺=381 (LCMS condition: Column: HALO 90A C18; Mobile phase A: water (containing 0.1% formic acid); Mobile phase B: MeCN (containing 0.1% formic acid); Flow rate: 1.2 mL/min; RT: 1.195 min)

### (j) Synthesis of Int. 81

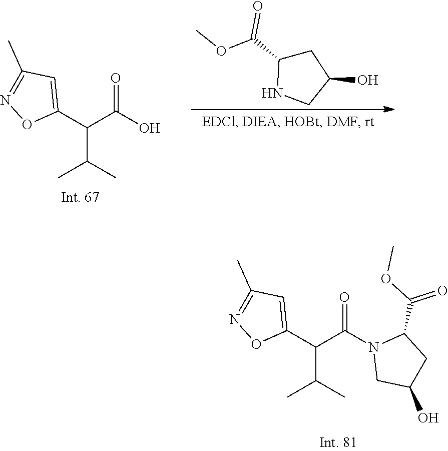

Into a 100 mL round-bottom flask were added 3-methyl-2-(3-methyl-1,2-oxazol-5-yl)butanoic acid (racemic) (3.3 g, 18.012 mmol, 1 eq.), DMF (30 mL), methyl (2S,4R)-4-hydroxypyrrolidine-2-carboxylate (2.6 g, 18.012 mmol, 1 eq.), EDCI (5.1 g, 27.018 mmol, 1.5 eq.), HOBt (3.6 g, 27.018 mmol, 1.5 eq.) and DIEA (11.6 g, 90.060 mmol, 5 eq.) at r.t.

The reaction was stirred for 2 h at r.t. The mixture was purified by reversed-phase flash chromatography on a C18 column using a 20 to 60% gradient of MeCN in water (containing 10 mmol/L NH₄HCO₃) over 35 min to afford methyl (2S,4R)-4-hydroxy-1-(3-methyl-2-(3-methylisoxazol-5-yl)butanoyl)pyrrolidine-2-carboxylate (mixture of diastereoisomers) (2.3 g, yield=51%) as a brown oil.

LCMS: (ES, m/z): [M+H]⁺=311 (LCMS condition: Column: Shim-pack Scepter C18-120; Mobile phase A: Water/MeCN (95:5, v:v) (containing 5 mM NH₄HCO₃); Mobile phase B: MeCN; Flow rate: 1.5 mL/min; RT: 1.225 min

### (k) Synthesis of Int. 82

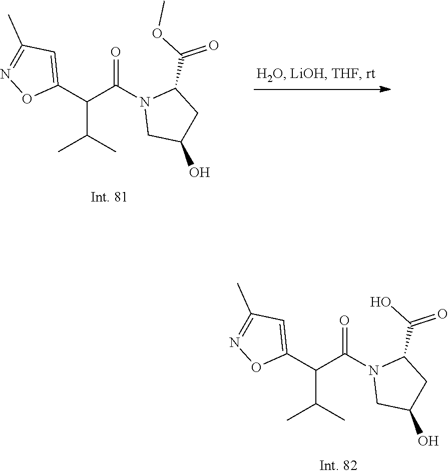 [SMILES: COC(=O)[C@@H]1C[C@@H](O)CN1C(=O)C(c1cc(C)no1)C(C)C ; Cc1cc(C(C(=O)N2C[C@H](O)C[C@H]2C(=O)O)C(C)C)on1]

Into a 100 mL round-bottom flask were added methyl (2S,4R)-4-hydroxy-1-[3-methyl-2-(3-methyl-1,2-oxazol-5-yl)butanoyl]pyrrolidine-2-carboxylate (mixture of diastereoisomers) (3.3 g, 0.322 mmol, 1 eq.), THF (30 mL), water (7.5 mL), and LIOH (2.5 g, 106.330 mmol, 10 eq.). The reaction was stirred for 2 h at r.t. The mixture was quenched with 1 N HCl (aq.) and extracted with EtOAc (3×300 mL). The combined organic extracts were washed with brine (3×100 mL) and dried over anhydrous Na₂SO₄. After filtration, the filtrate was concentrated under reduced pressure to afford (2S,4R)-4-hydroxy-1-(3-methyl-2-(3-methylisoxazol-5-yl)butanoyl)pyrrolidine-2-carboxylic acid (mixture of diastereoisomers) (3.0 g, yield=94%) as a light yellow solid.

LCMS: (ES, m/z): [M+H]⁺=297 (LCMS condition: Column: HALO 90A C18; Mobile phase A: water (containing 0.1% formic acid); Mobile phase B: MeCN (containing 0.1% formic acid); Flow rate: 1.2 mL/min.; RT: 0.572 min.)

### (l) Synthesis of Int. 83

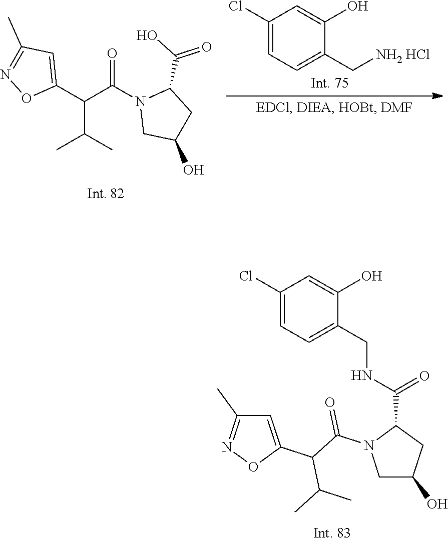 [SMILES: Cc1cc(C(C(=O)N2C[C@H](O)C[C@H]2C(=O)O)C(C)C)on1 ; NCc1ccc(Cl)cc1O ; Cl ; Cc1cc(C(C(=O)N2C[C@H](O)C[C@H]2C(=O)NCc2ccc(Cl)cc2O)C(C)C)on1]

Into a 250 mL round-bottom flask were added (2S,4R)-4-hydroxy-1-(3-methyl-2-(3-methylisoxazol-5-yl)butanoyl)pyrrolidine-2-carboxylic acid (mixture of diastereoisomers) (2.8 g, 9.449 mmol, 1 eq.), 2-(aminomethyl)-5-chlorophenol hydrochloride (1.6 g, 10.394 mmol, 1.1 eq.), HATU (5.3 g, 14.174 mmol, 1.5 eq.), DIEA (6.1 g, 47.245 mmol, 5 eq.) and DMF (30 mL). The reaction was stirred for 1 h at r.t. The mixture was purified by reversed-phase flash chromatography on a C18 column using a 50 to 60% gradient of MeCN in water (containing 0.1% formic acid) over 20 min to afford (2S,4R)—N-(4-chloro-2-hydroxybenzyl)-4-hydroxy-1-(3-methyl-2-(3-methylisoxazol-5-yl)butanoyl)pyrrolidine-2-carboxamide (mixture of diastereoisomers) (1.7 g, yield=43%) as a white solid.

LCMS: (ES, m/z): [M+H]⁺=436 (LCMS condition: Column: Shim-pack Scepter C18-120; Mobile phase A: Water/MeCN (95:5, v:v) (containing 5 mM NH₄HCO₃); Mobile phase B: MeCN; Flow rate: 1.5 mL/min; RT: 1.486 min)

### (m) Separation of Int. 83 Diastereoisomers

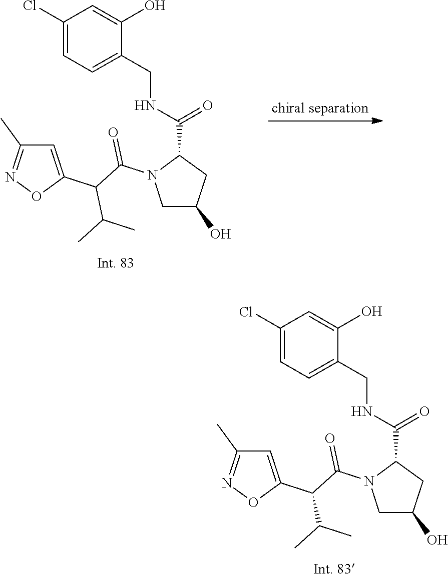 [SMILES: Cc1cc(C(C(=O)N2C[C@H](O)C[C@H]2C(=O)NCc2ccc(Cl)cc2O)C(C)C)on1 ; Cc1cc([C@H](C(=O)N2C[C@H](O)C[C@H]2C(=O)NCc2ccc(Cl)cc2O)C(C)C)on1]

(2S,4R)—N-(4-Chloro-2-hydroxybenzyl)-4-hydroxy-1-(3-methyl-2-(3-methylisoxazol-5-yl)butanoyl)pyrrolidine-2-carboxamide (mixture of diastereoisomers) was separated by preparative chiral HPLC (Column: Chiralpak IF, 2×25 cm, 5 μm; Mobile phase A: hexane, Mobile phase B: EtOH:DCM (1:1); Flow rate: 20 mL/min; Gradient: isocratic 30% B; Wave length: 254 and 220 nm; RT peak 1 (min): 4.35; RT peak 2 (min): 10.212; Sample Solvent: EtOH; Injection volume: 0.7 mL; Number of runs: 5) to afford (2S,4R)—N-(4-chloro-2-hydroxybenzyl)-4-hydroxy-1-((R)-3-methyl-2-(3-methylisoxazol-5-yl)butanoyl)pyrrolidine-2-carboxamide (absolute stereochemistry confirmed with a single crystal X-ray structure) (single diastereoisomer with unknown absolute stereochemistry at the centre indicated in the structure) (800 mg, yield=46%) as a white solid.

LCMS: (ES, m/z): [M+H]⁺=436 (LCMS condition: Column: HALO 90A C18; Mobile phase A: Water/MeCN (95:5, v:v) (containing 5 mM NH₄HCO₃); Mobile phase B: MeCN; Flow rate: 1.2 mL/min; RT: 1.466 min)

### (n) Synthesis of Int. 84

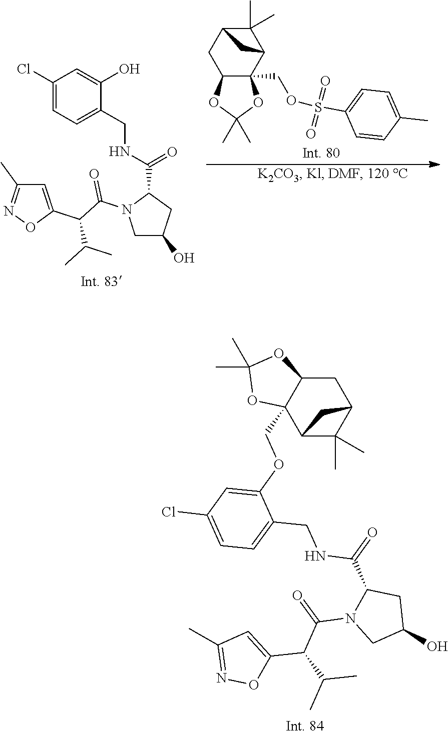 [SMILES: Cc1cc([C@H](C(=O)N2C[C@H](O)C[C@H]2C(=O)NCc2ccc(Cl)cc2O)C(C)C)on1 ; Cc1ccc(S(=O)(=O)OC[C@]23OC(C)(C)O[C@H]2C[C@H]2C[C@@H]3C2(C)C)cc1 ; Cc1cc([C@H](C(=O)N2C[C@H](O)C[C@H]2C(=O)NCc2ccc(Cl)cc2OC[C@]23OC(C)(C)O[C@H]2C[C@H]2C[C@@H]3C2(C)C)C(C)C)on1]

Into a 40 mL sealed tube were added (2S,4R)—N-(4-chloro-2-hydroxybenzyl)-4-hydroxy-1-((R)-3-methyl-2-(3-methylisoxazol-5-yl)butanoyl)pyrrolidine-2-carboxamide (250 mg, 0.574 mmol, 1 eq.), [(1R,2S,6S,8R)-4,4,9,9-tetramethyl-3,5-dioxatricyclo[6.1.1.0\hat{ }{2,6}]decan-2-yl]methyl 4-methylbenzenesulfonate (327 mg, 0.861 mmol, 1.5 eq.), K₂C03 (237 mg, 1.722 mmol, 3 eq.), KI (95 mg, 0.574 mmol, 1 eq.) and DMF (2 mL) at r.t. The reaction was stirred for 16 h at 120° C. The mixture was allowed to cool to r.t. and purified by reversed-phase flash chromatography on a C18 column using a 45 to 55% gradient of MeCN in water (containing 0.1% 10 mmol/L NH₄HCO₃) over 30 min. The crude product was purified by preparative HPLC (Column: XBridge Prep OBD C18, 30×150 mm, 5 μm; Mobile phase A: Water (10 mmol/L NH₄HCO₃), Mobile Phase B: MeCN; Flow rate: 60 mL/min; Gradient: 47 to 77% B over 10 min; Wave length: 254 and 220 nm; RT (min): 7.5) to afford (2S,4R)—N-(4-chloro-2-(((3aS,4R,6R,7aS)-2,2,5,5-tetramethyltetrahydro-4,6-methanobenzo[d][1,3]dioxol-3a(4H)-yl)methoxy)benzyl)-4-hydroxy-1-(3-methyl-2-(3-methylisoxazol-5-yl)butanoyl)pyrrolidine-2-carboxamide (mixture of diastereoisomers due to epimerisation in the reaction) (200 mg, yield=53%) as a white solid.

LCMS: (ES, m/z): [M−H]⁻=642 (LCMS condition: Column: HALO 90A C18; Mobile phase A: Water/MeCN (95:5, v:v) (containing 5 mM NH₄HCO₃); Mobile phase B: MeCN; Flow rate: 1.2 mL/min; RT: 0.933 min)

### (o) Separation of Int. 84 Diastereoisomers

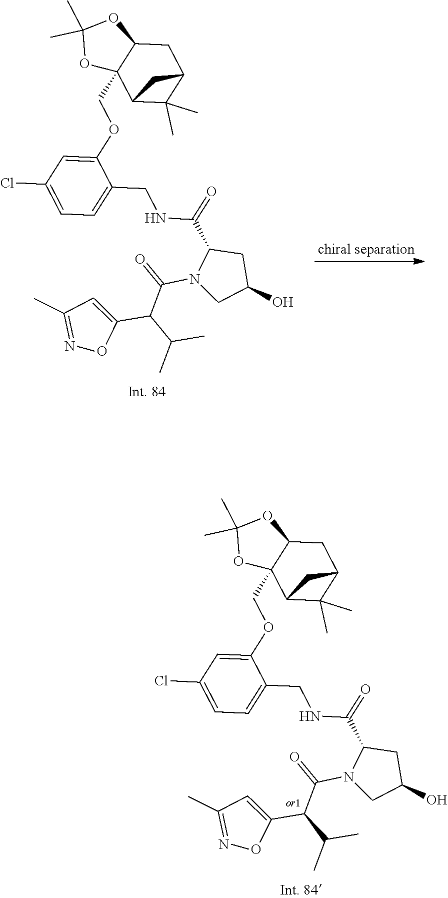 [SMILES: Cc1cc(C(C(=O)N2C[C@H](O)C[C@H]2C(=O)NCc2ccc(Cl)cc2OC[C@]23OC(C)(C)O[C@H]2C[C@H]2C[C@@H]3C2(C)C)C(C)C)on1 ; Cc1cc([C@@H](C(=O)N2C[C@H](O)C[C@H]2C(=O)NCc2ccc(Cl)cc2OC[C@]23OC(C)(C)O[C@H]2C[C@H]2C[C@@H]3C2(C)C)C(C)C)on1]

(2S,4R)—N-(4-Chloro-2-(((3aS,4R,6R,7aS)-2,2,5,5-tetramethyltetrahydro-4,6-methanobenzo[d][1,3]dioxol-3a(4H)-yl)methoxy)benzyl)-4-hydroxy-1-(3-methyl-2-(3-methylisoxazol-5-yl)butanoyl)pyrrolidine-2-carboxamide (mixture of diastereoisomers) was separated by preparative chiral SFC (Column: Chiralpak IG 3×25 cm, 5 μm; Mobile phase A: CO₂, Mobile Phase B: IPA (containing 1% of a 2 M solution of NH₃ in IPA); Flow rate: 85 mL/min; Gradient: isocratic 34% B; Column temperature (° C.): 35; Back pressure (bar): 100; Wave length: 220 nm; RT peak 1 (min): 3.27; RT peak 2 (min): 4.9; Sample solvent: MeOH; Injection volume: 1 mL; Number of runs: 6) to afford (2S,4R)—N-(4-chloro-2-(((3aS,4R,6R,7aS)-2,2,5,5-tetramethyltetrahydro-4,6-methanobenzo[d][1,3]dioxol-3a(4H)-yl)methoxy)benzyl)-4-hydroxy-1-(3-methyl-2-(3-methylisoxazol-5-yl)butanoyl)pyrrolidine-2-carboxamide (single diastereoisomer with unknown absolute stereochemistry at the centre indicated in the structure) (80 mg, yield=40%) as a white solid.

LCMS: (ES, m/z): [M+H]+=644 (LCMS condition: Column: Shim-pack Scepter C18-120; Mobile phase A: Water/MeCN (95:5, v:v) (containing 5 mM NH₄HCO₃); Mobile phase B: MeCN; Flow rate: 1.5 mL/min; RT: 0.946 min)

### (p) Synthesis of Int. 85

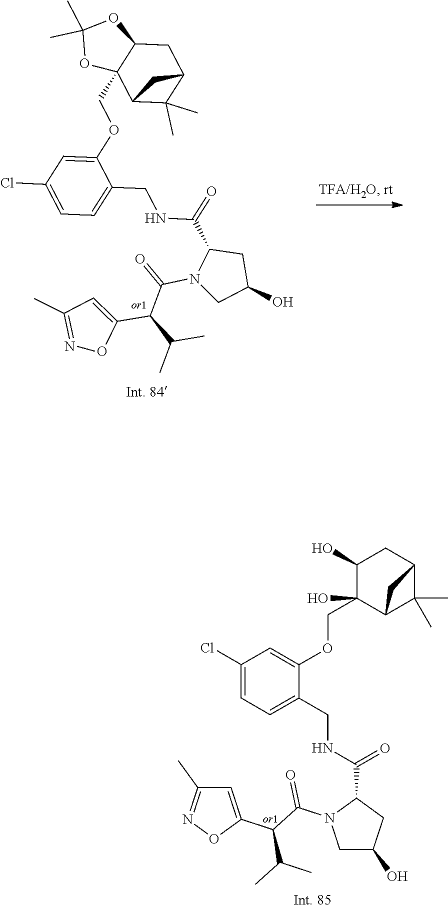

Into an 8 mL vial were added (2S,4R)—N-(4-chloro-2-(((3aS,4R,6R,7aS)-2,2,5,5-tetramethyltetrahydro-4,6-methanobenzo[d][1,3]dioxol-3a(4H)-yl)methoxy)benzyl)-4-hydroxy-1-(3-methyl-2-(3-methylisoxazol-5-yl)butanoyl)pyrrolidine-2-carboxamide (single diastereoisomer with unknown absolute stereochemistry at the centre indicated in the structure) (80 mg, 0.124 mmol, 1 eq.), TFA (2 mL), and water (2 mL) at r.t. The reaction was stirred for 2 h at r.t. The mixture was purified by reversed-phase flash chromatography on a C18 column using a 50 to 60% gradient of MeCN in water (containing 10 mmol/L NH₄HCO₃) over 30 min. The crude product was purified by preparative HPLC (Column: XBridge Prep OBD Shield RP18, 19×250 mm, 5 μm; Mobile phase A: water (containing 0.1% formic acid), Mobile phase B: MeOH; Flow rate: 25 mL/min; Gradient: 61 to 91% B over 8 min; Wave length: 254 and 220 nm; RT (min): 6.82) to afford (2S,4R)—N-(4-chloro-2-(((1R,2S,3S,5R)-2,3-dihydroxy-6,6-dimethylbicyclo[3.1.1]heptan-2-yl)methoxy)benzyl)-4-hydroxy-1-(3-methyl-2-(3-methylisoxazol-5-yl)butanoyl)pyrrolidine-2-carboxamide (second eluting peak) (single diastereoisomer with unknown absolute stereochemistry at the centre indicated in the structure) (34.4 mg, yield=45%) as a white solid.

LCMS: (ES, m/z): [M+H]⁺=604 (LCMS condition: Column: Shim-pack Scepter C18-120; Mobile phase A: Water/MeCN (95:5, v:v) (containing 5 mM NH₄HCO₃); Mobile phase B: MeCN; Flow rate: 1.5 mL/min; RT: 1.087 min)

### (q) Synthesis of Example 11

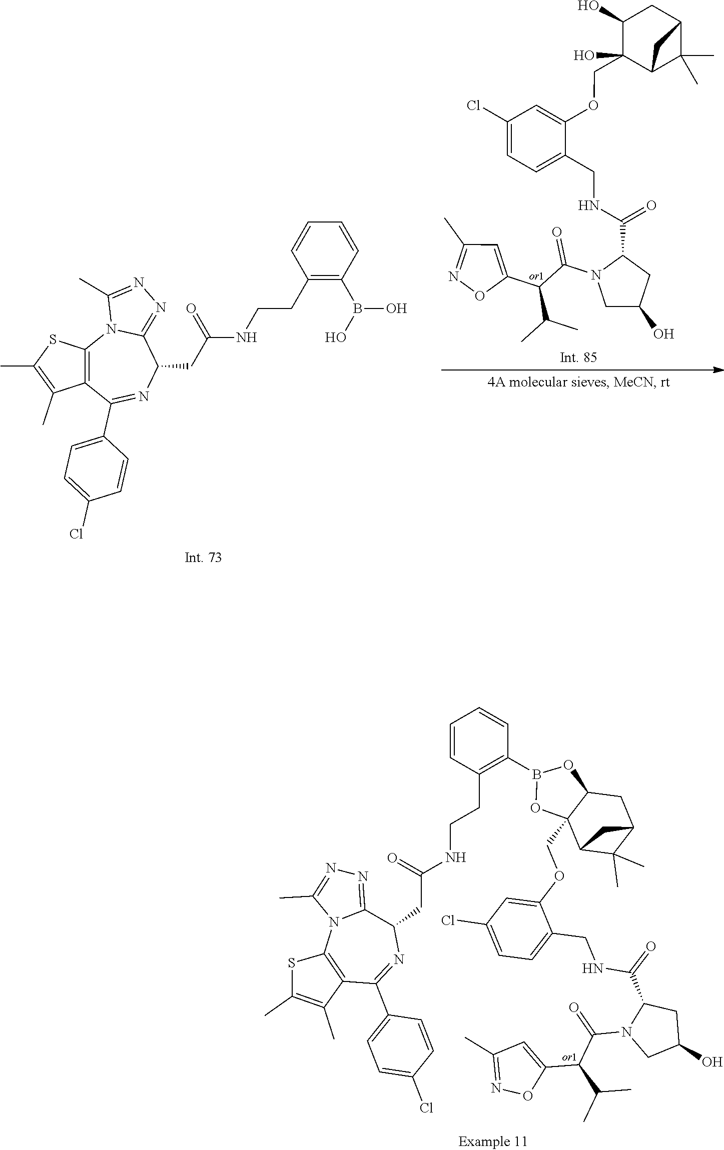

A solution of (S)-(2-(2-(2-(4-(4-chlorophenyl)-2,3,9-trimethyl-6H-thieno[3,2-f][1,2,4]triazolo[4,3-a][1,4]diazepin-6-yl)acetamido)ethyl)phenyl)boronic acid (single diastereoisomer with unknown absolute stereochemistry at the centre indicated in the structure) (17 mg, 0.029 mmol, 1 eq.) and molecular sieves (4 Å) (106 mg) in MeCN (3 mL) was stirred for 16 h at r.t. under nitrogen. The mixture was filtered, and the filter cake washed with MeOH (3×3 mL). The filtrate was concentrated under reduced pressure and the residue purified by preparative SFC (Column: Torus Diol OBD 3×25 cm, 5 μm; Mobile phase A: CO₂, Mobile phase B: MeOH (containing 1% of a 2 M solution of NH₃ in MeOH); Flow rate: 75 mL/min; Gradient: isocratic 24% B; Column temperature (° C.): 35; Back pressure (bar): 100; Wave length: 220 nm; RT (min): 5.41; Sample solvent: MeOH; Injection volume: 1 mL) to afford (2S,4R)—N-(4-chloro-2-(((3aS,4R,6R,7aS)-2-(2-(2-(2-((S)-4-(4-chlorophenyl)-2,3,9-trimethyl-6H-thieno[3,2-f][1,2,4]triazolo[4,3-a][1,4]diazepin-6-yl)acetamido)ethyl)phenyl)-5,5-dimethyltetrahydro-4,6-methanobenzo[d][1,3,2]dioxaborol-3a(4H)-yl)methoxy)benzyl)-4-hydroxy-1-(3-methyl-2-(3-methylisoxazol-5-yl)butanoyl)pyrrolidine-2-carboxamide (single diastereoisomer with unknown absolute stereochemistry at the centre indicated in the structure) (17.1 mg, yield=52%) as a white solid.

LCMS: (ES, m/z): [M+H]⁺=1115 (LCMS condition: Column: HALO 90A C18; Mobile phase A: water (containing 0.1% formic acid); Mobile phase B: MeCN (containing 0.1% formic acid); Flow rate: 1.2 mL/min; RT: 1.312 min)

### Example 12

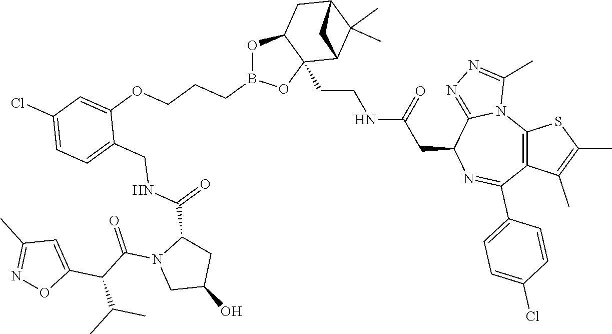

### (a) Synthesis of Int. 86

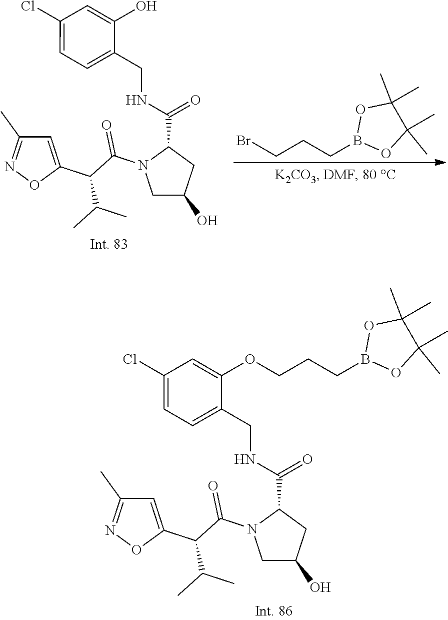

Into a 40 mL sealed tube were added (2S,4R)—N-(4-chloro-2-hydroxybenzyl)-4-hydroxy-1-((R)-3-methyl-2-(3-methylisoxazol-5-yl)butanoyl)pyrrolidine-2-carboxamide (250 mg, 0.574 mmol, 1 eq.), 2-(3-bromopropyl)-4,4,5,5-tetramethyl-1,3,2-dioxaborolane (713 mg, 2.870 mmol, 5 eq.), K₂CO₃ (237 mg, 1.722 mmol, 3 eq.), KI (95 mg, 0.574 mmol, 1 eq.) and DMF (5 mL) at r.t. The reaction was stirred for 3 h at 80° C. The mixture was allowed to cool to r.t. and the residue purified by reversed-phase flash chromatography on a C18 column using a 50 to 60% gradient of MeCN in water (containing 10 mmol/L NH₄HCO₃) over 20 min to afford (2S,4R)—N-(4-chloro-2-(3-(4,4,5,5-tetramethyl-1,3,2-dioxaborolan-2-yl)propoxy)benzyl)-4-hydroxy-1-((R)-3-methyl-2-(3-methylisoxazol-5-yl)butanoyl)pyrrolidine-2-carboxamide (200 mg, yield=40%) as a colourless solid.

LCMS: (ES, m/z): [M+H]⁺=604 (LCMS condition: Column: Kinetex EVO C18; Mobile phase A: water (containing 6.5 mM NH₄HCO₃ and ammonia hydroxide (pH=10)); Mobile phase B: MeCN; Flow rate: 1.5 mL/min; RT: 1.013 min)

### (b) Synthesis of Int. 87

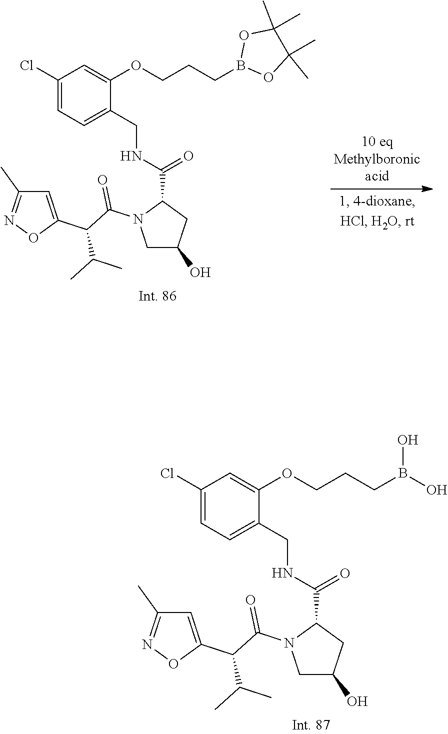

Into an 8 mL sealed tube were added ((2S,4R)—N-(4-chloro-2-(3-(4,4,5,5-tetramethyl-1,3,2-dioxaborolan-2-yl)propoxy)benzyl)-4-hydroxy-1-((R)-3-methyl-2-(3-methylisoxazol-5-yl)butanoyl)pyrrolidine-2-carboxamide (200 mg, 0.033 mmol, 1 eq.), methylboronic acid (198 mg, 3.310 mmol, 10 eq.), 4 M HCl in 1,4-dioxane (2 mL) and water (0.4 mL). The reaction was stirred for 1 h at r.t. The mixture was concentrated under reduced pressure and the residue purified by reversed-phase flash chromatography on a C18 column using a 50 to 60% gradient of MeCN in water (containing 10 mmol/L NH₄HCO₃) over 30 min. The crude product was purified by preparative HPLC (Column: XBridge Prep OBD Shield RP18, 19×250 mm, 5 μm; Mobile phase A: water (containing 0.1% formic acid), Mobile phase B: MeOH; Flow rate: 25 mL/min; Gradient: 55 to 71% B over 8 min; Wave length: 254 and 220 nm; RT (min): 7.2) to afford (3-(5-chloro-2-(((2S,4R)-4-hydroxy-1-((R)-3-methyl-2-(3-methylisoxazol-5-yl)butanoyl)pyrrolidine-2-carboxamido)methyl)phenoxy)propyl)boronic acid (54.6 mg, yield=31%) as a white solid.

LCMS: (ES, m/z): [M−H]⁻=520 (LCMS condition: Column: HALO 90A C18; Mobile phase A: water (containing 0.1% formic acid); Mobile phase B: MeCN (containing 0.1% formic acid) Flow rate: 1.2 mL/min; RT: 0.773 min)

### (c) Synthesis of Example 12

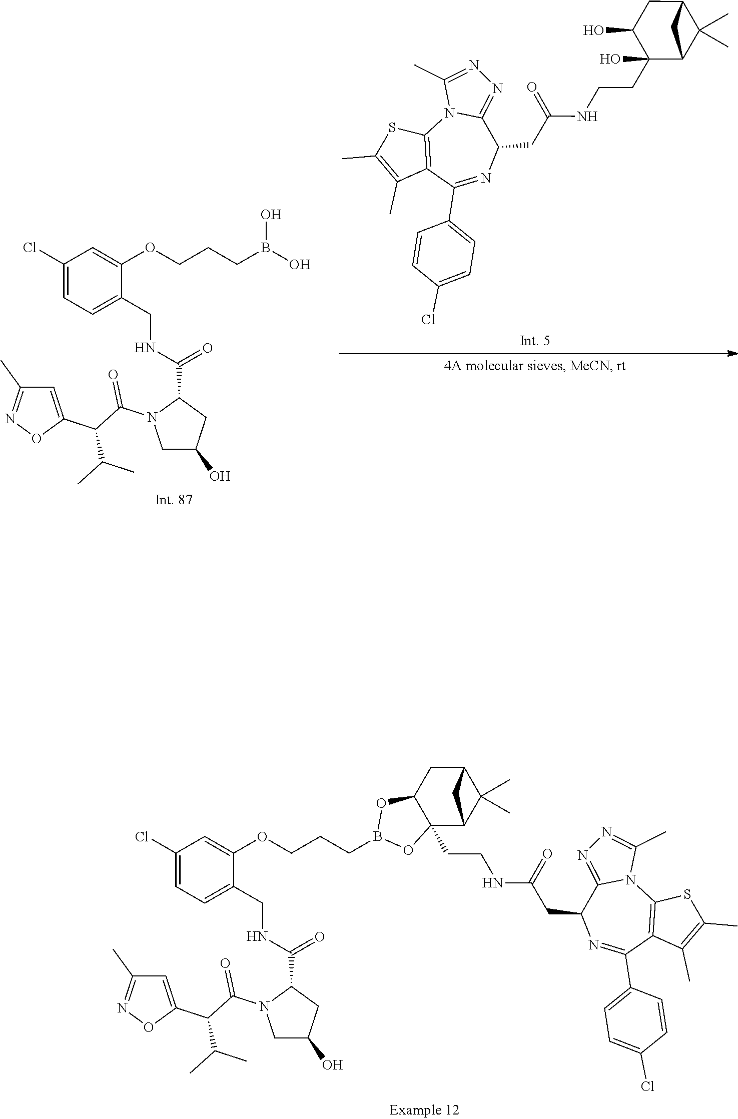

A solution of (3-(5-chloro-2-(((2S,4R)-4-hydroxy-1-((R)-3-methyl-2-(3-methylisoxazol-5-yl)butanoyl)pyrrolidine-2-carboxamido)methyl)phenoxy)propyl)boronic acid (23 mg, 0.044 mmol, 1 eq.), 2-[(9S)-7-(4-chlorophenyl)-4,5,13-trimethyl-3-thia-1,8,11,12-tetraazatricyclo[8.3.0.0\hat{ }{2,6}]trideca-2(6),4,7,10,12-pentaen-9-y]-N-{2-[(1R,2R,3S,5R)-2,3-dihydroxy-6,6-dimethylbicyclo[3.1.1]heptan-2-yl]ethyl}acetamide (25 mg, 0.044 mmol, 1 eq.) and molecular sieves (4 Å) (174 mg) in MeCN (3 mL) was stirred for 16 h at r.t. under nitrogen. The mixture was filtered, and the filter cake washed with MeOH (3×3 mL). The filtrate was concentrated under reduced pressure and the crude product (51 mg) purified by preparative SFC (Column: Torus Diol OBD 3×25 cm, 5 μm; Mobile Phase A: CO₂, Mobile phase B: MeOH (containing 1% of a 2 M solution of NH₃ in MeOH); Flow rate: 75 mL/min; Gradient: isocratic 24% B; Column temperature (° C.): 35; Back pressure (bar): 100; Wave length: 220 nm; RT (min): 5.41; Sample solvent: MeOH; Injection volume: 1 mL) to afford (2S,4R)—N-(4-chloro-2-(3-((3aR,4R,6R,7aS)-3a-(2-(2-((S)-4-(4-chlorophenyl)-2,3,9-trimethyl-6H-thieno[3,2-f][1,2,4]triazolo[4,3-a][1,4]diazepin-6-yl)acetamido)ethyl)-5,5-dimethylhexahydro-4,6-methanobenzo[d][1,3,2]dioxaborol-2-yl)propoxy)benzyl)-4-hydroxy-1-((R)-3-methyl-2-(3-methylisoxazol-5-yl)butanoyl)pyrrolidine-2-carboxamide (28 mg, yield=57%) as a white solid.

LCMS: (ES, m/z): [M+H]⁺=1067 (LCMS condition: Column: HALO 90A C18; Mobile phase A: water (containing 0.1% formic acid); Mobile phase B: MeCN (containing 0.1% formic acid); Flow rate: 1.2 mL/min.; RT: 1.226 min.)

### Example 13

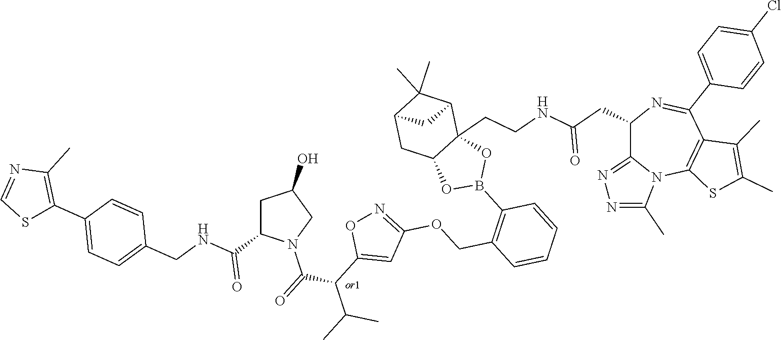 [SMILES: Cc1ncsc1-c1ccc(CNC(=O)[C@@H]2C[C@@H](O)CN2C(=O)[C@H](c2cc(OCc3ccccc3B3O[C@@H]4C[C@@H]5C[C@@H](C5(C)C)[C@]4(CCNC(=O)C[C@@H]4N=C(c5ccc(Cl)cc5)c5c(sc(C)c5C)-n5c(C)nnc54)O3)no2)C(C)C)cc1]

### (a) Synthesis of Int. 88

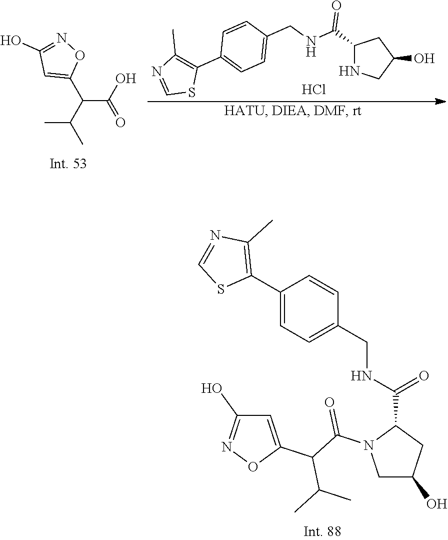

To a stirred solution of 2-(3-hydroxy-1,2-oxazol-5-yl)-3-methylbutanoic acid (racemic) (2.0 g, 10.800 mmol, 1.00 eq.), (2S,4R)-4-hydroxy-N-{[4-(4-methyl-1,3-thiazol-5-yl)phenyl]methyl}pyrrolidine-2-carboxamide hydrochloride (3.4 g, 10.800 mmol, 1.00 eq.) and HATU (6.2 g, 16.200 mmol, 1.5 eq.) in anhydrous DMF (20 mL) was added DIEA (9.4 mL, 54.000 mmol, 5.00 eq.) at 0° C. The reaction was stirred at 25° C. for 2 h. The mixture was purified by reversed-phase flash chromatography on a C18 column using a 0 to 50% gradient of MeCN in water (containing 0.05% NH₄HCO₃) over 10 min to afford (2S,4R)-4-hydroxy-1-[2-(3-hydroxy-1,2-oxazol-5-yl)-3-methylbutanoyl]-N-{[4-(4-methyl-1,3-thiazol-5-yl)phenyl]methyl}pyrrolidine-2-carboxamide (mixture of diastereoisomers) (4.27 g, yield=80%) as a light yellow solid.

LCMS: (ES, m/z): [M+H]⁺=485 (LCMS condition: Column: Shim-pack Scepter C18-120; Mobile phase A: water (containing 5 mM NH₄HCO₃); Mobile phase B: MeCN; Flow rate: 1.5 mL/min.; RT: 0.570 min.)

### (b) Synthesis of Int. 89

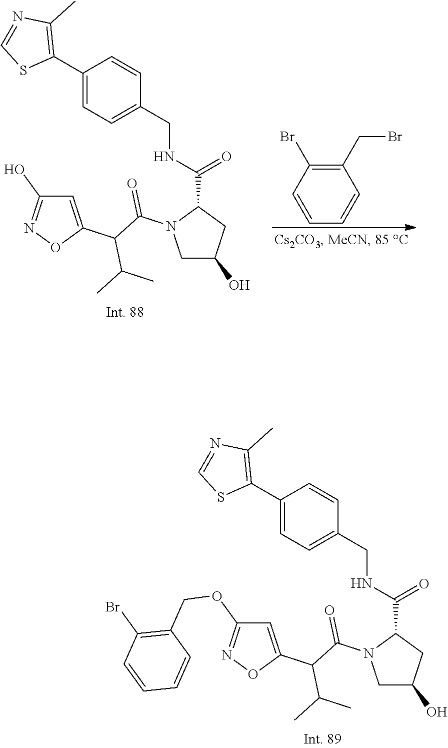 [SMILES: Cc1ncsc1-c1ccc(CNC(=O)[C@@H]2C[C@@H](O)CN2C(=O)C(c2cc(O)no2)C(C)C)cc1 ; BrCc1ccccc1Br ; Cc1ncsc1-c1ccc(CNC(=O)[C@@H]2C[C@@H](O)CN2C(=O)C(c2cc(OCc3ccccc3Br)no2)C(C)C)cc1]

To a stirred solution of (2S,4R)-4-hydroxy-1-[2-(3-hydroxy-1,2-oxazol-5-yl)-3-methylbutanoyl]-N-{[4-(4-methyl-1,3-thiazol-5-yl)phenyl]methyl}pyrrolidine-2-carboxamide (mixture of diastereoisomers) (4.2 g, 8.812 mmol, 1 eq.) and 1-bromo-2-(bromomethyl)benzene (3.7 g, 14.980 mmol, 1.7 eq.) in MeCN (50 mL) was added Cs₂CO₃ (8.6 g, 26.436 mmol, 3 eq.) at 25° C. The reaction was stirred at 85° C. for 4 h. The mixture was filtered and the filter cake washed with DCM (2×50 mL). The filtrate was concentrated under reduced pressure and the residue purified by silica gel column chromatography eluting with DCM/MeOH (10:1) to afford (2S,4R)-1-(2-{3-[(2-bromophenyl)methoxy]-1,2-oxazol-5-yl}-3-methylbutanoyl)-4-hydroxy-N-{[4-(4-methyl-1,3-thiazol-5-yl)phenyl]methyl}pyrrolidine-2-carboxamide (mixture of diastereoisomers) (2.37 g, yield=32%) as a yellow solid.

LCMS: (ES, m/z): [M+H]⁺=655 (LCMS condition: Column: HALO 90A C18; Mobile phase A: water (containing 0.1% formic acid); Mobile phase B: MeCN (containing 0.1% formic acid); Flow rate: 1.2 mL/min.; RT: 1.425 min.)

### (c) Synthesis of Int. 90

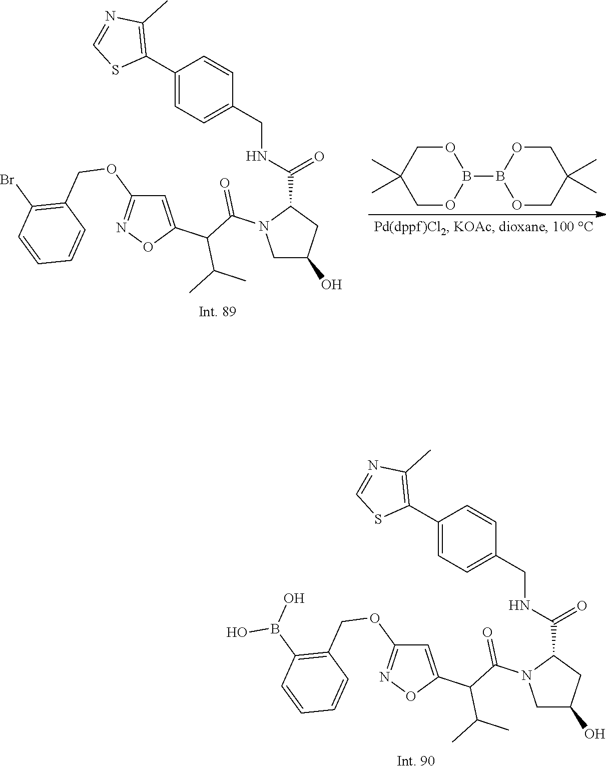

To a stirred solution of (2S,4R)-1-(2-{3-[(2-bromophenyl)methoxy]-1,2-oxazol-5-yl}-3-methylbutanoyl)-4-hydroxy-N-{[4-(4-methyl-1,3-thiazol-5-yl)phenyl]methyl}pyrrolidine-2-carboxamide (mixture of diastereoisomers) (2.37 g, 3.626 mmol, 1 eq.), 2-(5,5-dimethyl-1,3,2-dioxaborinan-2-yl)-5,5-dimethyl-1,3,2-dioxaborinane (4.10 g, 18.13 mmol, 5 eq.) and Pd(dppf)Cl₂·DCM (300 mg, 0.363 mmol, 0.1 eq.) in anhydrous 1,4-dioxane (24 mL) was added KOAc (1.78 g, 18.13 mmol, 5 eq.). The reaction was stirred at 90° C. for 2 h. The mixture was filtered, and the filter cake washed with DCM. The filtrate was concentrated under reduced pressure and the residue purified by reversed-phase flash chromatography on a CIS column using a 0 to 70% gradient of MeCN in water (containing 10 mmol/L NH₄HCO₃) over 40 min. The crude product (1.27 g) was purified by preparative SFC (Column: DAICEL DCpak P4VP 3×25 cm, 5 μm; Mobile Phase A: CO₂, Mobile Phase B: MeOH (containing 1% of a 2 M solution of NH₃ in MeOH); Flow rate: 65 mL/min; Gradient: isocratic 25% B; Column Temperature (° C.): 35; Back Pressure (bar): 100; Wave Length: 220 nm; RT peak 1 (min): 6.27; RT peak 2 (min): 6.78; Sample Solvent: MeOH; Injection Volume: 2 mL; Number of runs: 7) to afford (2-(((5-(1-((2S,4R)-4-hydroxy-2-((4-(4-methylthiazol-5-yl)benzyl)carbamoyl)pyrrolidin-1-yl)-3-methyl-1-oxobutan-2-yl)isoxazol-3-yl)oxy)methyl)phenyl)boronic acid (mixture of diastereoisomers) (810 mg, yield=35%) as a white solid.

LCMS: (ES, m/z): [M−OH]⁺=601 (LCMS condition: Column: Shim-pack Scepter C18-120; Mobile phase A: water (containing 5 mM NH₄HCO₃); Mobile phase B: MeCN; Flow rate: 1.5 mL/min.; RT: 0.840 min.)

### (d) Separation of Int 90 Diastereoisomers

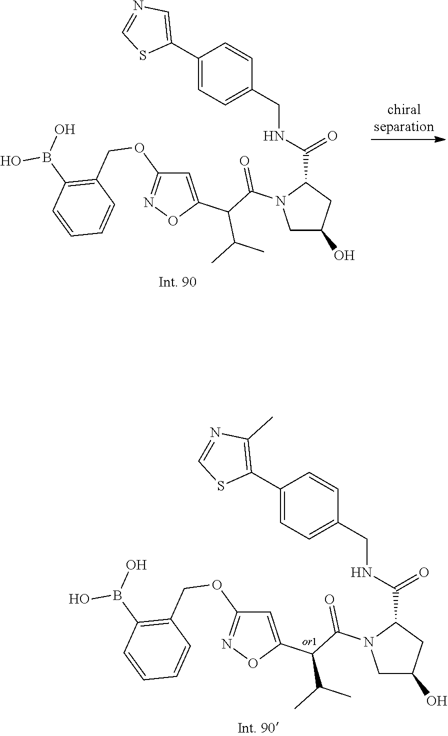 [SMILES: CC(C)C(C(=O)N1C[C@H](O)C[C@H]1C(=O)NCc1ccc(-c2cncs2)cc1)c1cc(OCc2ccccc2B(O)O)no1 ; Cc1ncsc1-c1ccc(CNC(=O)[C@@H]2C[C@@H](O)CN2C(=O)[C@H](c2cc(OCc3ccccc3B(O)O)no2)C(C)C)cc1]

(2-(((5-(1-((2S,4R)-4-Hydroxy-2-((4-(4-methylthiazol-5-yl)benzyl)carbamoyl)pyrrolidin-1-yl)-3-methyl-1-oxobutan-2-yl)isoxazol-3-yl)oxy)methyl)phenyl)boronic acid (mixture of diastereoisomers) (810 mg) was separated by preparative chiral SFC (Column: CHIRAL ART Cellulose-SZ 3×25 cm, 5 μm; Mobile Phase A: CO₂, Mobile Phase B: MeOH (containing 1% of a 2 M solution of NH₃ in MeOH); Flow rate: 100 mL/min; Gradient: isocratic 44% B; Column Temperature (° C.): 35; Back Pressure (bar): 100; Wave Length: 220 nm; RT peak 1 (min): 3.93; RT peak 2 (min): 6.77; Sample Solvent: MeOH; Injection Volume: 2 mL) to afford (2-(((5-(1-((2S,4R)-4-hydroxy-2-((4-(4-methylthiazol-5-yl)benzyl)carbamoyl)pyrrolidin-1-yl)-3-methyl-1-oxobutan-2-yl)isoxazol-3-yl)oxy)methyl)phenyl)boronic acid (second eluting peak) (single diastereoisomer with unknown absolute stereochemistry at the centre indicated in the structure) (410 mg, yield=50%).

LCMS: (ES, m/z): [M−OH]⁺=601 (LCMS condition: Column: Shim-pack Scepter C18-120; Mobile phase A: water (containing 5 mM NH₄HCO₃); Mobile phase B: MeCN; Flow rate: 1.5 mL/min.; RT: 0.818 min.)

### (e) Synthesis of Int. 91

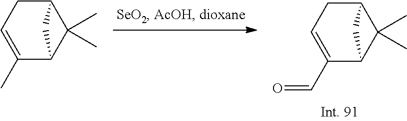

Into a 1 L round-bottom flask were added (+)-α-pinene (20.0 g, 147 mmol, 1 eq.), 1,4-dioxane (300 mL), SeO₂ (16.3 g, 147 mmol, 1 eq.), and AcOH (13.2 g, 220 mmol, 1.5 eq.) at 20° C. The reaction was stirred for 16 h at 60° C. The mixture was quenched with water and extracted with EtOAc (3×200 mL). The combined organic extracts were washed with brine (100 mL) and dried over anhydrous Na₂SO₄. After filtration, the filtrate was concentrated under reduced pressure and the residue purified by silica gel column chromatography eluting with PE/EtOAc (10:1) to afford (1S,5R)-6,6-dimethylbicyclo[3.1.1]hept-2-ene-2-carbaldehyde (15.6 g, yield=67%) as a brown oil.

LCMS: (ES, m/z): [M+H]⁺=151 (LCMS condition: Column: HALO 90A C18; Mobile phase A: water (containing 0.1% formic acid); Mobile phase B: MeCN (containing 0.1% formic acid); Flow rate: 1.2 mL/min.; RT: 0.937 min.)

### (f) Synthesis of Int. 92

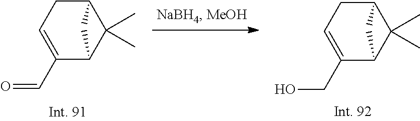

Into a 500 mL round-bottom flask were added (1S,5R)-6,6-dimethylbicyclo[3.1.1]hept-2-ene-2-carbaldehyde (8.0 g, 53 mmol, 1 eq.) and MeOH (160 mL) at 20° C. NaBH₄ (10.1 g, 266 mmol, 5 eq.) was added portion wise at 0° C. The reaction mixture was stirred for 16 h at 20° C. The mixture was quenched with water at 0° C. and extracted with EtOAc (3×150 mL). The combined organic extracts were washed with brine (50 mL) and dried over anhydrous Na₂SO₄. After filtration, the filtrate was concentrated under reduced pressure and the residue purified by silica gel column chromatography eluting with PE/EtOAc (10:1) to afford [(1S,5R)-6,6-dimethylbicyclo[3.1.1]hept-2-en-2-yl]methanol (6.4 g, yield=69%) as a brown oil.

LCMS: (ES, m/z): [M-OH]+=135 (LCMS condition: Column: HALO 90A C18; Mobile phase A: water (containing 0.1% formic acid); Mobile phase B: MeCN (containing 0.1% formic acid); Flow rate: 1.2 mL/min.; RT: 1.175 min.)

### (g) Synthesis of Int. 93

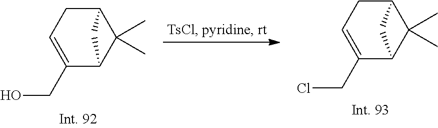

Into a 250 mL 3-necked round-bottom flask were added [(1S,5R)-6,6-dimethylbicyclo[3.1.1]hept-2-en-2-yl]methanol (6.0 g, 39 mmol, 1 eq.), DCM (60 mL), and pyridine (12.6 g, 78.8 mmol, 2 eq.) at 20° C. TsCl (9.0 g, 47 mmol, 1.2 eq.) was added to the mixture in portions at 0° C. The resulting mixture was stirred for 16 h at 20° C. The reaction was quenched with water and extracted with DCM (3×100 mL). The combined organic extracts were washed with brine (100 mL) and dried over anhydrous Na₂SO₄. After filtration, the filtrate was concentrated under reduced pressure and the residue purified by silica gel column chromatography eluting with PE/EtOAc (10:1) to afford (1S,5R)-2-(chloromethyl)-6,6-dimethylbicyclo[3.1.1]hept-2-ene (1.2 g, yield=17%) as a brown oil.

LCMS: (ES, m/z): no ionisation (LCMS condition: Column: HALO 90A C18; Mobile phase A: water (containing 0.1% formic acid); Mobile phase B: MeCN (containing 0.1% formic acid); Flow rate: 1.2 mL/min; RT: 1.261 min)

### (h) Synthesis of Int. 94

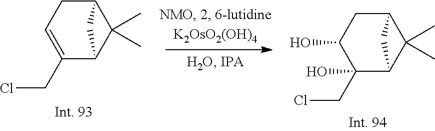

Into a 40 mL vial were added (1S,5R)-2-(chloromethyl)-6,6-dimethylbicyclo[3.1.1]hept-2-ene (600 mg, 3.52 mmol, 1 eq.), IPA (10 mL), water (2 mL), NMO (535 mg, 4.57 mmol, 1.3 eq.), 2,6-lutidine (452 mg, 4.22 mmol, 1.2 eq.) and K₂OsO₂(OH)₄ (130 mg, 0.352 mmol, 0.1 eq.). The reaction was stirred for 20 h at 50° C. under nitrogen. The mixture was allowed to cool to 20° C., quenched with water and extracted with EtOAc (3×100 mL). The combined organic extracts were washed with brine (30 mL) and dried over anhydrous Na₂SO₄. After filtration, the filtrate was concentrated under reduced pressure and the residue purified by silica gel column chromatography eluting with PE/EtOAc (5:1) to afford (1S,2S,3R,5S)-2-(chloromethyl)-6,6-dimethylbicyclo[3.1.1]heptane-2,3-diol (200 mg, yield=20%) as a colourless oil.

### (i) Synthesis of Int. 95

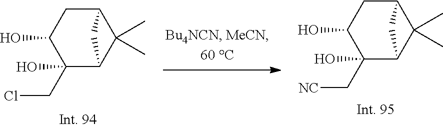

Into a 40 mL vial were added (1S,2S,3R,5S)-2-(chloromethyl)-6,6-dimethylbicyclo[3.1.1]heptane-2,3-diol (250 mg, 1.22 mmol, 1 eq.), MeCN (5.0 mL) and Bu₄NCN (492 mg, 1.83 mmol, 1.5 eq.) at 20° C. The reaction was stirred for 5 h at 60° C. The mixture was allowed to cool to 20° C., quenched with water and extracted with EtOAc (3×50 mL). The combined organic extracts were washed with brine (3×30 mL) and dried over anhydrous Na₂SO₄. After filtration, the filtrate was concentrated under reduced pressure to afford 2-[(15,25,3R,5S)-2,3-dihydroxy-6,6-dimethylbicyclo[3.1.1]heptan-2-yl]acetonitrile (150 mg, yield=44%) as a brown oil.

### (j) Synthesis of Int. 96

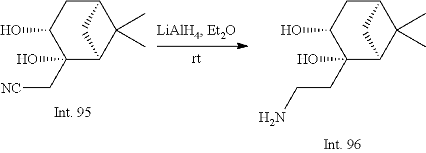

Into a 40 mL vial were added 2-[(1S,2S,3R,5S)-2,3-dihydroxy-6,6-dimethylbicyclo[3.1.1]heptan-2-yl]acetonitrile (650 mg, 3.33 mmol, 1 eq.) and THF (10 mL) at 20° C. LiAlH₄ (379 mg, 9.99 mmol, 3 eq.) was added to the mixture at 0° C. and the reaction stirred for 16 h at 20° C. The mixture was quenched with water at 0° C. and extracted with EtOAc (3×100 mL). The combined organic extracts were washed with brine (30 mL) and dried over anhydrous Na₂SO₄. After filtration, the filtrate was concentrated under reduced pressure to afford (1S,2S,3R,5S)-2-(2-aminoethyl)-6,6-dimethylbicyclo[3.1.1]heptane-2,3-diol (450 mg, yield=41%) as a colourless oil.

### (k) Synthesis of Int. 97

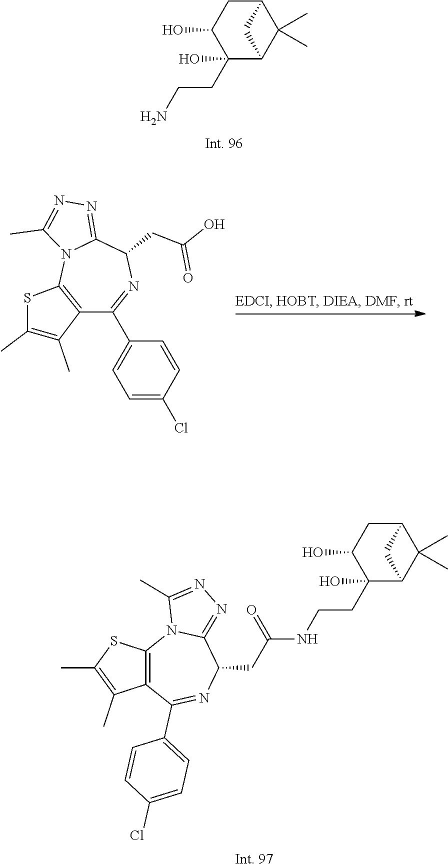

Into a 40 mL vial were added (1S,2S,3R,5S)-2-(2-aminoethyl)-6,6-dimethylbicyclo[3.1.1]heptane-2,3-diol (400 mg, 2.01 mmol, 1 eq.), DMF (8 mL), (S)-2-(4-(4-chlorophenyl)-2,3,9-trimethyl-6H-thieno[3,2-f][1,2,4]triazolo[4,3-a][1,4]diazepin-6-yl)acetic acid (402 mg, 1.00 mmol, 0.5 eq.), EDCI (500 mg, 2.61 mmol, 1.3 eq.), HOBT (353 mg, 2.61 mmol, 1.3 eq.) and DIEA (778 mg, 6.02 mmol, 3 eq.) at 20° C. The reaction was stirred for 1 h at 20° C. The mixture was quenched with water at 20° C. and extracted with EtOAc (3×100 mL). The combined organic extracts were washed with brine (3×50 mL) and dried over anhydrous Na₂SO₄. After filtration, the filtrate was concentrated under reduced pressure and the residue purified by reversed-phase flash chromatography on a C18 column using a 40 to 70% gradient of MeCN in water (containing 0.1% formic acid) over 10 min. The crude product was purified by preparative HPLC (Column: XBridge Prep Shield RP C18 Column, 19×250 mm, 5 μm; Mobile Phase A: water (containing 0.1% formic acid), Mobile Phase B: MeOH; Flow rate: 25 mL/min; Gradient: 50 to 80% B over 15 min; Wave Length: 254 and 220 nm; RT (min): 13.95) to afford 2-((S)-4-(4-chlorophenyl)-2,3,9-trimethyl-6H-thieno[3,2-f][1,2,4]triazolo[4,3-a][1,4]diazepin-6-yl)-N-(2-((1S,2S,3R,5S)-2,3-dihydroxy-6,6-dimethylbicyclo[3.1.1]heptan-2-yl)ethyl)acetamide (single diastereoisomer with unknown absolute stereochemistry at the centre indicated in the structure) (157.4 mg, yield=13%) as a white solid.

LCMS: (ES, m/z): [M+H]⁺=582 (LCMS condition: Column: HALO 90A C18; Mobile phase A: water (containing 0.1% formic acid); Mobile phase B: MeCN (containing 0.1% formic acid); Flow rate: 1.2 mL/min.; RT: 0.933 min.)

### (l) Synthesis of Example 13

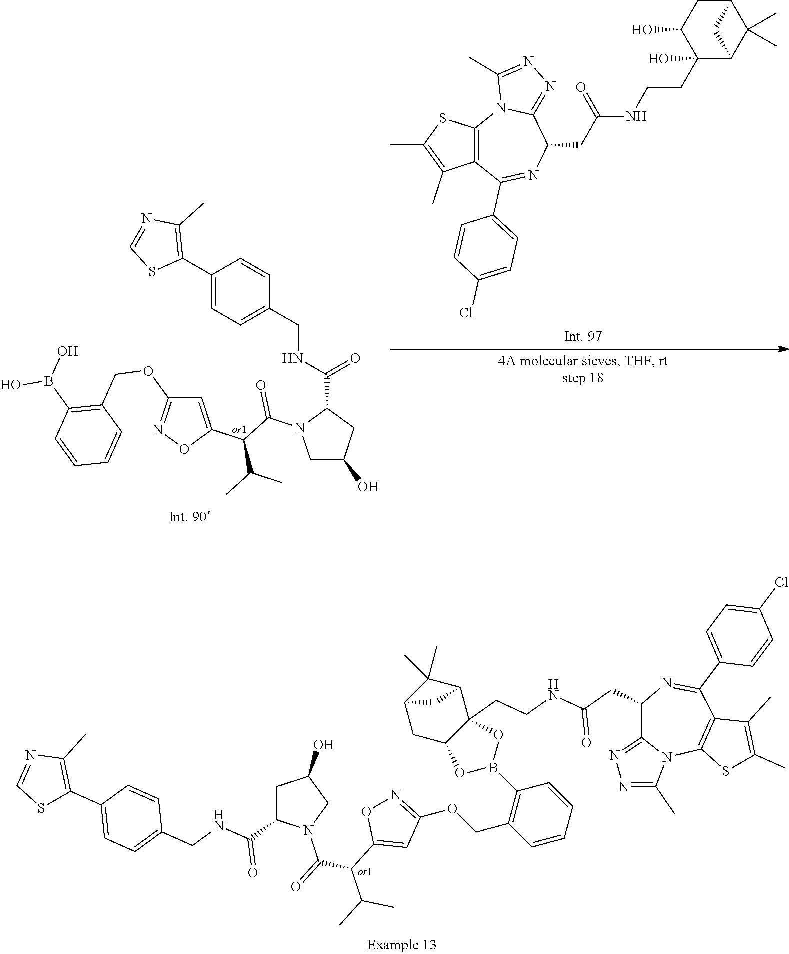

A solution of (2-(((5-(1-((2S,4R)-4-hydroxy-2-((4-(4-methylthiazol-5-yl)benzyl)carbamoyl)pyrrolidin-1-yl)-3-methyl-1-oxobutan-2-yl)isoxazol-3-yl)oxy)methyl)phenyl)boronic acid (single diastereoisomer with unknown absolute stereochemistry at the centre indicated in the structure) (75 mg, 0.121 mmol, 1 eq.), 2-((S)-4-(4-chlorophenyl)-2,3,9-trimethyl-6H-thieno[3,2-f][1,2,4]triazolo[4,3-a][1,4]diazepin-6-yl)-N-(2-((1S,2S,3R,5S)-2,3-dihydroxy-6,6-dimethylbicyclo[3.1.1]heptan-2-yl)ethyl)acetamide (71 mg, 0.121 mmol, 1 eq.) and molecular sieves (4×) (400 mg) in THF (10 mL) was stirred for 16 h at r.t. under nitrogen. The mixture was filtered, and the filter cake washed with THF (3×10 mL). The filtrate was concentrated under reduced pressure and the residue purified by preparative SFC (Column: Torus Diol OBD 3×25 cm, 5 μm; Mobile Phase A: CO₂, Mobile Phase B: IPA (containing 1% of a 2 M solution of NH₃ in IPA); Flow rate: 75 mL/min; Gradient: isocratic 30% B; Column Temperature (° C.): 35; Back Pressure (bar): 100; Wave Length: 220 nm; RT (min): 14.62; Sample Solvent: MeCN; Injection Volume: 2.5 mL; Number of runs: 2) to afford (2S,4R)-1-(2-(3-((2-((3aS,4S,6S,7aR)-3a-(2-(2-((S)-4-(4-chlorophenyl)-2,3,9-trimethyl-6H-thieno[3,2-f][1,2,4]triazolo[4,3-a][1,4]diazepin-6-yl)acetamido)ethyl)-5,5-dimethylhexahydro-4,6-methanobenzo[d][1,3,2]dioxaborol-2-yl)benzyl)oxy)isoxazol-5-yl)-3-methylbutanoyl)-4-hydroxy-N-(4-(4-methylthiazol-5-yl)benzyl)pyrrolidine-2-carboxamide (single diastereoisomer with unknown absolute stereochemistry at the centre indicated in the structure) (92.3 mg, yield=65%) as a white solid.

LCMS: (ES, m/z): [M+H]⁺=1164 (LCMS condition: Column: HALO 90A C18; Mobile phase A: water (containing 0.1% formic acid); Mobile phase B: MeCN (containing 0.1% formic acid); Flow rate: 1.2 mL/min.; RT: 1.222 min.)

### Synthesis of Example Compounds—BTK

### Example 14

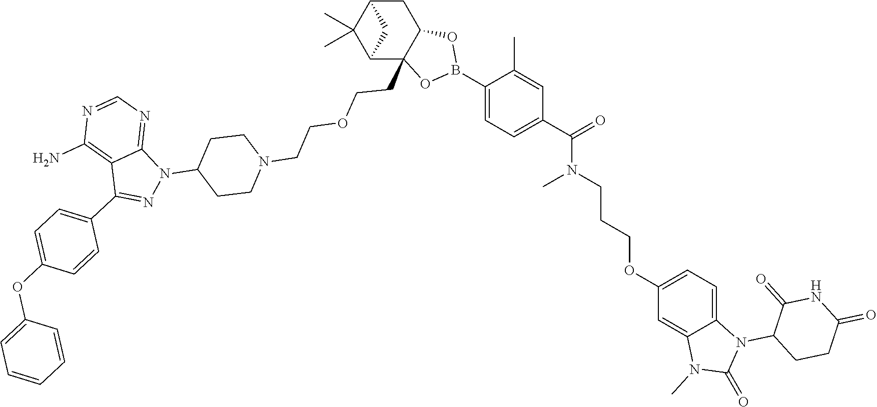 [SMILES: Cc1cc(C(=O)N(C)CCCOc2ccc3c(c2)n(C)c(=O)n3C2CCC(=O)NC2=O)ccc1B1O[C@H]2C[C@H]3C[C@H](C3(C)C)[C@@]2(CCOCCN2CCC(n3nc(-c4ccc(Oc5ccccc5)cc4)c4c(N)ncnc43)CC2)O1]

### (a) Synthesis of Int. 98

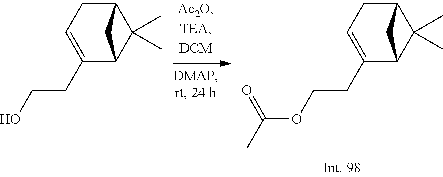

To a stirred solution of 2-[(1R,5S)-6,6-dimethylbicyclo[3.1.1]hept-2-en-2-yl]ethanol (6 g, 36.087 mmol, 1 eq.), Ac₂O (4.1 mL, 43.304 mmol, 1.2 eq.) and DMAP (440.9 mg, 3.609 mmol, 0.1 eq.) in DCM (30 mL) was added TEA (7.5 mL, 54.131 mmol, 1.50 eq.) and the reaction stirred for 4 h at 25° C. The mixture was concentrated under reduced pressure and the residue purified by silica gel column chromatography eluting with a gradient of 0 to 10% THF in PE to afford 2-[(1R,5S)-6,6-dimethylbicyclo[3.1.1]hept-2-en-2-yl]ethyl acetate (7.3 g, yield=95%) as a colourless oil.

LCMS condition: Column: HALO 90A C18; Mobile phase A: water (containing 0.1% formic acid); Mobile phase B: MeCN (containing 0.1% formic acid); Flow rate: 1.2 mL/min; RT: 1.189 min)

### (b) Synthesis of Int. 99

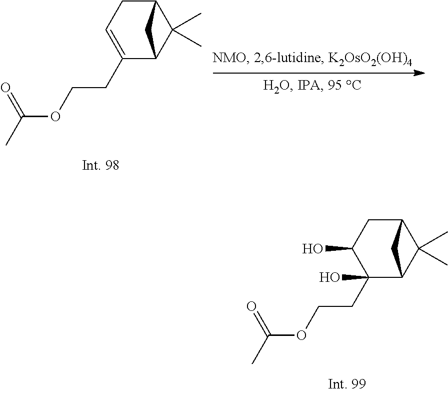 [SMILES: CC(=O)OCCC1=CC[C@H]2C[C@@H]1C2(C)C ; CC(=O)OCC[C@@]1(O)[C@@H]2C[C@H](C[C@@H]1O)C2(C)C]

To a stirred solution of 2-[(1R,5S)-6,6-dimethylbicyclo[3.1.1]hept-2-en-2-yl]ethyl acetate (7.3 g, 34.380 mmol, 1 eq.), NMO (4.83 g, 41.256 mmol, 1.2 eq.) and 2,6-lutidine (4.8 mL, 41.256 mmol, 1.20 eq.) in water (4 mL) and IPA (20 mL) was added K₂OsO₂(OH)₄ (1266.7 mg, 3.438 mmol, 0.1 eq.) at 25° C. and stirred for 16 h at 95° C. The mixture was quenched with water (150 mL) and extracted with EtOAc (3×200 mL). The combined organic extracts were washed with brine (200 mL) and dried over Na₂SO₄. After filtration, the filtrate was concentrated under reduced pressure and the residue purified by silica gel column chromatography eluting with a gradient of 0 to 60% EtOAc in PE to afford 2-[(1R,2R,3S,5R)-2,3-dihydroxy-6,6-dimethylbicyclo[3.1.1]heptan-2-yl]ethyl acetate (4.96 g, yield=60%) as a yellow oil.

### (c) Synthesis of Int. 100

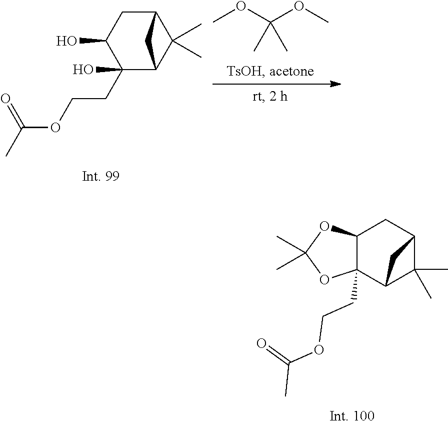

To a stirred solution of 2-[(1R,2R,3S,5R)-2,3-dihydroxy-6,6-dimethylbicyclo[3.1.1]heptan-2-yl]ethyl acetate (4.9 g, 20.222 mmol, 1 eq.) and 2,2-dimethoxypropane (4.97 g, 40.444 mmol, 2 eq.) in acetone (50 mL) was added TsOH (696.4 mg, 4.044 mmol, 0.2 eq.) and the reaction stirred for 2 h at 25° C. The mixture was concentrated under reduced pressure and the residue purified by silica gel column chromatography eluting with a gradient of 0 to 20%/o EtOAc in PE to afford 2-((3aR,4R,6R,7aS)-2,2,5,5-tetramethyltetrahydro-4,6-methanobenzo[d][1,3]dioxol-3a(4H)-yl)ethyl acetate (4.3 g, yield=72%) as a colourless oil.

### (d) Synthesis of Int. 101

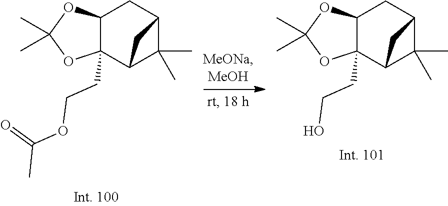

To a stirred solution of 2-((3aR,4R,6R,7aS)-2,2,5,5-tetramethyltetrahydro-4,6-methanobenzo[d][1,3]dioxol-3a(4H)-yl)ethyl acetate (4.3 g, 15.228 mmol, 1 eq.) in MeOH (28 mL) was added MeONa (987.2 mg, 18.274 mmol, 1.2 eq.) and the reaction stirred for 16 h at 25° C. The mixture was concentrated under reduced pressure and the residue purified by silica gel column chromatography eluting with PE/EtOAc (1:1) to afford 2-((3aR,4R,6R,7aS)-2,2,5,5-tetramethyltetrahydro-4,6-methanobenzo[d][1,3]dioxol-3a(4H)-yl)ethan-1-ol (3.4 g, yield=87%) as a colourless oil.

### (e) Synthesis of Int. 102

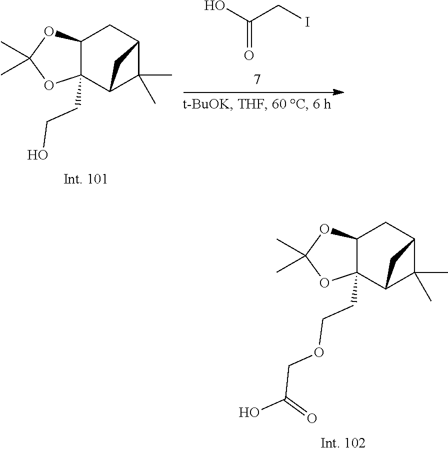

To a stirred solution of 2-[(1R,2R,6S,8R)-4,4,9,9-tetramethyl-3,5-dioxatricyclo[6.1.1.0\hat{ }{2,6}]decan-2-yl]ethanol (2.4 g, 9.986 mmol, 1 eq.), iodoacetic acid (2.04 g, 10.985 mmol, 1.1 eq.) in THF (25 mL) was added t-BuOK (2.24 g, 19.962 mmol, 2.00 eq.) at 25° C. The reaction was stirred for 16 h at 60° C. The mixture was quenched with water (100 mL) and extracted with EtOAc (3×150 mL). The aqueous layer was acidified to pH 3 with 6 N HCl (aq.) and extracted with EtOAc (3×150 mL). The organic extracts were combined, washed with brine (150 mL) and dried over Na₂SO₄. After filtration, the filtrate was concentrated under reduced pressure to afford crude 2-(2-((3aR,4R,6R,7aS)-2,2,5,5-tetramethyltetrahydro-4,6-methanobenzo[d][1,3]dioxol-3a(4H)-yl)ethoxy)acetic acid (2 g, yield=58%) which was used in the next step without purification.

### (f) Synthesis of Int. 103

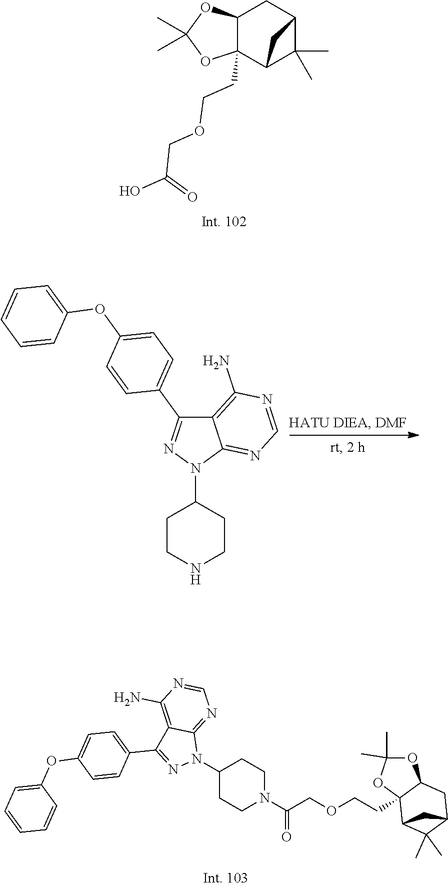 [SMILES: CC1(C)O[C@H]2C[C@H]3C[C@H](C3(C)C)[C@@]2(CCOCC(=O)O)O1 ; Nc1ncnc2c1c(-c1ccc(Oc3ccccc3)cc1)nn2C1CCNCC1 ; CC1(C)O[C@H]2C[C@H]3C[C@H](C3(C)C)[C@@]2(CCOCC(=O)N2CCC(n3nc(-c4ccc(Oc5ccccc5)cc4)c4c(N)ncnc43)CC2)O1]

To a stirred solution of 2-(2-((3aR,4R,6R,7a5)-2,2,5,5-tetramethyltetrahydro-4,6-methanobenzo[d][1,3]dioxol-3a(4H)-yl)ethoxy)acetic acid (1.07 g, 3.571 mmol, 1.15 eq.), 3-(4-phenoxyphenyl)-1-(piperidin-4-yl)pyrazolo[3,4-d]pyrimidin-4-amine (1.2 g, 3.105 mmol, 1.00 eq.) and HATU (1.77 g, 4.657 mmol, 1.50 eq.) in DMF (12 mL) was added DIEA (2.7 mL, 15.525 mmol, 5.00 eq.) and the reaction stirred for 2 h at 25° C. The mixture was quenched with water (80 mL) and extracted with EtOAc (3×100 mL). The combined organic extracts were washed with brine (100 mL) and dried over Na₂SO₄. After filtration, the filtrate was concentrated under reduced pressure and the residue purified by silica gel column chromatography eluting with DCM/MeOH (10:1) to afford 1-(4-(4-amino-3-(4-phenoxyphenyl)-1H-pyrazolo[3,4-d]pyrimidin-1-yl)piperidin-1-yl)-2-(2-((3aR,4R,6R,7aS)-2,2,5,5-tetramethyltetrahydro-4,6-methanobenzo[d][1,3]dioxol-3a(4H)-yl)ethoxy)ethan-1-one (780 mg, yield=31%) as a pink solid.

LCMS: (ES, m/z): [M+H]⁺=667 (LCMS condition: Column: HALO 90A C18; Mobile phase A: water (containing 0.1% formic acid); Mobile phase B: MeCN (containing 0.1%/o formic acid); Flow rate: 1.2 mL/min; RT: 1.440 min)

### (g) Synthesis of Int. 104

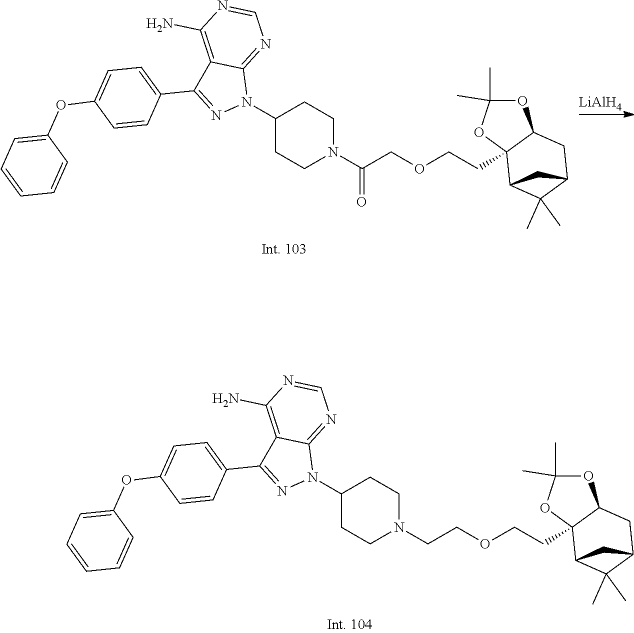 [SMILES: CC1(C)O[C@H]2C[C@H]3C[C@H](C3(C)C)[C@@]2(CCOCC(=O)N2CCC(n3nc(-c4ccc(Oc5ccccc5)cc4)c4c(N)ncnc43)CC2)O1 ; "LiAlH4" ; CC1(C)O[C@H]2C[C@H]3C[C@H](C3(C)C)[C@@]2(CCOCCN2CCC(n3nc(-c4ccc(Oc5ccccc5)cc4)c4c(N)ncnc43)CC2)O1]

Into a 40 mL sealed vial were added 1-(4-(4-amino-3-(4-phenoxyphenyl)-1H-pyrazolo[3,4-d]pyrimidin-1-yl)piperidin-1-yl)-2-(2-((3aR,4R,6R,7aS)-2,2,5,5-tetramethyltetrahydro-4,6-methanobenzo[d][1,3]dioxol-3a(4H)-yl)ethoxy)ethan-1-one (700 mg, 1.050 mmol, 1 eq.), and LiAlH₄ (79.7 mg, 2.100 mmol, 2 eq.) at 25° C. under nitrogen. THF (6 mL) was added and the reaction stirred for 2 h at 25° C. The mixture was quenched with water (20 mL). The resulting mixture was extracted with EtOAc (3×30 mL). The combined organic phase was washed with brine (50 mL) and dried over Na₂SO₄. After filtration, the filtrate was concentrated under reduced pressure and the residue purified by preparative TLC using a gradient of 0 to 10% MeOH in DCM to afford 3-(4-phenoxyphenyl)-1-(1-(2-(2-((3aR,4R,6R,7aS)-2,2,5,5-tetramethyltetrahydro-4,6-methanobenzo[d][1,3]dioxol-3a(4H)-yl)ethoxy)ethyl)piperidin-4-yl)-1H-pyrazolo[3,4-d]pyrimidin-4-amine (280 mg, yield=38%) as an off-white solid.

LCMS: (ES, m/z): [M+H]⁺=653 (LCMS condition: Column: Kinetex EVO C18; Mobile phase A: water (containing 6.5 mM NH₄HCO₃ and ammonia hydroxide (pH=10)); Mobile phase B: MeCN; Flow rate: 1.5 mL/min; RT: 1.218 min)

### (h) Synthesis of Int. 105

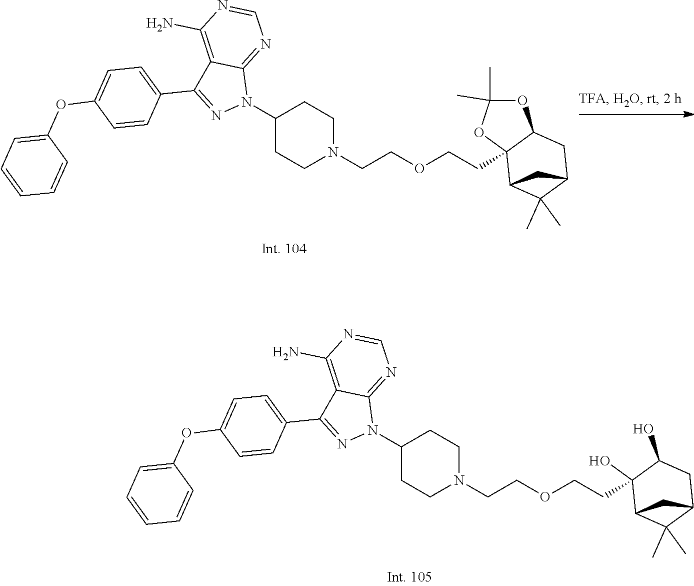 [SMILES: CC1(C)O[C@H]2C[C@H]3C[C@H](C3(C)C)[C@@]2(CCOCCN2CCC(n3nc(-c4ccc(Oc5ccccc5)cc4)c4c(N)ncnc43)CC2)O1 ; CC1(C)[C@@H]2C[C@H]1[C@](O)(CCOCCN1CCC(n3nc(-c4ccc(Oc5ccccc5)cc4)c4c(N)ncnc43)CC1)[C@@H](O)C2]

Into a 8 mL sealed vial were added 3-(4-phenoxyphenyl)-1-(1-(2-(2-((3aR,4R,6R,7aS)-2,2,5,5-tetramethyltetrahydro-4,6-methanobenzo[d][1,3]dioxol-3a(4H)-yl)ethoxy)ethyl)piperidin-4-yl)-1H-pyrazolo[3,4-d]pyrimidin-4-amine (280 mg, 0.429 mmol, 1 eq.), water (1.5 mL) and TFA (1.5 mL). The reaction was stirred for 3 h at 25° C. The mixture was concentrated under reduced pressure and the residue purified by reversed-phase flash chromatography on a C18 column using a 10 to 50% gradient of MeCN in water (containing 0.1% NH₄HCO₃) over 38 min. The crude product (100 mg) was purified by preparative HPLC (Column: XBridge Prep OBD C18 Column, 30×150 mm, 5 μm; Mobile Phase A: water (containing 10 nmol/L NH₄HCO₃), Mobile Phase B: MeCN; Flow rate: 60 mL/min; Gradient: 48 to 56% B over 10 min; Wave Length: 254 and 220 nm; RT (min): 8.72-9.22) to afford (1R,2R,3S,5R)-2-[2-(2-{4-[4-amino-3-(4-phenoxyphenyl)pyrazolo[3,4-d]pyrimidin-1-yl]piperidin-1-yl}ethoxy)ethyl]-6,6-dimethylbicyclo[3.1.1]heptane-2,3-diol (45.9 mg, yield=17%) as a white solid.

LCMS: (ES, m/z): [M+H]⁺=613 (LCMS condition: Column: HALO 90A C18; Mobile phase A: water (containing 0.1% formic acid); Mobile phase B: MeCN (containing 0.1% formic acid); Flow rate: 1.2 mL/min; RT: 0.745 min).

### (i) Synthesis of Int. 106

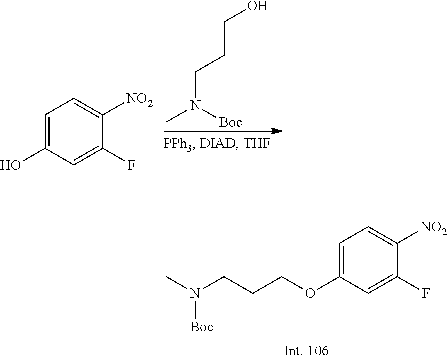

Into a 250 mL round-bottom flask were added 3-fluoro-4-nitrophenol (10 g, 63.654 mmol, 1 eq.), tert-butyl N-(3-hydroxypropyl)-N-methylcarbamate (14.46 g, 76.385 mmol, 1.2 eq.), tert-butyl N-(3-hydroxypropyl)-N-methylcarbamate (14.46 g, 76.385 mmol, 1.2 eq.), PPh₃ (21.70 g, 82.750 mmol, 1.3 eq.), THF (100 mL) and DIAD (19.31 g, 95.481 mmol, 1.5 eq.). The reaction was stirred 3 h at 25° C. under nitrogen. The mixture was quenched with water (100 mL) and extracted with EtOAc (3×200 mL). The combined organic extracts were washed with brine (200 mL) and dried over anhydrous Na₂SO₄. After filtration, the filtrate was concentrated under reduced pressure and the residue purified by silica gel column chromatography eluting with PE/EtOAc (1:1) to afford tert-butyl (3-(3-fluoro-4-nitrophenoxy)propyl)(methyl)carbamate (20 g, yield=96%) as a brown yellow oil.

LCMS: (ES, m/z): [M−C₄H₈]⁺=273 (LCMS condition: Column: Shim-pack Scepter C18-120; Mobile phase A: water (containing 5 mM NH₄HCO₃); Mobile phase B: MeCN; Flow rate: 1.5 mL/min)

### (j) Synthesis of Int. 107

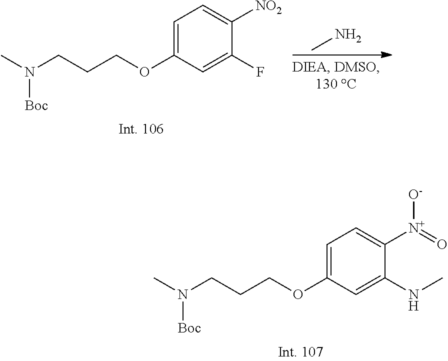

Into a 250 mL round-bottom flask were added tert-butyl (3-(3-fluoro-4-nitrophenoxy)propyl)(methyl)carbamate (6.5 g, 19.797 mmol, 1 eq.), DIEA (7.68 g, 59.391 mmol, 3 eq.), methanamine hydrochloride (1.60 g, 23.756 mmol, 1.2 eq.) and DMSO (65 mL) at 20° C. The reaction was stirred 2 h at 130° C. under nitrogen. The mixture was quenched with water (100 mL) and extracted with EtOAc (3×200 mL). The combined organic extracts were washed with brine (200 mL) and dried over anhydrous Na₂SO₄. After filtration, the filtrate was concentrated under reduced pressure and the residue purified by silica gel column chromatography eluting with PE/EtOAc (5:1) to afford tert-butyl methyl(3-(3-(methylamino)-4-nitrophenoxy)propyl)carbamate (6.5 g, yield=95%) as a yellow oil.

LCMS: (ES, m/z): [M−C₄H₈]⁺=284 (LCMS condition: Column: Shim-pack Scepter C18-120; Mobile phase A: water (containing 5 mM NH₄HCO₃); Mobile phase B: MeCN; Flow rate: 1.5 mL/min)

### (k) Synthesis of Int. 108

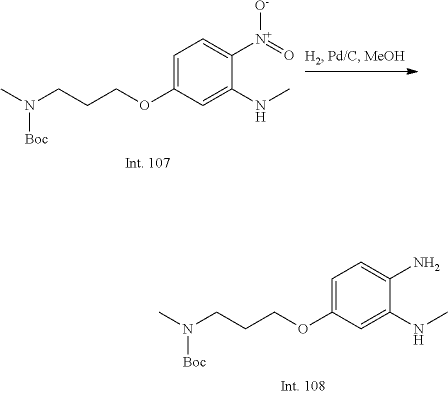

Into a 250 mL 3-necked round-bottom flask were added tert-butyl methyl(3-(3-(methylamino)-4-nitrophenoxy)propyl)carbamate (7.5 g, 22.098 mmol, 1 eq.), Pd/C (705.5 mg, 0.663 mmol, 0.03 eq., 10% wt.), MeOH (75 mL) and H₂ at r.t. The reaction was stirred for 16 h at r.t. The mixture filtered through Celite which was then washed with MeOH (300 mL). The filtrate was concentrated under reduced pressure to afford tert-butyl (3-(4-amino-3-(methylamino)phenoxy)propyl)(methyl)carbamate (6.8 g, yield=25%) as a black oil.

LCMS: (ES, m/z): [M+H]⁺=310 (LCMS condition: Column: Shim-pack Scepter C18-120; Mobile phase A: water (containing 5 mM NH₄HCO₃); Mobile phase B: MeCN; Flow rate: 1.5 mL/min; RT: 1.512 min)

### (l) Synthesis of Int. 109

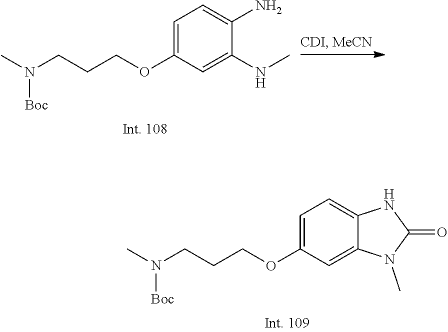

Into a 100 mL round-bottom flask were added tert-butyl (3-(4-amino-3-(methylamino)phenoxy)propyl)(methyl)carbamate (5 g, 16.160 mmol, 1 eq.), MeCN (50 mL) and CDI (2.62 g, 16.160 mmol, 1.00 eq.). The reaction was stirred for 2 h at 20° C. under nitrogen. The mixture was quenched with water (100 mL) and extracted with EtOAc (3×100 mL). The combined organic extracts were washed with brine (100 mL) and dried over anhydrous Na₂SO₄. After filtration, the filtrate was concentrated under reduced pressure and the residue purified by reversed-phase flash chromatography on a C18 column using a 40 to 50% gradient of MeCN in water (containing 0.1% NH₄HCO₃) over 10 min to afford tert-butyl methyl(3-((3-methyl-2-oxo-2,3-dihydro-1H-benzo[d]imidazol-5-yl)oxy)propyl)carbamate (3.14 g, yield=48%) as a light yellow solid.

LCMS: (ES, m/z): [M−C₄H₈]⁺=280 (LCMS condition: Column: Shim-pack Scepter C18-120; Mobile phase A: water (containing 5 mM NH₄HCO₃); Mobile phase B: MeCN; Flow rate: 1.5 mL/min; RT: 1.506 min)

### (m) Synthesis of Int. 111

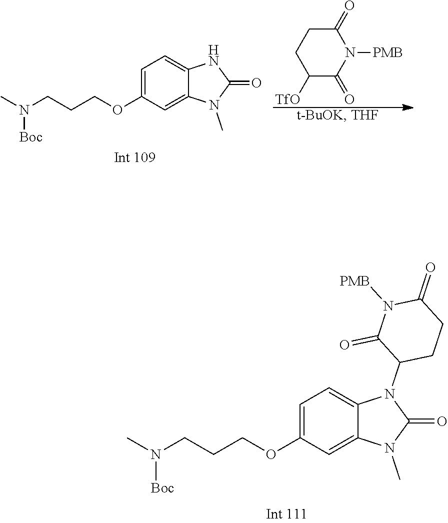

Into a 40 mL vial were added tert-butyl methyl(3-((3-methyl-2-oxo-2,3-dihydro-1H-benzo[d]imidazol-5-yl)oxy)propyl)carbamate (1 g, 2.981 mmol, 1 eq.), 1-[(4-methoxyphenyl)methyl]-2,6-dioxopiperidin-3-yl trifluoromethanesulfonate (racemic) (2.84 g, 7.452 mmol, 2.5 eq.), t-BuOK (0.50 g, 4.471 mmol, 1.5 eq.) and THF (10 mL) at 0° C. The resulting mixture was stirred for 0.1 h at 25° C. The resulting mixture was quenched with water (50 mL) and extracted with EtOAc (3×50 mL). The combined organic extracts were washed with brine (100 mL) and dried over anhydrous Na₂SO₄. After filtration, the filtrate was concentrated under reduced pressure and the residue purified by silica gel column chromatography eluting with DCM/MeOH (10:1) to afford tert-butyl (3-((1-(1-(4-methoxybenzyl)-2,6-dioxopiperidin-3-yl)-3-methyl-2-oxo-2,3-dihydro-1H-benzo[d]imidazol-5-yl)oxy)propyl)(methyl)carbamate (racemic) (1.2 g, yield=70%) as a white solid.

LCMS: (ES, m/z): [M−C₅H₇O₂]⁺=467 (LCMS condition: Column: Shim-pack Scepter C18-120; Mobile phase A: water (containing 5 mM NH₄HCO₃); Mobile phase B: MeCN; Flow rate: 1.5 mL/min; RT: 1.134 min)

### (n) Synthesis of Int. 112

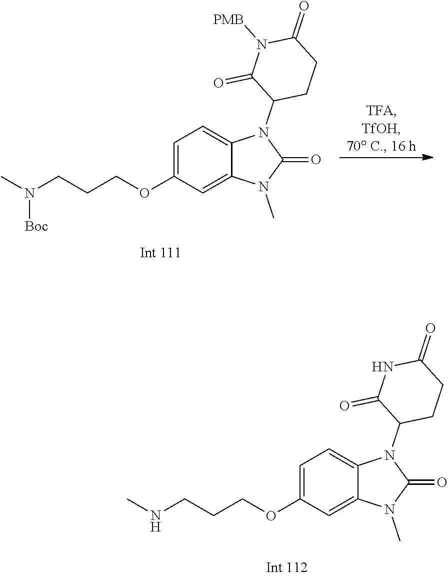

Into a 40 mL vial were added afford tert-butyl (3-((1-(1-(4-methoxybenzyl)-2,6-dioxopiperidin-3-yl)-3-methyl-2-oxo-2,3-dihydro-1H-benzo[d]imidazol-5-yl)oxy)propyl)(methyl)carbamate (racemic) (500 mg, 0.882 mmol, 1 eq.), TFA (25 mL) and TfOH (1.25 mL) at 20° C. The reaction was stirred for 16 h at 70° C. under air. The mixture was cooled to r.t. and concentrated under reduced pressure. The residue was purified by reversed-phase flash chromatography on a C18 column using a 10 to 50% gradient of MeCN in water (containing 0.1a formic acid) over 10 min to afford 3-{3-methyl-4-[3-(methylamino)propoxy]-2-oxo-1,3-benzodiazol-1-yl}piperidine-2,6-dione (racemic) (300 mg, yield=66%) as a light yellow oil.

LCMS: (ES, m/z): [M+H]⁺=347 (LCMS condition: Column: Shim-pack Scepter C18-120; Mobile phase A: water (containing 0.1% formic acid); Mobile phase B: MeCN (containing 0.1% formic acid); Flow rate: 1.5 mL/min)

### (o) Synthesis of Int. 113

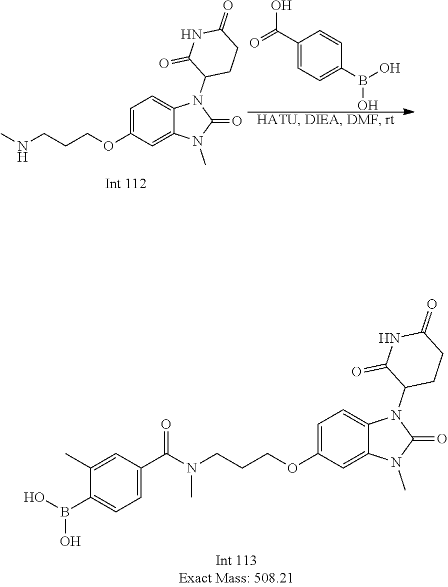

Into an 8 mL vial were added 3-{3-methyl-4-[3-(methylamino)propoxy]-2-oxo-1,3-benzodiazol-1-yl}piperidine-2,6-dione (racemic) (300 mg, 0.866 mmol, 1 eq.), 4-(dihydroxyboranyl)-3-methylbenzoic acid (233.8 mg, 1.299 mmol, 1.5 eq.), HOBt (175.6 mg, 1.299 mmol, 1.5 eq.), EDCI (249.0 mg, 1.299 mmol, 1.5 eq.), DMF (3 mL) and TEA (263 mg, 2.598 mmol, 3 eq.). The reaction was stirred 3 h at 20° C. under nitrogen. The resulting was quenched with water (30 mL) and extracted with EtOAc (3×30 mL). The combined organic extracts were washed with brine (50 mL) and dried over anhydrous Na₂SO₄. After filtration, the filtrate was concentrated under reduced pressure and the residue purified by preparative HPLC (Column: XBridge Prep Shield RP18 OBD C18, 30×150 mm, 5 μm; Mobile Phase A: water (containing 0.1% formic acid), Mobile Phase B: MeCN; Flow rate: 60 mL/min; Gradient: 7 to 37% B over 10 min; Wave Length: 254 and 220 nm; RT (min): 8.5) to afford 4-[(3-{[1-(2,6-dioxopiperidin-3-yl)-3-methyl-2-oxo-1,3-benzodiazol-5-yl]oxy}propyl)(methyl)carbamoyl]-2-methylphenylboronic acid (racemic) (55.7 mg, yield=12%) as a white solid.

LCMS: (ES, m/z): [M+H]⁺=509 (LCMS condition: Column: Shim-pack Scepter C18-120; Mobile phase A: water (containing 0.1% formic acid); Mobile phase B: MeCN (containing 0.1% formic acid); Flow rate: 1.5 mL/min; RT: 0.685 min)

### (p) Synthesis of Example 14

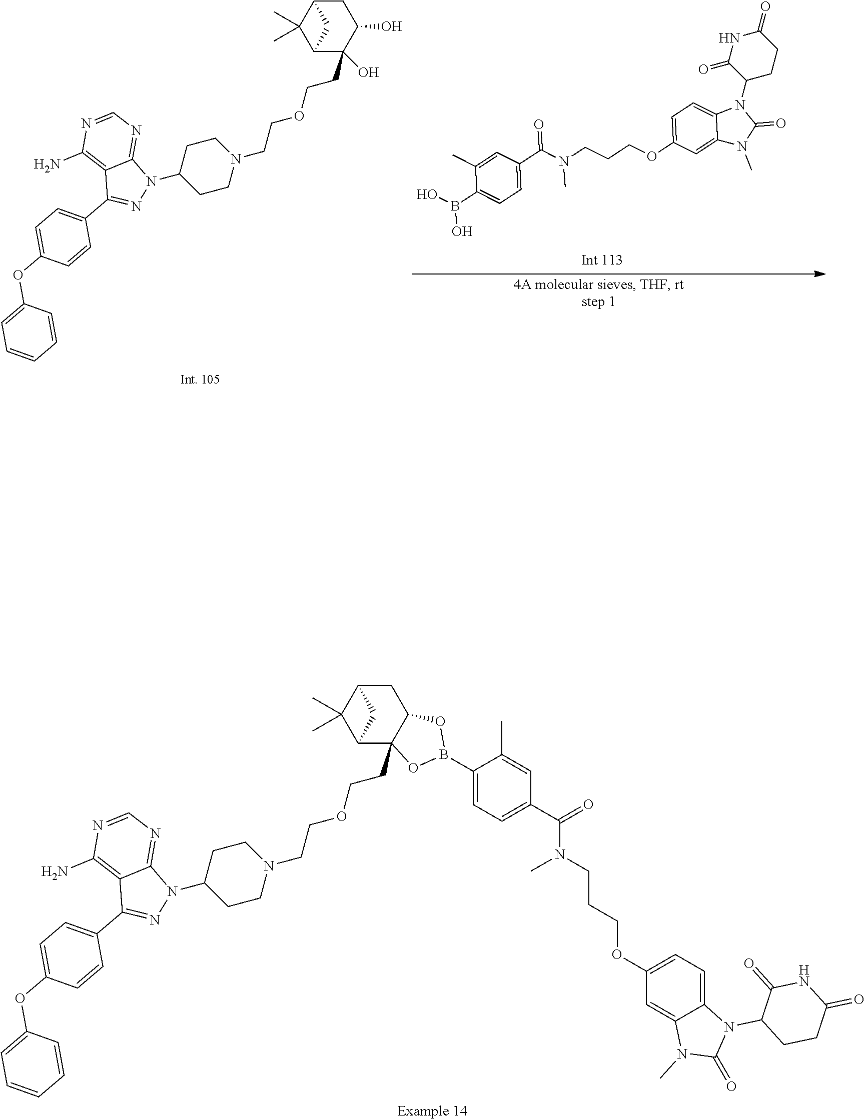 [SMILES: CC1(C)[C@@H]2C[C@H]1[C@](O)(CCOCCN1CCC(n3nc(-c4ccc(Oc5ccccc5)cc4)c4c(N)ncnc43)CC1)[C@@H](O)C2 ; Cc1cc(C(=O)N(C)CCCOc2ccc3c(c2)n(C)c(=O)n3C2CCC(=O)NC2=O)ccc1B(O)O ; Cc1cc(C(=O)N(C)CCCOc2ccc3c(c2)n(C)c(=O)n3C2CCC(=O)NC2=O)ccc1B1O[C@H]2C[C@H]3C[C@H](C3(C)C)[C@@]2(CCOCCN2CCC(n3nc(-c4ccc(Oc5ccccc5)cc4)c4c(N)ncnc43)CC2)O1]

Into an 8 mL sealed vial were added (1R,2R,3S,5R)-2-[2-(2-{4-[4-amino-3-(4-phenoxyphenyl)pyrazolo[3,4-d]pyrimidin-1-yl]piperidin-1-yl}ethoxy)ethyl]-6,6-dimethylbicyclo[3.1.1]heptane-2,3-diol (12 mg, 0.020 mmol, 1 eq.), 4-[(3-{[1-(2,6-dioxopiperidin-3-yl)-3-methyl-2-oxo-1,3-benzodiazol-5-yl]oxy}propyl)(methyl)carbamoyl]-2-methylphenylboronic acid (racemic) (10 mg, 0.020 mmol, 1 eq.) and molecular sieves (4 Å) (60 mg) at 25° C. EtOAc (0.5 mL) was added under nitrogen and the reaction stirred for 16 h at 25° C. The mixture was filtered and the filtrate concentrated under reduced pressure and the residue purified by preparative SFC (Column: Torus Diol OBD 3×25 cm, 5 μm; Mobile Phase A: CO₂, Mobile Phase B: IPA; Flow rate: 75 mL/min; Gradient: isocratic 40%/a B; Column Temperature (° C.): 35; Back Pressure (bar): 100; Wave Length: 220 nm; RT (min): 6.66; Sample Solvent: MeCN; Injection Volume: 1 mL; Number of runs: 5) to afford 4-((3aR,4R,6R,7aS)-3a-(2-(2-(4-(4-amino-3-(4-phenoxyphenyl)-1H-pyrazolo[3,4-d]pyrimidin-1-yl)piperidin-1-yl)ethoxy)ethyl)-5,5-dimethylhexahydro-4,6-methanobenzo[d][1,3,2]dioxaborol-2-yl)-N-(3-((1-(2,6-dioxopiperidin-3-yl)-3-methyl-2-oxo-2,3-dihydro-1H-benzo[d]imidazol-5-yl)oxy)propyl)-N,3-dimethylbenzamide (mixture of diastereoisomers) (12.8 mg, yield=49%) as a white solid.

LCMS: (ES, m/z): [M+H]⁺=1086 (LCMS condition: Column: Shim-pack Scepter C18-120; Mobile phase A: Water/MeCN (95:5, v:v) (containing 5 mM NH₄HCO₃); Mobile phase B: MeCN; Flow rate: 1.5 mL/min; RT: 0.998 min)

### Example 15

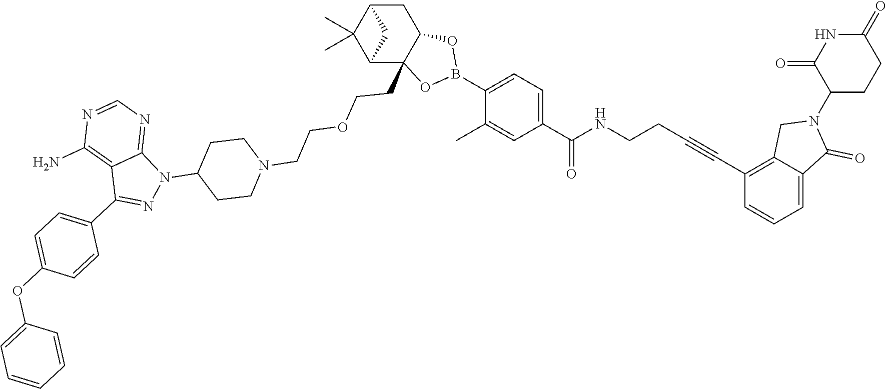 [SMILES: Cc1cc(C(=O)NCCC#Cc2cccc3c2CN(C2CCC(=O)NC2=O)C3=O)ccc1B1O[C@H]2C[C@H]3C[C@H](C3(C)C)[C@@]2(CCOCCN2CCC(n3nc(-c4ccc(Oc5ccccc5)cc4)c4c(N)ncnc43)CC2)O1]

### (a) Synthesis of Int. 114

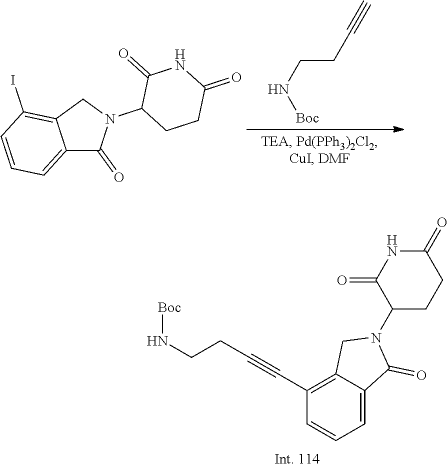 [SMILES: O=C1CCC(N2Cc3c(I)cccc3C2=O)C(=O)N1 ; C#CCCNC(=O)OC(C)(C)C ; CC(C)(C)OC(=O)NCCC#Cc1cccc2c1CN(C1CCC(=O)NC1=O)C2=O]

Into a 40 mL vial were added 3-(4-iodo-1-oxo-3H-isoindol-2-yl)piperidine-2,6-dione (racemic) (500 mg, 1.35 mmol, 1 eq.), DMF (5 mL), tert-butyl but-3-yn-1-ylcarbamate (274 mg, 1.62 mmol, 1.2 eq.), TEA (683 mg, 6.76 mmol, 5 eq.), Pd(PPh₃)₂Cl₂ (95 mg, 0.14 mmol, 0.1 eq.) and CuI (10 mg, 0.054 mmol, 0.04 eq.) at 18° C. The reaction was stirred for 5 h at 85° C. under nitrogen. The mixture was allowed to cool to 18° C., quenched with water (30 mL) and extracted with EtOAc (3×50 mL). The combined organic extracts were washed with brine (3×30 mL) and dried over anhydrous Na₂SO₄. After filtration, the filtrate was concentrated under reduced pressure and the residue purified by reversed-phase flash chromatography on a C18 column using a 40 to 70% gradient of MeCN in water (containing 0.1% formic acid) over 20 min to afford tert-butyl (4-(2-(2,6-dioxopiperidin-3-yl)-1-oxoisoindolin-4-yl)but-3-yn-1-yl)carbamate (racemic) (400 mg, yield=69%) as a brown solid.

LCMS: (ES, m/z): [M−C₄H₈]⁺=356 (LCMS condition: Column: HALO 90A C18; Mobile phase A: water (containing 0.1% formic acid); Mobile phase B: MeCN (containing 0.1% formic acid); Flow rate: 1.5 mL/min.; RT: 0.775 min)

### (b) Synthesis of Int. 115

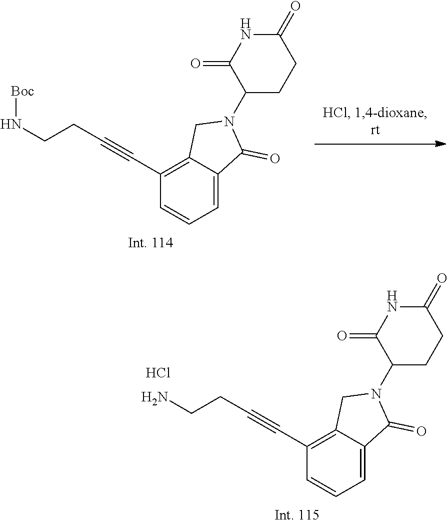 [SMILES: CC(C)(C)OC(=O)NCCC#Cc1cccc2c1CN(C1CCC(=O)NC1=O)C2=O ; Cl ; NCCC#Cc1cccc2c1CN(C1CCC(=O)NC1=O)C2=O]

Into a 40 mL vial were added tert-butyl (4-(2-(2,6-dioxopiperidin-3-yl)-1-oxoisoindolin-4-yl)but-3-yn-1-yl)carbamate (racemic) (380 mg, 0.924 mmol, 1 eq.), 1,4-dioxane (4 mL) and 4 M HCl in 1,4-dioxane (10 mL, 40.0 mmol). The reaction was stirred for 1 h at 18° C. The mixture was concentrated under reduced pressure to afford 3-[4-(4-aminobut-1-yn-1-yl)-1-oxo-3H-isoindol-2-yl]piperidine-2,6-dione hydrochloride (racemic) (280 mg, yield=93%) as a yellow solid.

LCMS: (ES, m/z): [M+H]⁺=312 (LCMS condition: Column: Shim-pack Scepter C18-120; Mobile phase A: water (containing 5 mM NH₄HCO₃); Mobile phase B: MeCN; Flow rate: 1.5 mL/min.; RT: 0.518 min)

### (c) Synthesis of Int. 116

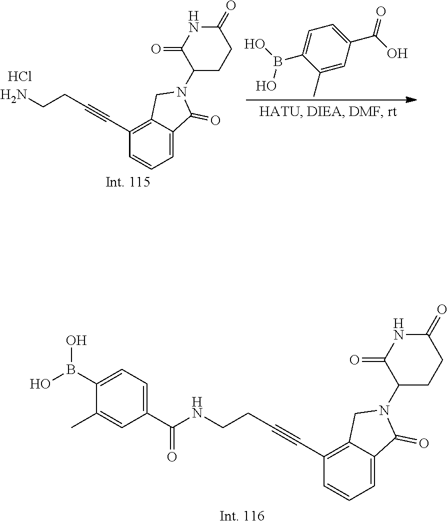

Into an 8 mL vial were added 3-[4-(4-aminobut-1-yn-1-yl)-1-oxo-3H-isoindol-2-yl]piperidine-2,6-dione hydrochloride (200 mg, 0.642 mmol, 1 eq.), DMF (2 mL), 4-(dihydroxyboranyl)-3-methylbenzoic acid (116 mg, 0.642 mmol, 1 eq.), HATU (366 mg, 0.963 mmol, 1.5 eq.) and DIEA (249 mg, 1.93 mmol, 3 eq.). The reaction was stirred for 1 h at 18° C. The mixture was purified by reversed-phase flash chromatography on a C18 column using a 20 to 50% gradient of MeCN in water (containing 0.1% formic acid) over 20 min. The crude product was purified by preparative HPLC (Column: Xselect CSH Prep OBD C18, 30×150 mm, 5 μm; Mobile Phase A: water (containing 0.1% formic acid), Mobile Phase B: MeCN; Flow rate: 60 mL/min; Gradient: 10 to 40% B over 10 min; Wave Length: 254 and 220 nm; RT (min): 7.88) to afford 4-({4-[2-(2,6-dioxopiperidin-3-yl)-1-oxo-3H-isoindol-4-yl]but-3-yn-1-yl}carbamoyl)-2-methylphenylboronic acid (racemic) (103.8 mg, yield=34%) as a white solid.

LCMS: (ES, m/z): [M+H]⁺=474 (LCMS condition: Column: HALO 90A C18; Mobile phase A: water (containing 0.1% formic acid); Mobile phase B: MeCN (containing 0.1% formic acid); Flow rate: 1.5 mL/min.; RT: 0.637 min)

### (d) Synthesis of Example 15

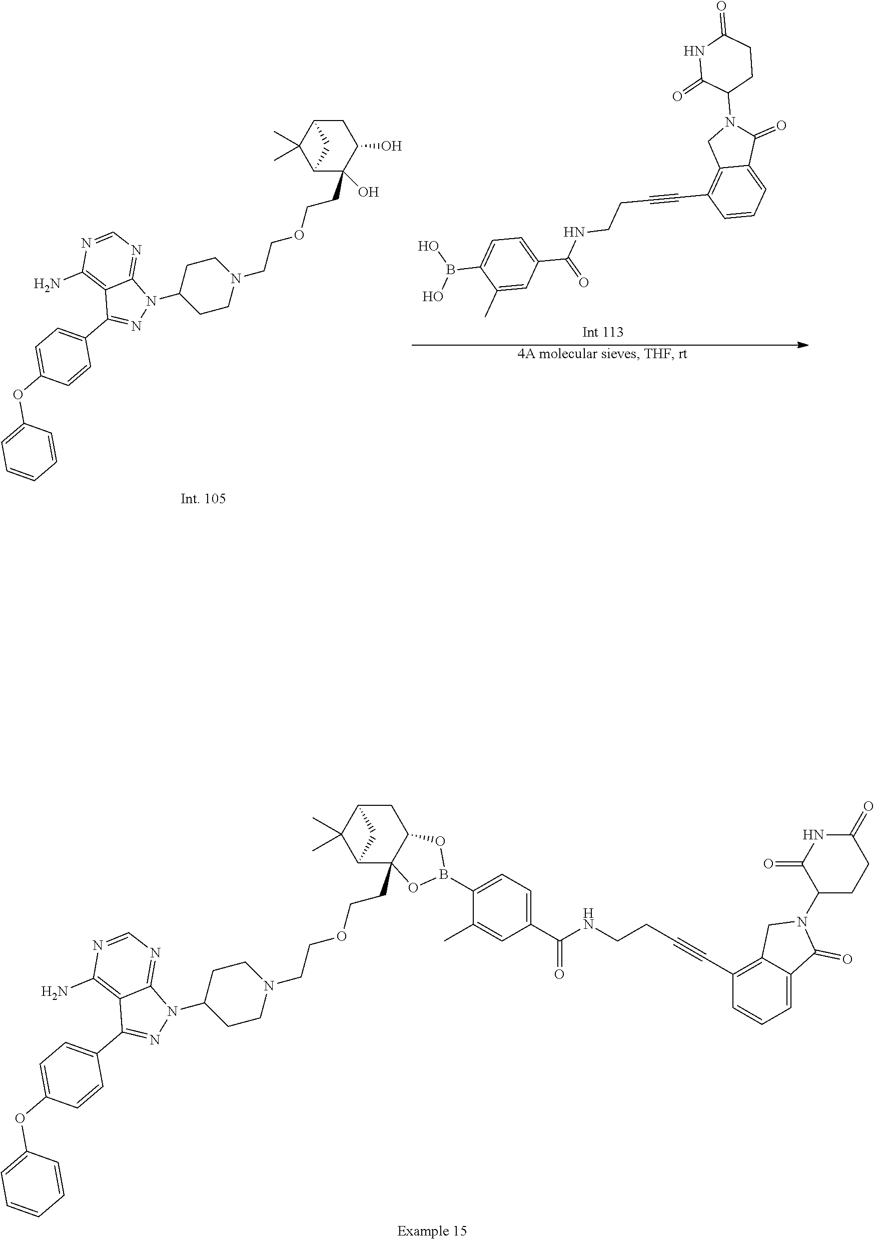 [SMILES: CC1(C)[C@@H]2C[C@H]1[C@](O)(CCOCCN1CCC(n3nc(-c4ccc(Oc5ccccc5)cc4)c4c(N)ncnc43)CC1)[C@@H](O)C2 ; Cc1cc(C(=O)NCCC#Cc2cccc3c2CN(C2CCC(=O)NC2=O)C3=O)ccc1B(O)O ; Cc1cc(C(=O)NCCC#Cc2cccc3c2CN(C2CCC(=O)NC2=O)C3=O)ccc1B1O[C@H]2C[C@H]3C[C@H](C3(C)C)[C@@]2(CCOCCN2CCC(n3nc(-c4ccc(Oc5ccccc5)cc4)c4c(N)ncnc43)CC2)O1]

Into an 8 mL sealed vial were added (1R,2R,3S,5R)-2-[2-(2-{4-[4-amino-3-(4-phenoxyphenyl)pyrazolo[3,4-d]pyrimidin-1-yl]piperidin-1-yl}ethoxy)ethyl]-6,6-dimethylbicyclo[3.1.1]heptane-2,3-diol (10 mg, 0.016 mmol, 1 eq.), 4-({4-[2-(2,6-dioxopiperidin-3-yl)-1-oxo-3H-isoindol-4-yl]but-3-yn-1-yl}carbamoyl)-2-methylphenylboronic acid (racemic) (7.72 mg, 0.016 mmol, 1 eq.) and molecular sieves (4 Å) (50 mg) at 25° C. Then THF (1 mL) was added under nitrogen. The reaction mixture was stirred for 16 h at 25° C. The mixture was concentrated under reduced pressure and the residue purified by preparative SFC (Column: Torus Diol OBD 3×25 cm, 5 μm; Mobile Phase A: CO₂, Mobile Phase B: MeOH; Flow rate: 75 mL/min; Gradient: isocratic 38% B; Column Temperature (° C.): 35; Back Pressure (bar): 100; Wave Length: 220 nm; RT (min): 5.09; Sample Solvent: MeOH; Injection Volume: 1.5 mL; Number of runs: 2) to afford 4-((3aR,4R,6R,7aS)-3a-(2-(2-(4-(4-amino-3-(4-phenoxyphenyl)-1H-pyrazolo[3,4-d]pyrimidin-1-yl)piperidin-1-yl)ethoxy)ethyl)-5,5-dimethylhexahydro-4,6-methanobenzo[d][1,3,2]dioxaborol-2-yl)-N-(4-(2-(2,6-dioxopiperidin-3-yl)-1-oxoisoindolin-4-yl)but-3-yn-1-yl)-3-methylbenzamide (mixture of diastereoisomers) (10.4 mg, yield=58%) as a white solid.

LCMS: (ES, m/z): [M+H]⁺=1050 (LCMS condition: Column: Shim-pack Scepter C18-120; Mobile phase A: Water/MeCN (95:5, v:v) (containing 5 mM NH₄HCO₃); Mobile phase B: MeCN (containing 0.1% formic acid); Flow rate: 1.5 mL/min.; RT: 1.033 min)

### Example 16

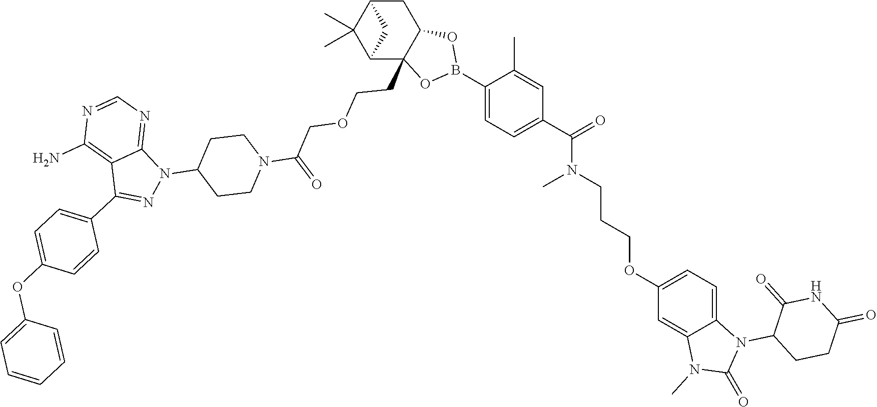 [SMILES: Cc1cc(C(=O)N(C)CCCOc2ccc3c(c2)n(C)c(=O)n3C2CCC(=O)NC2=O)ccc1B1O[C@H]2C[C@H]3C[C@H](C3(C)C)[C@@]2(CCOCC(=O)N2CCC(n3nc(-c4ccc(Oc5ccccc5)cc4)c4c(N)ncnc43)CC2)O1]

### (a) Synthesis of Int. 117

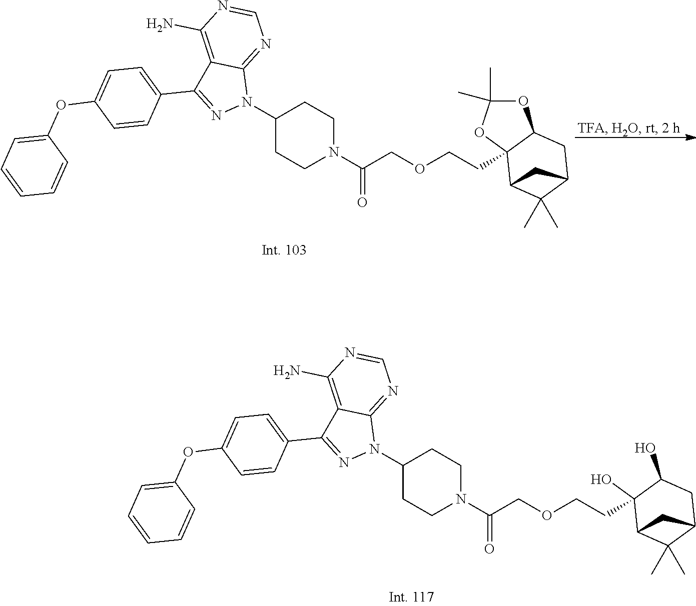 [SMILES: CC1(C)O[C@H]2C[C@H]3C[C@H](C3(C)C)[C@@]2(CCOCC(=O)N2CCC(n3nc(-c4ccc(Oc5ccccc5)cc4)c4c(N)ncnc43)CC2)O1 ; CC1(C)[C@@H]2C[C@H]1[C@](O)(CCOCC(=O)N1CCC(n3nc(-c4ccc(Oc5ccccc5)cc4)c4c(N)ncnc43)CC1)[C@@H](O)C2]

To a stirred solution of 1-(4-(4-amino-3-(4-phenoxyphenyl)-1H-pyrazolo[3,4-d]pyrimidin-1-yl)piperidin-1-yl)-2-(2-((3aR,4R,6R,7aS)-2,2,5,5-tetramethyltetrahydro-4,6-methanobenzo[d][1,3]dioxol-3a(4H)-yl)ethoxy)ethan-1-one (200 mg, 0.300 mmol, 1 eq.) in water (5 mL) was added TFA (5 mL) at 25° C. The reaction was stirred 2 h at 25° C. The mixture was concentrated under reduced pressure and the residue purified by preparative HPLC (Column: Xselect CSH Prep OBD C18 Column, 30×150 mm, 5 μm; Mobile Phase A: Water (containing 0.1%/a formic acid), Mobile Phase B: MeCN; Flow rate: 60 mL/min; Gradient: 47 to 55% B over 8 min; Wave Length: 254 and 220 nm; RT (min): 7.37) to afford 1-(4-(4-amino-3-(4-phenoxyphenyl)-1H-pyrazolo[3,4-d]pyrimidin-1-yl)piperidin-1-yl)-2-(2-((1R,2R,3S,5R)-2,3-dihydroxy-6,6-dimethylbicyclo[3.1.1]heptan-2-yl)ethoxy)ethan-1-one (62.2 mg, yield=32%) as a white solid.

LCMS: (ES, m/z): [M+H]⁺=627 (LCMS condition: Column: Shim-pack Scepter C18-120; Mobile phase A: water (containing 5 mM NH₄HCO₃); Mobile phase B: MeCN; Flow rate: 1.5 mL/min; RT: 1.130 min)

### (b) Synthesis of Example 16

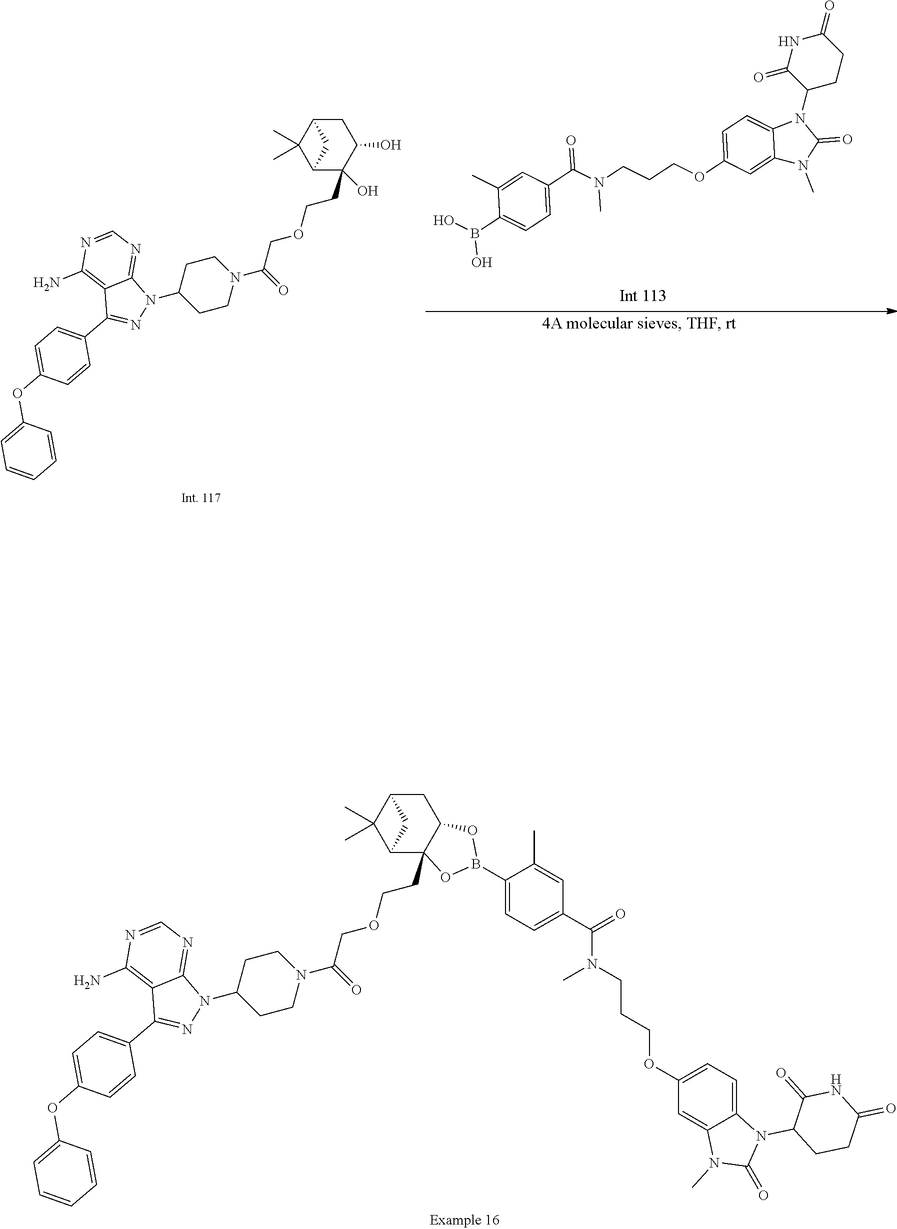 [SMILES: CC1(C)[C@@H]2C[C@H]1[C@](O)(CCOCC(=O)N1CCC(n3nc(-c4ccc(Oc5ccccc5)cc4)c4c(N)ncnc43)CC1)[C@@H](O)C2 ; Cc1cc(C(=O)N(C)CCCOc2ccc3c(c2)n(C)c(=O)n3C2CCC(=O)NC2=O)ccc1B(O)O ; Cc1cc(C(=O)N(C)CCCOc2ccc3c(c2)n(C)c(=O)n3C2CCC(=O)NC2=O)ccc1B1O[C@H]2C[C@H]3C[C@H](C3(C)C)[C@@]2(CCOCC(=O)N2CCC(n3nc(-c4ccc(Oc5ccccc5)cc4)c4c(N)ncnc43)CC2)O1]

Into a 20 mL vial were added 1-(4-(4-amino-3-(4-phenoxyphenyl)-1H-pyrazolo[3,4-d]pyrimidin-1-yl)piperidin-1-yl)-2-(2-((1R,2R,3S,5R)-2,3-dihydroxy-6,6-dimethylbicyclo[3.1.1]heptan-2-yl)ethoxy)ethan-1-one (20 mg, 0.032 mmol, 1 eq.), 4-[(3-{[1-(2,6-dioxopiperidin-3-yl)-3-methyl-2-oxo-1,3-benzodiazol-5-yl]oxy}propyl)(methyl)carbamoyl]-2-methylphenylboronic acid (racemic) (16 mg, 0.032 mmol, 1.00 eq.), molecular sieves (4 Å) (200 mg) and THF (5.00 mL). The reaction was stirred for 16 h at 25° C. under nitrogen. The mixture was filtered, and the filter cake washed with THF (3×20 mL). The filtrate was concentrated under reduced pressure and residue purified by preparative SFC (Column: Torus Diol OBD 3×25 cm, 5 μm; Mobile Phase A: CO₂, Mobile Phase B: IPA; Flow rate: 75 mL/min; Gradient: isocratic 42% B; Column Temperature (° C.): 35; Back Pressure (bar): 100; Wave Length: 220 nm; RT (min): 6.68; Sample Solvent: MeCN; Injection Volume: 2 mL) to afford 4-((3aR,4R,6R,7aS)-3a-(2-(2-(4-(4-amino-3-(4-phenoxyphenyl)-1H-pyrazolo[3,4-d]pyrimidin-1-yl)piperidin-1-yl)-2-oxoethoxy)ethyl)-5,5-dimethylhexahydro-4,6-methanobenzo[d][1,3,2]dioxaborol-2-yl)-N-(3-((1-(2,6-dioxopiperidin-3-yl)-3-methyl-2-oxo-2,3-dihydro-1H-benzo[d]imidazol-5-yl)oxy)propyl)-N,3-dimethylbenzamide (mixture of diastereoisomers) (8.3 mg, yield=23%) as a white solid.

LCMS: (ES, m/z): [M+H]⁺=1099 (LCMS condition: Column: HALO 90A C18; Mobile phase A: water (containing 0.1% formic acid); Mobile phase B: MeCN (containing 0.1% formic acid); Flow rate: 1.2 mL/min; RT: 1.046 min).

### Example 17

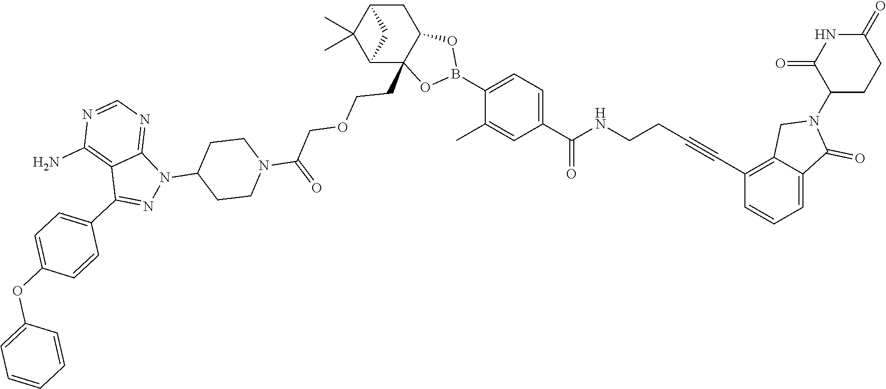 [SMILES: Cc1cc(C(=O)NCCC#Cc2cccc3c2CN(C2CCC(=O)NC2=O)C3=O)ccc1B1O[C@H]2C[C@H]3C[C@H](C3(C)C)[C@@]2(CCOCC(=O)N2CCC(n3nc(-c4ccc(Oc5ccccc5)cc4)c4c(N)ncnc43)CC2)O1]

### (a) Synthesis of Example 17

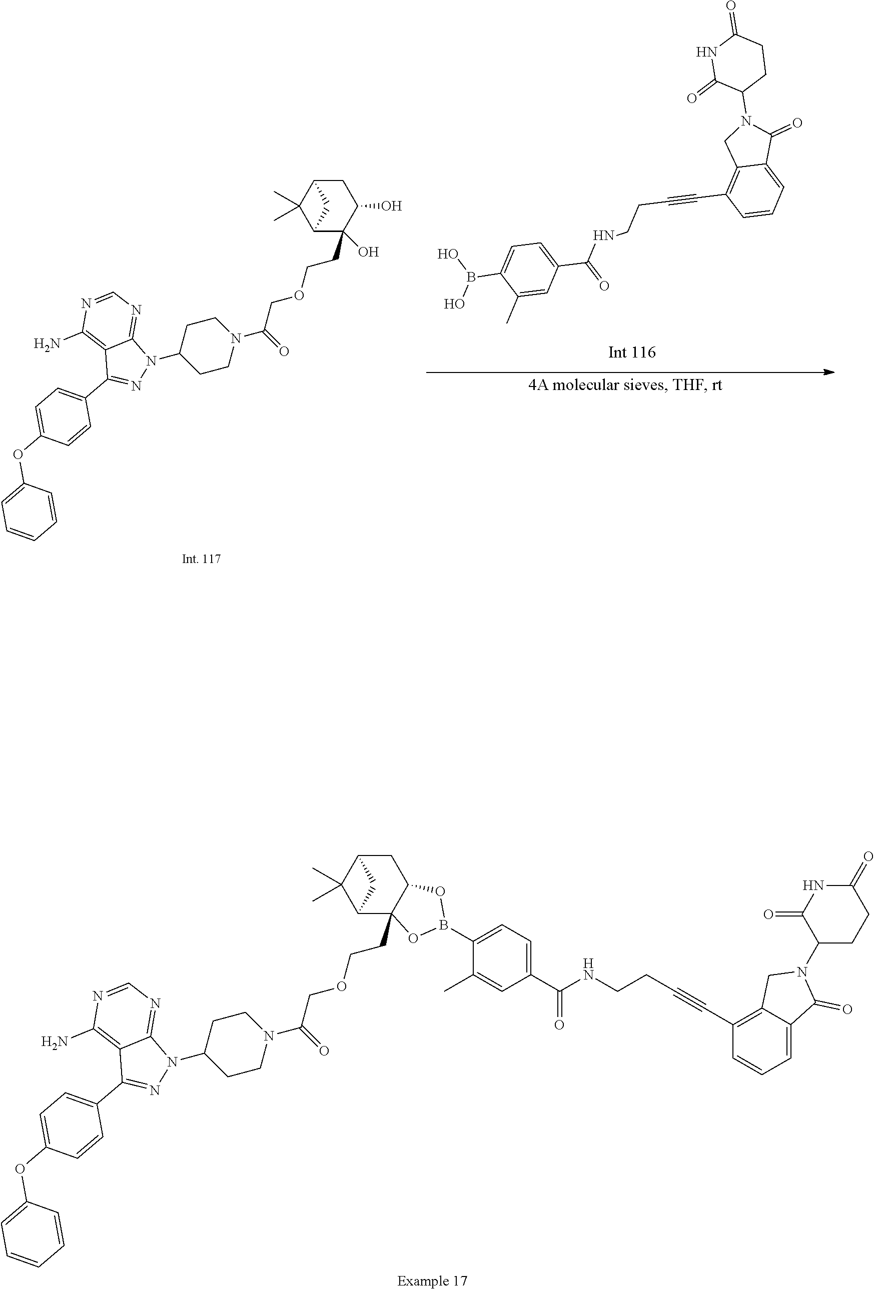 [SMILES: CC1(C)[C@@H]2C[C@H]1[C@](O)(CCOCC(=O)N1CCC(n3nc(-c4ccc(Oc5ccccc5)cc4)c4c(N)ncnc43)CC1)[C@@H](O)C2 ; Cc1cc(C(=O)NCCC#Cc2cccc3c2CN(C2CCC(=O)NC2=O)C3=O)ccc1B(O)O ; Cc1cc(C(=O)NCCC#Cc2cccc3c2CN(C2CCC(=O)NC2=O)C3=O)ccc1B1O[C@H]2C[C@H]3C[C@H](C3(C)C)[C@@]2(CCOCC(=O)N2CCC(n3nc(-c4ccc(Oc5ccccc5)cc4)c4c(N)ncnc43)CC2)O1]

Into a 10 mL sealed tube were added 1-(4-(4-amino-3-(4-phenoxyphenyl)-1H-pyrazolo[3,4-d]pyrimidin-1-yl)piperidin-1-yl)-2-(2-((1R,2R,3S,5R)-2,3-dihydroxy-6,6-dimethylbicyclo[3.1.1]heptan-2-yl)ethoxy)ethan-1-one (18 mg, 0.029 mmol, 1 eq.), 4-({4-[2-(2,6-dioxopiperidin-3-yl)-1-oxo-3H-isoindol-4-yl]but-3-yn-1-yl}carbamoyl)-2-methylphenylboronic acid (racemic) (13 mg, 0.029 mmol, 1.00 eq.), molecular sieves (4 Å) (90 mg) and THF (3 mL). The reaction was stirred for 16 h at 25° C. under nitrogen. The mixture was filtered and the filter cake washed with THF (3×50 mL). The filtrate was concentrated under reduced pressure and the residue purified by preparative SFC (Column: Torus Diol OBD 3×25 cm, 5 μm; Mobile Phase A: CO₂, Mobile Phase B: IPA; Flow rate: 75 mL/min; Gradient: isocratic 42% B; Column Temperature (° C.): 35; Back Pressure (bar): 100; Wave Length: 220 nm; RT (min): 8.22; Sample Solvent: MeCN/DCM; Injection Volume: 1.8 mL) to afford 4-((3aR,4R,6R,7aS)-3a-(2-(2-(4-(4-amino-3-(4-phenoxyphenyl)-1H-pyrazolo[3,4-d]pyrimidin-1-yl)piperidin-1-yl)-2-oxoethoxy)ethyl)-5,5-dimethylhexahydro-4,6-methanobenzo[d][1,3,2]dioxaborol-2-yl)-N-(4-(2-(2,6-dioxopiperidin-3-yl)-1-oxoisoindolin-4-yl)but-3-yn-1-yl)-3-methylbenzamide (mixture of diastereoisomers) (5.8 mg, yield=16%) as a white solid.

LCMS: (ES, m/z): [M+H]⁺=1064 (LCMS condition: Column: HALO 90A C18; Mobile phase A: water (containing 0.1% formic acid); Mobile phase B: MeCN (containing 0.1% formic acid); Flow rate: 1.2 mL/min; RT: 1.058 min)

### Example 18

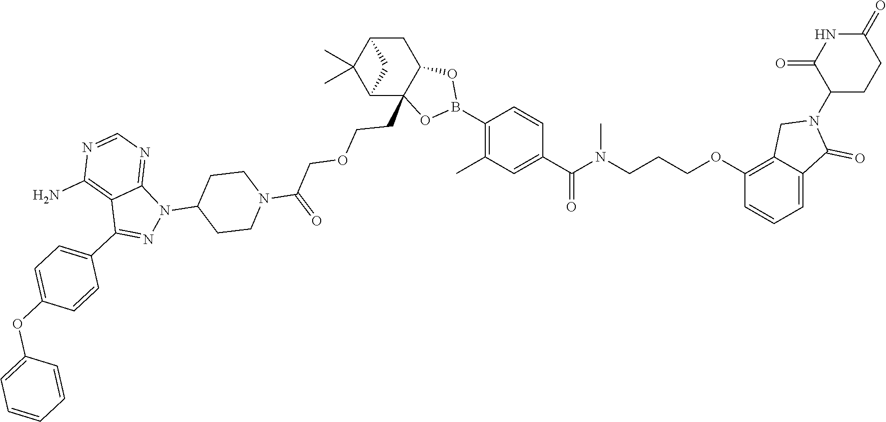 [SMILES: Cc1cc(C(=O)N(C)CCCOc2cccc3c2CN(C2CCC(=O)NC2=O)C3=O)ccc1B1O[C@H]2C[C@H]3C[C@H](C3(C)C)[C@@]2(CCOCC(=O)N2CCC(n3nc(-c4ccc(Oc5ccccc5)cc4)c4c(N)ncnc43)CC2)O1]

### (a) Synthesis of Int. 118

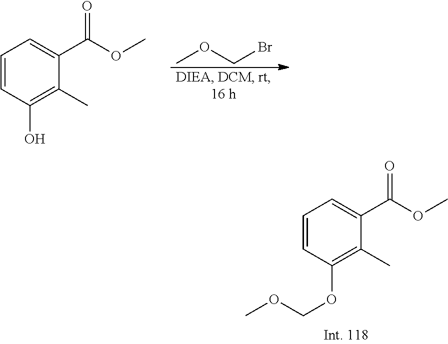

Into a 500 mL round-bottom flask were added methyl 3-hydroxy-2-methylbenzoate (20 g, 120.354 mmol, 1 eq.), DCM (200 mL) and DIEA (46.67 g, 361.062 mmol, 3.00 eq.) at r.t.

To the above mixture was added bromo(methoxy)methane (22.56 g, 180.531 mmol, 1.5 eq.) dropwise over 3 min at 0° C. The reaction was stirred overnight at r.t. The mixture was quenched with water (200 mL) and extracted with EtOAc (3×300 mL). The combined organic extracts were washed with brine (300 mL) and dried over anhydrous Na₂SO₄. After filtration, the filtrate was concentrated under reduced pressure and the residue purified by silica gel column chromatography eluting with PE/EtOAc (10:1) to afford methyl 3-(methoxymethoxy)-2-methylbenzoate (19.2 g, yield=75%) as a colourless oil.

LCMS: (ES, m/z): [M+H]⁺=211 (LCMS condition: Column: HALO 90A C18; Mobile phase A: water (containing 0.1% formic acid); Mobile phase B: MeCN (containing 0.1% formic acid); Flow rate: 1.5 mL/min; RT: 1.207 min)

### (b) Synthesis of Int. 119

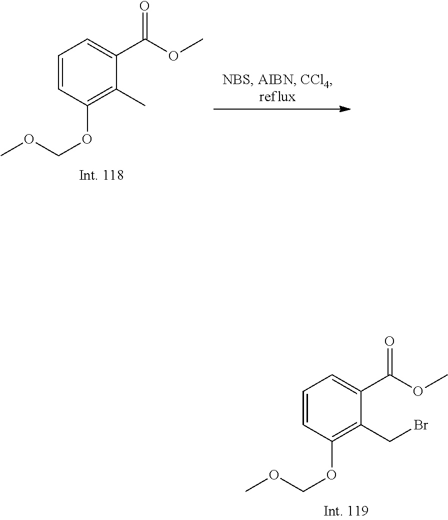

Into a 250 mL round-bottom flask were added methyl 3-(methoxymethoxy)-2-methylbenzoate (7.5 g, 35.675 mmol, 1 eq.), CCl₄ (115 mL), NBS (6.41 g, 36.032 mmol, 1.01 eq.) and AIBN (0.59 g, 3.567 mmol, 0.1 eq.) at r.t. The reaction was stirred overnight at 88° C. under nitrogen. The mixture was allowed to cool to r.t. and concentrated to afford methyl 2-(bromomethyl)-3-(methoxymethoxy)benzoate (10 g, yield=97%) as a light yellow solid.

### (c) Synthesis of Int. 120

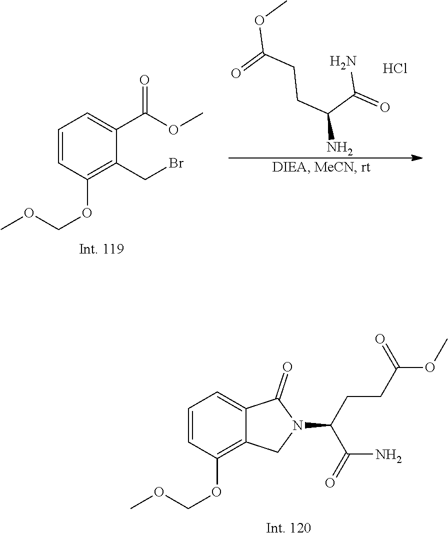

Into a 250 mL round-bottom flask were added methyl 2-(bromomethyl)-3-(methoxymethoxy)benzoate (5 g, 17.294 mmol, 1 eq.), methyl (4S)-4-amino-4-carbamoylbutanoate (12.46 g, 77.822 mmol, 1.5 eq.), DIEA (20.12 g, 155.643 mmol, 3.00 eq.) and MeCN (115 mL) at r.t. The reaction was stirred overnight at r.t. under nitrogen. The mixture was quenched with water (200 mL) and extracted with EtOAc (3×300 mL). The combined organic extracts were washed with brine (300 mL) and dried over anhydrous Na₂SO₄. After filtration, the filtrate was concentrated under reduced pressure and the residue purified by silica gel column chromatography eluting with DCM/MeOH (10:1) to afford methyl (4S)-4-carbamoyl-4-[4-(methoxymethoxy)-1-oxo-3H-isoindol-2-yl]butanoate (17 g, yield=97%) as a light yellow solid.

LCMS: (ES, m/z): [M+H]⁺=337 (LCMS condition: Column: Shim-pack Scepter C18-120; Mobile phase A: water (containing 5 mM NH₄HCO₃); Mobile phase B: MeCN; Flow rate: 1.5 mL/min.; RT: 0.650 min)

### (d) Synthesis of Int. 121

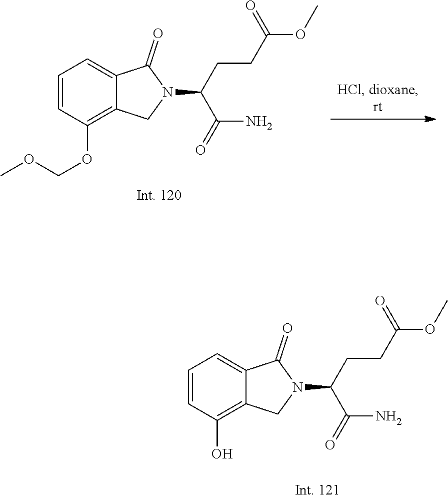

Into a 250 mL round-bottom flask were added methyl (4S)-4-carbamoyl-4-[4-(methoxymethoxy)-1-oxo-3H-isoindol-2-yl]butanoate (10 g, 29.731 mmol, 1 eq.) and 4 M HCl in 1,4-dioxane (100 mL) at 20° C. The reaction was stirred for 4 h at 20° C. under nitrogen. The mixture was concentrated under vacuum and the residue purified by reversed-phase flash chromatography on a C18 column using a 0 to 30% gradient of MeCN in water (containing 0.1% NH₄HCO₃) over 30 min to afford methyl (S)-5-amino-4-(4-hydroxy-1-oxoisoindolin-2-yl)-5-oxopentanoate (2 g, yield=23%) as a white solid.

LCMS: (ES, m/z): [M+H]⁺=293 (LCMS condition: Column: Shim-pack Scepter C18-120; Mobile phase A: water (containing 5 mM NH₄HCO₃); Mobile phase B: MeCN; Flow rate: 1.5 mL/min.; RT: 0.515 min).

### (e) Synthesis of Int. 122

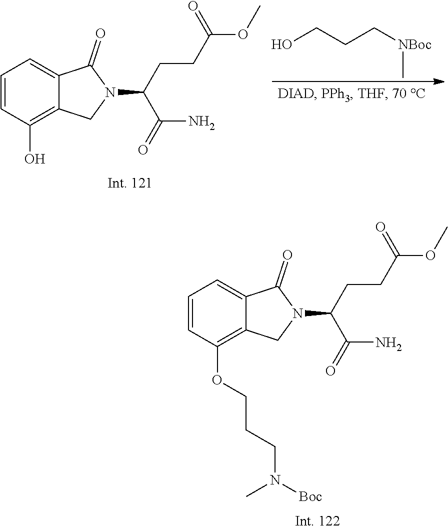

A solution of methyl (S)-5-amino-4-(4-hydroxy-1-oxoisoindolin-2-yl)-5-oxopentanoate (420 mg, 1.437 mmol, 1 eq.), tert-butyl N-(3-hydroxypropyl)-N-methylcarbamate (299 mg, 1.581 mmol, 1.1 eq.), DIAD (348 mg, 1.724 mmol, 1.2 eq.) and PPh₃ (565 mg, 2.155 mmol, 1.5 eq.) in THF (5 mL) was stirred for 2 min at 25° C. under nitrogen. The resulting mixture was stirred for 16 h at 70° C. The resulting mixture was concentrated under reduced pressure and the residue purified by silica gel column chromatography eluting with DCM/MeOH (10:1) to afford methyl (S)-5-amino-4-(4-(3-((tert-butoxycarbonyl)(methyl)amino)propoxy)-1-oxoisoindolin-2-yl)-5-oxopentanoate (290 mg, yield=42%) as a colourless semi-solid.

LCMS: (ES, m/z): [M+H]⁺=464 (LCMS condition: Column: Shim-pack Scepter C18-120; Mobile phase A: water (containing 5 mM NH₄HCO₃); Mobile phase B: MeCN; Flow rate: 1.5 mL/min; RT: 0.877 min)

### (f) Synthesis of Int. 123

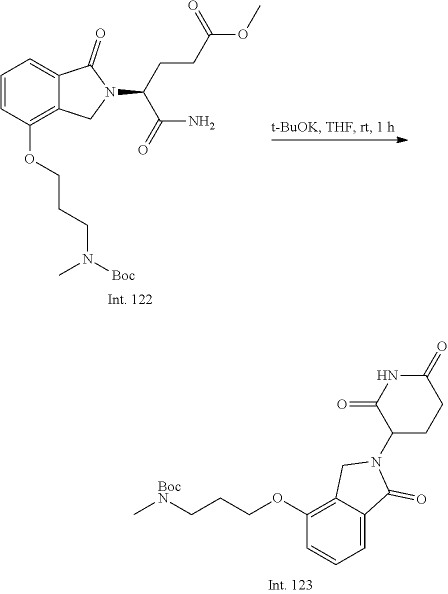 [SMILES: COC(=O)CC[C@@H](C(N)=O)N1Cc2c(OCCCN(C)C(=O)OC(C)(C)C)cccc2C1=O ; CN(CCCOc1cccc2c1CN(C1CCC(=O)NC1=O)C2=O)C(=O)OC(C)(C)C]

Into a 50 mL round-bottom flask were added methyl (S)-5-amino-4-(4-(3-((tert-butoxycarbonyl)(methyl)amino)propoxy)-1-oxoisoindolin-2-yl)-5-oxopentanoate (260 mg, 0.561 mmol, 1 eq.), t-BuOK (94 mg, 0.842 mmol, 1.5 eq.) and THF (10 mL). The reaction was stirred for 1 h at 25° C. The mixture was concentrated under reduced pressure and the residue purified by reversed-phase flash chromatography on a C18 column using a 30 to 60% gradient of MeCN in water (containing 0.1% formic acid) over 30 min to afford tert-butyl (3-((2-(2,6-dioxopiperidin-3-yl)-1-oxoisoindolin-4-yl)oxy)propyl)(methyl)carbamate (racemic due to racemisation in the reaction) (130 mg, yield=52%) as a white solid.

LCMS: (ES, m/z): [M−C₄H₈]⁺=376 (LCMS condition: Column: HALO 90A C18; Mobile phase A: water (containing 0.1% formic acid); Mobile phase B: MeCN (containing 0.1% formic acid); Flow rate: 1.2 mL/min.; RT: 0.797 min)

### (g) Synthesis of Int. 124

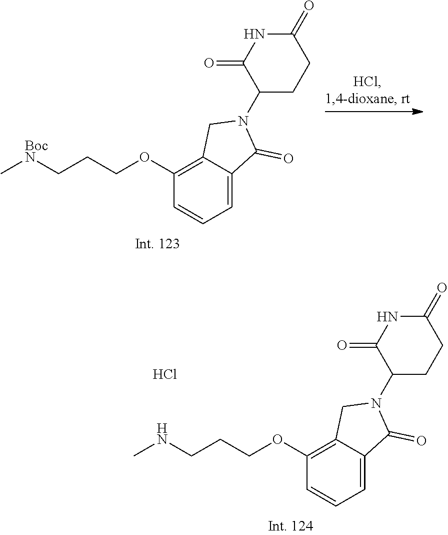 [SMILES: CN(CCCOc1cccc2c1CN(C1CCC(=O)NC1=O)C2=O)C(=O)OC(C)(C)C ; CNCCCOc1cccc2c1CN(C1CCC(=O)NC1=O)C2=O ; Cl]

Into a 100 mL round-bottom flask were added tert-butyl (3-((2-(2,6-dioxopiperidin-3-yl)-1-oxoisoindolin-4-yl)oxy)propyl)(methyl)carbamate (racemic) (1.1 g, 2.549 mmol, 1 eq.) and 4 M HCl in 1,4-dioxane (20 mL) at 25° C. The reaction was stirred for 1 h at 25° C. The mixture was concentrated under reduced pressure to afford crude 3-(4-(3-(methylamino)propoxy)-1-oxoisoindolin-2-yl)piperidine-2,6-dione hydrochloride (racemic) (832 mg) as a white solid which was used in the next step without purification.

LCMS: (ES, m/z): [M+H]⁻=332 (LCMS condition: Column: HALO 90A C18; Mobile phase A: water (containing 0.1% formic acid); Mobile phase B: MeCN (containing 0.1% formic acid) Flow rate: 1.2 mL/min; RT: 0.404 min)

### (h) Synthesis of Int. 125

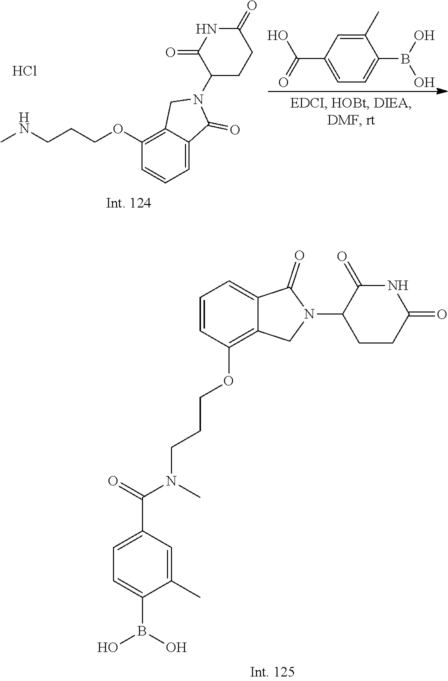

Into a 20 mL vial were added 3-(4-(3-(methylamino)propoxy)-1-oxoisoindolin-2-yl)piperidine-2,6-dione hydrochloride (racemic) (200 mg, 0.604 mmol, 1 eq.), 4-(dihydroxyboranyl)-3-methylbenzoic acid (217 mg, 1.208 mmol, 2 eq.), EDCI (347 mg, 1.812 nmol, 3 eq.), HOBT (244 mg, 1.812 mmol, 3 eq.), DIEA (780 mg, 6.040 mmol, 10 eq.) and DMF (5 mL). The reaction was stirred for 1 h at 25° C. The mixture was purified by reversed-phase flash chromatography on a C18 column using a 10 to 50% gradient of MeCN in water (containing 0.1% formic acid) over 10 min. The crude product (300 mg) was purified by preparative HPLC (Column: Xselect CSH Prep OBD C18, 30×150 mm, 5 μm; Mobile Phase A: water (containing 0.1% formic acid), Mobile Phase B: MeCN; Flow rate: 60 mL/min mL/min; Gradient: 17 to 37% B over 8 min; Wave Length: 254 and 220 nm; RT (min): 6.75) to afford (4-((3-((2-(2,6-dioxopiperidin-3-yl)-1-oxoisoindolin-4-yl)oxy)propyl)(methyl)carbamoyl)-2-methylphenyl)boronic acid (racemic) (267.3 mg, yield=89%) as a white solid.

LCMS: (ES, m/z): [M−H]⁻=492 (LCMS condition: Column: HALO 90A C18; Mobile phase A: water (containing 0.1% formic acid); Mobile phase B: MeCN (containing 0.1%/a formic acid); Flow rate: 1.2 mL/min.; RT: 0.619 min.)

### (i) Synthesis of Example 18

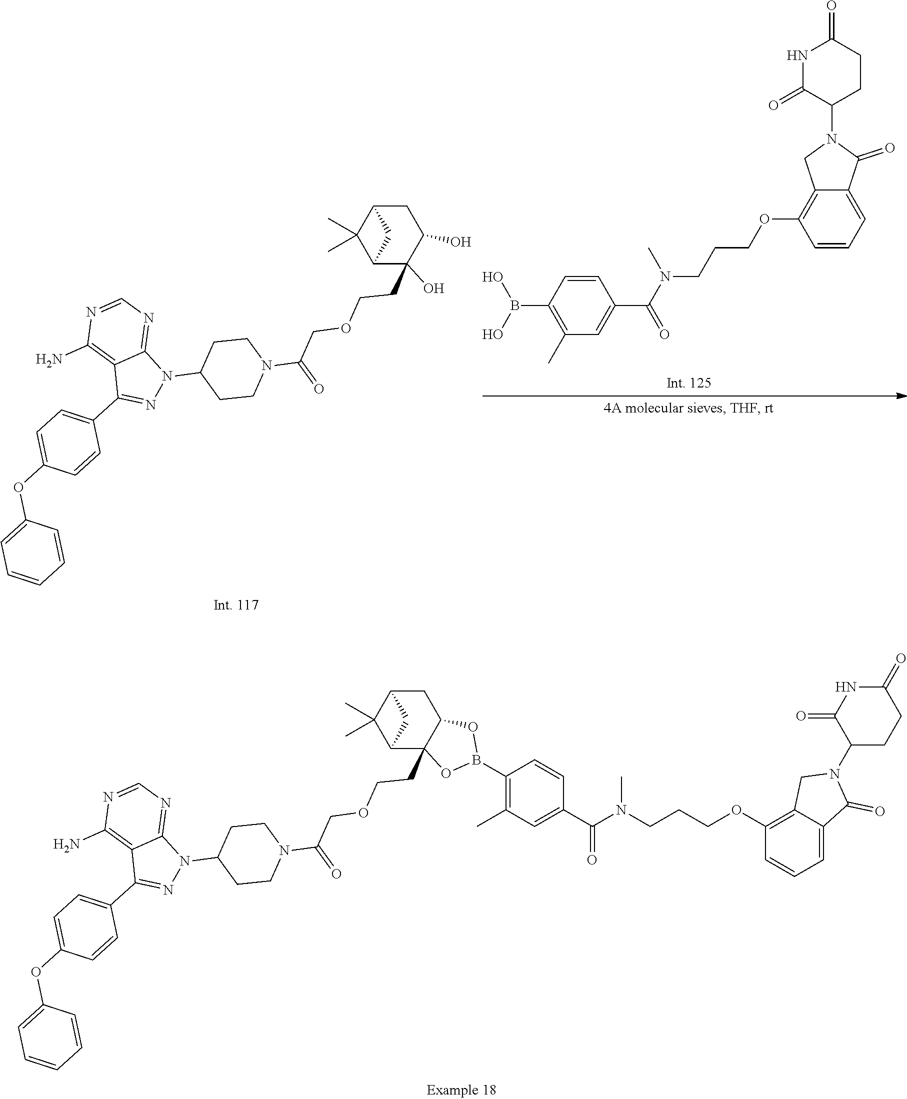 [SMILES: CC1(C)[C@@H]2C[C@H]1[C@](O)(CCOCC(=O)N1CCC(n3nc(-c4ccc(Oc5ccccc5)cc4)c4c(N)ncnc43)CC1)[C@@H](O)C2 ; Cc1cc(C(=O)N(C)CCCOc2cccc3c2CN(C2CCC(=O)NC2=O)C3=O)ccc1B(O)O ; Cc1cc(C(=O)N(C)CCCOc2cccc3c2CN(C2CCC(=O)NC2=O)C3=O)ccc1B1O[C@H]2C[C@H]3C[C@H](C3(C)C)[C@@]2(CCOCC(=O)N2CCC(n3nc(-c4ccc(Oc5ccccc5)cc4)c4c(N)ncnc43)CC2)O1]

Into a 10 mL sealed tube were added (4-((3-((2-(2,6-dioxopiperidin-3-yl)-1-oxoisoindolin-4-yl)oxy)propyl)(methyl)carbamoyl)-2-methylphenyl)boronic acid (18 mg, 0.029 mmol, 1 eq.), 4-[(3-{[2-(2,6-dioxopiperidin-3-yl)-1-oxo-3H-isoindol-4-yl]oxy}propyl)(methyl)carbamoyl]-2-methylphenylboronic acid (racemic) (14 mg, 0.029 mmol, 1 eq.), molecular sieves (4 Å) (100 mg) and THF (3 mL). The reaction was stirred for 16 h at 25° C. under nitrogen. The mixture was filtered, and the filter cake washed with THF (3×50 mL). The filtrate was concentrated under reduced pressure and the residue purified by preparative SFC (Column: Torus Diol OBD 3×25 cm, 5 μm; Mobile Phase A: CO₂, Mobile Phase B: IPA; Flow rate: 75 mL/min; Gradient: isocratic 42% B; Column Temperature (° C.): 35; Back Pressure (bar): 100; Wave Length: 220 nm; RT (min): 6.91; Sample Solvent: MeCN/DCM; Injection Volume: 2 mL) to afford 4-((3aR,4R,6R,7aS)-3a-(2-(2-(4-(4-amino-3-(4-phenoxyphenyl)-1H-pyrazolo[3,4-d]pyrimidin-1-yl)piperidin-1-yl)-2-oxoethoxy)ethyl)-5,5-dimethylhexahydro-4,6-methanobenzo[d][1,3,2]dioxaborol-2-yl)-N-(3-((2-(2,6-dioxopiperidin-3-yl)-1-oxoisoindolin-4-yl)oxy)propyl)-N,3-dimethylbenzamide (mixture of diastereoisomers (2.6 mg, yield=8%) as a white solid.

LCMS: (ES, m/z): [M+H]⁺=1085 (LCMS condition: Column: HALO 90A C18; Mobile phase A: water (containing 0.1%/a formic acid); Mobile phase B: MeCN (containing 0.1%/a formic acid); Flow rate: 1.2 mL/min; RT: 1.043 min)

### Example 19

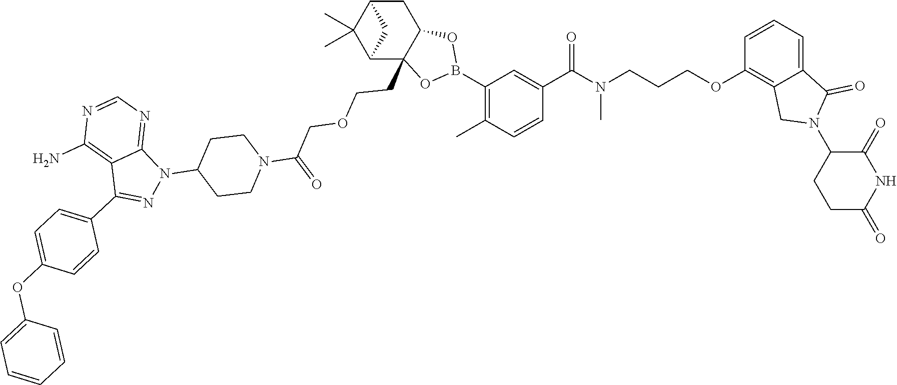 [SMILES: Cc1ccc(C(=O)N(C)CCCOc2cccc3c2CN(C2CCC(=O)NC2=O)C3=O)cc1B1O[C@H]2C[C@H]3C[C@H](C3(C)C)[C@@]2(CCOCC(=O)N2CCC(n3nc(-c4ccc(Oc5ccccc5)cc4)c4c(N)ncnc43)CC2)O1]

### (a) Synthesis of Int. 126

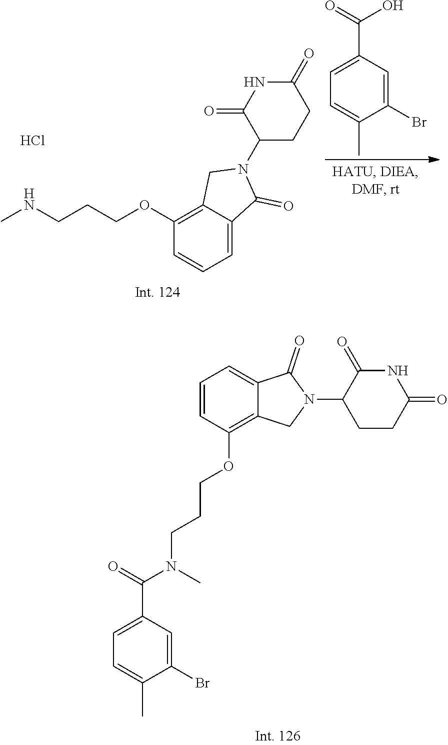

Into an 8 mL vial were added 3-(4-(3-(methylamino)propoxy)-1-oxoisoindolin-2-yl)piperidine-2,6-dione hydrochloride (racemic) (300 mg, 0.905 mmol, 1 eq.), DMF (3 mL), 3-bromo-4-methylbenzoic acid (195 mg, 0.905 mmol, 1 eq.), HATU (516 mg, 1.36 mmol, 1.5 eq.) and DIEA (351 mg, 2.72 mmol, 3 eq.). The reaction was stirred for 1 h at 18° C. The mixture was quenched with water (20 mL) and extracted with EtOAc (3×30 mL). The combined organic extracts were washed with brine (3×20 mL) and dried over anhydrous Na₂SO₄. After filtration, the filtrate was concentrated under reduced pressure to afford 3-bromo-N-(3-((2-(2,6-dioxopiperidin-3-yl)-1-oxoisoindolin-4-yl)oxy)propyl)-N,4-dimethylbenzamide (racemic) (400 mg, yield=76%) as an off-white solid.

LCMS: (ES, m/z): [M+H]⁺=528 (LCMS condition: Column: HALO 90A C18; Mobile phase A: water (containing 0.1% formic acid); Mobile phase B: MeCN (containing 0.1% formic acid); Flow rate: 1.2 mL/min.; RT: 1.485 min.)

### (b) Synthesis of Int. 127

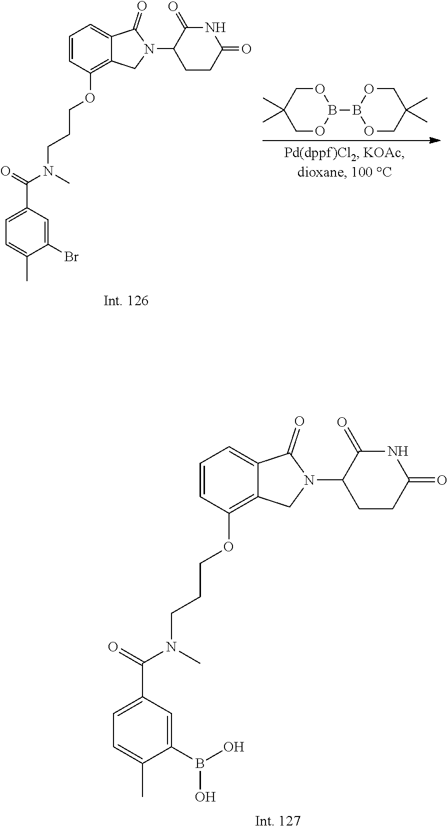

Into an 8 mL vial were added 3-bromo-N-(3-((2-(2,6-dioxopiperidin-3-yl)-1-oxoisoindolin-4-yl)oxy)propyl)-N,4-dimethylbenzamide (racemic) (200 mg, 0.378 mmol, 1 eq.), 2-(5,5-dimethyl-1,3,2-dioxaborinan-2-yl)-5,5-dimethyl-1,3,2-dioxaborinane (171 mg, 0.756 mmol, 2 eq.), Pd(dppf)Cl₂-DCM (28 mg, 0.038 mmol, 0.1 eq.), KOAc (111 mg, 1.13 mmol, 3 eq.) and 1,4-dioxane (2 mL). The reaction was stirred for 2 h at 100° C. under nitrogen. The mixture was quenched with water (20 mL) at 18° C. and extracted with EtOAc (3×30 mL). The combined organic extracts were washed with brine (30 mL) and dried over anhydrous Na₂SO₄. After filtration, the filtrate was concentrated under reduced pressure and the residue purified by reversed-phase flash chromatography on a C18 column using a 5 to 40% gradient of MeCN in water (containing 0.1% formic acid) over 30 min. The crude product was purified by preparative HPLC (Column: Xselect CSH Prep OBD C18 Column, 30×150 mm, 5 μm; Mobile Phase A: water (containing 0.1% formic acid), Mobile Phase B: MeCN; Flow rate: 60 mL/min; Gradient: 17 to 22% B over 15 min; Wave Length: 254 and 220 nm; RT (min): 14.37) to afford (5-((3-((2-(2,6-dioxopiperidin-3-yl)-1-oxoisoindolin-4-yl)oxy)propyl)(methyl)carbamoyl)-2-methylphenyl)boronic acid (racemic) (33.7 mg, yield=18%) as a white solid.

LCMS: (ES, m/z): [M+H]⁺=493 (LCMS condition: Column: HALO 90A C18; Mobile phase A: water (containing 0.1% formic acid); Mobile phase B: MeCN (containing 0.1% formic acid); Flow rate: 1.2 mL/min.; RT: 0.625 min.)

### (c) Synthesis of Example 19

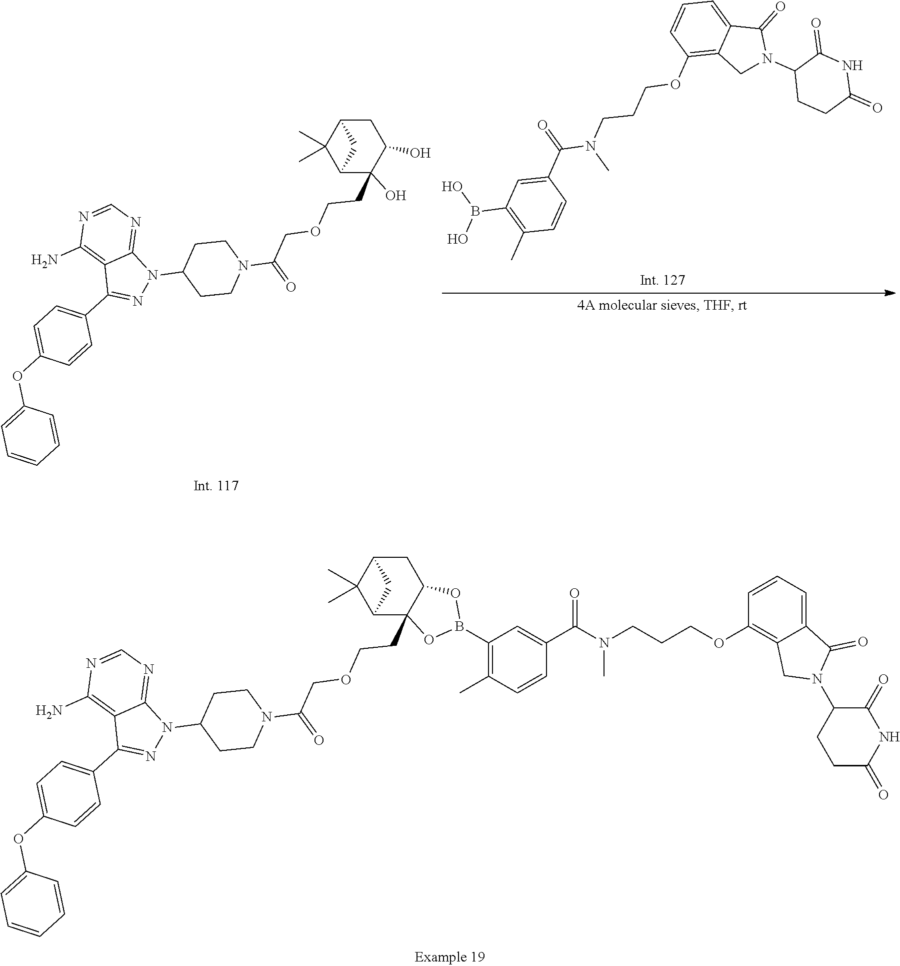 [SMILES: CC1(C)[C@@H]2C[C@H]1[C@](O)(CCOCC(=O)N1CCC(n3nc(-c4ccc(Oc5ccccc5)cc4)c4c(N)ncnc43)CC1)[C@@H](O)C2 ; Cc1ccc(C(=O)N(C)CCCOc2cccc3c2CN(C2CCC(=O)NC2=O)C3=O)cc1B(O)O ; Cc1ccc(C(=O)N(C)CCCOc2cccc3c2CN(C2CCC(=O)NC2=O)C3=O)cc1B1O[C@H]2C[C@H]3C[C@H](C3(C)C)[C@@]2(CCOCC(=O)N2CCC(n3nc(-c4ccc(Oc5ccccc5)cc4)c4c(N)ncnc43)CC2)O1]

Into a 10 mL sealed tube were added 1-(4-(4-amino-3-(4-phenoxyphenyl)-1H-pyrazolo[3,4-d]pyrimidin-1-yl)piperidin-1-yl)-2-(2-((1R,2R,3S,5R)-2,3-dihydroxy-6,6-dimethylbicyclo[3.1.1]heptan-2-yl)ethoxy)ethan-1-one (18 mg, 0.029 mmol, 1 eq.), (5-((3-((2-(2,6-dioxopiperidin-3-yl)-1-oxoisoindolin-4-yl)oxy)propyl)(methyl)carbamoyl)-2-methylphenyl)boronic acid (racemic) (14 mg, 0.029 mmol, 1 eq.), molecular sieves (4 Å) (90 mg) and THF (3 mL). The reaction was stirred for 16 h at 25° C. under nitrogen. The mixture was filtered and the filter cake washed with THF (3×50 mL). The filtrate was concentrated under reduced pressure and the residue purified by preparative SFC (Column: Torus Diol OBD 3×25 cm, 5 μm; Mobile Phase A: CO₂, Mobile Phase B: IPA; Flow rate: 75 mL/min; Gradient: isocratic 42% B; Column Temperature (° C.): 35; Back Pressure (bar): 100; Wave Length: 220 nm; RT (min): 6.77; Sample Solvent: MeCN/DCM; Injection Volume: 3 mL) to afford 3-((3aR,4R,6R,7aS)-3a-(2-(2-(4-(4-amino-3-(4-phenoxyphenyl)-1H-pyrazolo[3,4-d]pyrimidin-1-yl)piperidin-1-yl)-2-oxoethoxy)ethyl)-5,5-dimethylhexahydro-4,6-methanobenzo[d][1,3,2]dioxaborol-2-yl)-N-(3-((2-(2,6-dioxopiperidin-3-yl)-1-oxoisoindolin-4-yl)oxy)propyl)-N,4-dimethylbenzamide (mixture of diastereoisomers) (1.3 mg, yield=4%) as a white solid.

LCMS: (ES, m/z): [M+H]⁺=1084 (LCMS condition: Column: HALO 90A C18; Mobile phase A: water (containing 0.1% formic acid); Mobile phase B: MeCN (containing 0.1%/a formic acid); Flow rate: 1.2 mL/min.; RT: 1.045 min.)

### Example 20

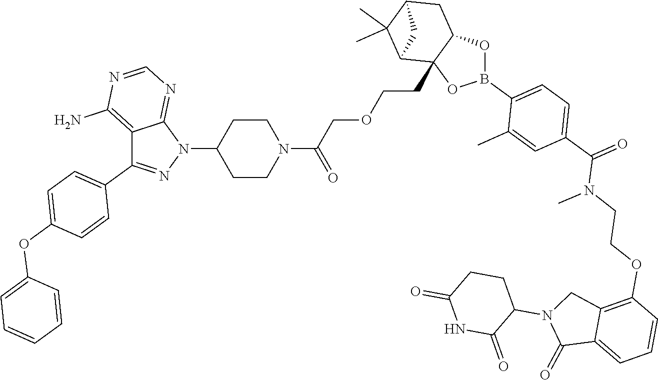 [SMILES: Cc1cc(C(=O)N(C)CCOc2cccc3c2CN(C2CCC(=O)NC2=O)C3=O)ccc1B1O[C@H]2C[C@H]3C[C@H](C3(C)C)[C@@]2(CCOCC(=O)N2CCC(n3nc(-c4ccc(Oc5ccccc5)cc4)c4c(N)ncnc43)CC2)O1]

### (a) Synthesis of Int. 128

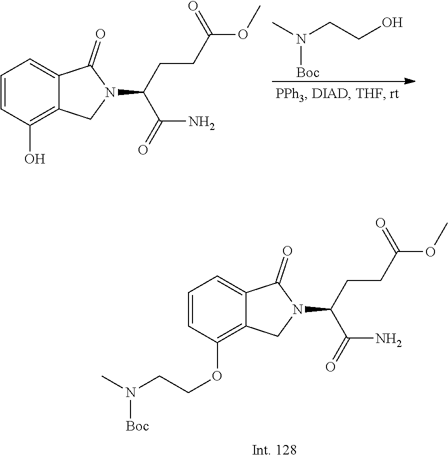

Into a 40 mL vial were added methyl (S)-5-amino-4-(4-hydroxy-1-oxoisoindolin-2-yl)-5-oxopentanoate (600 mg, 2.053 mmol, 1 eq.), PPh₃ (1.29 g, 4.927 mmol, 2.4 eq.), tert-butyl (2-hydroxyethyl)(methyl)carbamate (3.60 g, 20.530 mmol, 10 eq.) and THF (12 mL). The reaction was stirred for 10 min at 25° C. under nitrogen. DIAD (1.25 g, 6.159 mmol, 3 eq.) in THF (2 mL) was added at 0° C. and the reaction stirred for 3 h at r.t. under nitrogen. The mixture was quenched with water (20 mL) at 0° C. and extracted with EtOAc (3×30 mL). The combined organic layers were washed with brine (3×30 mL) and dried over anhydrous Na₂SO₄. After filtration, the filtrate was concentrated under reduced pressure and the residue was purified by reversed-phase flash chromatography on a C18 column using a 40 to 60% gradient of MeCN in water (containing 0.1% formic acid) over 10 min to afford methyl (S)-5-amino-4-(4-(2-((tert-butoxycarbonyl)(methyl)amino)ethoxy)-1-oxoisoindolin-2-yl)-5-oxopentanoate (360 mg, yield=36%) as a light yellow semi-solid.

LCMS: (ES, m/z): [M−C₅H₇O₂]⁺=350 (LCMS condition: Column: HALO 90A C18; Mobile phase A: water (containing 0.1% formic acid); Mobile phase B: MeCN (containing 0.1% formic acid)); Flow rate: 1.2 mL/min; RT: 0.771 min)

### (b) Synthesis of Int. 129

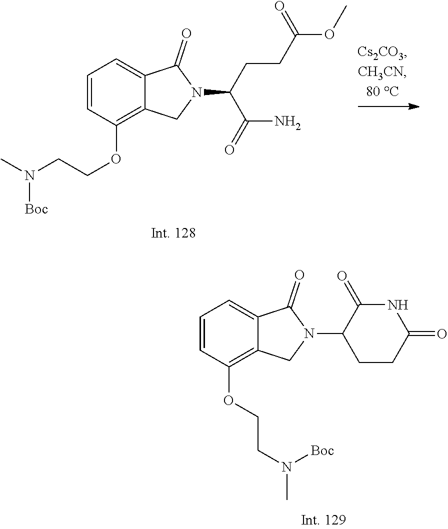

Into a 40 mL vial were added methyl (S)-5-amino-4-(4-(2-((tert-butoxycarbonyl)(methyl)amino)ethoxy)-1-oxoisoindolin-2-yl)-5-oxopentanoate (260 mg, 0.578 mmol, 1 eq.), MeCN (13 mL), and Cs₂CO₃ (565 mg, 1.734 mmol, 3 eq.). The reaction was stirred for 30 min at 80° C. The mixture was filtered, and the filter cake washed with DCM (3×100 mL). The filtrate was concentrated under reduced pressure and the residue purified by reversed-phase flash chromatography on a C18 column using a 10 to 50% gradient of MeCN in water (containing 0.1% formic acid) over 10 min to tert-butyl (2-((2-(2,6-dioxopiperidin-3-yl)-1-oxoisoindolin-4-yl)oxy)ethyl)(methyl)carbamate (racemic due to racemisation in the reaction) (138 mg, yield=55%) as a yellow semi-solid.

LCMS: (ES, m/z): [M−C₅H₇O₂]⁺=318 (LCMS condition: Column: HALO 90A C18; Mobile phase A: water (containing 0.1% formic acid); Mobile phase B: MeCN (containing 0.1% formic acid)); Flow rate: 1.2 mL/min.; RT: 1.506 min.)

### (c) Synthesis of Int. 130

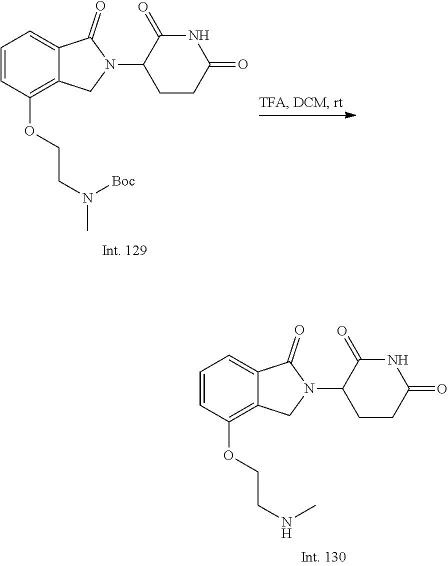 [SMILES: CN(CCOc1cccc2c1CN(C1CCC(=O)NC1=O)C2=O)C(=O)OC(C)(C)C ; CNCCOc1cccc2c1CN(C1CCC(=O)NC1=O)C2=O]

Into a 50 mL round-bottom flask were added tert-butyl (2-((2-(2,6-dioxopiperidin-3-yl)-1-oxoisoindolin-4-yl)oxy)ethyl)(methyl)carbamate (800 mg, 1.916 mmol, 1 eq.), DCM (12 mL) and TFA (4 mL). The reaction was stirred for 1 h at 25° C. The mixture was quenched with saturated NaHCO₃ (aq.) at 0° C., basified to pH=7 and extracted with EtOAc (3×100 mL). The combined organic layers were washed with brine (100 mL) and dried over anhydrous Na₂SO₄. After filtration, the filtrate was concentrated under reduced pressure to afford 3-(4-(2-(methylamino)ethoxy)-1-oxoisoindolin-2-yl)piperidine-2,6-dione (racemic) (650 mg, yield 100%) as an off-white semi-solid.

LCMS: (ES, m/z): [M+H]⁺=318 (LCMS condition: Column: HALO 90A C18; Mobile phase A: water (containing 0.1% formic acid); Mobile phase B: MeCN (containing 0.1% formic acid)); Flow rate: 1.2 mL/min.; RT: 0.363 min.)

### (d) Synthesis of Int. 131

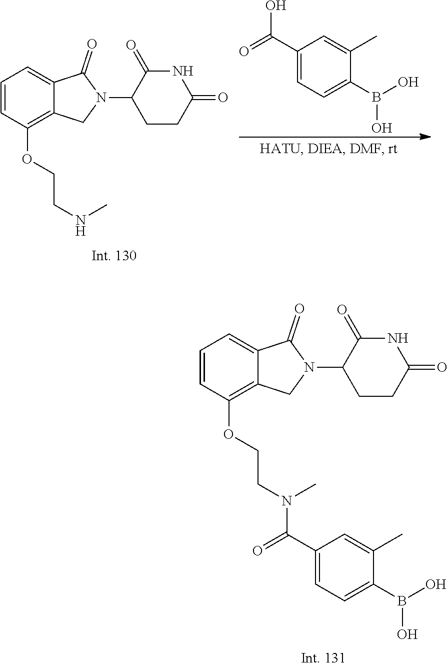 [SMILES: CNCCOc1cccc2c1CN(C1CCC(=O)NC1=O)C2=O ; Cc1cc(C(=O)O)ccc1B(O)O ; Cc1cc(C(=O)N(C)CCOc2cccc3c2CN(C2CCC(=O)NC2=O)C3=O)ccc1B(O)O]

Into a 40 mL vial were added 3-(4-(2-(methylamino)ethoxy)-1-oxoisoindolin-2-yl)piperidine-2,6-dione (600 mg, 0.848 mmol, 1.5 eq.), 4-(dihydroxyboranyl)-3-methylbenzoic acid (102 mg, 0.565 mmol, 1 eq.), HATU (322 mg, 0.848 mmol, 1.5 eq.), DIEA (328 mg, 2.544 mmol, 3 eq.) and DMF (6 mL). The reaction mixture was stirred for 1 h at 25° C. The mixture was purified by reversed-phase flash chromatography on a C18 column using a 10 to 50% gradient of MeCN in water (containing 0.1% formic acid) over 10 min. The crude product was purified by preparative HPLC (Column: Xselect CSH Prep OBD C18, 30×150 mm, 5 μm; Mobile Phase A: water (containing 0.1% formic acid), Mobile Phase B: MeCN; Flow rate: 60 mL/min mL/min; Gradient: 11 to 31% B in 8 min; Wave Length: 254 and 220 nm) to afford (4-((2-((2-(2,6-dioxopiperidin-3-yl)-1-oxoisoindolin-4-yl)oxy)ethyl)(methyl)carbamoyl)-2-methylphenyl)boronic acid (racemic) (134.4 mg, yield=49%) as an off-white solid.

LCMS: (ES, m/z): [M+H]⁺=480 (LCMS condition: Column: HALO 90A C18; Mobile phase A: water (containing 0.1% formic acid); Mobile phase B: MeCN (containing 0.1%/a formic acid)); Flow rate: 1.2 mL/min.; RT: 0.592 min.)

### (e) Synthesis of Example 20

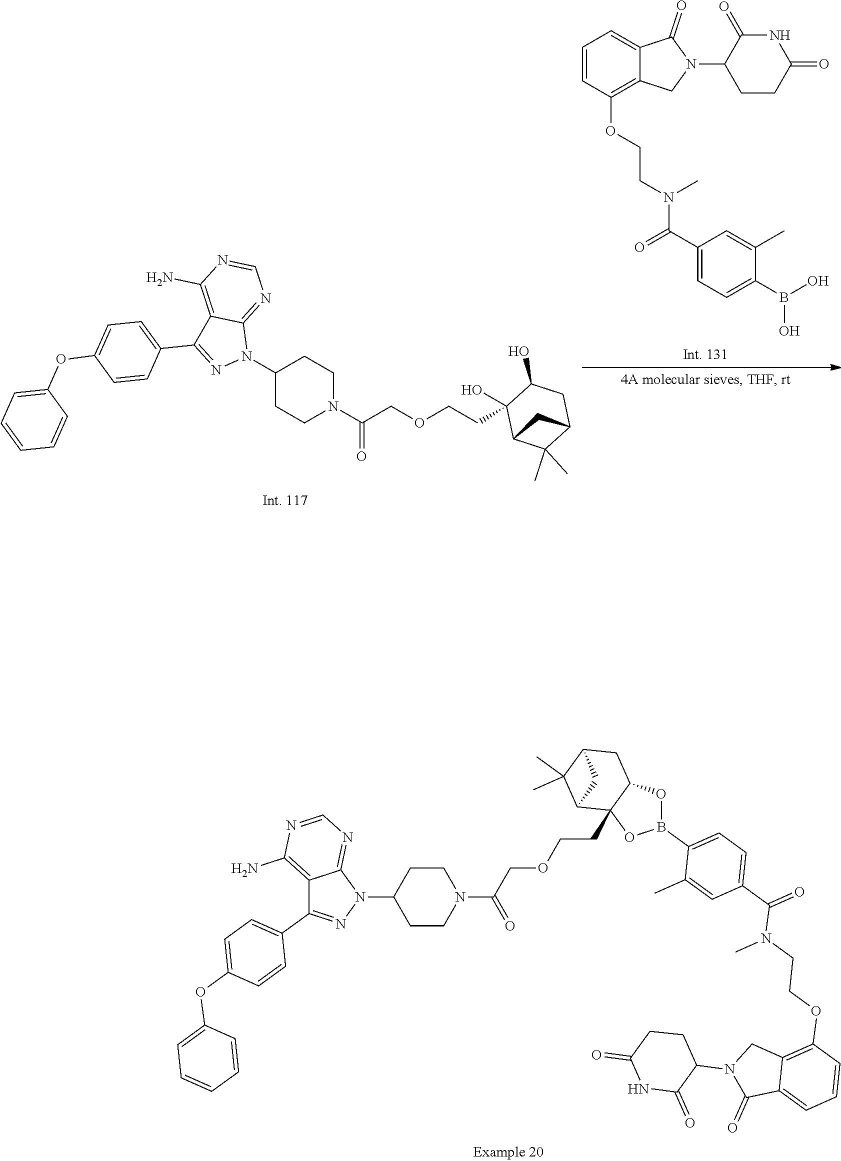 [SMILES: CC1(C)[C@@H]2C[C@H]1[C@](O)(CCOCC(=O)N1CCC(n3nc(-c4ccc(Oc5ccccc5)cc4)c4c(N)ncnc43)CC1)[C@@H](O)C2 ; Cc1cc(C(=O)N(C)CCOc2cccc3c2CN(C2CCC(=O)NC2=O)C3=O)ccc1B(O)O ; Cc1cc(C(=O)N(C)CCOc2cccc3c2CN(C2CCC(=O)NC2=O)C3=O)ccc1B1O[C@H]2C[C@H]3C[C@H](C3(C)C)[C@@]2(CCOCC(=O)N2CCC(n3nc(-c4ccc(Oc5ccccc5)cc4)c4c(N)ncnc43)CC2)O1]

To a stirred solution of 1-(4-(4-amino-3-(4-phenoxyphenyl)-1H-pyrazolo[3,4-d]pyrimidin-1-yl)piperidin-1-yl)-2-(2-((1R,2R,3S,5R)-2,3-dihydroxy-6,6-dimethylbicyclo[3.1.1]heptan-2-yl)ethoxy)ethan-1-one (30 mg, 0.048 mmol, 1 eq.) and (4-((2-((2-(2,6-dioxopiperidin-3-yl)-1-oxoisoindolin-4-yl)oxy)ethyl)(methyl)carbamoyl)-2-methylphenyl)boronic acid (racemic) (23 mg, 0.048 mmol, 1 eq.) in THF (2 mL) was added molecular sieves (4 Å) (150 mg). The reaction was stirred at 25° C. for 16 h. The mixture was filtered and the filtrate concentrated under reduced pressure. The residue was purified by preparative SFC (Column: Torus Diol OBD 3×25 cm, 5 μm; Mobile Phase A: CO₂, Mobile Phase B: MeOH (containing 1% of a 2 M solution of NH₃ in MeOH); Flow rate: 75 mL/min; Gradient: isocratic 38% B; Column Temperature (° C.): 35; Back Pressure (bar): 100; Wave Length: 220 nm; RT (min): 4.67; Sample Solvent: MeOH; Injection Volume: 3 mL; Number of runs: 5) to afford 4-((3aR,4R,6R,7aS)-3a-(2-(2-(4-(4-amino-3-(4-phenoxyphenyl)-1H-pyrazolo[3,4-d]pyrimidin-1-yl)piperidin-1-yl)-2-oxoethoxy)ethyl)-5,5-dimethylhexahydro-4,6-methanobenzo[d][1,3,2]dioxaborol-2-yl)-N-(2-((2-(2,6-dioxopiperidin-3-yl)-1-oxoisoindolin-4-yl)oxy)ethyl)-N,3-dimethylbenzamide (mixture of diastereoisomers) (19.9 mg, yield=54%) as a white solid.

LCMS: (ES, m/z): [M+H]⁺=1070 (LCMS condition: Column: HALO 90A C18; Mobile phase A: water (containing 0.1% formic acid); Mobile phase B: MeCN (containing 0.1% formic acid)); Flow rate: 1.2 mL/min.; RT: 1.018 min.)

### Synthesis of Example Compounds—AR

### Example 21

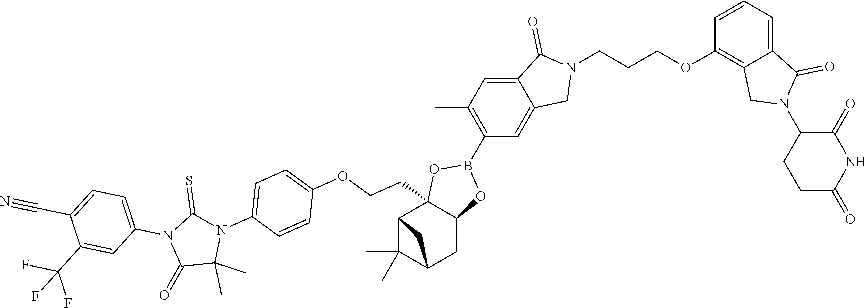 [SMILES: Cc1cc2c(cc1B1O[C@H]3C[C@H]4C[C@H](C4(C)C)[C@@]3(CCOc3ccc(N4C(=S)N(c5ccc(C#N)c(C(F)(F)F)c5)C(=O)C4(C)C)cc3)O1)CN(CCCOc1cccc3c1CN(C1CCC(=O)NC1=O)C3=O)C2=O]

### (a) Synthesis of Int. 132

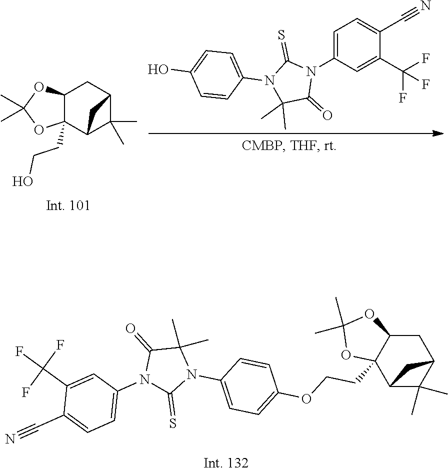

A mixture of 4-(3-(4-hydroxyphenyl)-4,4-dimethyl-5-oxo-2-thioxoimidazolidin-1-yl)-2-(trifluoromethyl)benzonitrile (300 mg, 0.740 mmol, 1 eq.), 2-((3aR,4R,6R,7aS)-2,2,5,5-tetramethyltetrahydro-4,6-methanobenzo[d][1,3]dioxol-3a(4H)-yl)ethan-1-ol (375.3 mg, 1.561 mmol, 2.11 eq.), and CMBP (446.5 mg, 1.850 mmol, 2.5 eq.) in THF (3 mL) was stirred for 16 h at r.t. under nitrogen. The mixture was quenched with water (50 mL) and extracted with EtOAc (3×100 mL). The organic extracts were combined and dried over Na₂SO₄. After filtration, the filtrate was concentrated under reduced pressure and the residue purified by reversed-phase flash chromatography on a C18 column using a 50 to 100% gradient of MeCN in water (containing 10 mmol/L NH₄HCO₃) over 20 min to afford 4-(4,4-dimethyl-5-oxo-3-(4-(2-((3aR,4R,6R,7aS)-2,2,5,5-tetramethyltetrahydro-4,6-methanobenzo[d][1,3]dioxol-3a(4H)-yl)ethoxy)phenyl)-2-thioxoimidazolidin-1-yl)-2-(trifluoromethyl)benzonitrile (210 mg, yield=45%) as a white solid.

LCMS condition: [M+H]⁺=628 (Column: Kinetex EVO C18; Mobile phase A: water (containing 6.5 mM NH₄HCO₃ and ammonia hydroxide (pH=10)); Mobile phase B: MeCN; Flow rate: 1.5 mL/min; RT: 1.610 min)

### (b) Synthesis of Int. 133

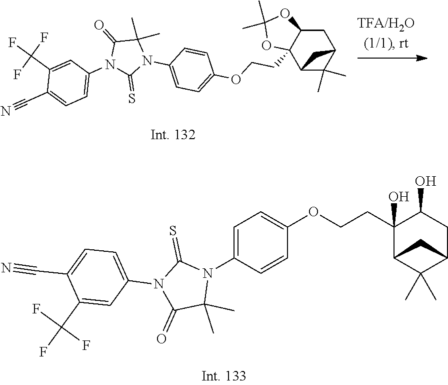 [SMILES: CC1(C)O[C@H]2C[C@H]3C[C@H](C3(C)C)[C@@]2(CCOc2ccc(N3C(=S)N(c4ccc(C#N)c(C(F)(F)F)c4)C(=O)C3(C)C)cc2)O1 ; CC1(C)[C@@H]2C[C@H]1[C@](O)(CCOc1ccc(N3C(=S)N(c4ccc(C#N)c(C(F)(F)F)c4)C(=O)C3(C)C)cc1)[C@@H](O)C2]

Into a 40 mL vial were added 4-(4,4-dimethyl-5-oxo-3-(4-(2-((3aR,4R,6R,7aS)-2,2,5,5-tetramethyltetrahydro-4,6-methanobenzo[d][1,3]dioxol-3a(4H)-yl)ethoxy)phenyl)-2-thioxoimidazolidin-1-yl)-2-(trifluoromethyl)benzonitrile (200 mg, 0.319 mmol, 1 eq.), TFA (3 mL) and water (3 mL). The reaction was stirred for 2 h at r.t. The mixture was concentrated under reduced pressure and the residue purified by preparative HPLC (Column: XBridge Prep OBD C18 Column, 30×150 mm, 5 μm; Mobile Phase A: water (10 mmol/L NH₄HCO₃), Mobile Phase B: MeCN; Flow rate: 60 mL/min; Gradient: 56 to 77% B over 10 min; Wave Length: 254 and 220 nm; RT (min): 8.68) to afford crude product. The crude product (70 mg) was separated by preparative chiral HPLC (Column: CHIRAL ART Amylose-SA, 2×25 cm, 5 μm; Mobile Phase A: hexane (containing 0.5% of a 2 M solution of NH₃ in MeOH), Mobile Phase B: EtOH:DCM (1:1); Flow rate: 20 mL/min; Gradient: isocratic 20% B; Wave Length: 254 and 220 nm; RT (min): 14.902; Sample Solvent: EtOH; Injection Volume: 0.5 mL; Number of runs: 7) to afford 4-(3-(4-(2-((1R,2R,3S,5R)-2,3-Dihydroxy-6,6-dimethylbicyclo[3.1.1]heptan-2-yl)ethoxy)phenyl)-4,4-dimethyl-5-oxo-2-thioxoimidazolidin-1-yl)-2-(trifluoromethyl)benzonitrile (50.1 mg, yield=78%) as a white solid.

LCMS condition: [M+H]⁺=588 (Column: HALO 90A C18; Mobile phase A: water (containing 0.1% formic acid); Mobile phase B: MeCN (containing 0.1% formic acid); Flow rate: 1.2 mL/min; RT: 1.130 min)

### (c) Synthesis of Int. 134

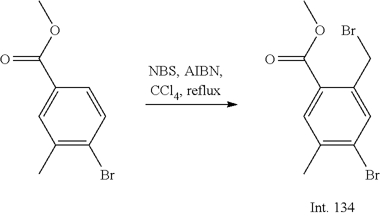

Into a 40 mL vial were added methyl 4-bromo-2,5-dimethylbenzoate (850 mg, 3.50 mmol, 1 eq.), CCl₄ (8.5 mL), NBS (560 mg, 3.15 mmol, 0.9 eq.) and AIBN (57 mg, 0.35 mmol, 0.1 eq.) at 18° C. The reaction was stirred for 4 h at 80° C. under nitrogen. The mixture was concentrated under vacuum to afford crude methyl 4-bromo-2-(bromomethyl)-5-methylbenzoate (1.1 g) as a yellow oil which was used in the next step without purification.

### (d) Synthesis of Int. 135

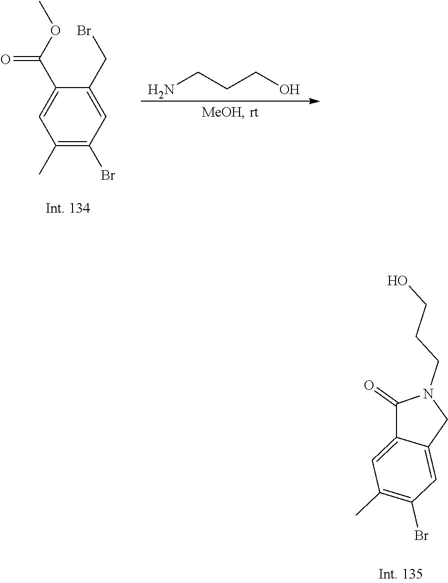

Into a 40 mL vial were added methyl 4-bromo-2-(bromomethyl)-5-methylbenzoate (1.1 g, 3.4 mmol, 1 eq.), MeOH (8.5 mL) and propanolamine (1.3 g, 17 mmol, 5 eq.). The reaction was stirred for 16 h at 18° C. The mixture was concentrated under vacuum and the residue purified by silica gel column chromatography eluting with DCM/MeOH (10:1) to afford 5-bromo-2-(3-hydroxypropyl)-6-methylisoindolin-1-one (370 mg, yield=34%) as a brown solid.

LCMS: (ES, m/z): [M+H]⁺=284 (LCMS condition: Column: HALO 90A C18; Mobile phase A: water (containing 0.1% formic acid); Mobile phase B: MeCN (containing 0.1% formic acid); Flow rate: 1.2 mL/min.; RT: 1.261 min.)

### (e) Synthesis of Int. 136

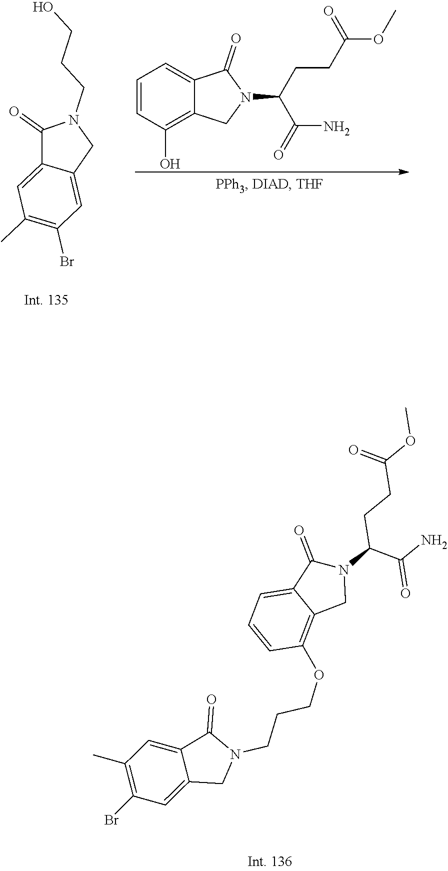

Into a 40 mL vial were added 5-bromo-2-(3-hydroxypropyl)-6-methylisoindolin-1-one (200 mg, 0.704 mmol, 1 eq.), THF (4 mL), methyl (S)-5-amino-4-(4-hydroxy-1-oxoisoindolin-2-yl)-5-oxopentanoate (247 mg, 0.845 mmol, 1.2 eq.) and PPh₃ (277 mg, 1.06 mmol, 1.5 eq.) at 18° C. DIAD (185 mg, 0.915 mmol, 1.3 eq.) in THF (1 mL) was added dropwise at 0° C. The reaction was stirred for 16 h at 18° C. under nitrogen. The mixture was purified by reversed-phase flash chromatography on a CIS column using a 40 to 60% gradient of MeCN in water (containing 10 mmol/L NH₄HCO₃) over 10 min to afford methyl (S)-5-amino-4-(4-(3-(5-bromo-6-methyl-1-oxoisoindolin-2-yl)propoxy)-1-oxoisoindolin-2-yl)-5-oxopentanoate (120 mg, yield=31%) as an off-white solid.

LCMS: (ES, m/z): [M+H]⁺=558 (LCMS condition: Column: HALO 90A C18; Mobile phase A: water (containing 0.1% formic acid); Mobile phase B: MeCN (containing 0.1% formic acid); Flow rate: 1.2 mL/min; RT: 1.393 min)

### (f) Synthesis of Int. 137

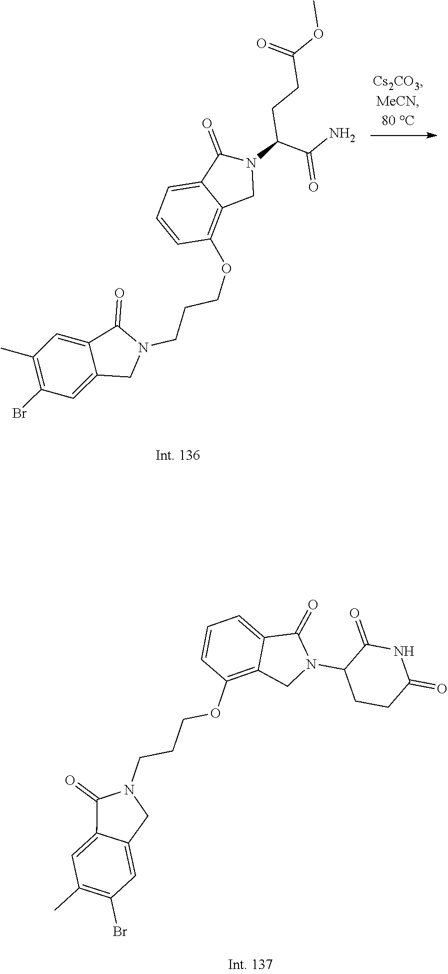

Into a 40 mL vial were added methyl (S)-5-amino-4-(4-(3-(5-bromo-6-methyl-1-oxoisoindolin-2-yl)propoxy)-1-oxoisoindolin-2-yl)-5-oxopentanoate (160 mg, 0.287 mmol, 1 eq.), MeCN (8 mL) and Cs₂CO₃ (280 mg, 0.861 mmol, 3 eq.). The reaction was stirred for 1 h at 80° C. The mixture was concentrated under reduced pressure and the residue purified by reversed-phase flash chromatography on a C18 column using a 40 to 70% gradient of MeCN in water (containing 0.1% formic acid) over 10 min to afford 3-(4-(3-(5-bromo-6-methyl-1-oxoisoindolin-2-yl)propoxy)-1-oxoisoindolin-2-yl)piperidine-2,6-dione (racemic due to racemisation in the reaction) (70 mg, yield=34%) as an off-white solid.

LCMS: (ES, m/z): [M−H]⁻=524 (LCMS condition: Column: HALO 90A C18; Mobile phase A: water (containing 0.1% formic acid); Mobile phase B: MeCN (containing 0.1% formic acid); Flow rate: 1.2 mL/min.; RT: 0.780 min.)

### (g) Synthesis of Int. 138

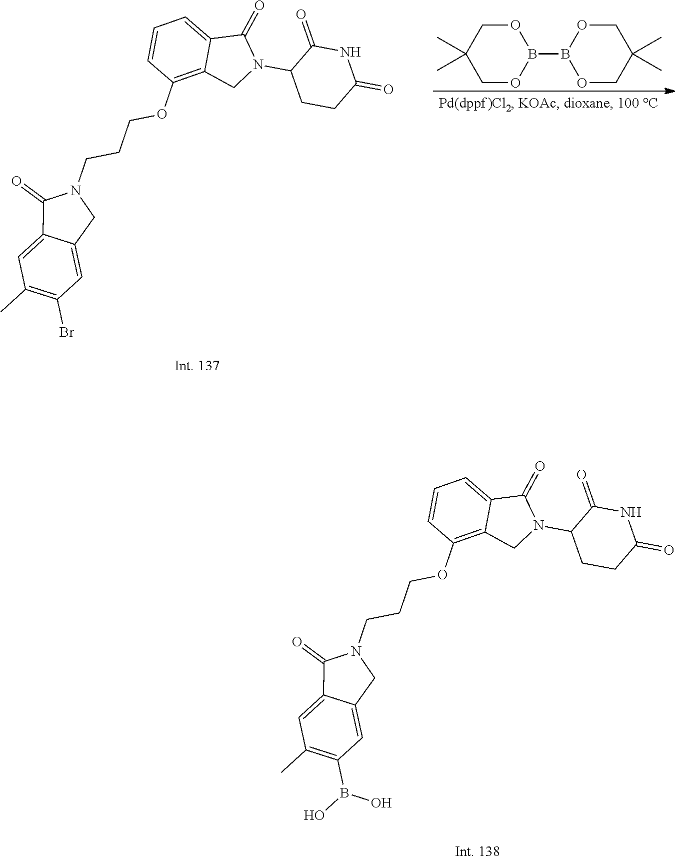

Into an 8 mL vial were added 3-(4-(3-(5-bromo-6-methyl-1-oxoisoindolin-2-yl)propoxy)-1-oxoisoindolin-2-yl)piperidine-2,6-dione (racemic) (75 mg, 0.14 mmol, 1 eq.), 1,4-dioxane (1.5 mL), 2-(5,5-dimethyl-1,3,2-dioxaborinan-2-yl)-5,5-dimethyl-1,3,2-dioxaborinane (64 mg, 0.28 mmol, 2 eq.), KOAc (42 mg, 0.43 mmol, 3 eq.) and Pd(dppf)Cl₂·DCM (10 mg, 0.014 mmol, 0.1 eq.). The reaction was stirred for 2 h at 100° C. under nitrogen. The mixture was purified by reversed-phase flash chromatography on a C18 column using a 40 to 70% gradient of MeCN in water (containing 0.1% formic acid) over 10 min. The crude product was purified by preparative HPLC (Column: Xselect CSH Prep C18 Column, 30×150 mm, 5 μm; Mobile Phase A: water (containing 0.1% formic acid), Mobile Phase B: MeCN; Flow rate: 60 mL/min; Gradient: 6 to 36% B over 10 min; Wave Length: 254 and 220 nm; RT (min): 8.18) to afford (2-(3-((2-(2,6-dioxopiperidin-3-y)-1-oxoisoindolin-4-yl)oxy)propyl)-6-methyl-1-oxoisoindolin-5-yl)boronic acid (racemic) (22.9 mg, yield=32%) as a white solid.

LCMS: (ES, m/z): [M+H]⁺=492 (LCMS condition: Column: HALO 90A C18; Mobile phase A: water (containing 0.1% formic acid); Mobile phase B: MeCN (containing 0.1% formic acid); Flow rate: 1.2 mL/min.; RT: 0.590 min.)

### (h) Synthesis of Example 21

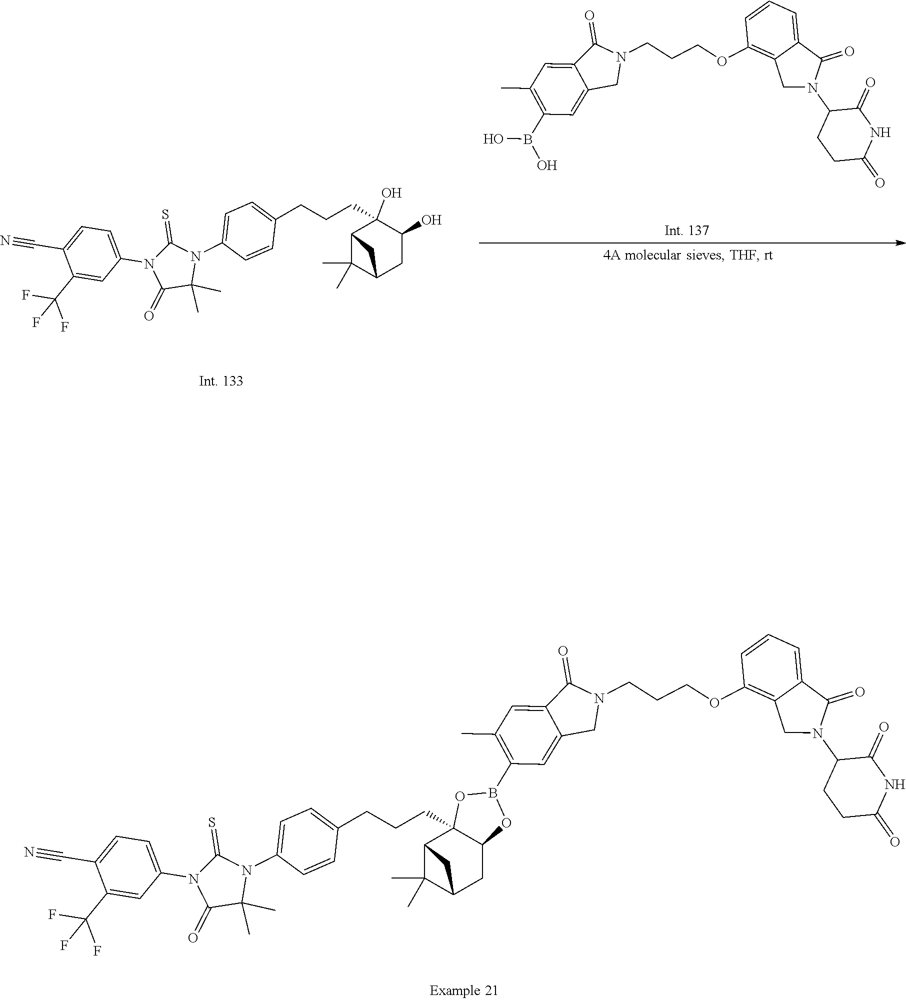

Into a 8 mL vial were added 4-(3-(4-(2-((1R,2R,3S,5R)-2,3-dihydroxy-6,6-dimethylbicyclo[3.1.1]heptan-2-yl)ethoxy)phenyl)-4,4-dimethyl-5-oxo-2-thioxoimidazolidin-1-yl)-2-(trifluoromethyl)benzonitrile (16 mg, 0.027 mmol, 1 eq.), (2-(3-((2-(2,6-dioxopiperidin-3-yl)-1-oxoisoindolin-4-yl)oxy)propyl)-6-methyl-1-oxoisoindolin-5-yl)boronic acid (racemic) (13.4 mg, 0.027 mmol, 1.00 eq.), molecular sieves (4 Å) (50 mg) and THF (3 mL). The reaction was stirred at 25° C. for 16 h. The mixture was filtered, and the filter cake washed with THF (3×30 mL). The filtrate was concentrated under reduced pressure and the crude product purified by preparative SFC (Column: Torus Diol OBD 3×25 cm, 5 μm; Mobile Phase A: CO₂, Mobile Phase B: IPA; Flow rate: 75 mL/min; Gradient: isocratic 35% B; Column Temperature (° C.): 35; Back Pressure (bar): 100; Wave Length: 220 nm; RT peak 1 (min): 2.32; RT peak 2 (min): 5.1; Sample Solvent: MeCN; Injection Volume: 2 mL; Number of runs: 3) to afford 4-(3-(4-(2-((3aR,4R,6R,7aS)-2-(2-(3-((2-(2,6-dioxopiperidin-3-yl)-1-oxoisoindolin-4-yl)oxy)propyl)-6-methyl-1-oxoisoindolin-5-yl)-5,5-dimethyltetrahydro-4,6-methanobenzo[d][1,3,2]dioxaborol-3a(4H)-yl)ethoxy)phenyl)-4,4-dimethyl-5-oxo-2-thioxoimidazolidin-1-yl)-2-(trifluoromethyl)benzonitrile (mixture of diastereoisomers) (6.9 mg, yield=24%) as a white solid.

LCMS: (ES, m/z): [M+H]⁺=1043 (LCMS condition: Column: Shim-pack Scepter C18-120; Mobile phase A: water (containing 5 mM NH₄HCO₃); Mobile phase B: MeCN; Flow rate: 1.5 mL/min.; RT: 1.148 min.)

### Example 22

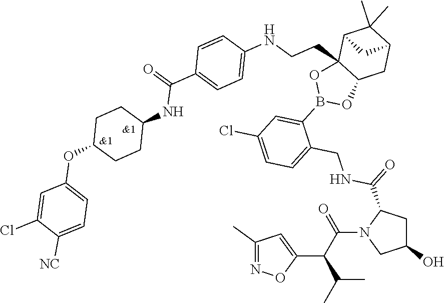 [SMILES: Cc1cc([C@@H](C(=O)N2C[C@H](O)C[C@H]2C(=O)NCc2ccc(Cl)cc2B2O[C@H]3C[C@H]4C[C@H](C4(C)C)[C@@]3(CCNc3ccc(C(=O)N[C@H]4CC[C@H](Oc5ccc(C#N)c(Cl)c5)CC4)cc3)O2)C(C)C)on1]

### (a) Synthesis of Int. 139

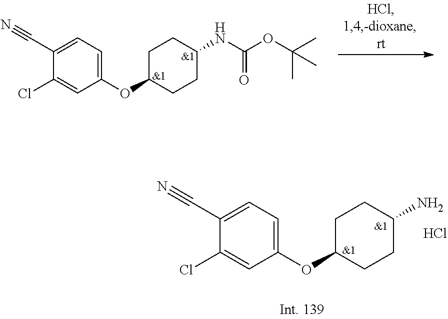

Into a 20 mL vial were added tert-butyl N-[trans-4-(3-chloro-4-cyanophenoxy)cyclohexyl]carbamate (500 mg, 1.425 mmol, 1 eq.) and 4 M HCl in 1,4-dioxane (5 mL) at 20° C. The reaction was stirred for 1 h at 25° C. under nitrogen. The mixture was concentrated under vacuum to afford 4-((trans-4-aminocyclohexyl)oxy)-2-chlorobenzonitrile hydrochloride (350 mg, yield=96%) as an off-white solid.

LCMS: (ES, m/z): [M+H]⁺=251 (LCMS condition: Column: HALO 90A C18; Mobile phase A: water (containing 0.1% formic acid); Mobile phase B: MeCN (containing 0.1% formic acid); Flow rate: 1.5 mL/min)

### (b) Synthesis of Int. 140

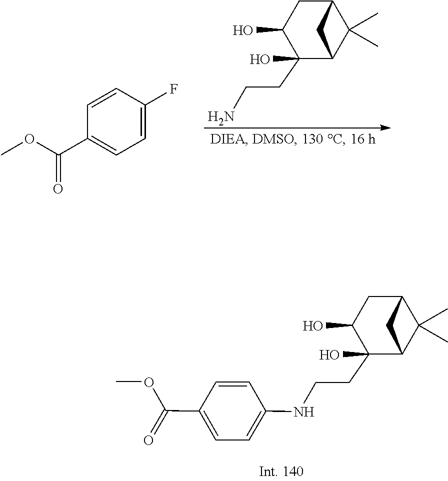

Into an 8 mL vial were added methyl 4-fluorobenzoate (300 mg, 1.946 mmol, 1 eq.), (1R,2R,3S,5R)-2-(2-aminoethyl)-6,6-dimethylbicyclo[3.1.1]heptane-2,3-diol (581 mg, 2.919 mmol, 1.50 eq.), DIEA (754 mg, 5.838 mmol, 3 eq.) and DMSO (3.00 mL, 42.228 mmol, 21.70 eq.). The reaction was stirred 16 h at 130° C. under nitrogen. The mixture was allowed to cool to 20° C., quenched with water and extracted with EtOAc (3×100 mL). The combined organic extracts were washed with brine (200 mL) and dried over anhydrous Na₂SO₄. After filtration, the filtrate was concentrated under reduced pressure and the residue purified by silica gel column chromatography eluting with PE/EtOAc (5:1) to afford methyl 4-({2-[(1R,2R,3S,5R)-2,3-dihydroxy-6,6-dimethylbicyclo[3.1.1]heptan-2-yl]ethyl}amino)benzoate (350 mg, yield=37%) as a red solid.

LCMS: (ES, m/z): [M+H]⁺=334 (LCMS condition: Column: Shim-pack Scepter C18-120; Mobile phase A: water (containing 5 mM NH₄HCO₃); Mobile phase B: MeCN; Flow rate: 1.5 mL/min)

### (c) Synthesis of Int. 141

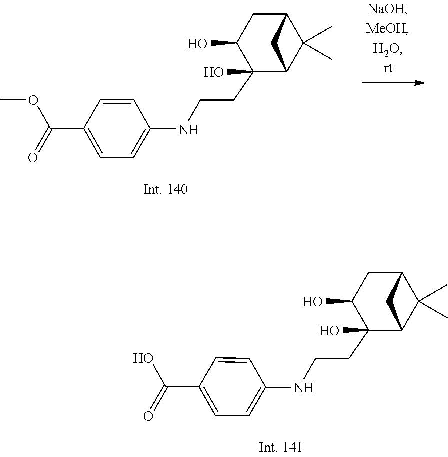

Into an 8 mL vial were added methyl 4-({2-[(1R,2R,3S,5R)-2,3-dihydroxy-6,6-dimethylbicyclo[3.1.1]heptan-2-yl]ethyl}amino)benzoate (300 mg, 0.900 mmol, 1 eq.), NaOH (179 mg, 4.500 mmol, 5 eq.), MeOH (3.00 mL) and water (3.00 mL). The reaction was stirred 2 h at 60° C. under air. The mixture was acidified to pH 1 with 1 M HCl (aq.) and extracted with EtOAc (3×100 mL). The combined organic extracts were washed with brine (100 mL) and dried over anhydrous Na₂SO₄. The residue was purified by reversed-phase flash chromatography on a C18 column using a 10 to 30% gradient of MeCN in water (containing 0.1% formic acid) over 10 min to afford 4-({2-[(1R,2R,3S,5R)-2,3-dihydroxy-6,6-dimethylbicyclo[3.1.1]heptan-2-yl]ethyl}amino)benzoic acid (100 mg, yield=35%) as a yellow solid.

LCMS: (ES, m/z): [M+H]⁺=320 (LCMS condition: Column: HALO 90A C18; Mobile phase A: water (containing 0.1% formic acid); Mobile phase B: MeCN (containing 0.1% formic acid); Flow rate: 1.5 mL/min)

### (d) Synthesis of Int. 142

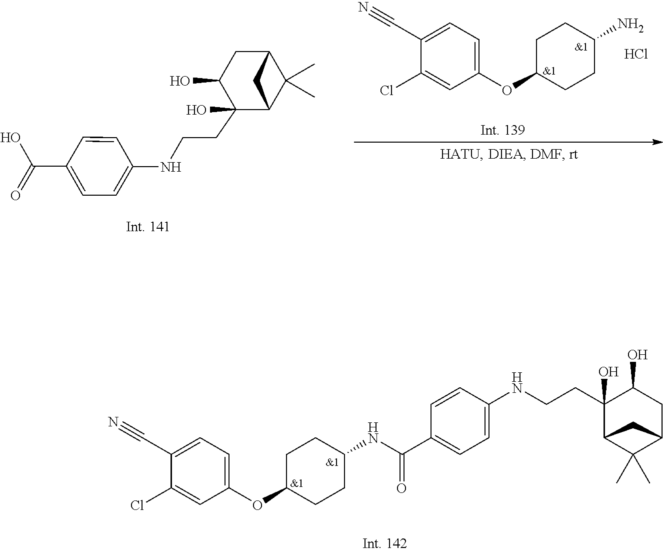

Into an 8 mL vial were added 4-({2-[(1R,2R,3S,5R)-2,3-dihydroxy-6,6-dimethylbicyclo[3.1.1]heptan-2-yl]ethyl}amino)benzoic acid (80 mg, 0.250 mmol, 1 eq.), 4-((trans-4-aminocyclohexyl)oxy)-2-chlorobenzonitrile hydrochloride (108 mg, 0.375 mmol, 1.5 eq.), HATU (142 mg, 0.375 mmol, 1.5 eq.), DMF (0.80 mL) and DIEA (97 mg, 0.750 mmol, 3 eq.). The reaction was stirred for 1 h at 20° C. under air. The mixture was diluted with water (30 mL) and extracted with EtOAc (3×30 mL). The combined organic extracts were washed with brine (20 mL) and dried over anhydrous Na₂SO₄. After filtration, the filtrate was concentrated under reduced pressure and purified by preparative HPLC (Column: Xselect CSH Prep OBD C18, 30×150 mm, 5 μm; Mobile Phase A: water (containing 0.1% formic acid), Mobile Phase B: MeCN; Flow rate: 60 mL/min; Gradient: 50 to 70% B over 8 min; Wave Length: 254 and 220 nm; RT (min): 6.75) to afford N-(trans-4-(3-chloro-4-cyanophenoxy)cyclohexyl)-4-((2-((1R,2R,3S,5R)-2,3-dihydroxy-6,6-dimethylbicyclo[3.1.1]heptan-2-yl)ethyl)amino)benzamide (26.5 mg, yield=190%) as a white solid.

LCMS: (ES, m/z): [M+H]⁺=552 (LCMS condition: Column: HALO 90A C18; Mobile phase A: water (containing 0.1% formic acid); Mobile phase B: MeCN (containing 0.1% formic acid); Flow rate: 1.5 mL/min; RT: 1.353 min.)

### (e) Synthesis of Example 22

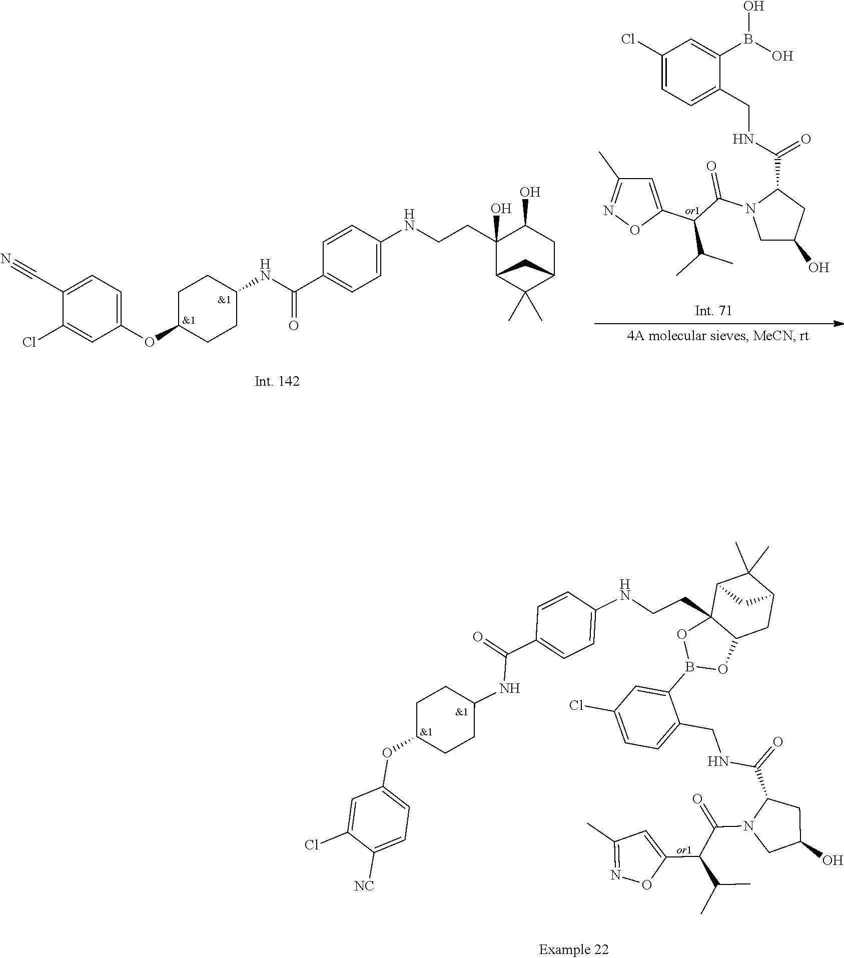

Into a 40 mL vial were added N-(trans-4-(3-chloro-4-cyanophenoxy)cyclohexyl)-4-((2-((1R,2R,3S,5R)-2,3-dihydroxy-6,6-dimethylbicyclo[3.1.1]heptan-2-yl)ethyl)amino)benzamide (80 mg, 0.145 mmol, 1 eq.), 5-chloro-2-({[(2S,4R)-4-hydroxy-1-[(2S)-3-methyl-2-(3-methyl-1,2-oxazol-5-yl)butanoyl]pyrrolidin-2-yl]formamido}methyl)phenylboronic acid (single diastereoisomer with unknown absolute stereochemistry at the centre indicated in the structure) (67.19 mg, 0.145 mmol, 1.00 eq.), molecular sieves (4 Å) (266.54 mg) and THF (10 mL). The reaction was stirred at 25° C. for 16 h. The mixture was filtered, and the filter cake washed with MeOH (3×30 mL). The filtrate was concentrated under reduced pressure and the residue purified by preparative SFC (Column: GreenSep Naphthyl 30×250 mm, 5 μm; Mobile Phase A: CO₂, Mobile Phase B: MeOH; Flow rate: 75 mL/min; Gradient: isocratic 46% B; Column Temperature (° C.): 35; Back Pressure (bar): 100; Wave Length: 220 nm; RT (min): 13.22; Sample Solvent: MeOH; Injection Volume: 2.5 mL) to afford (2S,4R)—N-(4-chloro-2-((3aR,4R,6R,7aS)-3a-(2-((4-((trans-4-(3-chloro-4-cyanophenoxy)cyclohexyl)carbamoyl)phenyl)amino)ethyl)-5,5-dimethylhexahydro-4,6-methanobenzo[d][1,3,2]dioxaborol-2-yl)benzyl)-4-hydroxy-1-((S)-3-methyl-2-(3-methylisoxazol-5-yl)butanoyl)pyrrolidine-2-carboxamide (single diastereoisomer with unknown absolute stereochemistry at the centre indicated in the structure) (95.5 mg, yield=67%) as a white solid.

LCMS: (ES, m/z): [M+H]⁺=980 (LCMS condition: Column: Shim-pack Scepter C18-120; Mobile phase A: Water/MeCN (95:5, v:v) (containing 5 mM NH₄HCO₃); Mobile phase B: MeCN; Flow rate: 1.5 mL/min; RT: 0.965 min.)

### Example 23

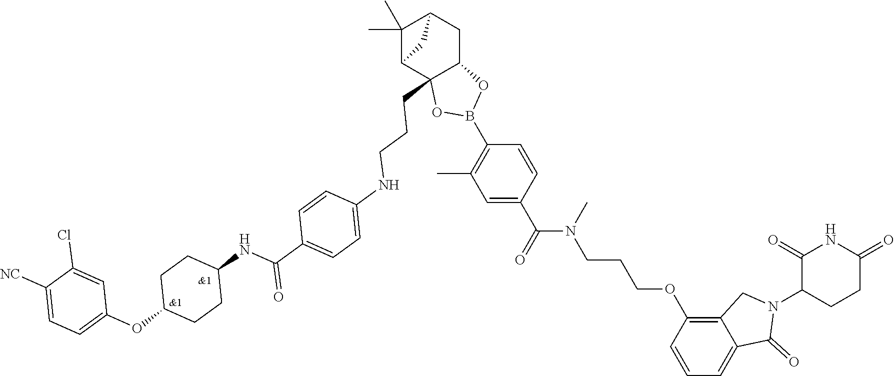 [SMILES: Cc1cc(C(=O)N(C)CCCOc2cccc3c2CN(C2CCC(=O)NC2=O)C3=O)ccc1B1O[C@H]2C[C@H]3C[C@H](C3(C)C)[C@@]2(CCCNc2ccc(C(=O)N[C@H]3CC[C@H](Oc4ccc(C#N)c(Cl)c4)CC3)cc2)O1]

### (a) Synthesis of Int. 143

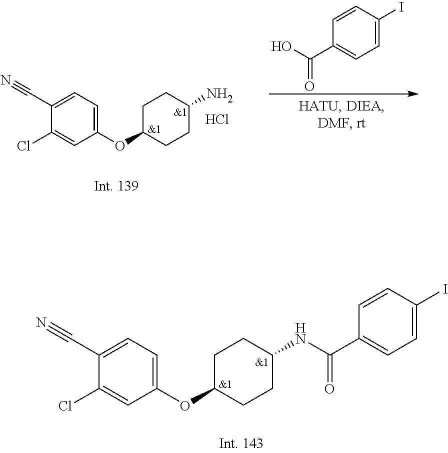

Into a 20 mL vial were added 4-iodobenzoic acid (400 mg, 1.613 mmol, 1 eq.), 4-((trans-4-aminocyclohexyl)oxy)-2-chlorobenzonitrile hydrochloride (606 mg, 2.12 mmol, 1.14 eq.), HATU (919 mg, 2.420 mmol, 1.5 eq.), DIEA (625 mg, 4.839 mmol, 3 eq.) and DMF (4.00 mL). The reaction was stirred for 2 h at r.t. The mixture was quenched with water and extracted with EtOAc (3×50 mL). The combined organic extracts were washed with brine (50 mL) and dried over anhydrous Na₂SO₄. After filtration, the filtrate was concentrated under reduced pressure to afford N-(trans-4-(3-chloro-4-cyanophenoxy)cyclohexyl)-4-iodobenzamide (750 mg, yield=96%) as a light brown solid.

LCMS: (ES, m/z): [M+H]⁺=481 (LCMS condition: Column: HALO 90A C18; Mobile phase A: water (containing 0.1% formic acid); Mobile phase B: MeCN (containing 0.1% formic acid); Flow rate: 1.5 mL/min)

### (b) Synthesis of Int. 144

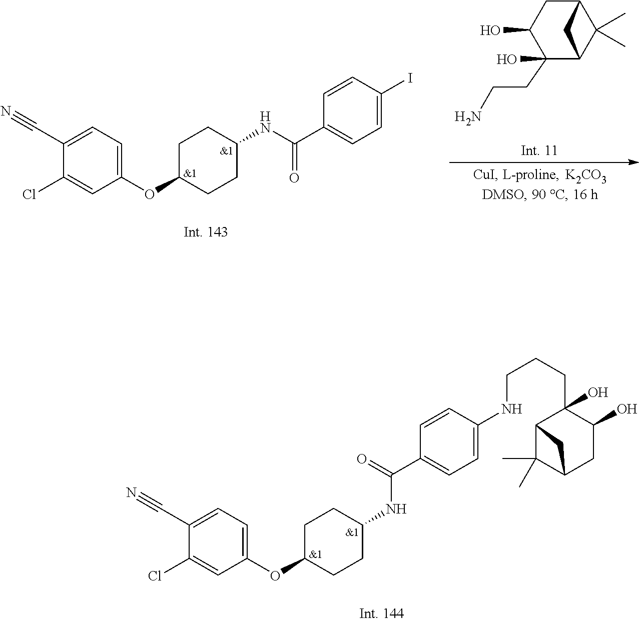

Into an 8 mL vial were added N-(trans-4-(3-chloro-4-cyanophenoxy)cyclohexyl)-4-iodobenzamide (110 mg, 0.229 mmol, 1 eq.), (1R,2R,3S,5R)-2-(3-aminopropyl)-6,6-dimethylbicyclo[3.1.1]heptane-2,3-diol (146 mg, 0.687 mmol, 3 eq.), CuI (8 mg, 0.046 mmol, 0.2 eq.), K₂CO₃ (63 mg, 0.458 mmol, 2 eq.), L-proline (5 mg, 0.046 mmol, 0.2 eq.) and DMSO (2 mL). The reaction was stirred for 16 h at 90° C. under nitrogen. The mixture was allowed to cool to 20° C., quenched with water, and extracted with EtOAc (3×50 mL). The combined organic extracts were washed with brine (50 mL) and dried over anhydrous Na₂SO₄. After filtration, the filtrate was concentrated under reduced pressure and purified by preparative HPLC (Column: Xselect CSH Prep OBD C18 Column, 30×150 mm, 5 μm; Mobile Phase A: water (containing 0.1% formic acid), Mobile Phase B: MeCN; Flow rate: 60 mL/min; Gradient: 53 to 61% B over 8 min; Wave Length: 254 and 220 nm; RT (min): 7.47. The resulting mixture was concentrated under reduced pressure to afford N-(trans-4-(3-chloro-4-cyanophenoxy)cyclohexyl)-4-((3-((1R,2R,3S,5R)-2,3-dihydroxy-6,6-dimethylbicyclo[3.1.1]heptan-2-yl)propyl)amino)benzamide (43.2 mg, yield=33%) as a white solid.

LCMS: (ES, m/z): [M+H]⁺=566 (LCMS condition: Column: HALO 90A C18; Mobile phase A: water (containing 6.5 mM NH₄HCO₃ and ammonia hydroxide (pH=10)); Mobile phase B: MeCN; Flow rate: 1.5 mL/min; RT: 1.346 min.)

### (c) Synthesis of Example 23

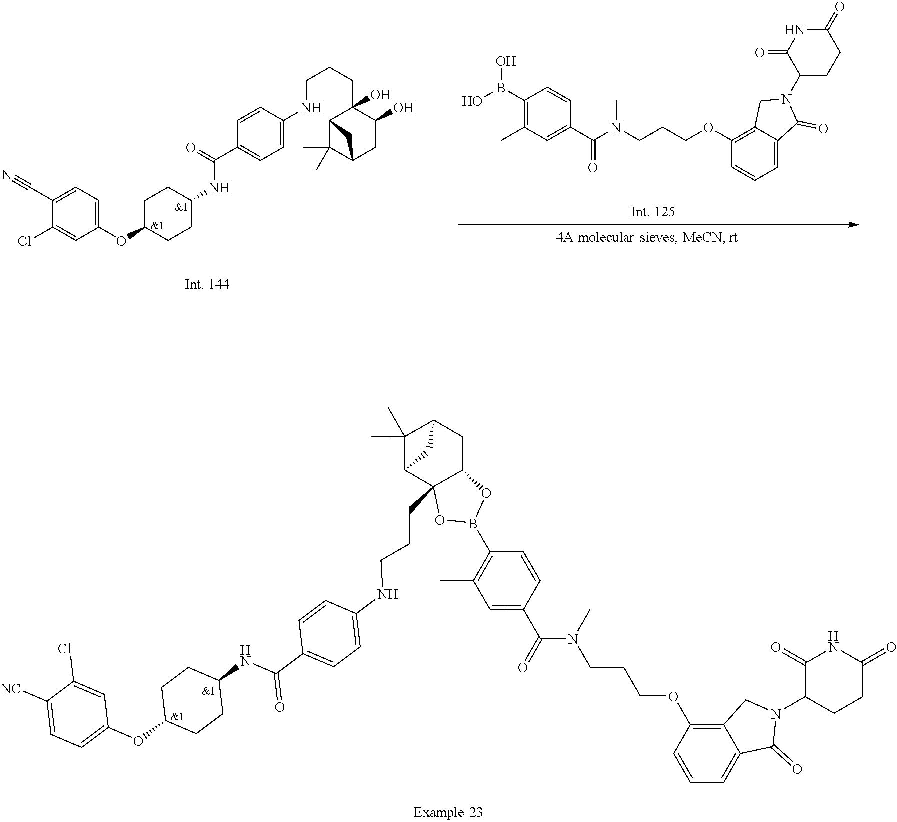

Into a 20 mL vial were added N-(trans-4-(3-chloro-4-cyanophenoxy)cyclohexyl)-4-((3-((1R,2R,3S,5R)-2,3-dihydroxy-6,6-dimethylbicyclo[3.1.1]heptan-2-yl)propyl)amino)benzamide (15 mg, 0.026 mmol, 1 eq.), (4-((3-((2-(2,6-dioxopiperidin-3-yl)-1-oxoisoindolin-4-yl)oxy)propyl)(methyl)carbamoyl)-2-methylphenyl)boronic acid (racemic) (13.07 mg, 0.026 mmol, 1 eq.), molecular sieves (4 Å) (74.99 mg) and MeCN (5 mL). The reaction was stirred at 25° C. for 16 h. The mixture was filtered, and the filter cake washed with MeOH (3×30 mL). The filtrate was concentrated under reduced pressure and the residue purified by preparative SFC (Column: GreenSep Basic 3×15 cm, 5 μm; Mobile Phase A: CO₂, Mobile Phase B: MeOH; Flow rate: 75 mL/min; Gradient: isocratic 32% B; Column Temperature (° C.): 35; Back Pressure (bar): 100; Wave Length: 220 nm; RT (min): 8.69; Sample Solvent: MeOH; Injection Volume: 2 mL; Number of runs: 3) to afford 4-((3aR,4R,6R,7aS)-3a-(3-((4-((trans-4-(3-chloro-4-cyanophenoxy)cyclohexyl)carbamoyl)phenyl)amino)propyl)-5,5-dimethylhexahydro-4,6-methanobenzo[d][1,3,2]dioxaborol-2-yl)-N-(3-((2-(2,6-dioxopiperidin-3-yl)-1-oxoisoindolin-4-yl)oxy)propyl)-N,3-dimethylbenzamide (mixture of diastereoisomers) (11.7 mg, yield=42%) as a white solid.

LCMS: (ES, m/z): [M+H]⁺=1024 (LCMS condition: Column: Shim-pack Scepter C18-120; Mobile phase A: Water/MeCN (95:5, v:v) (containing 5 mM NH₄HCO₃); Mobile phase B: MeCN; Flow rate: 1.5 mL/min; RT: 1.343 min)

### Example 24

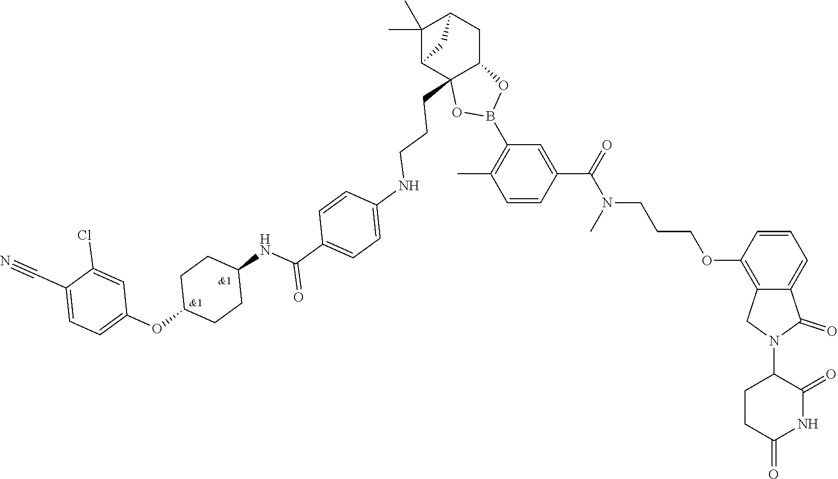 [SMILES: Cc1ccc(C(=O)N(C)CCCOc2cccc3c2CN(C2CCC(=O)NC2=O)C3=O)cc1B1O[C@H]2C[C@H]3C[C@H](C3(C)C)[C@@]2(CCCNc2ccc(C(=O)N[C@H]3CC[C@H](Oc4ccc(C#N)c(Cl)c4)CC3)cc2)O1]

### (a) Synthesis of Example 24

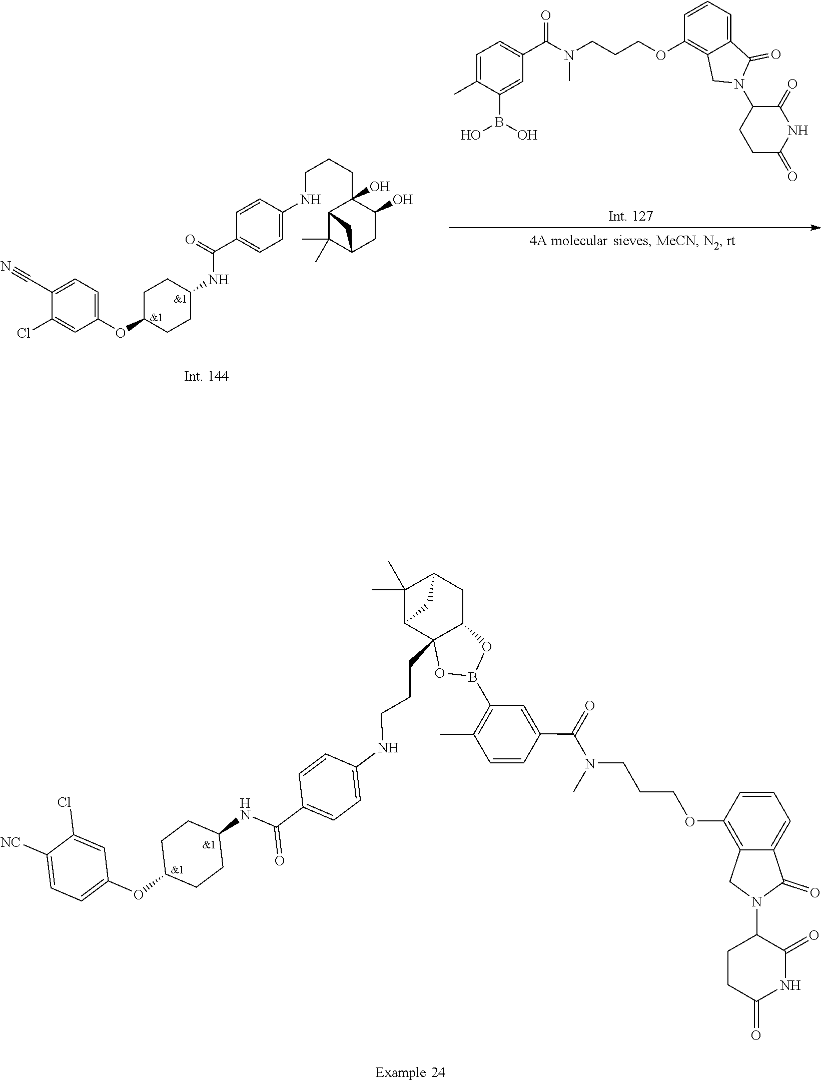

Into a 40 mL vial were added N-(trans-4-(3-chloro-4-cyanophenoxy)cyclohexyl)-4-((3-((1R,2R,3S,5R)-2,3-dihydroxy-6,6-dimethylbicyclo[3.1.1]heptan-2-yl)propyl)amino)benzamide (13 mg, 0.023 mmol, 1 eq.), (5-((3-((2-(2,6-dioxopiperidin-3-yl)-1-oxoisoindolin-4-yl)oxy)propyl)(methyl)carbamoyl)-2-methylphenyl)boronic acid (racemic) (11.33 mg, 0.023 mmol, 1 eq.), molecular sieves (4 Å) (64.99 mg) and MeCN (5 mL). The reaction was stirred at 25° C. for 16 h. The mixture was filtered, and the filter cake was washed with MeOH (3×30 mL). The filtrate was concentrated under reduced pressure and the residue purified by preparative SFC (Column: DAICEL DCpak P4VP 3×25 cm, 5 μm; Mobile Phase A: CO₂, Mobile Phase B: MeOH; Flow rate: 65 mL/min; Gradient: isocratic 48% B; Column Temperature (° C.): 35; Back Pressure (bar): 100; Wave Length: 220 nm; RT (min): 8.9; Sample Solvent: MeOH; Injection Volume: 1.5 mL) to afford 3-((3aR,4R,6R,7aS)-3a-(3-((4-((trans-4-(3-chloro-4-cyanophenoxy)cyclohexyl)carbamoyl)phenyl)amino)propyl)-5,5-dimethylhexahydro-4,6-methanobenzo[d][1,3,2]dioxaborol-2-yl)-N-(3-((2-(2,6-dioxopiperidin-3-yl)-1-oxoisoindolin-4-yl)oxy)propyl)-N,4-dimethylbenzamide (mixture of diastereoisomers) (15.1 mg, yield=64%) as a white solid.

LCMS: (ES, m/z): [M+H]⁺=1024 (LCMS condition: Column: Shim-pack Scepter C18-120: Mobile phase A: Water/MeCN (95:5, v:v) (containing 5 mM NH₄HCO₃): Mobile phase B: MeCN: Flow rate: 1.5 mL/min: RT: 1.012 min)

### BRD4 HiBiT Assays

The purpose of the BRD4 HiBiT Assay was to assess the binding affinity of the dimer to BRD4 and subsequent protein degradation.

### Materials

TABLE 1
Reagents, consumables and instruments of BRD4 HiBiT Assays

| | Vendor | Cat No. |
|---|---|---|
| Reagent | | |
| DMEM | Invitrogen | 11995 |
| Fetal bovine serum | AusGeneX | FBS500-S |
| DPBS (pH 7.4, 1×, sterile) | Invitrogen | 14190-144 |
| Penicillin-Streptomycin(10000 U/ml, 100 ml) | Invitrogen | 15140-122 |
| 1 × TrypLE ™ Express Enzyme, no phenol | Invitrogen | 12604021 |
| red(500 ml) | | |
| DMSO | Solarbio | D8371 |
| Nano Glo HiBiT Lytic Detection System | Promega | N3030 |
| Consumables | | |
| 225 cm² Flask | Corning | CLS431082-25EA |
| 75 cm² Flask | Corning | 354484 |
| 384 well plate | Corning | 3570 |
| 384-Well LDV Clear | Labcyte | LP-0200 |
| Instrument | | |
| Echo 550 Liquid Handler | Labcyte | Echo 550 |
| Multiplate reader | BMG | PHERAstar FSX |
| Centrifuge | Eppendorf | 5810R |
| CO₂ Incubator | ThermoFisher | 371 |
| Biological Safety Cabinet (Class II) | ThermoFisher | 1389 |

BRD4 HiBiT cells (Flp-In™ LgBiT and KI HiBiT) were generated by first creating a Flp-In™293 stable cell line, transfecting in the pFRT serial vector containing an integrated Flp recombination target (FRT) site (Thermo Fisher Scientific, Waltham, MA) to HEK293 cells (ATCC, Manassas, VA). The LgBiT expression sequence (Promega Corporation, Madison, WA) was then introduced into the genome via Flp recombinase-mediated DNA recombination at the FRT site. HiBiT sequence (Promega Corporation, Madison, WA) was then knocked in to the endogenous BRD4 protein in the Flp-In™ LgBiT HEK293 cells and successful clones verifying by sequencing. Work on BRD4 HiBiT cell line was performed under license from Promega Corporation (Madison, WA). Cells were maintained in DMEM (Invitrogen™, Waltham, MA) supplemented with 10% FBS (AusGeneX, Molendinar, Australia) and 1% penicillin/streptomycin (Invitrogen™, Waltham, MA) in a humidified incubator at 5% CO₂, 37° C.

Compounds were dissolved in DMSO (10 mM), serially diluted from 10 mM to 1.5 nM, and pre-incubated for 2 hours. Exponentially growing cells were seeded at 1×10⁴ cells per well of 384 well plate (Corning, New York #3570). Cells were treated with compound for 6 or 48 hours. Following treatment, Nano-Glo® HiBiT Lytic Detection reagent (Promega Corporation, Madison, WA) was added according to manufacturer's instructions, and signal read using BMG CLARIOstar plate reader (BMG Labtech, Ortenburg, Germany). Each data point was performed in duplicate for every experiment. A DC₅₀ value was calculated from linear regression analysis of the plots.

### BTK IFA Assays

The purpose of the BTK Assays were to assess the binding affinity of the dimer to BTK and subsequent protein degradation.

OCI-Ly10 cells were obtained from CoBioer Biosciences Co. Ltd, Shanghai, China, and maintained in IMDM (Invitrogen™, Waltham, MA) supplemented with 20% FBS (AusGeneX, Molendinar, Australia) and 1%, penicillin/streptomycin (Invitrogen™, Waltham, MA) in a humidified incubator at 5% CO₂, 37° C.

Compounds were dissolved in DMSO (10 mM), serially diluted from 10 mM to 1.5 nM, and pre-incubated for 2 hours. Exponentially growing cells were seeded at 8×10³ cells per well of 384 well plate (Corning, New York #356663). Cells were treated with compound for 48 hours. Following treatment, cells were fixed with 4% PFA (SolarBio Lifescience, Beijing, China) for 20 minutes, permeabilised in 100% ice-cold methanol (Merck, Darmstadt, Germany) for 10 minutes, and blocked with blocking buffer (LI-COR Biosciences Lincoln, NE) for 90 minutes.

Cells were incubated with primary BTK antibody (CST #8547 Danvers, MA) at 1:1000 dilution overnight in blocking buffer (LI-COR, Lincoln, NE) at 4° C. Cells were washed in DPBS (Thermo Fisher Scientific, Waltham, MA) and incubated with secondary antibody AlexaFluor 488 goat anti rabbit (Invitrogen™ #A-11008 Waltham, MA) 1:1000 dilution, with Hoechst stain (Invitrogen™ Waltham, MA) 1:5000 dilution in blocking buffer (LI-COR Biosciences, Lincoln, NE) for 90 minutes. After final DPBS wash, 200 ul of DPBS was added to the cells, and signal read on Operetta (Revvity, Waltham, MA). Each data point was performed in duplicate for every experiment. A DC₅₀ value was calculated from linear regression analysis of the plots.

### AR IFA Assays

The purpose of the AR Assays were to assess the binding affinity of the dimer to AR and subsequent protein degradation.

LNCaP, and VCaP cells were obtained from ATCC (ATCC, Manassas, VA). LNCaP cells were maintained in RPMI 1640 (Invitrogen™, Waltham, MA) supplemented with 10% FBS (AusGeneX, Molendinar, Australia) and 1% penicillin/streptomycin (Invitrogen™, Waltham, MA). VCaP cells were maintained in 1:1 ratio F-12(HAM) Nutrient mixture (AdvanCell Technology, Taichung city, Taiwan) and DMEM (Invitrogen™, Waltham, MA) supplemented with 10% FBS (AusGeneX, Molendinar, Australia) and 1% penicillin/streptomycin (Invitrogen™, Waltham, MA).

Compounds were dissolved in DMSO (10 mM), serially diluted from 10 mM to 1.5 nM, and pre-incubated for 2 hours. Exponentially growing cells were seeded at 8×10³ cells per well of 384 well plate (Corning, New York #356663). Cells were treated with compound for 48 hours. Following treatment, cells were fixed with 4% PFA (SolarBio Lifescience, Beijing, China) for 20 minutes, permeabilised in 100% ice-cold methanol (Merck, Darmstadt, Germany) for 10 minutes, and blocked with blocking buffer (LI-COR Biosciences Lincoln, NE) for 90 minutes. Cells were incubated with primary Androgen Receptor (D6F11) XP® Rabbit mAb antibody (CST #5153 Danvers, MA) at 1:1000 dilution overnight in blocking buffer (LI-COR, Lincoln, NE) at 4° C. Cells were washed in DPBS (Thermo Fisher Scientific, Waltham, MA) and incubated with secondary antibody AlexaFluor 488 goat anti rabbit (Invitrogen™ #A-11008 Waltham, MA) 1:1000 dilution, with Hoechst stain (Invitrogen™ Waltham, MA) 1:5000 dilution in blocking buffer (LI-COR Biosciences, Lincoln, NE) for 90 minutes. After final DPBS wash, 200 ul of DPBS was added to the cells, and signal read on Operetta (Revvity, Waltham, MA). Each data point was performed in duplicate for every experiment. A DC₅₀ value was calculated from linear regression analysis of the plots.

### AR CTG Assays

22RV-1, LNCaP, and VCaP cells were obtained from ATCC (ATCC, Manassas, VA). 22RV-1 and LNCaP cells were maintained in RPMI 1640 (Invitrogen™, Waltham, MA) supplemented with 10% FBS (AusGeneX, Molendinar, Australia) and 1% penicillin/streptomycin (Invitrogen™, Waltham, MA). VCaP cells were maintained in 1:1 ratio F-12(HAM) Nutrient mixture (AdvanCell Technology, Taichung city, Taiwan) and DMEM (Invitrogen™, Waltham, MA) supplemented with 10% FBS (AusGeneX, Molendinar, Australia) and 1% penicillin/streptomycin (Invitrogen™, Waltham, MA).

Compounds were dissolved in DMSO (10 mM), serially diluted from 10 mM to 1.5 nM, and pre-incubated for 2 hours. Exponentially growing cells were seeded at 8×10³ cells per well of 384 well plate (Corning, New York #3570). Cells were treated with compound for 24 hours. Following treatment, Cell Titer-Glo® Detection reagent (Promega Corporation, Madison, WA) was added according to manufacturer's instructions, and signal read using BMG CLARIOstar plate reader (BMG Labtech, Ortenburg, Germany). Each data point was performed in duplicate for every experiment. A GI₅₀ value was calculated from linear regression analysis of the plots.

### Results—BRD4 Assays

TABLE 2
HiBiT Assay data for Examples 1-13

| Example No. | HiBiT Assay DC₅₀/μM* |
|---|---|
| 1 | A |
| 2 | A |
| 3 | A |
| 4 | A |
| 5 | A |
| 6 | A |
| 7 | A |
| 8 | A |
| 9 | A |
| 10 | D** |
| 11 | D** |
| 12 | D** |
| 13 | A |

*Degradation in cells at 6-hour timepoint.
**Degradation in cells at 48-hour timepoint.
Data:
A <0.1 μM;
B 0.1-0.5 μM;
C 0.5-1.0 μM;
D 1-10 μM
E >10 μM

A value of less than 10 μM shows degradation. The Examples show degradation of the target protein in the HiBiT assay, as such they are expected to be useful in the treatment and prevention of various diseases.

### Results—BTK Assays

TABLE 3
Assay data for Examples 14-20

| Example No. | Assay DC₅₀/μM* |
|---|---|
| 14 | B |
| 15 | B |
| 16 | C |
| 17 | B |
| 18 | C |
| 19 | C |
| 20 | D |

*Degradation in cells at 48-hour timepoint.
Data:
A <0.1 μM;
B 0.1-0.5 μM;
C 0.5-1.0 μM;
D 1-10 μM
E >10 μM

A value of less than 10 μM shows degradation. The Examples show degradation of the target protein in the assay, as such they are expected to be useful in the treatment and prevention of various diseases.

### Results—AR Assays

TABLE 4
Assay data for Examples 21-24

| Example No. | Assay DC₅₀/μM* |
|---|---|
| 21 | B |
| 22 | B |
| 23 | B |
| 24 | B |

*Degradation in cells at 48-hour timepoint.
Data:
A <0.1 μM;
B 0.1-0.5 μM;
C 0.5-1.0 μM;
D 1-10 μM
E >10 μM

A value of less than 10 μM shows degradation. The Examples show degradation of the target protein in the assay, as such they are expected to be useful in the treatment and prevention of various diseases.

### Results—AR CTG Assays

TABLE 5
Assay data for Examples 21-24

| Example No. | Assay IC₅₀/μM* |
|---|---|
| 21 | E |
| 22 | E |
| 23 | E |
| 24 | E |

*Cell viability in cells at 24-hour timepoint.
Data:
A <0.1 μM;
B 0.1-0.5 μM;
C 0.5-1.0 μM;
D 1-10 μM
E >10 μM

A value above 10 μM indicate no general cytotoxicity. The Examples show no effect on cell viability at 24 h indicating that the degradation is the mechanism of target protein loss.

### Xenograft Study

MV4-11 tumor cell lines were maintained in vitro as a suspension in IMDM medium supplemented with 10% FBS and 1% PS at 37° C. in an atmosphere of 5% CO₂. At ≤5 passages, cells growing in an exponential growth phase were harvested and counted for tumor inoculation.

BALB/c nude female mice (6 weeks old, 19-21 g) were inoculated subcutaneously on the right flank with 10\hat{ }7 tumor cells (100 ul in matrigel). All study animals were monitored for mobility, food and water consumption, body weight, physical appearance and any other abnormal effects. Once tumors reached 250-400 mm3, mice were randomly assigned to groups of N=10. Treatments were formulated as a solution in either 20% HP-β-CD in water or 80% PEG 400 in saline and administered either i.p. (5 μl/g) or subcut (10 μl/g). Dose and frequency of the treatment are outlined in the figure legend. At the end of the study, animals were euthanised by carbon dioxide followed by cervical dislocation to ensure death. 30-60 μL of whole blood was collected into tubes containing anticoagulant with plasma harvested by centrifugation at 4,000 g×5 min and stored at −80° C. until further analysis. Tumor samples were cut into two pieces and snap frozen in liquid nitrogen and stored at −80° C. until analysis.

A compound of the invention has been found to be active in this xenograft study, as such, other compounds of the invention are also expected to be active.

### Western Blot

Tumor samples were cut into small pieces and washed with PBS and then homogenized using a Dounce homogeniser or a tissue grinder. Tissues were lysed on ice in a cytoplasmic and nuclear protein extraction buffer and centrifuged at maximum speed (~16000×g). Supernatants were then stored at −80° C. until further use.

20 ug of protein was loaded on to the gel and run at 120V. Proteins were then transferred to a nitrocellulose membrane using a Transblot (0.3 A, 1.5 h). After transfer, the membrane was blocked for 1 hr at room temperature before incubation with the primary antibody (Anti-Brd3 antibody Abcam Ab50818 or Recombinant Anti-Brd4 antibody Abcam Ab128874) overnight at 4° C. The membranes were then washed 6 times with TBS-tween and incubated for 1 hour with a secondary antibody. Densitometry of the proteins bands was analysed by Image Studio.

### CONCLUSIONS

A range of dimer compounds have been synthesized using linkers of varying size, structurally varied target protein ligands that bind to different intracellular target proteins (e.g., BRD4, BTK and AR) and structurally varied ligands that bind to different E3 ubiquitin ligases (e.g., CRBN, and VHL). The exemplified dimer compounds also show that the boronic acid derivative and the diol portions may be attached to either of the ligands. The assays show that the compounds demonstrate effective target protein degradation. As such, it is expected that the exemplified platform technology may be applied widely to other target proteins and ligases for the treatment and prevention of various diseases.

1. A method of manufacturing a dimer compound, wherein the dimer compound comprises:
a. a first portion comprising a target protein ligand that binds to an intracellular target protein;
b. a second portion comprising a ligand that binds to an E3 ubiquitin ligase; and
c. a linker portion covalently coupling the first and second portions;
wherein the linker portion comprises reversible covalent boronic ester bonds, the method comprising the step of:
i. reacting a first compound comprising the first portion with a second compound comprising the second portion, wherein the first and second compounds further comprise one of and different functional groups selected from a boronic acid derivative and a diol,
ii. isolating, and optionally characterising, the dimer compound.

2. The method according to claim 1, wherein the reaction of the first and second compounds of step (i) occurs in a solvent, preferably a polar solvent.

3. The method according to claim 1, wherein the dimer compound is a solid.

4. The method according to claim 1, wherein the reaction of the first and second compounds of step (i) occurs at a temperature between 1° and 100° C., preferably between 15 and 35° C., preferably between 2° and 30° C., and/or for at least 10 hours.

5. The method according to claim 1, further comprising the step of purifying the isolated dimer compound.

6. The method according to claim 1, further comprising the step of mixing the isolated or purified dimer compound with a pharmaceutically acceptable excipient to make a pharmaceutical composition.

7. The method according to claim 1, wherein the boronic acid derivative is a boronic acid, preferably selected from the list consisting of an alkyl boronic acid, haloalkyl boronic acid, cycloalkyl boronic acid, heterocycloalkyl boronic acid, aryl boronic acid, and a heteroaryl boronic acid, preferably an aryl boronic acid or an alkyl boronic acid.

8. The method according to claim 1, wherein the diol is:
a) cis-1,2-diol, 1,3-diol, preferably a cis-1,2-diol, and/or
b) an aromatic diol or preferably an aliphatic diol, optionally wherein the aromatic diol is an aryl diol or a heteroaryl diol and/or the aliphatic diol is an alkyl diol, a cycloalkyl diol, a heterocycloalkyl diol.

9. The method according to claim 1, wherein the target protein ligand is an inhibitor of a target protein or an antibody of a target protein.

10. The method according to claim 1, wherein the target protein ligand comprises a peptide, preferably an oligopeptide, and/or is non-peptidic, preferably a small molecule.

11. (canceled)

12. The method according to claim 1, wherein the target protein ligand is for an intracellular target protein selected from: a) a BET domain protein binding moiety, preferably selected from: BRD2, BRD3, BRD4, NRF2 and c-Myc, or b) a BTK protein, or c) an AR protein.

13. The method according to claim 1, wherein the ligand of the second portion is for an E3 ubiquitin ligase selected from Cereblon, (CRBN), Von Hippel-Lindau (VHL), mouse double minute 2 (mdm2), Cullin-RING, Cellular inhibitor of apoptosis protein 1 (cIAP1), RNF4 (RING finger protein 4), RNF114 (RING finger protein 114), DCAF16 (DDB1 and CUL4 associated factor 16), DCAF15 (DDB1 And CUL4 Associated Factor 15), DCAF11 (DDB1 And CUL4 Associated Factor 11), FEM1B (Fem-1 Homolog B) (KEAP1 (Kelch-like ECH-associated protein 1), preferably CRBN or VHL.

14. The method according to claim 1, wherein the ligand of the second portion comprises a peptide of an E3 ubiquitin ligase or is non-peptidic, preferably a small molecule.

15.-16. (canceled)

17. A pharmaceutical composition comprising a dimer compound, or a pharmaceutically acceptable salt or prodrug thereof, and a pharmaceutically acceptable excipient, wherein the dimer compound comprises:
a. a first portion comprising a target protein ligand that binds to an intracellular target protein;
b. a second portion comprising a ligand that binds to an E3 ubiquitin ligase; and
c. a linker portion covalently coupling the first and second portions;
wherein the linker portion comprises reversible covalent boronic ester bonds;
wherein the composition comprises substantially no monomer compound, wherein the monomer compound comprises:
d. the first portion or the second portion; and
e. a boronic acid derivative or a diol functional group.

18. The pharmaceutical composition according to claim 17, wherein
the composition comprises substantially no second dimer compound, or wherein the dimer compound is the only active pharmaceutical agent in the composition.

19. (canceled)

20. The method according to claim 1, wherein the linker portion is of a length:
a. sufficient to physically separate the first and second portions to an extent whereby binding of the ligands with their respective targets is not mutually exclusive as a result of steric inhibition; and/or
b. such that the E3 ubiquitin ligase bound to the ligand of the second portion is sufficiently close to the target protein bound to the ligand of the first portion to trigger ubiquitination of the target protein; and/or
c. of from 5 to 25 bonds.

21. The pharmaceutical composition according to claim 17, wherein the dimer compound is capable of eliciting ubiquitination and consequent proteasomal degradation of the target protein when delivered intracellularly.

22.-23. (canceled)

24. The pharmaceutical composition according to claim 17, wherein the composition is to be administered orally and/or the composition is in a solid form.

25. The method according to claim 1, wherein the dimer compound is selected from: (2S,4R)-1-((2S)-2-(3-(2-((3aR,4R,6R,7aS)-3a-(2-(2-((6S)-4-(4-chlorophenyl)-2,3,9-trimethyl-6H-thieno[3,2-f][1,2,4]triazolo[4,3-a][1,4]diazepin-6-yl)acetamido)ethyl)-5,5-dimethylhexahydro-4,6-methanobenzo[d][1,3,2]dioxaborol-2-yl)phenyl)propanamido)-3,3-dimethylbutanoyl)-4-hydroxy-N-(4-(4-methylthiazol-5-yl)benzyl)pyrrolidine-2-carboxamide,
4-(2-((3aR,4R,6R,7aR)-3a-(3-(2-((S)-4-(4-chlorophenyl)-2,3,9-trimethyl-6H-thieno[3,2-f][1,2,4]triazolo[4,3-a][1,4]diazepin-6-yl)acetamido)propyl)-5,5-dimethylhexahydro-4,6-methanobenzo[d][1,3,2]dioxaborol-2-yl)phenyl)-N-(2-(2,6-dioxopiperidin-3-yl)-1,3-dioxoisoindolin-4-yl)butanamide,
(2S,4R)-1-((2S)-2-(3-(2-((3aR,4R,6R,7aS)-3a-(2-(2-((6S)-4-(4-chlorophenyl)-2,3,9-trimethyl-6H-thieno[3,2-f][1,2,4]triazolo[4,3-a][1,4]diazepin-6-yl)acetamido)ethyl)-5,5-dimethylhexahydro-4,6-methanobenzo[d][1,3,2]dioxaborol-2-yl)-3-fluorophenyl)propanamido)-3,3-dimethylbutanoyl)-4-hydroxy-N-(4-(4-methylthiazol-5-yl)benzyl)pyrrolidine-2-carboxamide, 4-(2-((3aR,4R,6R,7aR)-3a-(2-(2-((S)-4-(4-chlorophenyl)-2,3,9-trimethyl-6H-thieno[3,2-f]][1,2,4]triazolo[4,3-a][1,4]diazepin-6-yl)acetamido)ethyl)-5,5-dimethylhexahydro-4,6-methanobenzo[d][1,3,2]dioxaborol-2-yl)phenyl)-N-(2-(2,6-dioxopiperidin-3-yl)-1,3-dioxoisoindolin-4-yl)butanamide,
(2S,4R)-1-((2S)-2-(3-(2-((3aR,4R,6R,7aS)-3a-(3-(2-((6S)-4-(4-chlorophenyl)-2,3,9-trimethyl-6H-thieno[3,2-f][1,2,4]triazolo[4,3-a][1,4]diazepin-6-yl)acetamido)propyl)-5,5-dimethylhexahydro-4,6-methanobenzo[d][1,3,2]dioxaborol-2-yl)phenyl)propanamido)-3,3-dimethylbutanoyl)-4-hydroxy-N-(4-(4-methylthiazol-5-yl)benzyl)pyrrolidine-2-carboxamide, (2S,4R)-1-((2S)-2-(3-(2-((3aR,4R,6R,7aS)-3a-(2-(2-((6S)-4-(4-chlorophenyl)-2,3,9-trimethyl-6H-thieno[3,2-f][1,2,4]triazolo[4,3-a][1,4]diazepin-6-yl)acetamido)ethyl)-5,5-dimethylhexahydro-4,6-methanobenzo[d][1,3,2]dioxaborol-2-yl)phenyl)propanamido)-3,3-dimethylbutanoyl)-4-hydroxy-N—((S)-1-(4-(4-methylthiazol-5-yl)phenyl)ethyl)pyrrolidine-2-carboxamide,
(2S,4R)-1-((2S)-2-(3-(2-((3aR,4R,6R,7aS)-3a-(3-(2-((6S)-4-(4-chlorophenyl)-2,3,9-trimethyl-6H-thieno[3,2-f][1,2,4]triazolo[4,3-a][1,4]diazepin-6-yl)acetamido)propyl)-5,5-dimethylhexahydro-4,6-methanobenzo[d][1,3,2]dioxaborol-2-yl)phenyl)propanamido)-3,3-dimethylbutanoyl)-4-hydroxy-N—((S)-1-(4-(4-methylthiazol-5-yl)phenyl)ethyl)pyrrolidine-2-carboxamide,
2-((S)-4-(4-chlorophenyl)-2,3,9-trimethyl-6H-thieno[3,2-f][1,2,4]triazolo[4,3-a][1,4]diazepin-6-yl)-N-(3-((3aS,4S,6S,7aR)-2-(2-(2-(1-(4-(2,4-dioxotetrahydropyrimidin-1(2H)-yl)phenyl)azetidin-3-yl)ethyl)phenyl)-5,5-dimethyltetrahydro-4,6-methanobenzo[d][1,3,2]dioxaborol-3a(4H)-yl)propyl)acetamide,
(2S,4R)-1-(2-(3-((2-(3a-(3-(2-((S)-4-(4-chlorophenyl)-2,3,9-trimethyl-6H-thieno[3,2-f][1,2,4]triazolo[4,3-a][1,4]diazepin-6-yl)acetamido)propyl)hexahydro-4,7-methanobenzo[d][1,3,2]dioxaborol-2-yl)benzyl)oxy)isoxazol-5-yl)-3-methylbutanoyl)-4-hydroxy-N—((S)-1-(4-(4-methylthiazol-5-yl)phenyl)ethyl)pyrrolidine-2-carboxamide,
(2S,4R)—N-(4-chloro-2-((3aR,4R,6R,7aS)-3a-(2-(2-((S)-4-(4-chlorophenyl)-2,3, 9-trimethyl-6H-thieno[3,2-f][1,2,4]triazolo[4,3-a][1,4]diazepin-6-yl)acetamido)ethyl)-5,5-dimethylhexahydro-4,6-methanobenzo[d][1,3,2]dioxaborol-2-yl)benzyl)-4-hydroxy-1-(3-methyl-2-(3-methylisoxazol-5-yl)butanoyl)pyrrolidine-2-carboxamide,
(2S,4R)—N-(4-chloro-2-(((3aS,4R,6R,7aS)-2-(2-(2-(2-((S)-4-(4-chlorophenyl)-2,3,9-trimethyl-6H-thieno[3,2-f][1,2,4]triazolo[4,3-a][1,4]diazepin-6-yl)acetamido)ethyl)phenyl)-5,5-dimethyltetrahydro-4,6-methanobenzo[d][1,3,2]dioxaborol-3a(4H)-yl)methoxy)benzyl)-4-hydroxy-1-(3-methyl-2-(3-methylisoxazol-5-yl)butanoyl)pyrrolidine-2-carboxamide,
(2S,4R)—N-(4-chloro-2-(3-((3aR,4R,6R,7aS)-3a-(2-(2-((S)-4-(4-chlorophenyl)-2,3,9-trimethyl-6H-thieno[3,2-f][1,2,4]triazolo[4,3-a][1,4]diazepin-6-yl)acetamido)ethyl)-5,5-dimethylhexahydro-4,6-methanobenzo[d][1,3,2]dioxaborol-2-yl)propoxy)benzyl)-4-hydroxy-1-((R)-3-methyl-2-(3-methylisoxazol-5-yl)butanoyl)pyrrolidine-2-carboxamide,
(2S,4R)-1-(2-(3-((2-((3aS,4S,6S,7aR)-3a-(2-(2-((S)-4-(4-chlorophenyl)-2,3,9-trimethyl-6H-thieno[3,2-f][1,2,4]triazolo[4,3-a][1,4]diazepin-6-yl)acetamido)ethyl)-5,5-dimethylhexahydro-4,6-methanobenzo[d][1,3,2]dioxaborol-2-yl)benzyl)oxy)isoxazol-5-yl)-3-methylbutanoyl)-4-hydroxy-N-(4-(4-methylthiazol-5-yl)benzyl)pyrrolidine-2-carboxamide,
4-((3aR,4R,6R,7aS)-3a-(2-(2-(4-(4-amino-3-(4-phenoxyphenyl)-1H-pyrazolo[3,4-d]pyrimidin-1-yl)piperidin-1-yl)ethoxy)ethyl)-5, 5-dimethylhexahydro-4,6-methanobenzo[d][1,3,2]dioxaborol-2-yl)-N-(3-((1-(2,6-dioxopiperidin-3-yl)-3-methyl-2-oxo-2,3-dihydro-1H-benzo[d]imidazol-5-yl)oxy)propyl)-N,3-dimethylbenzamide,
4-((3aR,4R,6R,7aS)-3a-(2-(2-(4-(4-amino-3-(4-phenoxyphenyl)-1H-pyrazolo[3,4-d]pyrimidin-1-yl)piperidin-1-yl)ethoxy)ethyl)-5, 5-dimethylhexahydro-4,6-methanobenzo[d][1,3,2]dioxaborol-2-yl)-N-(4-(2-(2,6-dioxopiperidin-3-yl)-1-oxoisoindolin-4-yl)but-3-yn-1-yl)-3-methylbenzamide,
4-((3aR,4R,6R,7aS)-3a-(2-(2-(4-(4-amino-3-(4-phenoxyphenyl)-1H-pyrazolo[3,4-d]pyrimidin-1-yl)piperidin-1-yl)-2-oxoethoxy)ethyl)-5,5-dimethylhexahydro-4,6-methanobenzo[d][1,3,2]dioxaborol-2-yl)-N-(3-((1-(2,6-dioxopiperidin-3-yl)-3-methyl-2-oxo-2,3-dihydro-1H-benzo[d]imidazol-5-yl)oxy)propyl)-N,3-dimethylbenzamide,
4-((3aR,4R,6R,7aS)-3a-(2-(2-(4-(4-amino-3-(4-phenoxyphenyl)-1H-pyrazolo[3,4-d]pyrimidin-1-yl)piperidin-1-yl)-2-oxoethoxy)ethyl)-5,5-dimethylhexahydro-4,6-methanobenzo[d][1,3,2]dioxaborol-2-yl)-N-(4-(2-(2,6-dioxopiperidin-3-yl)-1-oxoisoindolin-4-yl)but-3-yn-1-yl)-3-methylbenzamide,
4-((3aR,4R,6R,7aS)-3a-(2-(2-(4-(4-amino-3-(4-phenoxyphenyl)-1H-pyrazolo[3,4-d]pyrimidin-1-yl)piperidin-1-yl)-2-oxoethoxy)ethyl)-5,5-dimethylhexahydro-4,6-methanobenzo[d][1,3,2]dioxaborol-2-yl)-N-(3-((2-(2,6-dioxopiperidin-3-yl)-1-oxoisoindolin-4-yl)oxy)propyl)-N,3-dimethylbenzamide,
3-((3aR,4R,6R,7aS)-3a-(2-(2-(4-(4-amino-3-(4-phenoxyphenyl)-1H-pyrazolo[3,4-d]pyrimidin-1-yl)piperidin-1-yl)-2-oxoethoxy)ethyl)-5,5-dimethylhexahydro-4,6-methanobenzo[d][1,3,2]dioxaborol-2-yl)-N-(3-((2-(2,6-dioxopiperidin-3-yl)-1-oxoisoindolin-4-yl)oxy)propyl)-N,4-dimethylbenzamide,
4-((3aR,4R,6R,7aS)-3a-(2-(2-(4-(4-amino-3-(4-phenoxyphenyl)-1H-pyrazolo[3,4-d]pyrimidin-1-yl)piperidin-1-yl)-2-oxoethoxy)ethyl)-5,5-dimethylhexahydro-4,6-methanobenzo[d][1,3,2]dioxaborol-2-yl)-N-(2-((2-(2,6-dioxopiperidin-3-yl)-1-oxoisoindolin-4-yl)oxy)ethyl)-N,3-dimethylbenzamide,
4-(3-(4-(2-((3aR,4R,6R,7aS)-2-(2-(3-((2-(2,6-dioxopiperidin-3-yl)-1-oxoisoindolin-4-yl)oxy)propyl)-6-methyl-1-oxoisoindolin-5-yl)-5, 5-dimethyltetrahydro-4,6-methanobenzo[d][1,3,2]dioxaborol-3a(4H)-yl)ethoxy)phenyl)-4,4-dimethyl-5-oxo-2-thioxoimidazolidin-1-yl)-2-(trifluoromethyl)benzonitrile,
(2S,4R)—N-(4-chloro-2-((3aR,4R,6R,7aS)-3a-(2-((4-((trans-4-(3-chloro-4-cyanophenoxy)cyclohexyl)carbamoyl)phenyl)amino)ethyl)-5,5-dimethylhexahydro-4,6-methanobenzo[d][1,3,2]dioxaborol-2-yl)benzyl)-4-hydroxy-1-((S)-3-methyl-2-(3-methylisoxazol-5-yl)butanoyl)pyrrolidine-2-carboxamide,
4-((3aR,4R,6R,7aS)-3a-(3-((4-((trans-4-(3-chloro-4-cyanophenoxy)cyclohexyl)carbamoyl)phenyl)amino)propyl)-5,5-dimethylhexahydro-4,6-methanobenzo[d][1,3,2]dioxaborol-2-yl)-N-(3-((2-(2,6-dioxopiperidin-3-yl)-1-oxoisoindolin-4-yl)oxy)propyl)-N,3-dimethylbenzamide, and
3-((3aR,4R,6R,7aS)-3a-(3-((4-((trans-4-(3-chloro-4-cyanophenoxy)cyclohexyl)carbamoyl)phenyl)amino)propyl)-5,5-dimethylhexahydro-4,6-methanobenzo[d][1,3,2]dioxaborol-2-yl)-N-(3-((2-(2,6-dioxopiperidin-3-yl)-1-oxoisoindolin-4-yl)oxy)propyl)-N,4-dimethylbenzamide,
or a pharmaceutically acceptable salt or prodrug thereof.

26. A method of treating or preventing a disease comprising administering the pharmaceutical composition according to claim 1, wherein the disease is selected from cancer, neurological disorders, hepatitis, ulcerative colitis, gastritis, autoimmunity, restenosis, stroke, heart failure, neurodegenerative conditions such as Alzheimer's disease, Parkinson's disease, Huntington's disease, myotonic dystrophy, and amyotrophic lateral sclerosis, AIDS, ischemia such as traumatic brain injury, spinal cord injury, cerebral ischemia, cerebral ischemia/reperfusion (l/R) injury, acute and chronic CNS injury ischemia, stroke or myocardial infarction, degenerative diseases of the musculoskeletal system such as osteoporosis, autoimmune diseases, connective tissue disorders, inflammatory diseases or proteostatic disease.

27. (canceled)
